(19) United States
(12) Patent Application Publication
(10) Pub. No.: US 2009/0170097 A1
(43) Pub. Date: Jul. 2, 2009

(54) GENE EXPRESSION IN BIOLOGICAL CONDITIONS

(75) Inventors: Torben Falck Orntoft, Aabyhoj (DK); Thomas Thykjaer Andersen, Brabrand (DK); Lars Dyrskjot Andersen, Risskov (DK); Jens Ledet Jensen, Lystrup (DK)

Correspondence Address: EDWARDS ANGELL PALMER & DODGE LLP, P.O. BOX 55874, BOSTON, MA, 02205, US

(73) Assignee: Aros Applied Biotechnology APS

(21) Appl. No.: 12/180,321

(22) Filed: Jul. 25, 2008

Related U.S. Application Data
(63) Continuation of application No. 10/533,547, filed on Nov. 16, 2005, now abandoned.

(30) Foreign Application Priority Data
Nov. 1, 2002 (DK) PA200201685

(51) Int. Cl. C12Q 1/68

(52) U.S. Cl. 435/6

(57) ABSTRACT

The present invention relates to a method of predicting the prognosis of a biological condition in animal tissue, wherein the expression of genes is examined and correlated to standards. The invention further relates to the treatment of the biological condition and an assay for predicting the prognosis. In particular, the invention concerns gene expression in epithelial tissue, such as urinary bladder under both normal and abnormal conditions.

This application is a continuation of U.S. patent application Ser. No. 10/533,547 filed Nov. 16, 2005, which is a US National Phase application of PCT/DK03/00750 filed Nov. 3, 2003, both of which are incorporated herein in their entirety.

### TECHNICAL FIELD OF THE INVENTION

The present invention relates to a method of predicting the prognosis of a biological condition in animal tissue, wherein the expression of genes is examined and correlated to standards. The invention further relates to the treatment of the biological condition and an assay for predicting the prognosis.

### BACKGROUND

The building of large databases containing human genome sequences is the basis for studies of gene expressions in various tissues during normal physiological and pathological conditions. Constantly (constitutively) expressed sequences as well as sequences whose expression is altered during disease processes are important for our understanding of cellular properties, and for the identification of candidate genes for future therapeutic intervention. As the number of known genes and ESTs build up in the databases, array-based simultaneous screening of thousands of genes is necessary to obtain a profile of transcriptional behaviour, and to identify key genes that either alone or in combination with other genes, control various aspects of cellular life. One cellular behaviour that has been a mystery for many years is the malignant behaviour of cancer cells. It is now known that for example defects in DNA repair can lead to cancer but the cancer-creating mechanism in heterozygous individuals is still largely unknown as is the malignant cell's ability to repeat cell cycles to avoid apoptosis to escape the immune system to invade and metastasize and to escape therapy. There are indications in these areas and excellent progress has been made, both the myriad of genes interacting with each other in a highly complex multidimensional network is making the road to insight long and contorted.

Similar appearing tumors—morphologically, histochemically, microscopically—can be profoundly different. They can have different invasive and metastasizing properties, as well as respond differently to therapy. There is thus a need in the art for methods which distinguish tumors and tissues on factors different than those currently in clinical use. The malignant transformation from normal tissue to cancer is believed to be a multistep process, in which tumorsuppressor genes, that normally repress cancer growth show reduced gene expression and in which other genes that encode tumor promoting proteins (oncogenes) show an increased expression level. Several tumor suppressor genes have been identified up till now, as e.g. p16, Rb, p53 (Nesrin Özören and Wafik S. El-Deiry, Introduction to cancer genes and growth control, In: DNA alterations in cancer, genetic and epigenetic changes, Eaton publishing, Melanie Ehrlich (ed) p. 1-43, 2000; and references therein). They are usually identified by their lack of expression or their mutation in cancer tissue.

Other examinations have shown this downregulation of transcripts to be partly due to loss of genomic material (loss of heterozygosity), partly to methylation of promotorregions, and partly due to unknown factors (Nesrin Özören and Wafik S. El-Deiry, Introduction to cancer genes and growth control, In: DNA alterations in cancer, genetic and epigenetic changes, Eaton publishing, Melanie Ehrlich (ed) p. 1-43, 2000; and references therein).

Several oncogenes are known, e.g. cyclinD1/PRAD1/BCL1, FGFs, c-MYC, BCL-2 all of which are genes that are amplified in cancer showing an increased level of transcript (Nesrin Özören and Wafik S. El-Deiry, Introduction to cancer genes and growth control, In: DNA alterations in cancer, genetic and epigenetic changes, Eaton publishing, Melanie Ehrlich (ed) p. 1-43, 2000; and references therein). Many of these genes are related to cell growth and directs the tumor cells to uninhibited growth. Others may be related to tissue degradation as they e.g. encode enzymes that break down the surrounding connective tissue.

Bladder cancer is the fourth most common malignancy in males in the western countries (Pisani). The disease basically takes two different courses: one where patients have multiple recurrences of superficial tumors (Ta and T1), and one where the disease from the beginning is muscle invasive (T2+) and leads to metastasis. About 5-10% of patients with Ta tumors and 20-30% of the patients with T1 tumors will eventually develop a higher stage tumor (Wolf). Patients with superficial bladder tumors represent 75% of all bladder cancer patients and no clinical useful markers identifying patients with a poor prognosis exists at present.

The patients presenting isolated or concomitant Carcinoma in situ (CIS) lesions have a high risk of disease progression to a muscle invasive stage (Althausen). The CIS lesions may have a widespread manifestation in the bladder (field disease) and are believed to be the most common precursors of invasive carcinomas (Spruck, Rosin). The ability to predict which tumours are likely to recur or progress would have great impact on the clinical management of patients with superficial disease, as it would be possible to treat high-risk patients more aggressively (e.g. radical cystectomy or adjuvant therapy). This approach is currently not possible, as no clinical useful markers exist that identify these patients.

Although many prognostic markers have been investigated, the most important prognostic factors are still disease stage, dysplasia grade and especially the presence of areas with CIS (Anderstrom, Cummings, Cheng). The gold standard for detection of CIS is urine cytology and histopathologic analysis of a set of selected site biopsies removed during routine cytsocopy examinations; however these procedures are not sufficient sensitive.

Implementing routine cytoscopy examinations with 5-ALA fluorescence imaging of the tumours and pre-cancerous lesions (CIS lesions and moderate dysplasia lesions) may increase the sensitivity of the procedure (Kriegmar), however, increased detection sensitivity is still necessary in order to offer better treatment regiments to the individual patients.

### SUMMARY OF THE INVENTION

The present invention relates to prediction of prognosis of a biological condition, in particular to the prognosis of cancer such as bladder cancer. It is known that individuals suffering from cancer, although their tumors macroscopically and microscopically are identical, may have very different outcome. The present inventors have identified new predictor genes to classify macroscopically and microscopically identical tumors into two or more groups, wherein in each group has a separate risk profile of recurrence, invasive growth, metastasis etc. as compared to the other group(s). The present invention relates to genotyping of the tissue, and correlating the result to standard expression level(s) to predict the prognosis of the biological condition.

Accordingly, in one aspect the present invention relates to a method of predicting the prognosis of a biological condition in animal tissue,
comprising collecting a sample comprising cells from the tissue and/or expression products from the cells,
determining an expression level of at least one gene in said sample, said gene being selected from the group of genes consisting of gene No. 1 to gene No. 562,
correlating the expression level to at least one standard expression level to predict the prognosis of the biological condition in the animal tissue.

The genes No. 1—gene No. 562 are found in table A described below herein.

Animal tissue may be tissue from any animal, preferably from a mammal, such as a horse, a cow, a dog, a cat, and more preferably the tissue is human tissue. The biological condition may be any condition exhibiting gene expression different from normal tissue. In particular the biological condition relates to a malignant or premalignant condition, such as a tumor or cancer, in particular bladder cancer. By the term “collecting a sample comprising cells” is meant the sample is provided in a manner, so that the expression level of the genes may be determined.

Furthermore, the invention relates to a method of determining the stage of a biological condition in animal tissue,
comprising collecting a sample comprising cells from the tissue,
determining an expression level of at least one gene in said sample, said gene being selected from the group of genes consisting of gene No 1 to gene No. 562,
correlating the expression level of the assessed genes to at least one standard level of expression determining the stage of the condition.

The determination of the stage of the biological condition may be conducted prior to the method of predicting the method, or the stage of the biological condition may as such contain the information about the prognosis.

The methods above may be used for determining single gene expressions, however the invention also relates to a method of determining an expression pattern of a bladder cell sample, comprising:
collecting sample comprising bladder cells and/or expression products from bladder cells,
determining the expression level of at least one gene in the sample, said gene being selected from the group of genes consisting of gene No. 1 to gene No. 562, and obtaining an expression pattern of the bladder cell sample.

Further, the invention relates to a method of determining an expression pattern of a bladder cell sample independent of the proportion of submucosal, muscle, or connective tissue cells present, comprising:
determining the expression of one or more genes in a sample comprising cells, wherein the one or more genes exclude genes which are expressed in the submucosal, muscle, or connective tissue, whereby a pattern of expression is formed for the sample which is independent of the proportion of submucosal, muscle, or connective tissue cells in the sample.

The expression pattern may be used in a method according to this information, and accordingly, the invention also relates to a method of predicting the prognosis a biological condition in human bladder tissue comprising, collecting a sample comprising cells from the tissue, determining an expression pattern of the cells as defined in any of claims 43-54, correlating the determined expression pattern to a standard pattern, predicting the prognosis of the biological condition of said tissue as well as a method for determining the stage of a biological condition in animal tissue, comprising collecting a sample comprising cells from the tissue, determining an expression pattern of the cells as defined above, correlating the determined expression pattern to a standard pattern, determining the stage of the biological condition is said tissue.

The invention further relates to a method for reducing cell tumorigenicity or malignancy of a cell, said method comprising contacting a tumor cell with at least one peptide expressed by at least one gene selected from the group of genes consisting of gene Nos. 200-214, 233, 234, 235, 236, 244, 249, 251, 252, 255, 256, 259, 261, 262, 266, 268, 269, 273, 274, 275, 276, 277, 279, 280, 281, 282, 285, 286, 289, 293, 295, 296, 299, 301, 304, 306, 307, 308, 311, 312, 313, 314, 320, 322, 323, 325, 326, 327, 328, 330, 331, 332, 333, 334, 338, 341, 342, 343, 345, 348, 349, 350, 351, 352, 353, 355, 357, 360, 361, 363, 366, 367, 370, 373, 374, 375, 376, 385, 386, 387, 389, 390, 392, 394, 398, 400, 401, 405, 406, 407, 408, 410, 411, 412, 414, 415, 416, 418, 424, 426, 428, 433, 434, 435, 436, 438, 439, 440, 441, 442, 443, 445, 446, 453, 460, 461, 463, 464, 465, 466, 467, 469, 470, 471, 472, 473, 475, 476, 477, 479, 480, 481, 482, 483, 485, 486, 487, 488, 490, 492, 494, 496, 497, 498, 499, 503, 515, 516, 517, 521, 526, 527, 528, 530, 532, 533, 537, 539, 540, 541, 542, 543, 545, 554, 557, 560 or obtaining at least one gene selected from the group of genes consisting of gene Nos 200-214, 233, 234, 235, 236, 244, 249, 251, 252, 255, 256, 259, 261, 262, 266, 268, 269, 273, 274, 275, 276, 277, 279, 280, 281, 282, 285, 286, 289, 293, 295, 296, 299, 301, 304, 306, 307, 308, 311, 312, 313, 314, 320, 322, 323, 325, 326, 327, 328, 330, 331, 332, 333, 334, 338, 341, 342, 343, 345, 348, 349, 350, 351, 352, 353, 355, 357, 360, 361, 363, 366, 367, 370, 373, 374, 375, 376, 385, 386, 387, 389, 390, 392, 394, 398, 400, 401, 405, 406, 407, 408, 410, 411, 412, 414, 415, 416, 418, 424, 426, 428, 433, 434, 435, 436, 438, 439, 440, 441, 442, 443, 445, 446, 453, 460, 461, 463, 464, 465, 466, 467, 469, 470, 471, 472, 473, 475, 476, 477, 479, 480, 481, 482, 483, 485, 486, 487, 488, 490, 492, 494, 496, 497, 498, 499, 503, 515, 516, 517, 521, 526, 527, 528, 530, 532, 533, 537, 539, 540, 541, 542, 543, 545, 554, 557, 560, and introducing said at least one gene into the tumor cell in a manner allowing expression of said gene(s), or

obtaining at least one nucleotide probe capable of hybridising with at least one gene of a tumor cell, said at least one gene being selected from the group of genes consisting of gene Nos. 1-199, 215-232, 237, 238, 239, 240, 241, 242, 243, 245, 246, 247, 248, 250, 253, 254, 257, 258, 260, 263, 264, 265, 267, 270, 271, 272, 278, 283, 284, 287, 288, 290, 291, 292, 294, 297, 298, 300, 302, 303, 305, 309, 310, 315, 316, 317, 318, 319, 321, 324, 329, 335, 336, 337, 339, 340, 344, 346, 347, 354, 356, 358, 359, 362, 364, 365, 368, 369, 371, 372, 377, 378, 379, 380, 381, 382, 383, 384, 388, 391, 393, 395, 396, 397, 399, 402, 403, 404, 409, 413, 417, 419, 420, 421, 422, 423, 425, 427, 429, 430, 431, 432, 437, 444, 447, 448, 449, 450, 451, 452, 454, 455, 456, 457, 458, 459, 462, 468, 474, 478, 484, 489, 491, 493, 495, 500, 501, 502, 504, 505, 506, 507, 508, 509, 510, 511, 512, 513, 514, 518, 519, 520, 522, 523, 524, 525, 529, 531, 534, 535, 536, 538, 544, 546, 547, 548, 549, 550, 551, 552, 553, 555, 556, 558, 559, 561, 562, and introducing said at least one nucleotide probe into the tumor cell in a manner allowing the probe to hybridise to the at least one gene, thereby inhibiting expression of said at least one gene.

In a further aspect the invention relates to a method for producing antibodies against an expression product of a cell from a biological tissue, said method comprising the steps of

obtaining expression product(s) from at least one gene said gene being expressed as defined above,
immunising a mammal with said expression product(s) obtaining antibodies against the expression product.

The antibodies produced may be used for producing a pharmaceutical composition. Further, the invention relates to a vaccine capable of eliciting an immune response against at least one expression product from at least one gene said gene being expressed as defined above.

The invention furthermore relates to the use of any of the methods discussed above for producing an assay for diagnosing a biological condition in animal tissue.

Also, the invention relates to the use of a peptide as defined above as an expression product and/or the use of a gene as defined above and/or the use of a probe as defined above for preparation of a pharmaceutical composition for the treatment of a biological condition in animal tissue.

In yet a further aspect the invention relates to an assay for determining the presence or absence of a biological condition in animal tissue, comprising
at least one first marker capable of detecting an expression level of at least one gene selected from the group of genes consisting of gene No. 1 to gene No. 562,

In another aspect the invention relates to an assay for determining an expression pattern of a bladder cell, comprising at least a first marker and/or a second marker, wherein the first marker is capable of detecting a gene from a first gene group as defined above, and the second marker is capable of detecting a gene from a second gene group as defined above.

### DRAWINGS

Description of figures:

FIG. 1 Hierarchical cluster analysis of tumor samples based on 3,197 genes that show large variation across all tumor samples. Samples with progression are marked Prog.

FIG. 2 Delineation of the 200 best marker genes. Genes that show higher levels of expression in the non-progression group are shown in the top and genes that show higher levels of expression in the progression group is shown in the bottom. Each column in the diagram represents a tumor sample and each row a gene. The 13 non-progressing samples are shown to the left and the 16 progressing samples are shown to the right in the diagram. The color saturation indicates differences in gene expression across the tumor samples; light color indicates up regulation compared the median expression and down regulation compared to the median expression of the gene is shown in dark color. Gene names of particular interesting genes are listed. Notable, non-group expression patterns were observed for two tumors (arrows). The tumor in the no progression group (150-6) showed a solid growth pattern, which is associated with a poor prognosis. No special tumor characteristics can help explain the gene expression pattern observed for the tumor in the progression group (825-3).

FIG. 3. Cross-validation performance using from 1 to 200 genes.

FIG. 4. Predicting progression in early stage bladder tumors. a, The 45-gene expression signature found to be optimal for progression prediction. Genes showing high expression in progressing samples are show in the top and genes showing high expression in the non-progressing samples are shown in the bottom. Genes are listed according to how many cross-validation loops included the genes. b, The 45-gene expression signature in the 19 tumor test-set. The samples are listed according to the correlation to the average non-progression signature from the training set samples. The read punctuated line separates samples with positive (left) and negative (right) correlation values. The white lines separates samples above and below the correlation cutoff values of 0.1 and −0.1. The sample legend indicates no-progression (N) samples and progression (P) samples.

FIG. 5 Hierarchical cluster analysis of the metachronous tumor samples. Tight clustering tumors of different stage from the same patients are colored in grey.

FIG. 6 Two-way hierarchical clustering and multidimensional scaling analysis of gene expression data from 40 bladder tumour biopsies. a, Tumour cluster dendrogram based on the 1767 gene-set. CIS annotations following the sample names indicate concomitant carcinoma in situ. Tumour recurrence rates are shown to the right of the dendrogram as + and ++ indicating moderate and high recurrence rates, respectively, while no sign indicates no or moderate recurrence. b, Tumour cluster dendrogram based on 88 cancer related genes. c, 2D plot of multidimensional scaling analysis of the 40 tumours based on the 1767 gene-set. The colour code identifies the tumour samples from the cluster dendrogram (FIG. 1a). d, Two-way cluster analysis diagram of the 1767 gene-set. Each row in the diagram represents a gene and each column a tumour sample. The colour saturation represents differences in gene expression across the tumour samples; light color indicates higher expression of the gene compared to the median expression and lower expression of the gene compared to the median expression shown in dark color. The colour intensities indicate degrees of gene-regulation. The sidebars to the right of the diagram represent gene clusters a-j and normal 1-3 in the left side indicate the three normal biopsies and normal 4 indicates the pool of biopsies from 37 patients.

FIG. 7 Enlarged view of the gene clusters a, c, f, and g. The dendrogram at the top is identical to FIG. 6a. a, Cluster of transcription factors and other nuclear associated genes. c, Cluster of genes involved in proliferation and cell cycle control. f, Gene expression pattern and corresponding area with squamous metaplasia in urothelial carcinoma. The light colour indicates genes up-regulated in samples 1178-1 and 875-1, the only two samples with squamous cell metaplasia. g, Cluster of genes involved in angiogenesis and matrix remodelling.

FIG. 8. Hierarchical Cluster Analysis Results

Here we show expanded views of clusters a-j as identified in the 1767 gene-cluster. The tumour cluster dendrogram and colour bars on top of the clusters represents the same tumour cluster as shown in the paper. The four samples to the left are normal biopsies (normal 1-3) and a pool of 37 normal biopsies (normal 4).

FIG. 8a. Molecular classification of tumour samples using 80 predictive genes in each cross-validation loop. Each classification is based on the closeness to the mean in the three classes. Samples marked with * were not used to build the classifier. The scale indicates the distance from the samples to the classes in the classifier, measured in weighted squared Euclidean distance.

FIG. 9 Number of classification errors vs. number of genes used in cross-validation loops.

FIG. 10 Expression profiles of the 71 genes used in the final classifier model. The tumors shown are the 33 tumors used in the cross validation scheme. The Ta tumors are shown to the left, the T1 tumors in the middle, and the T2 tumors to the right.

FIG. 11 Number of prediction errors vs. number of genes used in cross-validation loops.

FIG. 12 The expression profiles of the 26 genes that constitute our final prediction model. The genes are listed according to the degree of correlation with the recurrence and non-recurrence groups. Genes with highest correlations are found in the top and the bottom of the list.

FIG. 13. Hierarchical cluster analysis of the gene expression in 41 TCC, 9 normal samples and 10 samples from cystectomy specimens with CIS lesions. a, Cluster dendrogram of all 41 TCC biopsies based on the expression of 5,491 genes. b, Cluster dendrogram of all superficial TCC biopsies based on the expression of 5,252 genes. c, Two-way cluster analysis diagram of the 41 TCC biopsies together with gene expressions in the normal and cystectomy samples (left columns). Each row represents a gene and each column represent a biopsy sample. Yellow indicates up-regulation compared to the median expression (black) of the gene and blue indicates down-regulation compared to the median expression. The colour saturation indicates degree of gene regulation. The sidebars to the right of the diagram represent gene-clusters 1-4; enlarged views of cluster 1 and 4 are shown to the right, with all gene symbols listed.

FIG. 14. Delineation of the 100 best markers that separate TCC without CIS from TCC with concomitant CIS. a, The 50 best up-regulated marker genes in TCC without CIS are shown in the top and the 50 best up-regulated marker genes in TCC with CIS are shown in the bottom. The gene symbols are listed to the right of the diagram. b, Expression profiles of the 100 marker genes in 9 normal biopsies (left column), 5 histologically normal samples adjacent to CIS lesions (middle column), and 5 biopsies with CIS lesions detected. (right column).

FIG. 15 Cross validation performance using all samples

FIG. 16 Expression profiles of the 16 genes in the CIS classifier. a, the expression of the 16 classifier genes in TCC with no surrounding CIS (left) and in TCC with surrounding CIS (right). The gene symbols of the classifier genes are listed together with the number of the times used in cross-validation loops. b, the expression of the 16 classifier genes in normal samples, in histologically normal samples adjacent to CIS lesions, and in biopsies with CIS lesions. The top dendrogram shows the sample clustering from hierarchical cluster analysis based on the 16 classifier genes. The genes appear in the same order as in 3a.

FIG. 17 Cross validation performance using half of the samples

FIG. 18 shows table B

FIG. 19 shows table C

FIG. 20 shows table D

FIG. 21 shows table E

FIG. 22 shows table F

FIG. 23 shows table G

FIG. 24 shows table H

### DETAILED DESCRIPTION OF THE INVENTION

As discussed above the present invention relates to the finding that it is possible to predict the prognosis of a biological condition by determining the expression level of one or more genes from a specified group of genes and comparing the expression level to at least one standard for expression levels. The present inventors have identified 562 genes relevant for predicting the prognosis of a biological condition, in particular a cancer disease, such as bladder cancer.

The following table A shows the genes relevant in this context. Whenever a gene is cited herein with reference to a gene No. the numbering refers to the genes of Table A.

TABLE A

| | | | Unigene | | | |
|---|---|---|---|---|---|---|
| Gene # | GeneChip | Probeset | Build | Unigene | description | Classifier |
| 1 | HUGeneFL | AB000220_at | 168 | Hs.171921 | sema domain, immunoglobulin domain | stage |
| | | | | | (Ig), short basic domain, secreted, | |
| | | | | | (semaphorin) 3C | |
| 2 | HUGeneFL | AF000231_at | 168 | Hs.75618 | RAB11A, member RAS oncogene | stage |
| | | | | | family | |
| 3 | HUGeneFL | D10922_s_at | 168 | Hs.99855 | formyl peptide receptor-like 1 | stage |
| 4 | HUGeneFL | D10925_at | 168 | Hs.301921 | chemokine (C-C motif) receptor 1 | stage |
| 5 | HUGeneFL | D11086_at | 168 | Hs.84 | interleukin 2 receptor, gamma (severe | stage |
| | | | | | combined immunodeficiency) | |
| 6 | HUGeneFL | D11151_at | 168 | Hs.211202 | endothelin receptor type A | stage |
| 7 | HUGeneFL | D13435_at | 168 | Hs.426142 | phosphatidylinositol glycan, class F | stage |
| 8 | HUGeneFL | D13666_s_at | 168 | Hs.136348 | osteoblast specific factor 2 (fasciclin I- | stage |
| | | | | | like) | |
| 9 | HUGeneFL | D14520_at | 168 | Hs.84728 | Kruppel-like factor 5 (intestinal) | stage |
| 10 | HUGeneFL | D21878_at | 168 | Hs.169998 | bone marrow stromal cell antigen 1 | stage |
| 11 | HUGeneFL | D26443_at | 168 | Hs.371369 | solute carrier family 1 (glial high affinity | stage |
| | | | | | glutamate transporter), member 3 | |
| 12 | HUGeneFL | D42046_at | 168 | Hs.194665 | DNA2 DNA replication helicase 2-like | stage |
| | | | | | (yeast) | |
| 13 | HUGeneFL | D45370_at | 168 | Hs.74120 | adipose specific 2 | stage |
| 14 | HUGeneFL | D49372_s_at | 168 | Hs.54460 | chemokine (C-C motif) ligand 11 | stage |
| 15 | HUGeneFL | D50495_at | 168 | Hs.224397 | transcription elongation factor A (SII), 2 | stage |
| 16 | HUGeneFL | D63135_at | 168 | Hs.27935 | tweety homolog 2 (Drosophila) | stage |
| 17 | HUGeneFL | D64053_at | 168 | Hs.198288 | protein tyrosine phosphatase, receptor | stage |
| | | | | | type, R | |
| 18 | HUGeneFL | D83920_at | 168 | Hs.440898 | ficolin (collagen/fibrinogen domain | stage |
| | | | | | containing) 1 | |
| 19 | HUGeneFL | D85131_s_at | 168 | Hs.433881 | MYC-associated zinc finger protein | stage |
| | | | | | (purine-binding transcription factor) | |
| 20 | HUGeneFL | D86062_s_at | 168 | Hs.413482 | chromosome 21 open reading frame | stage |
| | | | | | 33 | |
| 21 | HUGeneFL | D86479_at | 168 | Hs.439463 | AE binding protein 1 | stage |
| 22 | HUGeneFL | D86957_at | 168 | Hs.307944 | likely ortholog of mouse septin 8 | stage |
| 23 | HUGeneFL | D86959_at | 168 | Hs.105751 | Ste20-related serine/threonine kinase | stage |
| 24 | HUGeneFL | D86976_at | 168 | Hs.196914 | minor histocompatibility antigen HA-1 | stage |
| 25 | HUGeneFL | D87433_at | 168 | Hs.301989 | stabilin 1 | stage |
| 26 | HUGeneFL | D87443_at | 168 | Hs.409862 | sorting nexin 19 | stage |
| 27 | HUGeneFL | D87682_at | 168 | Hs.134792 | KIAA0241 protein | stage |
| 28 | HUGeneFL | D89077_at | 168 | Hs.75367 | Src-like-adaptor | stage |
| 29 | HUGeneFL | D89377_at | 168 | Hs.89404 | msh homeo box homolog 2 (Drosophila) | stage |
| 30 | HUGeneFL | D90279_s_at | 168 | Hs.433695 | collagen, type V, alpha 1 | stage |
| 31 | HUGeneFL | HG1996-HT2044_at | 168 | — | — | stage |
| 32 | HUGeneFL | HG2090-HT2152_s_at | 168 | — | — | stage |
| 33 | HUGeneFL | HG2463-HT2559_at | 168 | — | — | stage |
| 34 | HUGeneFL | HG3044-HT3742_s_at | 168 | — | — | stage |
| 35 | HUGeneFL | HG3187-HT3366_s_at | 168 | — | — | stage |
| 36 | HUGeneFL | HG3342-HT3519_s_at | 168 | — | — | stage |
| 37 | HUGeneFL | HG371-HT26388_s_at | 168 | — | — | stage |
| 38 | HUGeneFL | HG4069-HT4339_s_at | 168 | — | — | stage |
| 39 | HUGeneFL | HG67-HT67_f_at | 168 | — | — | stage |
| 40 | HUGeneFL | HG907-HT907_at | 168 | — | — | stage |
| 41 | HUGeneFL | J02871_s_at | 168 | Hs.436317 | cytochrome P450, family 4, subfamily | stage |
| | | | | | B, polypeptide 1 | |
| 42 | HUGeneFL | J03040_at | 168 | Hs.111779 | secreted protein, acidic, cysteine-rich | stage |
| | | | | | (osteonectin) | |
| 43 | HUGeneFL | J03060_at | 168 | — | — | stage |
| 44 | HUGeneFL | J03068_at | 168 | — | — | stage |
| 45 | HUGeneFL | J03241_s_at | 168 | Hs.2025 | transforming growth factor, beta 3 | stage |
| 46 | HUGeneFL | J03278_at | 168 | Hs.307783 | platelet-derived growth factor receptor, | stage |
| | | | | | beta polypeptide | |
| 47 | HUGeneFL | J03909_at | 168 | — | — | stage |
| 48 | HUGeneFL | J03925_at | 168 | Hs.172631 | integrin, alpha M (complement component | stage |
| | | | | | receptor 3, alpha; also known as | |
| | | | | | CD11b (p170), macrophage antigen | |
| | | | | | alpha polypeptide) | |
| 49 | HUGeneFL | J04056_at | 168 | Hs.88778 | carbonyl reductase 1 | stage |
| 50 | HUGeneFL | J04058_at | 168 | Hs.169919 | electron-transfer-flavoprotein, alpha | stage |
| | | | | | polypeptide (glutaric aciduria II) | |
| 51 | HUGeneFL | J04130_s_at | 168 | Hs.75703 | chemokine (C-C motif) ligand 4 | stage |
| 52 | HUGeneFL | J04152_rna1_s_at | 168 | — | — | stage |
| 53 | HUGeneFL | J04162_at | 168 | Hs.372679 | Fc fragment of IgG, low affinity IIIa, | stage |
| | | | | | receptor for (CD16) | |
| 54 | HUGeneFL | J04456_at | 168 | Hs.407909 | lectin, galactoside-binding, soluble, 1 | stage |
| | | | | | (galectin 1) | |
| 55 | HUGeneFL | J05032_at | 168 | Hs.32393 | aspartyl-tRNA synthetase | stage |
| 56 | HUGeneFL | J05070_at | 168 | Hs.151738 | matrix metalloproteinase 9 (gelatinase | stage |
| | | | | | B, 92 kDa gelatinase, 92 kDa type IV | |
| | | | | | collagenase) | |
| 57 | HUGeneFL | J05448_at | 168 | Hs.79402 | polymerase (RNA) II (DNA directed) | stage |
| | | | | | polypeptide C, 33 kDa | |
| 58 | HUGeneFL | K01396_at | 168 | Hs.297681 | serine (or cysteine) proteinase inhibitor, | stage |
| | | | | | clade A (alpha-1 antiproteinase, | |
| | | | | | antitrypsin), member 1 | |
| 59 | HUGeneFL | K03430_at | 168 | — | — | stage |
| 60 | HUGeneFL | L06797_s_at | 168 | Hs.421986 | chemokine (C—X—C motif) receptor 4 | stage |
| 61 | HUGeneFL | L10343_at | 168 | Hs.112341 | protease inhibitor 3, skin-derived | stage |
| | | | | | (SKALP) | |
| 62 | HUGeneFL | L13391_at | 168 | Hs.78944 | regulator of G-protein signalling 2, | stage |
| | | | | | 24 kDa | |
| 63 | HUGeneFL | L13698_at | 168 | Hs.65029 | growth arrest-specific 1 | stage |
| 64 | HUGeneFL | L13720_at | 168 | Hs.437710 | growth arrest-specific 6 | stage |
| 65 | HUGeneFL | L13923_at | 168 | Hs.750 | fibrillin 1 (Marfan syndrome) | stage |
| 66 | HUGeneFL | L15409_at | 168 | Hs.421597 | von Hippel-Lindau syndrome | stage |
| 67 | HUGeneFL | L17325_at | 168 | Hs.195825 | RNA binding protein with multiple | stage |
| | | | | | splicing | |
| 68 | HUGeneFL | L19872_at | 168 | Hs.170087 | aryl hydrocarbon receptor | stage |
| 69 | HUGeneFL | L27476_at | 168 | Hs.75608 | tight junction protein 2 (zona occludens | stage |
| | | | | | 2) | |
| 70 | HUGeneFL | L33799_at | 168 | Hs.202097 | procollagen C-endopeptidase enhancer | stage |
| 71 | HUGeneFL | L40388_at | 168 | Hs.30212 | thyroid receptor interacting protein 15 | stage |
| 72 | HUGeneFL | L40904_at | 168 | Hs.387667 | peroxisome proliferative activated | stage |
| | | | | | receptor, gamma | |
| 73 | HUGeneFL | L41919_rna1_at | 168 | — | — | stage |
| 74 | HUGeneFL | M11433_at | 168 | Hs.101850 | retinol binding protein 1, cellular | stage |
| 75 | HUGeneFL | M11718_at | 168 | Hs.283393 | collagen, type V, alpha 2 | stage |
| 76 | HUGeneFL | M12125_at | 168 | Hs.300772 | tropomyosin 2 (beta) | stage |
| 77 | HUGeneFL | M14218_at | 168 | Hs.442047 | argininosuccinate lyase | stage |
| 78 | HUGeneFL | M15395_at | 168 | Hs.375957 | integrin, beta 2 (antigen CD18 (p95), | stage |
| | | | | | lymphocyte function-associated antigen | |
| | | | | | 1; macrophage antigen 1 (mac-1) | |
| | | | | | beta subunit) | |
| 79 | HUGeneFL | M16591_s_at | 168 | Hs.89555 | hemopoietic cell kinase | stage |
| 80 | HUGeneFL | M17219_at | 168 | Hs.203862 | guanine nucleotide binding protein (G | stage |
| | | | | | protein), alpha inhibiting activity poly- | |
| | | | | | peptide 1 | |
| 81 | HUGeneFL | M20530_at | 168 | — | — | stage |
| 82 | HUGeneFL | M23178_s_at | 168 | Hs.73817 | chemokine (C-C motif) ligand 3 | stage |
| 83 | HUGeneFL | M28130_rna1_s_at | 168 | — | — | stage |
| 84 | HUGeneFL | M29550_at | 168 | Hs.187543 | protein phosphatase 3 (formerly 2B), | stage |
| | | | | | catalytic subunit, beta isoform (calcineurin | |
| | | | | | A beta) | |
| 85 | HUGeneFL | M31165_at | 168 | Hs.407546 | tumor necrosis factor, alpha-induced | stage |
| | | | | | protein 6 | |
| 86 | HUGeneFL | M32011_at | 168 | Hs.949 | neutrophil cytosolic factor 2 (65 kDa, | stage |
| | | | | | chronic granulomatous disease, autosomal | |
| | | | | | 2) | |
| 87 | HUGeneFL | M33195_at | 168 | Hs.433300 | Fc fragment of IgE, high affinity I, | stage |
| | | | | | receptor for; gamma polypeptide | |
| 88 | HUGeneFL | M37033_at | 168 | Hs.443057 | CD53 antigen | stage |
| 89 | HUGeneFL | M37766_at | 168 | Hs.901 | CD48 antigen (B-cell membrane | stage |
| | | | | | protein) | |
| 90 | HUGeneFL | M55998_s_at | 168 | Hs.172928 | collagen, type I, alpha 1 | stage |
| 91 | HUGeneFL | M57731_s_at | 168 | Hs.75765 | chemokine (C—X—C motif) ligand 2 | stage |
| 92 | HUGeneFL | M62840_at | 168 | Hs.82542 | acyloxyacyl hydrolase (neutrophil) | stage |
| 93 | HUGeneFL | M63262_at | 168 | — | — | stage |
| 94 | HUGeneFL | M68840_at | 168 | Hs.183109 | monoamine oxidase A | stage |
| 95 | HUGeneFL | M69203_s_at | 168 | Hs.75703 | chemokine (C-C motif) ligand 4 | stage |
| 96 | HUGeneFL | M72885_rna1_s_at | 168 | — | — | stage |
| 97 | HUGeneFL | M77349_at | 168 | Hs.421496 | transforming growth factor, beta- | stage |
| | | | | | induced, 68 kDa | |
| 98 | HUGeneFL | M82882_at | 168 | Hs.124030 | E74-like factor 1 (ets domain transcription | stage |
| | | | | | factor) | |
| 99 | HUGeneFL | M83822_at | 168 | Hs.209846 | LPS-responsive vesicle trafficking, | stage |
| | | | | | beach and anchor containing | |
| 100 | HUGeneFL | M92934_at | 168 | Hs.410037 | connective tissue growth factor | stage |
| 101 | HUGeneFL | M95178_at | 168 | Hs.119000 | actinin, alpha 1 | stage |
| 102 | HUGeneFL | S69115_at | 168 | Hs.10306 | natural killer cell group 7 sequence | stage |
| 103 | HUGeneFL | S77393_at | 168 | Hs.145754 | kruppel-like factor 3 (basic) | stage |
| 104 | HUGeneFL | S78187_at | 168 | Hs.153752 | cell division cycle 25B | stage |
| 105 | HUGeneFL | U01833_at | 168 | Hs.81469 | nucleotide binding protein 1 (MinD | stage |
| | | | | | homolog, E. coli) | |
| 106 | HUGeneFL | U07231_at | 168 | Hs.309763 | G-rich RNA sequence binding factor 1 | stage |
| 107 | HUGeneFL | U09278_at | 168 | Hs.436852 | fibroblast activation protein, alpha | stage |
| 108 | HUGeneFL | U09937_rna1_s_at | 168 | — | — | stage |
| 109 | HUGeneFL | U10550_at | 168 | Hs.79022 | GTP binding protein overexpressed in | stage |
| | | | | | skeletal muscle | |
| 110 | HUGeneFL | U12424_s_at | 168 | Hs.108646 | glycerol-3-phosphate dehydrogenase | stage |
| | | | | | 2 (mitochondrial) | |
| 111 | HUGeneFL | U16306_at | 168 | Hs.434488 | chondroitin sulfate proteoglycan 2 | stage |
| | | | | | (versican) | |
| 112 | HUGeneFL | U20158_at | 168 | Hs.2488 | lymphocyte cytosolic protein 2 (SH2 | stage |
| | | | | | domain containing leukocyte protein of | |
| | | | | | 76 kDa) | |
| 113 | HUGeneFL | U20536_s_at | 168 | Hs.3280 | caspase 6, apoptosis-related cysteine | stage |
| | | | | | protease | |
| 114 | HUGeneFL | U24266_at | 168 | Hs.77448 | aldehyde dehydrogenase 4 family, | stage |
| | | | | | member A1 | |
| 115 | HUGeneFL | U28249_at | 168 | Hs.301350 | FXYD domain containing ion transport | stage |
| | | | | | regulator 3 | |
| 116 | HUGeneFL | U28488_s_at | 168 | Hs.155935 | complement component 3a receptor 1 | stage |
| 117 | HUGeneFL | U29680_at | 168 | Hs.227817 | BCL2-related protein A1 | stage |
| 118 | HUGeneFL | U37143_at | 168 | Hs.152096 | cytochrome P450, family 2, subfamily | stage |
| | | | | | J, polypeptide 2 | |
| 119 | HUGeneFL | U38864_at | 168 | Hs.108139 | zinc finger protein 212 | stage |
| 120 | HUGeneFL | U39840_at | 168 | Hs.163484 | forkhead box A1 | stage |
| 121 | HUGeneFL | U41315_rna1_s_at | 168 | — | — | stage |
| 122 | HUGeneFL | U44111_at | 168 | Hs.42151 | histamine N-methyltransferase | stage |
| 123 | HUGeneFL | U47414_at | 168 | Hs.13291 | cyclin G2 | stage |
| 124 | HUGeneFL | U49352_at | 168 | Hs.414754 | 2,4-dienoyl CoA reductase 1, mitochondrial | stage |
| 125 | HUGeneFL | U50708_at | 168 | Hs.1265 | branched chain keto acid dehydrogenase | stage |
| | | | | | E1, beta polypeptide (maple | |
| | | | | | syrup urine disease) | |
| 126 | HUGeneFL | U52101_at | 168 | Hs.9999 | epithelial membrane protein 3 | stage |
| 127 | HUGeneFL | U59914_at | 168 | Hs.153863 | MAD, mothers against decapentaplegic | stage |
| | | | | | homolog 6 (Drosophila) | |
| 128 | HUGeneFL | U60205_at | 168 | Hs.393239 | sterol-C4-methyl oxidase-like | stage |
| 129 | HUGeneFL | U61981_at | 168 | Hs.42674 | mutS homolog 3 (E. coli) | stage |
| 130 | HUGeneFL | U64520_at | 168 | Hs.66708 | vesicle-associated membrane protein | stage |
| | | | | | 3 (cellubrevin) | |
| 131 | HUGeneFL | U65093_at | 168 | Hs.82071 | Cbp/p300-interacting transactivator, | stage |
| | | | | | with Glu/Asp-rich carboxy-terminal | |
| | | | | | domain, 2 | |
| 132 | HUGeneFL | U66619_at | 168 | Hs.444445 | SWI/SNF related, matrix associated, | stage |
| | | | | | actin dependent regulator of chromatin, | |
| | | | | | subfamily d, member 3 | |
| 133 | HUGeneFL | U68019_at | 168 | Hs.288261 | MAD, mothers against decapentaplegic | stage |
| | | | | | homolog 3 (Drosophila) | |
| 134 | HUGeneFL | U68385_at | 168 | Hs.380923 | likely ortholog of mouse myeloid ecotropic | stage |
| | | | | | viral integration site-related gene 2 | |
| 135 | HUGeneFL | U68485_at | 168 | Hs.193163 | bridging integrator 1 | stage |
| 136 | HUGeneFL | U74324_at | 168 | Hs.90875 | RAB interacting factor | stage |
| 137 | HUGeneFL | U77970_at | 168 | Hs.321164 | neuronal PAS domain protein 2 | stage |
| 138 | HUGeneFL | U83303_cds2_at | 168 | Hs.164021 | chemokine (C—X—C motif) ligand 6 | stage |
| | | | | | (granulocyte chemotactic protein 2) | |
| 139 | HUGeneFL | U88871_at | 168 | Hs.79993 | peroxisomal biogenesis factor 7 | stage |
| 140 | HUGeneFL | U90549_at | 168 | Hs.236774 | high mobility group nucleosomal binding | stage |
| | | | | | domain 4 | |
| 141 | HUGeneFL | U90716_at | 168 | Hs.79187 | coxsackie virus and adenovirus receptor | stage |
| 142 | HUGeneFL | V00594_at | 168 | Hs.118786 | metallothionein 2A | stage |
| 143 | HUGeneFL | V00594_s_at | 168 | Hs.118786 | metallothionein 2A | stage |
| 144 | HUGeneFL | X02761_s_at | 168 | Hs.418138 | fibronectin 1 | stage |
| 145 | HUGeneFL | X04011_at | 168 | Hs.88974 | cytochrome b-245, beta polypeptide | stage |
| | | | | | (chronic granulomatous disease) | |
| 146 | HUGeneFL | X04085_rna1_at | 168 | — | — | stage |
| 147 | HUGeneFL | X07438_s_at | 168 | — | — | stage |
| 148 | HUGeneFL | X07743_at | 168 | Hs.77436 | pleckstrin | stage |
| 149 | HUGeneFL | X13334_at | 168 | Hs.75627 | CD14 antigen | stage |
| 150 | HUGeneFL | X14046_at | 168 | Hs.153053 | CD37 antigen | stage |
| 151 | HUGeneFL | X14813_at | 168 | Hs.166160 | acetyl-Coenzyme A acyltransferase 1 | stage |
| | | | | | (peroxisomal 3-oxoacyl-Coenzyme A | |
| | | | | | thiolase) | |
| 152 | HUGeneFL | X15880_at | 168 | Hs.415997 | collagen, type VI, alpha 1 | stage |
| 153 | HUGeneFL | X15882_at | 168 | Hs.420269 | collagen, type VI, alpha 2 | stage |
| 154 | HUGeneFL | X51408_at | 168 | Hs.380138 | chimerin (chimaerin) 1 | stage |
| 155 | HUGeneFL | X53800_s_at | 168 | Hs.89690 | chemokine (C—X—C motif) ligand 3 | stage |
| 156 | HUGeneFL | X54489_rna1_at | 168 | — | — | stage |
| 157 | HUGeneFL | X57351_s_at | 168 | Hs.174195 | interferon induced transmembrane | stage |
| | | | | | protein 2 (1-8D) | |
| 158 | HUGeneFL | X57579_s_at | 168 | — | — | stage |
| 159 | HUGeneFL | X58072_at | 168 | Hs.169946 | GATA binding protein 3 | stage |
| 160 | HUGeneFL | X62048_at | 168 | Hs.249441 | WEE1 homolog (S. pombe) | stage |
| 161 | HUGeneFL | X64072_s_at | 168 | Hs.375957 | integrin, beta 2 (antigen CD18 (p95), | stage |
| | | | | | lymphocyte function-associated antigen | |
| | | | | | 1; macrophage antigen 1 (mac-1) | |
| | | | | | beta subunit) | |
| 162 | HUGeneFL | X65614_at | 168 | Hs.2962 | S100 calcium binding protein P | stage |
| 163 | HUGeneFL | X66945_at | 168 | Hs.748 | fibroblast growth factor receptor 1 | stage |
| | | | | | (fms-related tyrosine kinase 2, Pfeiffer | |
| | | | | | syndrome) | |
| 164 | HUGeneFL | X67491_f_at | 168 | Hs.355697 | glutamate dehydrogenase 1 | stage |
| 165 | HUGeneFL | X68194_at | 168 | Hs.80919 | synaptophysin-like protein | stage |
| 166 | HUGeneFL | X73882_at | 168 | Hs.254605 | microtubule-associated protein 7 | stage |
| 167 | HUGeneFL | X78520_at | 168 | Hs.372528 | chloride channel 3 | stage |
| 168 | HUGeneFL | X78549_at | 168 | Hs.51133 | PTK6 protein tyrosine kinase 6 | stage |
| 169 | HUGeneFL | X78565_at | 168 | Hs.98998 | tenascin C (hexabrachion) | stage |
| 170 | HUGeneFL | X78669_at | 168 | Hs.79088 | reticulocalbin 2, EF-hand calcium | stage |
| | | | | | binding domain | |
| 171 | HUGeneFL | X83618_at | 168 | Hs.59889 | 3-hydroxy-3-methylglutaryl-Coenzyme | stage |
| | | | | | A synthase 2 (mitochondrial) | |
| 172 | HUGeneFL | X84908_at | 168 | Hs.78060 | phosphorylase kinase, beta | stage |
| 173 | HUGeneFL | X90908_at | 168 | Hs.147391 | fatty acid binding protein 6, ileal (gastrotropin) | stage |
| 174 | HUGeneFL | X91504_at | 168 | Hs.389277 | ADP-ribosylation factor related protein 1 | stage |
| 175 | HUGeneFL | X95632_s_at | 168 | Hs.387906 | abl-interactor 2 | stage |
| 176 | HUGeneFL | X97267_rna1_s_at | 168 | — | — | stage |
| 177 | HUGeneFL | Y00705_at | 168 | Hs.407856 | serine protease inhibitor, Kazal type 1 | stage |
| 178 | HUGeneFL | Y00787_s_at | 168 | Hs.624 | interleukin 8 | stage |
| 179 | HUGeneFL | Y00815_at | 168 | Hs.75216 | protein tyrosine phosphatase, receptor | stage |
| | | | | | type, F | |
| 180 | HUGeneFL | Y08374_rna1_at | 168 | — | — | stage |
| 181 | HUGeneFL | Z12173_at | 168 | Hs.334534 | glucosamine (N-acetyl)-6-sulfatase | stage |
| | | | | | (Sanfilippo disease IIID) | |
| 182 | HUGeneFL | Z19554_s_at | 168 | Hs.435800 | vimentin | stage |
| 183 | HUGeneFL | Z26491_s_at | 168 | Hs.240013 | catechol-O-methyltransferase | stage |
| 184 | HUGeneFL | Z29331_at | 168 | Hs.372758 | ubiquitin-conjugating enzyme E2H | stage |
| | | | | | (UBC8 homolog, yeast) | |
| 185 | HUGeneFL | Z35491_at | 168 | Hs.377484 | BCL2-associated athanogene | stage |
| 186 | HUGeneFL | Z48199_at | 168 | Hs.82109 | syndecan 1 | stage |
| 187 | HUGeneFL | Z48605_at | 168 | Hs.421825 | inorganic pyrophosphatase 2 | stage |
| 188 | HUGeneFL | Z74615_at | 168 | Hs.172928 | collagen, type I, alpha 1 | stage |
| 189 | HUGeneFL | D87437_at | 168 | Hs.43660 | chromosome 1 open reading frame 16 | recurrence |
| 190 | HUGeneFL | L49169_at | 168 | Hs.75678 | FBJ murine osteosarcoma viral oncogene | recurrence |
| | | | | | homolog B | |
| 191 | HUGeneFL | AF006041_at | 168 | Hs.336916 | death-associated protein 6 | recurrence |
| 192 | HUGeneFL | D83780_at | 168 | Hs.437991 | KIAA0196 gene product | recurrence |
| 193 | HUGeneFL | D64154_at | 168 | Hs.90107 | adhesion regulating molecule 1 | recurrence |
| 194 | HUGeneFL | D21337_at | 168 | Hs.408 | collagen, type IV, alpha 6 | recurrence |
| 195 | HUGeneFL | M16938_s_at | 168 | Hs.820 | homeo box C6 | recurrence |
| 196 | HUGeneFL | D87258_at | 168 | Hs.75111 | protease, serine, 11 (IGF binding) | recurrence |
| 197 | HUGeneFL | U58516_at | 168 | Hs.3745 | milk fat globule-EGF factor 8 protein | recurrence |
| 198 | HUGeneFL | U45973_at | 168 | Hs.178347 | skeletal muscle and kidney enriched | recurrence |
| | | | | | inositol phosphatase | |
| 199 | HUGeneFL | U62015_at | 168 | Hs.8867 | cysteine-rich, angiogenic inducer, 61 | recurrence |
| 200 | HUGeneFL | U94855_at | 168 | Hs.381255 | eukaryotic translation initiation factor 3, | recurrence |
| | | | | | subunit 5 epsilon, 47 kDa | |
| 201 | HUGeneFL | L34155_at | 168 | Hs.83450 | laminin, alpha 3 | recurrence |
| 202 | HUGeneFL | U70439_s_at | 168 | Hs.84264 | acidic (leucine-rich) nuclear phospho- | recurrence |
| | | | | | protein 32 family, member B | |
| 203 | HUGeneFL | U66702_at | 168 | Hs.74624 | protein tyrosine phosphatase, receptor | recurrence |
| | | | | | type, N polypeptide 2 | |
| 204 | HUGeneFL | HG511-HT511_at | 168 | — | — | recurrence |
| 205 | HUGeneFL | HG3076-HT3238_s_at | 168 | — | — | recurrence |
| 206 | HUGeneFL | M98528_at | 168 | Hs.79404 | DNA segment on chromosome 4 | recurrence |
| | | | | | (unique) 234 expressed sequence | |
| 207 | HUGeneFL | M63175_at | 168 | Hs.295137 | autocrine motility factor receptor | recurrence |
| 208 | HUGeneFL | D49387_at | 168 | Hs.294584 | leukotriene B4 12- | recurrence |
| | | | | | hydroxydehydrogenase | |
| 209 | HUGeneFL | HG1879-HT1919_at | 168 | — | — | recurrence |
| 210 | HUGeneFL | Z23064_at | 168 | Hs.380118 | RNA binding motif protein, X chromosome | recurrence |
| 211 | HUGeneFL | X63469_at | 168 | Hs.77100 | general transcription factor IIE, poly- | recurrence |
| | | | | | peptide 2, beta 34 kDa | |
| 212 | HUGeneFL | L38928_at | 168 | Hs.118131 | 5,10-methenyltetrahydrofolate synthetase | recurrence |
| | | | | | (5-formyltetrahydrofolate | |
| | | | | | cyclo-ligase) | |
| 213 | HUGeneFL | U21858_at | 168 | Hs.60679 | TAF9 RNA polymerase II, TATA box | recurrence |
| | | | | | binding protein (TBP)-associated | |
| | | | | | factor, 32 kDa | |
| 214 | HUGeneFL | M64572_at | 168 | Hs.405666 | protein tyrosine phosphatase, non- | recurrence |
| | | | | | receptor type 3 | |
| 215 | HUGeneFL | D83657_at | 168 | Hs.19413 | S100 calcium binding protein A12 | SCC |
| | | | | | (calgranulin C) | |
| 216 | HUGeneFL | HG3945-HT4215_at | 168 | — | — | SCC |
| 217 | HUGeneFL | J00124_at | 168 | — | — | SCC |
| 218 | HUGeneFL | L05187_at | 168 | — | — | SCC |
| 219 | HUGeneFL | L42583_f_at | 168 | Hs.367762 | keratin 6A | SCC |
| 220 | HUGeneFL | L42601_f_at | 168 | Hs.367762 | keratin 6A | SCC |
| 221 | HUGeneFL | L42611_f_at | 168 | Hs.446417 | keratin 6E | SCC |
| 222 | HUGeneFL | M19888_at | 168 | Hs.1076 | small proline-rich protein 1B (cornifin) | SCC |
| 223 | HUGeneFL | M20030_f_at | 168 | Hs.505352 | Human small proline rich protein (sprII) | SCC |
| | | | | | mRNA, clone 930. | |
| 224 | HUGeneFL | M21005_at | 168 | — | — | SCC |
| 225 | HUGeneFL | M21302_at | 168 | Hs.505327 | Human small proline rich protein (sprII) | SCC |
| | | | | | mRNA, clone 174N. | |
| 226 | HUGeneFL | M21539_at | 168 | Hs.2421 | small proline-rich protein 2C | SCC |
| 227 | HUGeneFL | M86757_s_at | 168 | Hs.112408 | S100 calcium binding protein A7 (psoriasin | SCC |
| | | | | | 1) | |
| 228 | HUGeneFL | S72493_s_at | 168 | Hs.432448 | keratin 16 (focal non-epidermolytic | SCC |
| | | | | | palmoplantar keratoderma) | |
| 229 | HUGeneFL | U70981_at | 168 | Hs.336046 | interleukin 13 receptor, alpha 2 | SCC |
| 230 | HUGeneFL | V01516_f_at | 168 | Hs.367762 | keratin 6A | SCC |
| 231 | HUGeneFL | X53065_f_at | 168 | — | — | SCC |
| 232 | HUGeneFL | X57766_at | 168 | Hs.143751 | matrix metalloproteinase 11 (stromelysin | SCC |
| | | | | | 3) | |
| 233 | EOS Hu03 | 400773 | 133 | — | NM_003105*: Homo sapiens sortilin- | progression |
| | | | | | related receptor, L(DLR class) A | |
| | | | | | repeats-containing (SORL1), mRNA. | |
| 234 | EOS Hu03 | 400843 | 133 | — | NM_003105*: Homo sapiens sortilin- | progression |
| | | | | | related receptor, L(DLR class) A | |
| | | | | | repeats-containing (SORL1), mRNA. | |
| 235 | EOS Hu03 | 400844 | 133 | — | NM_003105*: Homo sapiens sortilin- | progression |
| | | | | | related receptor, L(DLR class) A | |
| | | | | | repeats-containing (SORL1), mRNA. | |
| 236 | EOS Hu03 | 400846 | 133 | — | sortilin-related receptor, L(DLR class) | progression |
| | | | | | A repeats-containing (SORL1) | |
| 237 | EOS Hu03 | 402328 | 133 | — | Target Exon | progression |
| 238 | EOS Hu03 | 402384 | 133 | — | NM_007181*: Homo sapiens mitogen- | progression |
| | | | | | activated protein kinase kinase kinase | |
| | | | | | kinase 1 (MAP4K1), mRNA. | |
| 239 | EOS Hu03 | 404208 | 133 | — | C6001282: gi\|4504223\|ref\|NP_000172.1\| | progression |
| | | | | | glucuronidase, beta [Homo sapiens] | |
| | | | | | gi\|114963\|sp\|P082 | |
| 240 | EOS Hu03 | 404606 | 133 | — | Target Exon | progression |
| 241 | EOS Hu03 | 404826 | 133 | — | Target Exon | progression |
| 242 | EOS Hu03 | 404875 | 133 | — | NM_022819*: Homo sapiens phospholipase | progression |
| | | | | | A2, group IIF (PLA2G2F), | |
| | | | | | mRNA. VERSION NM_020245.2 GI | |
| 243 | EOS Hu03 | 404913 | 133 | — | NM_024408*: Homo sapiens Notch | progression |
| | | | | | (Drosophila) homolog 2 (NOTCH2), | |
| | | | | | mRNA. VERSION NM_024410.1 GI | |
| 244 | EOS Hu03 | 404977 | 133 | — | Insulin-like growth factor 2 (somatomedin | progression |
| | | | | | A) (IGF2) | |
| 245 | EOS Hu03 | 405036 | 133 | — | NM_021628*: Homo sapiens arachidonate | progression |
| | | | | | lipoxygenase 3 (ALOXE3), | |
| | | | | | mRNA. VERSION NM_020229.1 GI | |
| 246 | EOS Hu03 | 405371 | 133 | — | NM_005569*: Homo sapiens LIM | progression |
| | | | | | domain kinase 2 (LIMK2), transcript | |
| | | | | | variant 2a, mRNA. | |
| 247 | EOS Hu03 | 405667 | 133 | — | Target Exon | progression |
| 248 | EOS Hu03 | 406002 | 133 | — | Target Exon | progression |
| 249 | EOS Hu03 | 407955 | 133 | Hs.9343 | ESTs | progression |
| 250 | EOS Hu03 | 408049 | 133 | Hs.345588 | desmoplakin (DPI, DPII) | progression |
| 251 | EOS Hu03 | 408288 | 133 | Hs.16886 | gb: zI73d06.r1 Stratagene colon | progression |
| | | | | | (937204) Homo sapiens cDNA clone | |
| | | | | | 5′, mRNA sequence | |
| 252 | EOS Hu03 | 409513 | 133 | Hs.54642 | methionine adenosyltransferase II, | progression |
| | | | | | beta | |
| 253 | EOS Hu03 | 409556 | 133 | Hs.54941 | phosphorylase kinase, alpha 2 (liver) | progression |
| 254 | EOS Hu03 | 409586 | 133 | Hs.55044 | DKFZP586H2123 protein | progression |
| 255 | EOS Hu03 | 409632 | 133 | Hs.55279 | serine (or cysteine) proteinase inhibitor, | progression |
| | | | | | clade B (ovalbumin), member 5 | |
| 256 | EOS Hu03 | 410047 | 133 | Hs.379753 | zinc finger protein 36 (KOX 18) | progression |
| 257 | EOS Hu03 | 411817 | 133 | Hs.72241 | mitogen-activated protein kinase | progression |
| | | | | | kinase 2 | |
| 258 | EOS Hu03 | 412649 | 133 | Hs.74369 | integrin, alpha 7 | progression |
| 259 | EOS Hu03 | 412841 | 133 | Hs.101395 | hypothetical protein MGC11352 | progression |
| 260 | EOS Hu03 | 413564 | 133 | — | gb: 601146990F1 NIH_MGC_19 Homo | progression |
| | | | | | sapiens cDNA clone 5′, mRNA sequence | |
| 261 | EOS Hu03 | 413786 | 133 | Hs.13500 | ESTs | progression |
| 262 | EOS Hu03 | 413840 | 133 | Hs.356228 | RNA binding motif protein, X chromosome | progression |
| 263 | EOS Hu03 | 413929 | 133 | Hs.75617 | collagen, type IV, alpha 2 | progression |
| 264 | EOS Hu03 | 414223 | 133 | Hs.238246 | hypothetical protein FLJ22479 | progression |
| 265 | EOS Hu03 | 414732 | 133 | Hs.77152 | minichromosome maintenance deficient | progression |
| | | | | | (S. cerevisiae) 7 | |
| 266 | EOS Hu03 | 414762 | 133 | Hs.77257 | KIAA0068 protein | progression |
| 267 | EOS Hu03 | 414840 | 133 | Hs.23823 | hairy/enhancer-of-split related with | progression |
| | | | | | YRPW motif-like | |
| 268 | EOS Hu03 | 414843 | 133 | Hs.77492 | heterogeneous nuclear ribonucleoprotein | progression |
| | | | | | A0 | |
| 269 | EOS Hu03 | 414895 | 133 | Hs.116278 | Homo sapiens cDNA FLJ13571 fis, | progression |
| | | | | | clone PLACE1008405 | |
| 270 | EOS Hu03 | 414907 | 133 | Hs.77597 | polo (Drosophia)-like kinase | progression |
| 271 | EOS Hu03 | 414918 | 133 | Hs.72222 | hypothetical protein FLJ13459 | progression |
| 272 | EOS Hu03 | 415200 | 133 | Hs.78202 | SWI/SNF related, matrix associated, | Progression |
| | | | | | actin dependent regulator of chromatin, | |
| | | | | | subfamily a, member 4 | |
| 273 | EOS Hu03 | 416640 | 133 | Hs.79404 | neuron-specific protein | Progression |
| 274 | EOS Hu03 | 416815 | 133 | Hs.80120 | UDP-N-acetyl-alpha-D- | Progression |
| | | | | | galactosamine: polypeptide N- | |
| | | | | | acetylgalactosaminyltransferase 1 | |
| | | | | | (GalNAc-T1) | |
| 275 | EOS Hu03 | 416977 | 133 | Hs.406103 | hypothetical protein FKSG44 | Progression |
| 276 | EOS Hu03 | 417615 | 133 | Hs.82314 | hypoxanthine phosphoribosyltransferase | Progression |
| | | | | | 1 (Lesch-Nyhan syndrome) | |
| 277 | EOS Hu03 | 417839 | 133 | Hs.82712 | fragile X mental retardation, autosomal | Progression |
| | | | | | homolog 1 | |
| 278 | EOS Hu03 | 417900 | 133 | Hs.82906 | CDC20 (cell division cycle 20, S. cerevisiae, | Progression |
| | | | | | homolog) | |
| 279 | EOS Hu03 | 417924 | 133 | Hs.82932 | cyclin D1 (PRAD1: parathyroid adenomatosis | Progression |
| | | | | | 1) | |
| 280 | EOS Hu03 | 418127 | 133 | Hs.83532 | membrane cofactor protein (CD46, | Progression |
| | | | | | trophoblast-lymphocyte cross-reactive | |
| | | | | | antigen) | |
| 281 | EOS Hu03 | 418321 | 133 | Hs.84087 | KIAA0143 protein | Progression |
| 282 | EOS Hu03 | 418504 | 133 | Hs.85335 | Homo sapiens mRNA; cDNA | Progression |
| | | | | | DKFZp564D1462 (from clone | |
| | | | | | DKFZp564D1462) | |
| 283 | EOS Hu03 | 418629 | 133 | Hs.86859 | growth factor receptor-bound protein 7 | Progression |
| 284 | EOS Hu03 | 419602 | 133 | Hs.91521 | hypothetical protein | Progression |
| 285 | EOS Hu03 | 419847 | 133 | Hs.184544 | Homo sapiens, clone IMAGE: 3355383, | Progression |
| | | | | | mRNA, partial cds | |
| 286 | EOS Hu03 | 420079 | 133 | Hs.94896 | PTD011 protein | Progression |
| 287 | EOS Hu03 | 420116 | 133 | Hs.95231 | FH1/FH2 domain-containing protein | Progression |
| 288 | EOS Hu03 | 420307 | 133 | Hs.66219 | ESTs | Progression |
| 289 | EOS Hu03 | 420613 | 133 | Hs.406637 | ESTs, Weakly similar to A47582 B-cell | Progression |
| | | | | | growth factor precursor [H. sapiens] | |
| 290 | EOS Hu03 | 420732 | 133 | Hs.367762 | ESTs | Progression |
| 291 | EOS Hu03 | 421026 | 133 | Hs.101067 | GCN5 (general control of amino-acid | Progression |
| | | | | | synthesis, yeast, homolog)-like 2 | |
| 292 | EOS Hu03 | 421075 | 133 | Hs.101474 | KIAA0807 protein | Progression |
| 293 | EOS Hu03 | 421101 | 133 | Hs.101840 | major histocompatibility complex, class | Progression |
| | | | | | I-like sequence | |
| 294 | EOS Hu03 | 421186 | 133 | Hs.270563 | ESTs, Moderately similar to T12512 | Progression |
| | | | | | hypothetical protein DKFZp434G232.1 | |
| | | | | | [H. sapiens] | |
| 295 | EOS Hu03 | 421311 | 133 | Hs.283609 | hypothetical protein PRO2032 progression | |
| 296 | EOS Hu03 | 421475 | 133 | Hs.104640 | HIV-1 inducer of short transcripts | progression |
| | | | | | binding protein; lymphoma related | |
| | | | | | factor | |
| 297 | EOS Hu03 | 421505 | 133 | Hs.285641 | KIAA1111 protein | progression |
| 298 | EOS Hu03 | 421595 | 133 | Hs.301685 | KIAA0620 protein | progression |
| 299 | EOS Hu03 | 421628 | 133 | Hs.106210 | hypothetical protein FLJ10813 | progression |
| 300 | EOS Hu03 | 421649 | 133 | Hs.106415 | peroxisome proliferative activated | progression |
| | | | | | receptor, delta | |
| 301 | EOS Hu03 | 421733 | 133 | Hs.1420 | fibroblast growth factor receptor 3 | progression |
| | | | | | (achondroplasia, thanatophoric dwarfism) | |
| 302 | EOS Hu03 | 421782 | 133 | Hs.108258 | actin binding protein; macrophin | progression |
| | | | | | (microfilament and actin filament | |
| | | | | | cross-linker protein) | |
| 303 | EOS Hu03 | 421989 | 133 | Hs.110457 | Wolf-Hirschhorn syndrome candidate 1 | progression |
| 304 | EOS Hu03 | 422043 | 133 | Hs.110953 | retinoic acid induced 1 | progression |
| 305 | EOS Hu03 | 422068 | 133 | Hs.104520 | Homo sapiens cDNA FLJ13694 fis, | progression |
| | | | | | clone PLACE2000115 | |
| 306 | EOS Hu03 | 422506 | 133 | Hs.300741 | sorcin | progression |
| 307 | EOS Hu03 | 422913 | 133 | Hs.121599 | CGI-18 protein | progression |
| 308 | EOS Hu03 | 422929 | 133 | Hs.94011 | ESTs, Weakly similar to | progression |
| | | | | | MGB4_HUMAN MELANOMA- | |
| | | | | | ASSOCIATED ANTIGEN B4 | |
| | | | | | [H. sapiens] | |
| 309 | EOS Hu03 | 422959 | 133 | Hs.349256 | paired immunoglobulin-like receptor | progression |
| | | | | | beta | |
| 310 | EOS Hu03 | 423138 | 133 | — | gb: EST385571 MAGE resequences, | progression |
| | | | | | MAGM Homo sapiens cDNA, mRNA | |
| | | | | | sequence | |
| 311 | EOS Hu03 | 423185 | 133 | Hs.380062 | ornithine decarboxylase antizyme 1 | progression |
| 312 | EOS Hu03 | 423599 | 133 | Hs.31731 | peroxiredoxin 5 | progression |
| 313 | EOS Hu03 | 423810 | 133 | Hs.132955 | BCL2/adenovirus E1B 19 kD- | progression |
| | | | | | interacting protein 3-like | |
| 314 | EOS Hu03 | 423960 | 133 | Hs.136309 | SH3-containing protein SH3GLB1 | progression |
| 315 | EOS Hu03 | 424244 | 133 | Hs.143601 | hypothetical protein hCLA-iso | progression |
| 316 | EOS Hu03 | 424415 | 133 | Hs.146580 | enolase 2, (gamma, neuronal) | progression |
| 317 | EOS Hu03 | 424909 | 133 | Hs.153752 | cell division cycle 25B | progression |
| 318 | EOS Hu03 | 424959 | 133 | Hs.153937 | activated p21cdc42Hs kinase | progression |
| 319 | EOS Hu03 | 425093 | 133 | Hs.154525 | KIAA1076 protein | progression |
| 320 | EOS Hu03 | 425097 | 133 | Hs.154545 | PDZ domain containing guanine nucleotide | progression |
| | | | | | exchange factor(GEF)1 | |
| 321 | EOS Hu03 | 425205 | 133 | Hs.155106 | receptor (calcitonin) activity modifying | progression |
| | | | | | protein 2 | |
| 322 | EOS Hu03 | 425221 | 133 | Hs.155188 | TATA box binding protein (TBP)- | progression |
| | | | | | associated factor, RNA polymerase II, | |
| | | | | | F, 55 kD | |
| 323 | EOS Hu03 | 425243 | 133 | Hs.155291 | KIAA0005 gene product | progression |
| 324 | EOS Hu03 | 425380 | 133 | Hs.32148 | AD-015 protein | progression |
| 325 | EOS Hu03 | 426028 | 133 | Hs.172028 | a disintegrin and metalloproteinase | progression |
| | | | | | domain 10 (ADAM10) | |
| 326 | EOS Hu03 | 426125 | 133 | Hs.166994 | FAT tumor suppressor (Drosophila) | progression |
| | | | | | homolog | |
| 327 | EOS Hu03 | 426177 | 133 | Hs.167700 | Homo sapiens cDNA FLJ10174 fis, | progression |
| | | | | | clone HEMBA1003959 | |
| 328 | EOS Hu03 | 426252 | 133 | Hs.28917 | ESTs | progression |
| 329 | EOS Hu03 | 426468 | 133 | Hs.117558 | ESTs | progression |
| 330 | EOS Hu03 | 426469 | 133 | Hs.363039 | methylmalonate-semialdehyde dehydrogenase | progression |
| 331 | EOS Hu03 | 426508 | 133 | Hs.170171 | glutamate-ammonia ligase (glutamine | progression |
| | | | | | synthase) | |
| 332 | EOS Hu03 | 426682 | 133 | Hs.2056 | UDP glycosyltransferase 1 family, | progression |
| | | | | | polypeptide A9 | |
| 333 | EOS Hu03 | 426799 | 133 | Hs.303154 | popeye protein 3 | progression |
| 334 | EOS Hu03 | 426982 | 133 | Hs.173091 | ubiquitin-like 3 | progression |
| 335 | EOS Hu03 | 427239 | 133 | Hs.356512 | ubiquitin carrier protein | progression |
| 336 | EOS Hu03 | 427351 | 133 | Hs.123253 | hypothetical protein FLJ22009 | progression |
| 337 | EOS Hu03 | 427681 | 133 | Hs.284232 | tumor necrosis factor receptor super- | progression |
| | | | | | family, member 12 (translocating | |
| | | | | | chain-association membrane protein) | |
| 338 | EOS Hu03 | 427722 | 133 | Hs.180479 | hypothetical protein FLJ20116 | progression |
| 339 | EOS Hu03 | 427747 | 133 | Hs.180655 | serine/threonine kinase 12 | progression |
| 340 | EOS Hu03 | 427999 | 133 | Hs.181369 | ubiquitin fusion degradation 1-like | progression |
| 341 | EOS Hu03 | 428115 | 133 | Hs.300855 | KIAA0977 protein | progression |
| 342 | EOS Hu03 | 428284 | 133 | Hs.183435 | NM_004545: Homo sapiens NADH | progression |
| | | | | | dehydrogenase (ubiquinone) 1 beta | |
| | | | | | subcomplex, 1 (7 kD, MNLL) | |
| | | | | | (NDUFB1), mRNA. | |
| 343 | EOS Hu03 | 428318 | 133 | Hs.356190 | ubiquitin B | progression |
| 344 | EOS Hu03 | 428712 | 133 | Hs.190452 | KIAA0365 gene product | progression |
| 345 | EOS Hu03 | 428901 | 133 | Hs.146668 | KIAA1253 protein | progression |
| 346 | EOS Hu03 | 429124 | 133 | Hs.196914 | minor histocompatibility antigen HA-1 | progression |
| 347 | EOS Hu03 | 429187 | 133 | Hs.163872 | ESTs, Weakly similar to S65657 alpha- | progression |
| | | | | | 1C-adrenergic receptor splice | |
| | | | | | form 2 [H. sapiens] | |
| 348 | EOS Hu03 | 429311 | 133 | Hs.198998 | conserved helix-loop-helix ubiquitous | progression |
| | | | | | kinase | |
| 349 | EOS Hu03 | 429561 | 133 | Hs.250646 | baculoviral IAP repeat-containing 6 | progression |
| 350 | EOS Hu03 | 429802 | 133 | Hs.5367 | ESTs, Weakly similar to I38022 hypothetical | progression |
| | | | | | protein [H. sapiens] | |
| 351 | EOS Hu03 | 429953 | 133 | Hs.226581 | COX15 (yeast) homolog, cytochrome c | progression |
| | | | | | oxidase assembly protein | |
| 352 | EOS Hu03 | 430604 | 133 | Hs.247309 | succinate-CoA ligase, GDP-forming, | progression |
| | | | | | beta subunit | |
| 353 | EOS Hu03 | 430677 | 133 | Hs.359784 | desmoglein 2 | progression |
| 354 | EOS Hu03 | 430746 | 133 | Hs.406256 | ESTs | progression |
| 355 | EOS Hu03 | 431604 | 133 | Hs.264190 | vacuolar protein sorting 35 (yeast | progression |
| | | | | | homolog) | |
| 356 | EOS Hu03 | 431842 | 133 | Hs.271473 | epithelial protein up-regulated in carcinoma, | progression |
| | | | | | membrane associated protein | |
| | | | | | 17 | |
| 357 | EOS Hu03 | 431857 | 133 | Hs.271742 | ADP-ribosyltransferase (NAD; poly | progression |
| | | | | | (ADP-ribose) polymerase)-like 3 | |
| 358 | EOS Hu03 | 432258 | 133 | Hs.293039 | ESTs | progression |
| 359 | EOS Hu03 | 432327 | 133 | Hs.274363 | neuroglobin | progression |
| 360 | EOS Hu03 | 432554 | 133 | Hs.278411 | NCK-associated protein 1 | progression |
| 361 | EOS Hu03 | 432864 | 133 | Hs.359682 | calpastatin | progression |
| 362 | EOS Hu03 | 433052 | 133 | Hs.293003 | ESTs, Weakly similar to PC4259 | progression |
| | | | | | ferritin associated protein [H. sapiens] | |
| 363 | EOS Hu03 | 433282 | 133 | Hs.49007 | hypothetical protein | progression |
| 364 | EOS Hu03 | 433844 | 133 | Hs.179647 | Homo sapiens cDNA FLJ12195 fis, | progression |
| | | | | | clone MAMMA1000865 | |
| 365 | EOS Hu03 | 433914 | 133 | Hs.112160 | Homo sapiens DNA helicase homolog | progression |
| | | | | | (PIF1) mRNA, partial cds | |
| 366 | EOS Hu03 | 434055 | 133 | Hs.3726 | x 003 protein | progression |
| 367 | EOS Hu03 | 434263 | 133 | Hs.79187 | ESTs | progression |
| 368 | EOS Hu03 | 434547 | 133 | Hs.106124 | ESTs | progression |
| 369 | EOS Hu03 | 434831 | 133 | Hs.273397 | KIAA0710 gene product | progression |
| 370 | EOS Hu03 | 434978 | 133 | Hs.4310 | eukaryotic translation initiation factor | progression |
| | | | | | 1A | |
| 371 | EOS Hu03 | 435158 | 133 | Hs.65588 | DAZ associated protein 1 | progression |
| 372 | EOS Hu03 | 435320 | 133 | Hs.117864 | ESTs | progression |
| 373 | EOS Hu03 | 435521 | 133 | Hs.6361 | mitogen-activated protein kinase | progression |
| | | | | | kinase 1 interacting protein 1 | |
| 374 | EOS Hu03 | 436472 | 133 | Hs.46366 | KIAA0948 protein | progression |
| 375 | EOS Hu03 | 436576 | 133 | Hs.77542 | ESTs | progression |
| 376 | EOS Hu03 | 437223 | 133 | Hs.330716 | Homo sapiens cDNA FLJ14368 fis, | progression |
| | | | | | clone HEMBA1001122 | |
| 377 | EOS Hu03 | 437256 | 133 | Hs.97871 | Homo sapiens, clone IMAGE: 3845253, | progression |
| | | | | | mRNA, partial cds | |
| 378 | EOS Hu03 | 437524 | 133 | Hs.385719 | ESTs | progression |
| 379 | EOS Hu03 | 438013 | 133 | Hs.15670 | ESTs | progression |
| 380 | EOS Hu03 | 438644 | 133 | Hs.129037 | ESTs | progression |
| 381 | EOS Hu03 | 438818 | 133 | Hs.30738 | ESTs | progression |
| 382 | EOS Hu03 | 438942 | 133 | Hs.6451 | PRO0659 protein | progression |
| 383 | EOS Hu03 | 439010 | 133 | Hs.75216 | Homo sapiens cDNA FLJ13713 fis, | progression |
| | | | | | clone PLACE2000398, moderately | |
| | | | | | similar to LAR PROTEIN PRECURSOR | |
| | | | | | (LEUKOCYTE ANTIGEN RELATED) | |
| | | | | | (EC 3.1.3.48) | |
| 384 | EOS Hu03 | 439130 | 133 | Hs.375195 | ESTs | progression |
| 385 | EOS Hu03 | 439578 | 133 | Hs.350547 | nuclear receptor co-repressor/HDAC3 | progression |
| | | | | | complex subunit | |
| 386 | EOS Hu03 | 439632 | 133 | Hs.334437 | hypothetical protein MGC4248 | progression |
| 387 | EOS Hu03 | 440014 | 133 | Hs.6856 | ash2 (absent, small, or homeotic, | progression |
| | | | | | Drosophila, homolog)-like | |
| 388 | EOS Hu03 | 440100 | 133 | Hs.158549 | ESTs, Weakly similar to | progression |
| | | | | | T2D3_HUMAN TRANSCRIPTION | |
| | | | | | INITIATION FACTOR TFIID 135 KDA | |
| | | | | | SUBUNIT [H. sapiens] | |
| 389 | EOS Hu03 | 440197 | 133 | Hs.317714 | pallid (mouse) homolog, pallidin | progression |
| 390 | EOS Hu03 | 440357 | 133 | Hs.20950 | phospholysine phosphohistidine inorganic | progression |
| | | | | | pyrophosphate phosphatase | |
| 391 | EOS Hu03 | 441650 | 133 | Hs.132545 | ESTs | progression |
| 392 | EOS Hu03 | 442220 | 133 | Hs.8148 | selenoprotein T | progression |
| 393 | EOS Hu03 | 442549 | 133 | Hs.8375 | TNF receptor-associated factor 4 | progression |
| 394 | EOS Hu03 | 443407 | 133 | Hs.348514 | ESTs, Moderately similar to 2109260A | progression |
| | | | | | B cell growth factor [H. sapiens] | |
| 395 | EOS Hu03 | 443471 | 133 | Hs.398102 | Homo sapiens clone FLB3442 | progression |
| | | | | | PRO0872 mRNA, complete cds | |
| 396 | EOS Hu03 | 443679 | 133 | Hs.9670 | hypothetical protein FLJ10948 | progression |
| 397 | EOS Hu03 | 443893 | 133 | Hs.115472 | ESTs, Weakly similar to 2004399A | progression |
| | | | | | chromosomal protein [H. sapiens] | |
| 398 | EOS Hu03 | 444037 | 133 | Hs.380932 | CHMP1.5 protein | progression |
| 399 | EOS Hu03 | 444312 | 133 | Hs.351142 | ESTs | progression |
| 400 | EOS Hu03 | 444336 | 133 | Hs.10882 | HMG-box containing protein 1 | progression |
| 401 | EOS Hu03 | 444604 | 133 | Hs.11441 | chromosome 1 open reading frame 8 | progression |
| 402 | EOS Hu03 | 445084 | 133 | Hs.250848 | hypothetical protein FLJ14761 | progression |
| 403 | EOS Hu03 | 445462 | 133 | Hs.288649 | hypothetical protein MGC3077 | progression |
| 404 | EOS Hu03 | 445692 | 133 | Hs.182099 | ESTs | progression |
| 405 | EOS Hu03 | 445831 | 133 | Hs.13351 | LanC (bacterial lantibiotic synthetase | progression |
| | | | | | component C)-like 1 | |
| 406 | EOS Hu03 | 446556 | 133 | Hs.15303 | KIAA0349 protein | progression |
| 407 | EOS Hu03 | 446847 | 133 | Hs.82845 | Homo sapiens cDNA: FLJ21930 fis, | progression |
| | | | | | clone HEP04301, highly similar to | |
| | | | | | HSU90916 Human clone 23815 | |
| | | | | | mRNA sequence | |
| 408 | EOS Hu03 | 447343 | 133 | Hs.236894 | ESTs, Highly similar to S02392 alpha- | progression |
| | | | | | 2-macroglobulin receptor precursor | |
| | | | | | [H. sapiens] | |
| 409 | EOS Hu03 | 447400 | 133 | Hs.18457 | hypothetical protein FLJ20315 | progression |
| 410 | EOS Hu03 | 448357 | 133 | Hs.108923 | RAB38, member RAS oncogene family | progression |
| 411 | EOS Hu03 | 448524 | 133 | Hs.21356 | hypothetical protein DKFZp762K2015 | progression |
| 412 | EOS Hu03 | 448625 | 133 | Hs.178470 | hypothetical protein FLJ22662 | progression |
| 413 | EOS Hu03 | 448780 | 133 | Hs.267749 | Human DNA sequence from clone | progression |
| | | | | | 366N23 on chromosome 6q27. Contains | |
| | | | | | two genes similar to consecutive | |
| | | | | | parts of the C. elegans UNC-93 (protein | |
| | | | | | 1, C46F11.1) gene, a KIAA0173 | |
| | | | | | and Tubulin-Tyrosine Ligase LIKE | |
| | | | | | gene, a Mitotic Feedback Control | |
| | | | | | Protein MADP2 H | |
| 414 | EOS Hu03 | 448813 | 133 | Hs.22142 | cytochrome b5 reductase b5R.2 | progression |
| 415 | EOS Hu03 | 449268 | 133 | Hs.23412 | hypothetical protein FLJ20160 | progression |
| 416 | EOS Hu03 | 449626 | 133 | Hs.112860 | zinc finger protein 258 | progression |
| 417 | EOS Hu03 | 450893 | 133 | Hs.25625 | hypothetical protein FLJ11323 | progression |
| 418 | EOS Hu03 | 450997 | 133 | Hs.35254 | hypothetical protein FLB6421 | progression |
| 419 | EOS Hu03 | 451164 | 133 | Hs.60659 | ESTs, Weakly similar to T46471 hypothetical | progression |
| | | | | | protein DKFZp434L0130.1 | |
| | | | | | [H. sapiens] | |
| 420 | EOS Hu03 | 451225 | 133 | Hs.57655 | ESTs | progression |
| 421 | EOS Hu03 | 451867 | 133 | Hs.27192 | hypothetical protein dJ1057B20.2 | progression |
| 422 | EOS Hu03 | 451970 | 133 | Hs.211046 | ESTs | progression |
| 423 | EOS Hu03 | 452012 | 133 | Hs.279766 | kinesin family member 4A | progression |
| 424 | EOS Hu03 | 452170 | 133 | Hs.28285 | patched related protein translocated in | progression |
| | | | | | renal cancer | |
| 425 | EOS Hu03 | 452517 | 133 | — | gb: RC-BT068-130399-068 BT068 | progression |
| | | | | | Homo sapiens cDNA, mRNA sequence | |
| 426 | EOS Hu03 | 452829 | 133 | Hs.63368 | ESTs, Weakly similar to | progression |
| | | | | | TRHY_HUMAN TRICHOHYALI | |
| | | | | | [H. sapiens] | |
| 427 | EOS Hu03 | 452929 | 133 | Hs.172816 | neuregulin 1 | progression |
| 428 | EOS Hu03 | 453395 | 133 | Hs.377915 | mannosidase, alpha, class 2A, member 1 | progression |
| 429 | EOS Hu03 | 454639 | 133 | — | gb: RC2-ST0158-091099-011-d05 | progression |
| | | | | | ST0158 Homo sapiens cDNA, mRNA | |
| | | | | | sequence | |
| 430 | EOS Hu03 | 456332 | 133 | Hs.399939 | gb: nc39d05.r1 NCI_CGAP_Pr2 Homo | progression |
| | | | | | sapiens cDNA clone, mRNA sequence | |
| 431 | EOS Hu03 | 457228 | 133 | Hs.195471 | Human cosmid CRI-JC2015 at | progression |
| | | | | | D10S289 in 10sp13 | |
| 432 | EOS Hu03 | 458132 | 133 | Hs.103267 | hypothetical protein FLJ22548 similar | progression |
| | | | | | to gene trap PAT 12 | |
| 433 | EOS Hu03 | 408688 | 133 | Hs.152925 | KIAA1268 protein | progression |
| 434 | EOS Hu03 | 410691 | 133 | Hs.65450 | reticulon 4 | progression |
| 435 | EOS Hu03 | 420269 | 133 | Hs.96264 | alpha thalassemia/mental retardation | progression |
| | | | | | syndrome X-linked (RAD54 (S. cerevisiae) | |
| | | | | | homolog) | |
| 436 | EOS Hu03 | 422119 | 133 | Hs.111862 | KIAA0590 gene product | progression |
| 437 | EOS Hu03 | 422765 | 133 | Hs.1578 | baculoviral IAP repeat-containing 5 | progression |
| | | | | | (survivin) | |
| 438 | EOS Hu03 | 422984 | 133 | Hs.351597 | ESTs | progression |
| 439 | EOS Hu03 | 428016 | 133 | Hs.181461 | ariadne homolog, ubiquitin-conjugating | progression |
| | | | | | enzyme E2 binding protein, 1 (Drosophila) | |
| 440 | EOS Hu03 | 437325 | 133 | Hs.5548 | F-box and leucine-rich repeat protein 5 | progression |
| 441 | EOS Hu03 | 444773 | 133 | Hs.11923 | hypothetical protein DJ167A19.1 | progression |
| 442 | EOS Hu03 | 445926 | 133 | Hs.334826 | splicing factor 3b, subunit 1, 155 kDa | progression |
| 443 | EOS Hu03 | 452714 | 133 | Hs.30340 | KIAA1165: likely ortholog of mouse | progression |
| | | | | | Nedd4 WW domain-binding protein 5A | |
| 444 | EOS Hu03 | 452866 | 133 | Hs.268016 | ESTs | progression |
| 445 | EOS Hu03 | 453963 | 133 | Hs.28959 | cDNA FLJ36513 fis, clone | progression |
| | | | | | TRACH2001523 | |
| 446 | EOS Hu03 | 457329 | 133 | Hs.359682 | calpastatin | progression |
| 447 | U133A | 200600_at | 168 | Hs.170328 | NM_001910; cathepsin E isoform a | CIS |
| | | | | | preproprotein NM_148964; cathepsin | |
| | | | | | E isoform b preproprotein | |
| 448 | U133A | 200762_at | 168 | Hs.173381 | NM_019894; transmembrane protease, | CIS |
| | | | | | serine 4 isoform 1 NM_183247; | |
| | | | | | transmembrane protease, serine 4 | |
| | | | | | isoform 2 | |
| 449 | U133A | 201088_at | 168 | Hs.159557 | NM_000228; laminin subunit beta 3 | CIS |
| | | | | | precursor | |
| 450 | U133A | 201291_s_at | 168 | Hs.156346 | NM_030570; uroplakin 3B isoform a | CIS |
| | | | | | NM_182683; uroplakin 3B isoform c | |
| | | | | | NM_182684; uroplakin 3B isoform b | |
| 451 | U133A | 201560_at | 168 | Hs.25035 | NM_005547; involucrin | CIS |
| 452 | U133A | 201616_s_at | 168 | Hs.443811 | NM_004692; NM_032727; internexin | CIS |
| | | | | | neuronal intermediate filament protein, | |
| | | | | | alpha | |
| 453 | U133A | 201641_at | 168 | Hs.118110 | NM_016233; peptidylarginine deiminase | CIS |
| | | | | | type III | |
| 454 | U133A | 201744_s_at | 168 | Hs.406475 | NM_014417; BCL2 binding component 3 | CIS |
| 455 | U133A | 201842_s_at | 168 | Hs.76224 | NM_020142; NADH: ubiquinone oxidoreductase | CIS |
| | | | | | MLRQ subunit homolog | |
| 456 | U133A | 201858_s_at | 168 | Hs.1908 | NM_018058; cartilage acidic protein 1 | CIS |
| 457 | U133A | 201859_at | 168 | Hs.1908 | NM_000497; cytochrome P450, sub- | CIS |
| | | | | | family XIB (steroid 11-beta- | |
| | | | | | hydroxylase), polypeptide 1 precursor | |
| 458 | U133A | 202746_at | 168 | Hs.17109 | NM_007193; annexin A10 | CIS |
| 459 | U133A | 202917_s_at | 168 | Hs.416073 | NM_001958; eukaryotic translation | CIS |
| | | | | | elongation factor 1 alpha 2 | |
| 460 | U133A | 203009_at | 168 | Hs.155048 | NM_005581; Lutheran blood group | CIS |
| | | | | | (Auberger b antigen included) | |
| 461 | U133A | 203287_at | 168 | Hs.18141 | NM_005581; Lutheran blood group | CIS |
| | | | | | (Auberger b antigen included) | |
| 462 | U133A | 203477_at | 168 | Hs.409034 | NM_030570; uroplakin 3B isoform a | CIS |
| | | | | | NM_182683; uroplakin 3B isoform c | |
| | | | | | NM_182684; uroplakin 3B isoform b | |
| 463 | U133A | 203649_s_at | 168 | Hs.76422 | NM_000300; phospholipase A2, group | CIS |
| | | | | | IIA (platelets, synovial fluid) | |
| 464 | U133A | 203759_at | 168 | Hs.75268 | NM_007193; annexin A10 | CIS |
| 465 | U133A | 203792_x_at | 168 | Hs.371617 | NM_007144; ring finger protein 110 | CIS |
| 466 | U133A | 203842_s_at | 168 | Hs.172740 | NM_014417; BCL2 binding component 3 | CIS |
| 467 | U133A | 203980_at | 168 | Hs.391561 | NM_001442; fatty acid binding protein | CIS |
| | | | | | 4, adipocyte | |
| 468 | U133A | 204141_at | 168 | Hs.300701 | NM_017689; hypothetical protein | CIS |
| | | | | | FLJ20151 | |
| 469 | U133A | 204380_s_at | 168 | Hs.1420 | NM_007144; ring finger protein 110 | CIS |
| 470 | U133A | 204465_s_at | 168 | Hs.76888 | NM_004692; NM_032727; internexin | CIS |
| | | | | | neuronal intermediate filament protein, | |
| | | | | | alpha | |
| 471 | U133A | 204487_s_at | 168 | Hs.367809 | NM_001248; ectonucleoside triphosphate | CIS |
| | | | | | diphosphohydrolase 3 | |
| 472 | U133A | 204508_s_at | 168 | Hs.279916 | NM_017689; hypothetical protein | CIS |
| | | | | | FLJ20151 | |
| 473 | U133A | 204540_at | 168 | Hs.433839 | NM_001958; eukaryotic translation | CIS |
| | | | | | elongation factor 1 alpha 2 | |
| 474 | U133A | 204688_at | 168 | Hs.409798 | NM_016233; peptidylarginine deiminase | CIS |
| | | | | | type III | |
| 475 | U133A | 204952_at | 168 | Hs.377028 | NM_000445; plectin 1, intermediate | CIS |
| | | | | | filament binding protein 500 kDa | |
| 476 | U133A | 204990_s_at | 168 | Hs.85266 | NM_000213; integrin, beta 4 | CIS |
| 477 | U133A | 205073_at | 168 | Hs.152096 | NM_019894; transmembrane protease, | CIS |
| | | | | | serine 4 isoform 1 NM_183247; | |
| | | | | | transmembrane protease, serine 4 | |
| | | | | | isoform 2 | |
| 478 | U133A | 205382_s_at | 168 | Hs.155597 | NM_000213; integrin, beta 4 | CIS |
| 479 | U133A | 205453_at | 168 | Hs.290432 | NM_002145; homeo box B2 | CIS |
| 480 | U133A | 205455_at | 168 | Hs.2942 | NM_006760; uroplakin 2 | CIS |
| 481 | U133A | 205927_s_at | 168 | Hs.1355 | NM_001910; cathepsin E isoform a | CIS |
| | | | | | preproprotein NM_148964; cathepsin | |
| | | | | | E isoform b preproprotein | |
| 482 | U133A | 206122_at | 168 | Hs.95582 | NM_006942; SRY-box 15 | CIS |
| 483 | U133A | 206191_at | 168 | Hs.47042 | NM_001248; ectonucleoside triphosphate | CIS |
| | | | | | diphosphohydrolase 3 | |
| 484 | U133A | 206392_s_at | 168 | Hs.82547 | NM_005522; homeobox A1 protein | CIS |
| | | | | | isoform a NM_153620; homeobox A1 | |
| | | | | | protein isoform b | |
| 485 | U133A | 206393_at | 168 | Hs.83760 | NM_003282; troponin I, skeletal, fast | CIS |
| 486 | U133A | 206465_at | 168 | Hs.277543 | NM_015162; lipidosin | CIS |
| 487 | U133A | 206561_s_at | 168 | Hs.116724 | NM_015162; lipidosin | CIS |
| 488 | U133A | 206658_at | 168 | Hs.284211 | NM_030570; uroplakin 3B isoform a | CIS |
| | | | | | NM_182683; uroplakin 3B isoform c | |
| | | | | | NM_182684; uroplakin 3B isoform b | |
| 489 | U133A | 207173_x_at | 168 | Hs.443435 | NM_000213; integrin, beta 4 | CIS |
| 490 | U133A | 207862_at | 168 | Hs.379613 | NM_006760; uroplakin 2 | CIS |
| 491 | U133A | 209138_x_at | 168 | Hs.505407 | NM_015162; lipidosin | CIS |
| 492 | U133A | 209270_at | 168 | Hs.436983 | NM_000228; laminin subunit beta 3 | CIS |
| | | | | | precursor | |
| 493 | U133A | 209340_at | 168 | Hs.21293 | NM_007144; ring finger protein 110 | CIS |
| 494 | U133A | 209591_s_at | 168 | Hs.170195 | NM_000228; laminin subunit beta 3 | CIS |
| | | | | | precursor | |
| 495 | U133A | 209732_at | 168 | Hs.85201 | NM_001248; ectonucleoside triphosphate | CIS |
| | | | | | diphosphohydrolase 3 | |
| 496 | U133A | 210143_at | 168 | Hs.188401 | NM_007193; annexin A10 | CIS |
| 497 | U133A | 210735_s_at | 168 | Hs.5338 | NM_017689; hypothetical protein | CIS |
| | | | | | FLJ20151 | |
| 498 | U133A | 210761_s_at | 168 | Hs.86859 | NM_020142; NADH: ubiquinone oxidoreductase | CIS |
| | | | | | MLRQ subunit homolog | |
| 499 | U133A | 211002_s_at | 168 | Hs.82237 | NM_001958; eukaryotic translation | CIS |
| | | | | | elongation factor 1 alpha 2 | |
| 500 | U133A | 211161_s_at | 168 | | NM_000300; phospholipase A2, group | CIS |
| | | | | | IIA (platelets, synovial fluid) | |
| 501 | U133A | 211430_s_at | 168 | Hs.413826 | NM_001910; cathepsin E isoform a | CIS |
| | | | | | preproprotein NM_148964; cathepsin | |
| | | | | | E isoform b preproprotein | |
| 502 | U133A | 211671_s_at | 168 | Hs.126608 | NM_007144; ring finger protein 110 | CIS |
| 503 | U133A | 211692_s_at | 168 | Hs.87246 | NM_014417; BCL2 binding component 3 | CIS |
| 504 | U133A | 211896_s_at | 168 | Hs.156316 | NM_005581; Lutheran blood group | CIS |
| | | | | | (Auberger b antigen included) | |
| 505 | U133A | 212077_at | 168 | Hs.443811 | NM_003282; troponin I, skeletal, fast | CIS |
| 506 | U133A | 212192_at | 168 | Hs.109438 | NM_020142; NADH: ubiquinone oxidoreductase | CIS |
| | | | | | MLRQ subunit homolog | |
| 507 | U133A | 212195_at | 168 | Hs.71968 | NM_000445; plectin 1, intermediate | CIS |
| | | | | | filament binding protein 500 kDa | |
| 508 | U133A | 212386_at | 168 | Hs.359289 | NM_005547; involucrin | CIS |
| 509 | U133A | 212667_at | 168 | Hs.111779 | NM_000299; plakophilin 1 | CIS |
| 510 | U133A | 212671_s_at | 168 | Hs.387679 | NM_002145; homeo box B2 | CIS |
| 511 | U133A | 212998_x_at | 168 | Hs.375115 | NM_000497; cytochrome P450, sub- | CIS |
| | | | | | family XIB (steroid 11-beta- | |
| | | | | | hydroxylase), polypeptide 1 precursor | |
| 512 | U133A | 213891_s_at | 168 | Hs.359289 | NM_007193; annexin A10 | CIS |
| 513 | U133A | 213975_s_at | 168 | Hs.234734 | NM_005522; homeobox A1 protein | CIS |
| | | | | | isoform a NM_153620; homeobox A1 | |
| | | | | | protein isoform b | |
| 514 | U133A | 214352_s_at | 168 | Hs.412107 | NM_006760; uroplakin 2 | CIS |
| 515 | U133A | 214599_at | 168 | Hs.157091 | NM_005547; involucrin | CIS |
| 516 | U133A | 214630_at | 168 | Hs.184927 | NM_000497; cytochrome P450, sub- | CIS |
| | | | | | family XIB (steroid 11-beta- | |
| | | | | | hydroxylase), polypeptide 1 precursor | |
| 517 | U133A | 214639_s_at | 168 | Hs.67397 | NM_005522; homeobox A1 protein | CIS |
| | | | | | isoform a NM_153620; homeobox A1 | |
| | | | | | protein isoform b | |
| 518 | U133A | 214651_s_at | 168 | Hs.127428 | NM_002145; homeo box B2 | CIS |
| 519 | U133A | 214669_x_at | 168 | Hs.377975 | NM_001442; fatty acid binding protein | CIS |
| | | | | | 4, adipocyte | |
| 520 | U133A | 214677_x_at | 168 | Hs.449601 | NM_006942; SRY-box 15 | CIS |
| 521 | U133A | 214752_x_at | 168 | Hs.195464 | NM_006942; SRY-box 15 | CIS |
| 522 | U133A | 215076_s_at | 168 | Hs.443625 | NM_016233; peptidylarginine deiminase | CIS |
| | | | | | type III | |
| 523 | U133A | 215121_x_at | 168 | Hs.356861 | NM_018058; cartilage acidic protein 1 | CIS |
| 524 | U133A | 215176_x_at | 168 | Hs.503443 | NM_001248; ectonucleoside triphosphate | CIS |
| | | | | | diphosphohydrolase 3 | |
| 525 | U133A | 215379_x_at | 168 | Hs.449601 | NM_006760; uroplakin 2 | CIS |
| 526 | U133A | 215812_s_at | 168 | Hs.499113 | NM_018058; cartilage acidic protein 1 | CIS |
| 527 | U133A | 216641_s_at | 168 | Hs.18141 | NM_005547; involucrin | CIS |
| 528 | U133A | 216971_s_at | 168 | Hs.79706 | NM_000445; plectin 1, intermediate | CIS |
| | | | | | filament binding protein 500 kDa | |
| 529 | U133A | 217028_at | 168 | Hs.421986 | NM_003282; troponin I, skeletal, fast | CIS |
| 530 | U133A | 217040_x_at | 168 | Hs.95582 | NM_001910; cathepsin E isoform a | CIS |
| | | | | | preproprotein NM_148964; cathepsin | |
| | | | | | E isoform b preproprotein | |
| 531 | U133A | 217388_s_at | 168 | Hs.444471 | NM_000228; laminin subunit beta 3 | CIS |
| | | | | | precursor | |
| 532 | U133A | 217626_at | 168 | Hs.201967 | NM_000299; plakophilin 1 | CIS |
| 533 | U133A | 218484_at | 168 | Hs.221447 | NM_020142; NADH: ubiquinone oxidoreductase | CIS |
| | | | | | MLRQ subunit homolog | |
| 534 | U133A | 218656_s_at | 168 | Hs.93765 | NM_001442; fatty acid binding protein | CIS |
| | | | | | 4, adipocyte | |
| 535 | U133A | 218718_at | 168 | Hs.43080 | NM_000445; plectin 1, intermediate | CIS |
| | | | | | filament binding protein 500 kDa | |
| 536 | U133A | 218918_at | 168 | Hs.8910 | NM_000300; phospholipase A2, group | CIS |
| | | | | | IIA (platelets, synovial fluid) | |
| 537 | U133A | 218960_at | 168 | Hs.414005 | NM_019894; transmembrane protease, | CIS |
| | | | | | serine 4 isoform 1 NM_183247; | |
| | | | | | transmembrane protease, serine 4 | |
| | | | | | isoform 2 | |
| 538 | U133A | 219410_at | 168 | Hs.104800 | NM_004692; NM_032727; internexin | CIS |
| | | | | | neuronal intermediate filament protein, | |
| | | | | | alpha | |
| 539 | U133A | 219922_s_at | 168 | Hs.289019 | NM_030570; uroplakin 3B isoform a | CIS |
| | | | | | NM_182683; uroplakin 3B isoform c | |
| | | | | | NM_182684; uroplakin 3B isoform b | |
| 540 | U133A | 220026_at | 168 | Hs.227059 | NM_001442; fatty acid binding protein | CIS |
| | | | | | 4, adipocyte | |
| 541 | U133A | 220779_at | 168 | Hs.149195 | NM_016233; peptidylarginine deiminase | CIS |
| | | | | | type III | |
| 542 | U133A | 221204_s_at | 168 | Hs.326444 | NM_018058; cartilage acidic protein 1 | CIS |
| 543 | U133A | 221660_at | 168 | Hs.247831 | NM_000300; phospholipase A2, group | CIS |
| | | | | | IIA (platelets, synovial fluid) | |
| 544 | U133A | 221671_x_at | 168 | Hs.377975 | NM_000299; plakophilin 1 | CIS |
| 545 | U133A | 221854_at | 168 | Hs.313068 | NM_000299; plakophilin 1 | CIS |
| 546 | U133A | 221872_at | 168 | Hs.82547 | NM_001958; eukaryotic translation | CIS |
| | | | | | elongation factor 1 alpha 2 | |
| 547 | U133A | 200958_s_at | 168 | Hs.164067 | NM_005625; syndecan binding protein | CIS |
| | | | | | (syntenin) | |
| 548 | U133A | 201877_s_at | 168 | Hs.249955 | NM_002719; gamma isoform of regulatory | CIS |
| | | | | | subunit B56, protein phosphatase | |
| | | | | | 2A isoform a NM_178586; | |
| | | | | | gamma isoform of regulatory subunit | |
| | | | | | B56, protein phosphatase 2A isoform b | |
| | | | | | NM_178587; gamma isoform of regulatory | |
| | | | | | subunit B56, protein phosphatase | |
| | | | | | 2A isoform c NM_178588; | |
| | | | | | gamma isoform of regulatory subunit | |
| | | | | | B56, protein phosphatase 2A isoform d | |
| 549 | U133A | 201887_at | 168 | Hs.285115 | NM_001560; interleukin 13 receptor, | CIS |
| | | | | | alpha 1 precursor | |
| 550 | U133A | 202076_at | 168 | Hs.289107 | NM_001166; baculoviral IAP repeat- | CIS |
| | | | | | containing protein 2 | |
| 551 | U133A | 202777_at | 168 | Hs.104315 | NM_007373; soc-2 suppressor of clear | CIS |
| | | | | | homolog | |
| 552 | U133A | 204640_s_at | 168 | Hs.129951 | NM_003563; speckle-type POZ protein | CIS |
| 553 | U133A | 209004_s_at | 168 | Hs.5548 | NM_012161; F-box and leucine-rich | CIS |
| | | | | | repeat protein 5 isoform 1 | |
| | | | | | NM_033535; F-box and leucine-rich | |
| | | | | | repeat protein 5 isoform 2 | |
| 554 | U133A | 209241_x_at | 168 | Hs.112028 | NM_015716; misshapen/NIK-related | CIS |
| | | | | | kinase isoform 1 NM_153827; misshapen/ | |
| | | | | | NIK-related kinase isoform 3 | |
| | | | | | NM_170663; misshapen/NIK-related | |
| | | | | | kinase isoform 2 | |
| 555 | U133A | 209579_s_at | 168 | Hs.35947 | NM_003925; methyl-CpG binding | CIS |
| | | | | | domain protein 4 | |
| 556 | U133A | 209630_s_at | 168 | Hs.444354 | NM_012164; F-box and WD-40 domain | CIS |
| | | | | | protein 2 | |
| 557 | U133A | 212784_at | 168 | Hs.388236 | NM_015125; capicua homolog | CIS |
| 558 | U133A | 212802_s_at | 168 | Hs.287266 | | CIS |
| 559 | U133A | 212899_at | 168 | Hs.129836 | NM_015076; cyclin-dependent kinase | CIS |
| | | | | | (CDC2-like) 11 | |
| 560 | U133A | 213633_at | 168 | Hs.97858 | NM_018957; SH3-domain binding | CIS |
| | | | | | protein 1 | |
| 561 | U133A | 217941_s_at | 168 | Hs.8117 | NM_018695; erbb2 interacting protein | CIS |
| 562 | U133A | 218150_at | 168 | Hs.342849 | NM_012097; ADP-ribosylation factor- | CIS |
| | | | | | like 5 isoform 1 NM_177985; ADP- | |
| | | | | | ribosylation factor-like 5 isoform 2 | |

The expression level of at least one gene in the sample is determined, wherein at least one of said genes is selected from the genes of Table A. The samples according to the present invention may be any tissue sample or body fluid sample, it is however often preferred to conduct the methods according to the invention on epithelial tissue, such as epithelial tissue from the bladder. In particular the epithelial tissue may be mucosa. In another embodiment the sample is a urine sample comprising the tissue cells.

The sample may be obtained by any suitable manner known to the man skilled in the art, such as a biopsy of the tissue, or a superficial sample scraped from the tissue. The sample may be prepared by forming a cell suspension made from the tissue, or by obtaining an extract from the tissue.

In one embodiment it is preferred that the sample comprises substantially only cells from said tissue, such as substantially only cells from mucosa of the bladder.

The methods according to the invention may be used for determining any biological condition, wherein said condition leads to a change in the expression of at least one gene, and preferably a change in a variety of genes.

Thus, the biological condition may be any malignant or premalignant condition, in particular in bladder, such as a tumor or an adenocarcinoma, a carcinoma, a teratoma, a sarcoma, and/or a lymphoma, and/or carcinoma-in-situ, and/or dysplasia-in-situ.

The expression level may be determined as single gene approaches, i.e. wherein the determination of expression from one or two or a few genes is conducted. It is however preferred that information is obtained from several genes, so that an expression pattern is obtained.

In a preferred embodiment expression from at least one gene from a first group is determined, said first gene group representing genes being expressed at a higher level in one type of tissue, i.e. tissue in one stage or one risk group, in combination with determination of expression of at least one gene from a second group, said second group representing genes being expressed at a higher level in tissue from another stage or from another risk group. Thereby the validity of the prediction increases, since expression levels from genes from more than one group are determined.

However, determination of the expression of a single gene whether belonging to the first group or second group is also within the scope of the present invention. In this case it is preferred that the single gene is selected among genes having a high change in expression level from normal cells to biological condition cells.

Another approach is determination of an expression pattern from a variety of genes, wherein the determination of the biological condition in the tissue relies on information from a variety of gene expression, i.e. rather on the combination of expressed genes than on the information from single genes.

The following data presented herein relates to bladder tumors, and therefore the description has focused on the gene expression level as one way of identifying genes that lose or gain function in cancer tissue. Genes showing a remarkable downregulation (or complete loss) or upregulation (gene expression gained de novo) of the expression level—measured as the mRNA transcript, during the malignant progression in bladder from normal mucosa through Ta superficial tumors, and Carcinoa in situ (CIS) to T1, slightly invasive tumors, to T2, T3 and T4 which have spread to muscle or even further into lymph nodes or other organs are within the scope of the invention, as well as genes gaining importance during the differentiation from normal towards malignancy.

The present invention relates to a variety of genes identified either by an EST identification number and/or by a gene identification number. Both type of identification numbers relates to identification numbers of UniGene database, NCBI, build 18.

The various genes have been identified using Affymetrix arrays of the following product numbers:

HUGeneFL (sold in 2000-2002)
EOS Hu03 (customized Affymetric array)
U133A (product #900367 sold in 2003)

Stage of a bladder tumor indicates how deep the tumor has penetrated. Superficial tumors are termed Ta, and Carcinoma in situ (CIS), and T1, T2, T3 and T4 are used to describe increasing degrees of penetration into the muscle. The grade of a bladder tumor is expressed on a scale of I-IV (1-4) according to Bergkvist, A.; Ijungquist, A.; Moberger, B. “Classification of bladder tumours basedf on the cellular pattern. Preliminary report of a clinical-pathological study of 300 cases with a minimum follow-up of eight years”, Acta Chir Scand., 1965, 130(4):371-8). The grade reflects the cytological appearance of the cells. Grade I cells are almost normal. Grade II cells are slightly deviant. Grade III cells are clearly abnormal. And Grade IV cells are highly abnormal. A special form of bladder malignancy is carcinoma-in-situ or dyplasia-in-situ in which the altered cells are located in-situ.

It is important to predict the prognosis of a cancer disease, as superficial tumors may require a less intensive treatment than invasive tumors. According to the invention the expression level of genes may be used to identify genes whose expression can be used to identify a certain stage and/or the prognosis of the disease. These “Classifiers” are divided into those which can be used to identify Ta, Carcinoma in situ (CIS), T1, and T2 stages as well as those identifying risk of recurrence or progression. In one aspect of the invention measuring the transcript level of one or more of these genes may lead to a classifier that can add supplementary information to the information obtained from the pathological classification. For example gene expression levels that signify a T2 stage will be unfavourable to detect in a Ta tumor, as they may signal that the Ta tumor has the potential to become a T2 tumor. The opposite is probably also true, that an expression level that signify Ta will be favorable to have in a T2 tumor. In that way independent information may be obtained from pathological classification and a classification based on gene expression levels is made.

In the present context a standard expression level is the level of expression of a gene in a standard situation, such as a standard Ta tumor or a standard T2 tumor. For use in the pre-sent invention standard expression levels is determined for each stage as well as for each group of progression, recurrence, and other prognostic indices. It is then possible to compare the result of a determination of the expression level from a gene of a given biological condition with a standard for each stage, progression, recurrence and other indices to obtain a classification of the biological condition.

Furthermore, in the present context a reference pattern refers to the pattern of expression levels seen in standard situations as discussed above, and reference patterns may be used as discussed above for standard expression levels.

It is known from the histopathological classification of bladder tumors that some information is obtained from merely classifying into stage and grade of tumor. Accordingly, in one aspect, the invention relates to a method of predicting the prognosis of the biological condition by determining the stage of the biological condition, by determining an expression level of at least one gene, wherein said gene is selected from the group of genes consisting of gene No 1 to gene No. 562. In this aspect information about the stage reveils directly information about the prognosis as well. An example hereof is when a bladder tumor is classified as for example stage T2, then the prognosis for the bladder tumor is obtained directly from the prognosis related generally to stage T2 tumors. In a preferred embodiment the genes for predicting the prognosis by establishing the stage of the tumor may be selected from gene selected from the group of genes consisting of gene No. 1 to gene No. 188. More preferably the genes for predicting the prognosis by establishing the stage of the tumor may be selected from gene selected from the group of genes consisting of gene Nos. 18, 39, 40, 55, 58, 79, 86, 87, 88, 91, 93, 103, 105, 106, 121, 123, 125, 126, 136, 137, 140, 149, 156, 158, 161, 165, 166, 167, 175, 184, 187, 188.

It is preferred that the expression level of more one gene is determined, such as the expression level of at least two genes, such as the expression level of at least three genes, such as the expression level of at least four genes, such as the expression level of at least five genes, such as the expression level of at least six genes, such as the expression level of at least seven genes, such as the expression level of at least eight genes, such as the expression level of at least nine genes, such as the expression level of at least ten genes, such as the expression level of at least 15 genes, such as the expression level of at least 20 genes, such as the expression levels of at least 25 genes, such as the expression levels of at least 30 genes, such as the expression level of 32 genes.

As discussed above, in relation to bladder cancer the stages of a bladder tumor are selected from bladder cancer stages Ta, Carcinoma in situ, T1, T2, T3 and T4. In an embodiment the determination of a stage comprises

assaying at least the expression of Ta stage gene from a Ta stage gene group, at least one expression of a CIS gene, at least one expression of T1 stage gene from a T1 stage gene group, at least the expression of T2 stage gene from a T2 stage gene group, and more preferably assaying at least the expression of Ta stage gene from a Ta stage gene group, at least one expression of a CIS gene, at least one expression of T1 stage gene from a T1 stage gene group, at least the expression of T2 stage gene from a T2 stage gene group, at least the expression of T3 stage gene from a T3 stage gene group, at least the expression of T4 stage gene from a T4 stage gene group wherein at least one gene from each gene group is expressed in a significantly different amount in that stage than in one of the other stages.

Preferably, the genes selected may be a gene from each gene group being expressed in a significantly higher amount in that stage than in one of the other stages as compared to normal controls, see for example Table B below.

The genes selected may be a gene from each gene group being expressed in a significantly lower amount in that stage than in one of the other stages.

In another embodiment the present invention relates to a method of predicting the prognosis of a biological condition by obtaining information in addition to the stage classification as such. As described above, by determining gene expression levels that signify a T2 stage in a tumor otherwise classified as a Ta tumor, the expression levels signal that the Ta tumor has the potential to become a T2 tumor. The opposite is also true, that an expression level that signify Ta will be favorable to have in a T2 tumor. In the present invention the inventors have shown that some genes are relevant for obtaining this additional information.

Also, in one embodiment the present invention relates to a further method of predicting the prognosis of a biological condition by obtaining information in addition to the stage classification as such. Determination of squamous metaplasia in a tumor, in particular in a T2 stage tumor, is indicative of risk of progression. In particular the genes may be selected from gene selected from the group of genes consisting of gene No. 215 to gene No. 232, see also table H.

It is preferred that the expression level of more one gene is determined, such as the expression level of at least two genes, such as the expression level of at least three genes, such as the expression level of at least four genes, such as the expression level of at least five genes, such as the expression level of at least six genes, such as the expression level of at least seven genes, such as the expression level of at least eight genes, such as the expression level of at least nine genes, such as the expression level of at least ten genes, such as the expression level of at least 15 genes, such as the expression level of 18 genes.

In another embodiment the invention relates to genes bearing information of recurrence of the biological condition as such. In particular the genes may be selected from gene selected from the group of genes consisting of gene No. 189 to gene No. 214. It is preferred to determine a first expression level of at least one gene from a first gene group, wherein the gene from the first gene group is selected from the group of genes wherein expression is increased in case of recurrence, genes No. 189 to gene No. 199 (recurrence genes), and determined a second expression level of at least one gene from a second gene group, wherein the second gene group is selected from the group of genes wherein expression is increased in case of no recurrence, genes No. 200 to No. 214 (non-recurrence genes), and correlate the first expression level to a standard expression level for progressors, and/or the second expression level to a standard expression level for non-progressors to predict the prognosis of the biological condition in the animal tissue, see also table C.

It is preferred that the expression level of more one gene is determined, such as the expression level of at least two genes, such as the expression level of at least three genes, such as the expression level of at least four genes, such as the expression level of at least five genes, such as the expression level of at least six genes, such as the expression level of at least seven genes, such as the expression level of at least eight genes, such as the expression level of at least nine genes, such as the expression level of at least ten genes, such as the expression level of at least 15 genes, such as the expression level of at least 20 genes, such as the expression level of at least 25 genes, such as the expression level of 26 genes.

Furthermore, in another embodiment the invention relates to genes bearing information of progression as such. In particular the genes may be selected from the group of genes of table D, more preferably selected from the group of genes consisting of gene No. 233 to gene No. 446. More preferably the genes may be selected from the group of genes Nos. 255, 273, 279, 280, 281, 282, 287, 295, 300, 311, 317, 320, 333, 346, 347, 349, 352, 364, 365, 373, 383, 386, 390, 394, 401, 407, 414, 417, 426, 427, 428, 433, 434, 435, 436, 437, 438, 439, 440, 441, 442, 443, 444, 445, 446, see table E.

It is preferred that the expression level of more one gene is determined, such as the expression level of at least two genes, such as the expression level of at least three genes, such as the expression level of at least four genes, such as the expression level of at least five genes, such as the expression level of at least six genes, such as the expression level of at least seven genes, such as the expression level of at least eight genes, such as the expression level of at least nine genes, such as the expression level of at least ten genes, such as the expression level of at least 15 genes, such as the expression level of at least 20 genes, such as the expression levels of at least 25 genes, such as the expression levels of at least 30 genes, such as the expression level of at least 35 genes, such as the expression level of at least 40 genes, such as the expression level of 45 genes.

Furthermore, it is within the scope of the invention to predict the prognosis of a biological condition in animal tissue by determining the expression level of at least two genes, by
determining a first expression level of at least one gene from a first gene group, wherein the gene from the first gene group is selected from the group of gene Nos. 237, 238, 239, 240, 241, 242, 243, 245, 246, 247, 248, 250, 253, 254, 257, 258, 260, 263, 264, 265, 267, 270, 271, 272, 278, 283, 284, 287, 288, 290, 291, 292, 294, 297, 298, 300, 302, 303, 305, 309, 310, 315, 316, 317, 318, 319, 321, 324, 329, 335, 336, 337, 339, 340, 344, 346, 347, 354, 356, 358, 359, 362, 364, 365, 368, 369, 371, 372, 377, 378, 379, 380, 381, 382, 383, 384, 388, 391, 393, 395, 396, 397, 399, 402, 403, 404, 409, 413, 417, 419, 420, 421, 422, 423, 425, 427, 429, 430, 431, 432, 437, 444 (progressor genes), and
determining a second expression level of at least one gene from a second gene group, wherein the second gene group is selected from the group of genes Nos. 233, 234, 235, 236, 244, 249, 251, 252, 255, 256, 259, 261, 262, 266, 268, 269, 273, 274, 275, 276, 277, 279, 280, 281, 282, 285, 286, 289, 293, 295, 296, 299, 301, 304, 306, 307, 308, 311, 312, 313, 314, 320, 322, 323, 325, 326, 327, 328, 330, 331, 332, 333, 334, 338, 341, 342, 343, 345, 348, 349, 350, 351, 352, 353, 355, 357, 360, 361, 363, 366, 367, 370, 373, 374, 375, 376, 385, 386, 387, 389, 390, 392, 394, 398, 400, 401, 405, 406, 407, 408, 410, 411, 412, 414, 415, 416, 418, 424, 426, 428, 433, 434, 435, 436, 438, 439, 440, 441, 442, 443, 445, 446 (non-progressor genes), and
correlating the first expression level to a standard expression level for progressors, and/or the second expression level to a standard expression level for non-progressors to predict the prognosis of the biological condition in the animal tissue.

In particular the genes of the first group and the second group for predicting the prognosis of a Ta stage tumor may be selected from gene selected from the group of progression/non-progression genes described above.

In yet another embodiment the present invention offers the possibility to predict the presence or absence of Carcinoma in situ in the same organ as the primary biological condition. An example hereof is for a Ta bladder tumor to predict, whether the bladder in addition to the Ta tumor comprises Carcinoma in situ (CIS). The presence of carcinoma in situ in a bladder containing a superficial Ta tumor is a signal that the Ta tumor has the potential of recurrence and invasiveness. Accordingly, by predicting the presence of carcinoma in situ important information about the prognosis is obtained. In the present context, genes for predicting the presence of carcinoma in situ for a Ta stage tumor may be selected from gene selected from the group of genes consisting of gene No. 447 to gene No. 562. More preferably the genes are selected from the group of genes consisting of gene Nos 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, see table F, or from the group of genes consisting of gene Nos. 547, 548, 549, 550, 551, 552, 553, 554, 555, 556, 557, 558, 559, 560, 561, 562, see table G.

It is preferred that the expression level of more one gene is determined, such as the expression level of at least two genes, such as the expression level of at least three genes, such as the expression level of at least four genes, such as the expression level of at least five genes, such as the expression level of at least six genes, such as the expression level of at least seven genes, such as the expression level of at least eight genes, such as the expression level of at least nine genes, such as the expression level of at least ten genes, such as the expression level of at least 15 genes, such as the expression level of at least 20 genes, such as the expression levels of at least 25 genes, such as the expression levels of at least 30 genes, such as the expression level of at least 35 genes, such as the expression level of at least 40 genes, such as the expression level of at least 45 genes, such as the expression level of at least 50 genes, such as 100 genes. In another embodiment the expression level of 16 genes are determined.

It is also preferred to determine a first expression level of at least one gene from a first gene group, wherein the gene from the first gene group is selected from the group of genes wherein expression is increased in case of CIS, genes Nos. 447, 448, 449, 450, 451, 452, 454, 455, 456, 457, 458, 459, 462, 468, 474, 478, 484, 489, 491, 493, 495, 500, 501, 502, 504, 505, 506, 507, 508, 509, 510, 511, 512, 513, 514, 518, 519, 520, 522, 523, 524, 525, 529, 531, 534, 535, 536, 538, 544, 546, 547, 548, 549, 550, 551, 552, 553, 555, 556, 558, 559, 561, 562 (CIS genes), and determined a second expression level of at least one gene from a second gene group, wherein the second gene group is selected from the group of genes wherein expression is increased in case of no CIS, genes Nos. 453, 460, 461, 463, 464, 465, 466, 467, 469, 470, 471, 472, 473, 475, 476, 477, 479, 480, 481, 482, 483, 485, 486, 487, 488, 490, 492, 494, 496, 497, 498, 499, 503, 515, 516, 517, 521, 526, 527, 528, 530, 532, 533, 537, 539, 540, 541, 542, 543, 545, 554, 557, 560 (non-CIS genes), and correlate the first expression level to a standard expression level for CIS, and/or the second expression level to a standard expression level for non-CIS to predict the prognosis of the biological condition in the animal tissue.

It is preferred when determining the expression level of at least one gene from a first group and at least one gene from a second group that the expression level of more than one genes from each group is determined. Thus, it is preferred that the expression level of more one gene is determined, such as the expression level of at least two genes, such as the expression level of at least three genes, such as the expression level of at least four genes, such as the expression level of at least five genes, such as the expression level of at least six genes, such as the expression level of at least seven genes, such as the expression level of at least eight genes, such as the expression level of at least nine genes, such as the expression level of at least ten genes in each group.

In one embodiment of the invention the stage of the biological condition has been determined before the prediction of prognosis. The stage may be determined by any suitable means such as determined by histological examination of the tissue or by genotyping of the tissue, preferably by genotyping of the tissue such as described herein or as described in WO 02/02804 incorporated herein by reference.

In another aspect the invention relates to a method of determining the stage of a biological condition in animal tissue,
comprising collecting a sample comprising cells from the tissue,
determining an expression level of at least one gene selected from the group of genes consisting of gene No. 1 to gene No. 562,
correlating the expression level of the assessed genes to at least one standard level of expression determining the stage of the condition.

In particular the expression level of at least one gene selected from the group of genes consisting of gene Nos. 1-457 and gene Nos. 459-535 and gene Nos. 537-562.

Specific embodiments of determining the stage is as described above for predicting prognosis by determination of stage.

In a preferred embodiment the expression level of at least two genes is determined by
determining the expression of at least a first stage gene from a first stage gene group and at least a second stage gene from a second stage gene group, wherein at least one of said genes is expressed in said first stage of the condition in a higher amount than in said second stage, and the other gene is a expressed in said first stage of the condition in a lower amount than in said second stage of the condition, and
correlating the expression level of the assessed genes to a standard level of expression determining the stage of the condition.

In general, genes being downregulated for higher stage tumors as well as for progression and recurrence may be of importance as predictive markers for the disease as loss of one or more of these may signal a poor outcome or an aggressive disease course. Furthermore, they may be important targets for therapy as restoring their expression level, e.g. by gene therapy, or substitution with those peptide products or small molecules with a similar biological effect may suppress the malignant growth.

Genes that are up-regulated (or gained de novo) during the malignant progression of bladder cancer from normal tissue through Ta, T1, T2, T3 and T4 is also within the scope of the invention. These genes are potential oncogenes and may be those genes that create or enhance the malignant growth of the cells. The expression level of these genes may serve as predictive markers for the disease course and treatment response, as a high level may signal an aggressive disease course, and they may serve as targets for therapy, as blocking these genes by e.g. anti-sense therapy, or by biochemical means could inhibit, or slow the tumor growth.

The genes used according to the invention show a sufficient difference in expression from one group to another and/or from one stage to another to use the gene as a classifier for the group and/or stage. Thus, comparison of an expression pattern to another may score a change from expressed to non-expressed, or the reverse. Alternatively, changes in intensity of expression may be scored, either increases or decreases. Any significant change can be used. Typical changes which are more than 2-fold are suitable. Changes which are greater than 5-fold are highly suitable.

The present invention in particular relates to methods using genes wherein at least a two-fold change in expression, such as at least a three-fold change, for example at least a four fold change, such as at least a five fold change, for example at least a six fold change, such as at least a ten fold change, for example at least a fifteen fold change, such as at least a twenty fold change is seen between two groups.

As described above the invention relates to the use of information of expression levels. In one embodiment the expression patterns is obtained, thus, the invention relates to a method of determining an expression pattern of a bladder cell sample, comprising:
collecting sample comprising bladder cells and/or expression products from bladder cells,
determining the expression level of at least one gene in the sample, said gene being selected from the group of genes consisting of gene No. 1 to gene No. 562, and obtaining an expression pattern of the bladder cell sample.

The invention preferably include more than one gene in the pattern, according it is preferred to include the expression level of at least two genes, such as the expression level of at least three genes, such as the expression level of at least four genes, such as the expression level of at least five genes, such as the expression level of more than six genes.

The expression pattern preferably relates to one or more of the group of genes discussed above with respect to prognosis relating to stage, SSC, progression, recurrence and/or CIS.

In order to predict prognosis and/or stages it is preferred to determine an expression pattern of a cell sample preferably independent of the proportion of submucosal, muscle and connective tissue cells present. Expression is determined of one or more genes in a sample comprising cells, said genes being selected from the same genes as discussed above and shown in the tables.

It is an object of the present invention that characteristic patterns of expression of genes can be used to characterize different types of tissue. Thus, for example gene expression patterns can be used to characterize stages and grades of bladder tumors. Similarly, gene expression patterns can be used to distinguish cells having a bladder origin from other cells. Moreover, gene expression of cells which routinely contaminate bladder tumor biopsies has been identified, and such gene expression can be removed or subtracted from patterns obtained from bladder biopsies. Further, the gene expression patterns of single-cell solutions of bladder tumor cells have been found to be substantially without interfering expression of contaminating muscle, submucosal, and connective tissue cells than biopsy samples.

The one or more genes exclude genes which are expressed in the submucosal, muscle, and connective tissue. A pattern of expression is formed for the sample which is independent of the proportion of submucosal, muscle, and connective tissue cells in the sample.

In another aspect of the invention a method of determining an expression pattern of a cell sample is provided. Expression is determined of one or more genes in a sample comprising cells. A first pattern of expression is thereby formed for the sample. Genes which are expressed in submucosal, muscle, and connective tissue cells are removed from the first pattern of expression, forming a second pattern of expression which is independent of the proportion of submucosal, muscle, and connective tissue cells in the sample.

Another embodiment of the invention provides a method for determining an expression pattern of a bladder mucosa or bladder cancer cell. Expression is determined of one or more genes in a sample comprising bladder mucosa or bladder cancer cells; the expression determined forms a first pattern of expression. A second pattern of expression which was formed using the one or more genes and a sample comprising predominantly submucosal, muscle, and connective tissue cells, is subtracted from the first pattern of expression, forming a third pattern of expression. The third pattern of expression reflects expression of the bladder mucosa or bladder cancer cells independent of the proportion of submucosal, muscle, and connective tissue cells present in the sample.

In one embodiment the invention provides a method to predict the prognosis of a bladder tumor as described above. A first pattern of expression is determined of one or more genes in a bladder tumor sample. The first pattern is compared to one or more reference patterns of expression determined for bladder tumors at different stages and/or in different groups. The reference pattern which shares maximum similarity with the first pattern is identified. The stage of the reference pattern with the maximum similarity is assigned to the bladder tumor sample.

Yet another embodiment the invention provides a method to determine the stage of a bladder tumor as described above. A first pattern of expression is determined of one or more genes in a bladder tumor sample. The first pattern is compared to one or more reference patterns of expression determined for bladder tumors at different stages. The reference pattern which shares maximum similarity with the first pattern is identified. The stage of the reference pattern with the maximum similarity is assigned to the bladder tumor sample.

Since a biopsy of the tissue often contains more tissue material such as connective tissue than the tissue to be examined, when the tissue to be examined is epithelial or mucosa, the invention also relates to methods, wherein the expression pattern of the tissue is independent of the amount of connective tissue in the sample.

Biopsies contain epithelial cells that most often are the targets for the studies, and in addition many other cells that contaminate the epithelial cell fraction to a varying extent. The contaminants include histiocytes, endothelial cells, leukocytes, nerve cells, muscle cells etc. Micro dissection is the method of choice for DNA examination, but in the case of expression studies this procedure is difficult due to RNA degradation during the procedure. The epithelium may be gently removed and the expression in the remaining submucosa and underlying connective tissue (the bladder wall) monitored. Genes expressed at high or low levels in the bladder wall should be interrogated when performing expression monitoring of the mucosa and tumors. A similar approach could be used for studies of epithelia in other organs.

In one embodiment of the invention normal mucosa lining the bladder lumen from bladders for cancer is scraped off. Then biopsies is taken from the denuded submucosa and connective tissue, reaching approximately 5 mm into the bladder wall, and immediately disintegrated in guanidinium isothiocyanate. Total RNA may be extracted, pooled, and poly(A)⁺ mRNA may be prepared from the pool followed by conversion to double-stranded cDNA and in vitro transcription into cRNA containing biotin-labeled CTP and UTP.

Genes that are expressed and genes that are not expressed in bladder wall can both interfere with the interpretation of the expression in a biopsy, and should be considered when interpreting expression intensities in tumor biopsies, as the bladder wall component of a biopsy varies in amount from biopsy to biopsy.

When having determined the pattern of genes expressed in bladder wall components said pattern may be subtracted from a pattern obtained from the sample resulting in a third pattern related to the mucosa (epithelial) cells.

In another embodiment of the invention a method is provided for determining an expression pattern of a bladder tissue sample independent of the proportion of submucosal, muscle and connective tissue cells present. A single-cell suspension of disaggregated bladder tumor cells is isolated from a bladder tissue sample comprising bladder tumor cells is isolated from a bladder tissue sample comprising bladder cells, submucosal cells, muscle cells, and connective tissue cells. A pattern of expression is thus formed for the sample which is independent of the proportion of submucosal, muscle, and connective tissue cells in the bladder tissue sample.

Yet another method relates to the elimination of mRNA from bladder wall components before determining the pattern, e.g. by filtration and/or affinity chromatography to remove mRNA related to the bladder wall.

Working with tumor material requires biopsies or body fluids suspected to comprise relevant cells. Working with RNA requires freshly frozen or immediately processed biopsies, or chemical pretreatment of the biopsy. Apart from the cancer tissue, biopsies do inevitably contain many different cell types, such as cells present in the blood, connective and muscle tissue, endothelium etc. In the case of DNA studies, microdissection or laser capture are methods of choice, however the time-dependent degradation of RNA makes it difficult to perform manipulation of the tissue for more than a few minutes. Furthermore, studies of expressed sequences may be difficult on the few cells obtained via microdissection or laser capture, as these cells may have an expression pattern that deviates from the predominant pattern in a tumor due to large intratumoral heterogeneity.

In the present context high density expression arrays may be used to evaluate the impact of bladder wall components in bladder tumor biopsies, and tested preparation of single cell solutions as a means of eliminating the contaminants. The results of these evaluations permit for the design of methods of evaluating bladder samples without the interfering background noise caused by ubiquitous contaminating submucosal, muscle, and connective tissue cells. The evaluating assays of the invention may be of any type.

While high density expression arrays can be used, other techniques are also contemplated. These include other techniques for assaying for specific mRNA species, including RT-PCR and Northern Blotting, as well as techniques for assaying for particular protein products, such as ELISA, Western blotting, and enzyme assays. Gene expression patterns according to the present invention are determined by measuring any gene product of a particular gene, including mRNA and protein. A pattern may be for one or more genes.

RNA or protein can be isolated and assayed from a test sample using any techniques known in the art. They can for example be isolated from a fresh or frozen biopsy, from formalin-fixed tissue, from body fluids, such as blood, plasma, serum, urine, or sputum.

Expression of genes may in general be detected by either detecting mRNA from the cells and/or detecting expression products, such as peptides and proteins.

The detection of mRNA of the invention may be a tool for determining the developmental stage of a cell type which may be definable by its pattern of expression of messenger RNA. For example, in particular stages of cells, high levels of ribosomal RNA are found whereas relatively low levels of other types of messenger RNAs may be found. Where a pattern is shown to be characteristic of a stage, said stage may be defined by that particular pattern of messenger RNA expression. The mRNA population is a good determinant of a developmental stage, and may be correlated with other structural features of the cell. In this manner, cells at specific developmental stages will be characterized by the intracellular environment, as well as the extracellular environment. The present invention also allows the combination of definitions based in part upon antigens and in part upon mRNA expression. In one embodiment, the two may be combined in a single incubation step. A particular incubation condition may be found which is compatible with both hybridization recognition and non-hybridization recognition molecules. Thus, e.g. an incubation condition may be selected which allows both specificity of antibody binding and specificity of nucleic acid hybridization. This allows simultaneous performance of both types of interactions on a single matrix. Again, where developmental mRNA patterns are correlated with structural features, or with probes which are able to hybridize to intracellular mRNA populations, a cell sorter may be used to sort specifically those cells having desired mRNA population patterns.

It is within the general scope of the present invention to provide methods for the detection of mRNA. Such methods often involve sample extraction, PCR amplification, nucleic acid fragmentation and labeling, extension reactions, and transcription reactions.

The nucleic acid (either genomic DNA or mRNA) may be isolated from the sample according to any of a number of methods well known to those of skill in the art. One of skill will appreciate that where alterations in the copy number of a gene are to be detected genomic DNA is preferably isolated. Conversely, where expression levels of a gene or genes are to be detected, preferably RNA (mRNA) is isolated.

Methods of isolating total mRNA are well known to those of skill in the art. In one embodiment, the total nucleic acid is isolated from a given sample using, for example, an acid guanidinium-phenol-chloroform extraction method and polyA.sup. and mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

The sample may be from tissue and/or body fluids, as defined elsewhere herein. Before analyzing the sample, e.g., on an oligonucleotide array, it will often be desirable to perform one or more sample preparation operations upon the sample. Typically, these sample preparation operations will include such manipulations as extraction of intracellular material, e.g., nucleic acids from whole cell samples, viruses, amplification of nucleic acids, fragmentation, transcription, labeling and/or extension reactions. One or more of these various operations may be readily incorporated into the device of the present invention.

DNA extraction may be relevant under circumstances where possible mutations in the genes are to be determined in addition to the determination of expression of the genes.

For those embodiments where whole cells, or other tissue samples are being analyzed, it will typically be necessary to extract the nucleic acids from the cells or viruses, prior to continuing with the various sample preparation operations. Accordingly, following sample collection, nucleic acids may be liberated from the collected cells, viral coat etc. into a crude extract followed by additional treatments to prepare the sample for subsequent operations, such as denaturation of contaminating (DNA binding) proteins, purification, filtration and desalting.

Liberation of nucleic acids from the sample cells, and denaturation of DNA binding proteins may generally be performed by physical or chemical methods. For example, chemical methods generally employ lysing agents to disrupt the cells and extract the nucleic acids from the cells, followed by treatment of the extract with chaotropic salts such as guanidinium isothiocyanate or urea to denature any contaminating and potentially interfering proteins.

Alternatively, physical methods may be used to extract the nucleic acids and denature DNA binding proteins, such as physical protrusions within microchannels or sharp edged particles piercing cell membranes and extract their contents. Combinations of such structures with piezoelectric elements for agitation can provide suitable shear forces for lysis.

More traditional methods of cell extraction may also be used, e.g., employing a channel with restricted cross-sectional dimension which causes cell lysis when the sample is passed through the channel with sufficient flow pressure. Alternatively, cell extraction and denaturing of contaminating proteins may be carried out by applying an alternating electrical current to the sample. More specifically, the sample of cells is flowed through a microtubular array while an alternating electric current is applied across the fluid flow. Subjecting cells to ultrasonic agitation, or forcing cells through microgeometry apertures, thereby subjecting the cells to high shear stress resulting in rupture are also possible extraction methods.

Following extraction, it will often be desirable to separate the nucleic acids from other elements of the crude extract, e.g. denatured proteins, cell membrane particles and salts. Removal of particulate matter is generally accomplished by filtration or flocculation. Further, where chemical denaturing methods are used, it may be desirable to desalt the sample prior to proceeding to the next step. Desalting of the sample and isolation of the nucleic acid may generally be carried out in a single step, e.g. by binding the nucleic acids to a solid phase and washing away the contaminating salts, or performing gel filtration chromatography on the sample passing salts through dialysis membranes. Suitable solid supports for nucleic acid binding include e.g. diatomaceous earth or silica (i.e., glass wool). Suitable gel exclusion media also well known in the art may be readily incorporated into the devices of the present invention and is commercially available from, e.g., Pharmacia and Sigma Chemical.

Alternatively, desalting methods may generally take advantage of the high electrophoretic mobility and negativity of DNA compared to other elements. Electrophoretic methods may also be utilized in the purification of nucleic acids from other cell contaminants and debris. Upon application of an appropriate electric field, the nucleic acids present in the sample will migrate toward the positive electrode and become trapped on the capture membrane. Sample impurities remaining free of the membrane are then washed away by applying an appropriate fluid flow. Upon reversal of the voltage, the nucleic acids are released from the membrane in a substantially purer form. Further, coarse filters may also be overlaid on the barriers to avoid any fouling of the barriers by particulate matter, proteins or nucleic acids, thereby permitting repeated use.

In a similar aspect, the high electrophoretic mobility of nucleic acids with their negative charges, may be utilized to separate nucleic acids from contaminants by utilizing a short column of a gel or other appropriate matrices or gels which will slow or retard the flow of other contaminants while allowing the faster nucleic acids to pass.

This invention provides nucleic acid affinity matrices that bear a large number of different nucleic acid affinity ligands allowing the simultaneous selection and removal of a large number of preselected nucleic acids from the sample. Methods of producing such affinity matrices are also provided. In general the methods involve the steps of a) providing a nucleic acid amplification template array comprising a surface to which are attached at least 50 oligonucleotides having different nucleic acid sequences, and wherein each different oligonucleotide is localized in a predetermined region of said surface, the density of said oligonucleotides is greater than about 60 different oligonucleotides per 1 cm.sup.2, and all of said different oligonucleotides have an identical terminal 3′ nucleic acid sequence and an identical terminal 5′ nucleic acid sequence. b) amplifying said multiplicity of oligonucleotides to provide a pool of amplified nucleic acids; and c) attaching the pool of nucleic acids to a solid support.

For example, nucleic acid affinity chromatography is based on the tendency of complementary, single-stranded nucleic acids to form a double-stranded or duplex structure through complementary base pairing. A nucleic acid (either DNA or RNA) can easily be attached to a solid substrate (matrix) where it acts as an immobilized ligand that interacts with and forms duplexes with complementary nucleic acids present in a solution contacted to the immobilized ligand. Unbound components can be washed away from the bound complex to either provide a solution lacking the target molecules bound to the affinity column, or to provide the isolated target molecules themselves. The nucleic acids captured in a hybrid duplex can be separated and released from the affinity matrix by denaturation either through heat, adjustment of salt concentration, or the use of a destabilizing agent such as formamide, TWEEN™-20 denaturing agent, or sodium dodecyl sulfate (SDS).

Affinity columns (matrices) are typically used either to isolate a single nucleic acid typically by providing a single species of affinity ligand. Alternatively, affinity columns bearing a single affinity ligand (e.g. oligo dt columns) have been used to isolate a multiplicity of nucleic acids where the nucleic acids all share a common sequence (e.g. a polyA).

The type of affinity matrix used depends on the purpose of the analysis. For example, where it is desired to analyze mRNA expression levels of particular genes in a complex nucleic acid sample (e.g., total mRNA) it is often desirable to eliminate nucleic acids produced by genes that are constitutively overexpressed and thereby tend to mask gene products expressed at characteristically lower levels. Thus, in one embodiment, the affinity matrix can be used to remove a number of preselected gene products (e.g., actin, GAPDH, etc.). This is accomplished by providing an affinity matrix bearing nucleic acid affinity ligands complementary to the gene products (e.g., mRNAs or nucleic acids derived therefrom) or to subsequences thereof. Hybridization of the nucleic acid sample to the affinity matrix will result in duplex formation between the affinity ligands and their target nucleic acids. Upon elution of the sample from the affinity matrix, the matrix will retain the duplexes nucleic acids leaving a sample depleted of the overexpressed target nucleic acids.

The affinity matrix can also be used to identify unknown mRNAs or cDNAs in a sample. Where the affinity matrix contains nucleic acids complementary to every known gene (e.g., in a cDNA library, DNA reverse transcribed from an mRNA, mRNA used directly or amplified, or polymerized from a DNA template) in a sample, capture of the known nucleic acids by the affinity matrix leaves a sample enriched for those nucleic acid sequences that are unknown. In effect, the affinity matrix is used to perform a subtractive hybridization to isolate unknown nucleic acid sequences. The remaining “unknown” sequences can then be purified and sequenced according to standard methods.

The affinity matrix can also be used to capture (isolate) and thereby purify unknown nucleic acid sequences. For example, an affinity matrix can be prepared that contains nucleic acid (affinity ligands) that are complementary to sequences not previously identified, or not previously known to be expressed in a particular nucleic acid sample. The sample is then hybridized to the affinity matrix and those sequences that are retained on the affinity matrix are “unknown” nucleic acids. The retained nucleic acids can be eluted from the matrix (e.g. at increased temperature, increased destabilizing agent concentration, or decreased salt) and the nucleic acids can then be sequenced according to standard methods.

Similarly, the affinity matrix can be used to efficiently capture (isolate) a number of known nucleic acid sequences. Again, the matrix is prepared bearing nucleic acids complementary to those nucleic acids it is desired to isolate. The sample is contacted to the matrix under conditions where the complementary nucleic acid sequences hybridize to the affinity ligands in the matrix. The non-hybridized material is washed off the matrix leaving the desired sequences bound. The hybrid duplexes are then denatured providing a pool of the isolated nucleic acids. The different nucleic acids in the pool can be subsequently separated according to standard methods (e.g. gel electrophoresis).

As indicated above the affinity matrices can be used to selectively remove nucleic acids from virtually any sample containing nucleic acids (e.g. in a cDNA library, DNA reverse transcribed from an mRNA, mRNA used directly or amplified, or polymerized from a DNA template, and so forth). The nucleic acids adhering to the column can be removed by washing with a low salt concentration buffer, a buffer containing a destabilizing agent such as formamide, or by elevating the column temperature.

In one particularly preferred embodiment, the affinity matrix can be used in a method to enrich a sample for unknown RNA sequences (e.g. expressed sequence tags (ESTs)). The method involves first providing an affinity matrix bearing a library of oligonucleotide probes specific to known RNA (e.g., EST) sequences. Then, RNA from undifferentiated and/or unactivated cells and RNA from differentiated or activated or pathological (e.g., transformed) or otherwise having a different metabolic state are separately hybridized against the affinity matrices to provide two pools of RNAs lacking the known RNA sequences.

In a preferred embodiment, the affinity matrix is packed into a columnar casing. The sample is then applied to the affinity matrix (e.g. injected onto a column or applied to a column by a pump such as a sampling pump driven by an autosampler). The affinity matrix (e.g. affinity column) bearing the sample is subjected to conditions under which the nucleic acid probes comprising the affinity matrix hybridize specifically with complementary target nucleic acids. Such conditions are accomplished by maintaining appropriate pH, salt and temperature conditions to facilitate hybridization as discussed above.

For a number of applications, it may be desirable to extract and separate messenger RNA from cells, cellular debris, and other contaminants. As such, the device of the present invention may, in some cases, include a mRNA purification chamber or channel. In general, such purification takes advantage of the poly-A tails on mRNA. In particular and as noted above, poly-T oligonucleotides may be immobilized within a chamber or channel of the device to serve as affinity ligands for mRNA. Poly-T oligonucleotides may be immobilized upon a solid support incorporated within the chamber or channel, or alternatively, may be immobilized upon the surface(s) of the chamber or channel itself. Immobilization of oligonucleotides on the surface of the chambers or channels may be carried out by methods described herein including, e.g., oxidation and silanation of the surface followed by standard DMT synthesis of the oligonucleotides.

In operation, the lysed sample is introduced to a high salt solution to increase the ionic strength for hybridization, whereupon the mRNA will hybridize to the immobilized poly-T. The mRNA bound to the immobilized poly-T oligonucleotides is then washed free in a low ionic strength buffer. The poly-T oligonucleotides may be immobilized upon poroussurfaces, e.g., porous silicon, zeolites silica xerogels, scintered particles, or other solid supports.

Following sample preparation, the sample can be subjected to one or more different analysis operations. A variety of analysis operations may generally be performed, including size based analysis using, e.g., microcapillary electrophoresis, and/or sequence based analysis using, e.g., hybridization to an oligonucleotide array.

In the latter case, the nucleic acid sample may be probed using an array of oligonucleotide probes. Oligonucleotide arrays generally include a substrate having a large number of positionally distinct oligonucleotide probes attached to the substrate. These arrays may be produced using mechanical or light directed synthesis methods which incorporate a combination of photolithographic methods and solid phase oligonucleotide synthesis methods.

The basic strategy for light directed synthesis of oligonucleotide arrays is as follows. The surface of a solid support, modified with photosensitive protecting groups is illuminated through a photolithographic mask, yielding reactive hydroxyl groups in the illuminated regions. A selected nucleotide, typically in the form of a 3′-O-phosphoramidite-activated deoxynucleoside (protected at the 5′ hydroxyl with a photosensitive protecting group), is then presented to the surface and coupling occurs at the sites that were exposed to light. Following capping and oxidation, the substrate is rinsed and the surface is illuminated through a second mask to expose additional hydroxyl groups for coupling. A second selected nucleotide (e.g., 5′-protected, 3′-O-phosphoramidite-activated deoxynucleoside) is presented to the surface. The selective deprotection and coupling cycles are repeated until the desired set of products is obtained. Since photolithography is used the process can be readily miniaturized to generate high density arrays of oligonucleotide probes. Furthermore, the sequence of the oligonucleotides at each site is known. See Pease et al. Mechanical synthesis methods are similar to the light directed methods except involving mechanical direction of fluids for deprotection and addition in the synthesis steps.

For some embodiments, oligonucleotide arrays may be prepared having all possible probes of a given length. The hybridization pattern of the target sequence on the array may be used to reconstruct the target DNA sequence. Hybridization analysis of large numbers of probes can be used to sequence long stretches of DNA or provide an oligonucleotide array which is specific and complementary to a particular nucleic acid sequence. For example, in particularly preferred aspects, the oligonucleotide array will contain oligonucleotide probes which are complementary to specific target sequences, and individual or multiple mutations of these. Such arrays are particularly useful in the diagnosis of specific disorders which are characterized by the presence of a particular nucleic acid sequence.

Following sample collection and nucleic acid extraction, the nucleic acid portion of the sample is typically subjected to one or more preparative reactions. These preparative reactions include in vitro transcription, labeling, fragmentation, amplification and other reactions. Nucleic acid amplification increases the number of copies of the target nucleic acid sequence of interest. A variety of amplification methods are suitable for use in the methods and device of the present invention, including for example, the polymerase chain reaction method or (PCR), the ligase chain reaction (LCR), self sustained sequence replication (3SR), and nucleic acid based sequence amplification (NASBA).

The latter two amplification methods involve isothermal reactions based on isothermal transcription, which produce both single stranded RNA (ssRNA) and double stranded DNA (dsDNA) as the amplification products in a ratio of approximately 30 or 100 to 1, respectively. As a result, where these latter methods are employed, sequence analysis may be carried out using either type of substrate, i.e. complementary to either DNA or RNA.

Frequently, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids.

### PCR

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high density array may then include probes specific to the internal standard for quantification of the amplified nucleic acid.

Thus, in one embodiment, this invention provides for a method of optimizing a probe set for detection of a particular gene. Generally, this method involves providing a high density array containing a multiplicity of probes of one or more particular length(s) that are complementary to subsequences of the mRNA transcribed by the target gene. In one embodiment the high density array may contain every probe of a particular length that is complementary to a particular mRNA. The probes of the high density array are then hybridized with their target nucleic acid alone and then hybridized with a high complexity, high concentration nucleic acid sample that does not contain the targets complementary to the probes. Thus, for example, where the target nucleic acid is an RNA, the probes are first hybridized with their target nucleic acid alone and then hybridized with RNA made from a cDNA library (e.g., reverse transcribed polyA.sup.+ mRNA) where the sense of the hybridized RNA is opposite that of the target nucleic acid (to insure that the high complexity sample does not contain targets for the probes). Those probes that show a strong hybridization signal with their target and little or no cross-hybridization with the high complexity sample are preferred probes for use in the high density arrays of this invention.

PCR amplification generally involves the use of one strand of the target nucleic acid sequence as a template for producing a large number of complements to that sequence. Generally, two primer sequences complementary to different ends of a segment of the complementary strands of the target sequence hybridize with their respective strands of the target sequence, and in the presence of polymerase enzymes and nucleoside triphosphates, the primers are extended along the target sequence. The extensions are melted from the target sequence and the process is repeated, this time with the additional copies of the target sequence synthesized in the preceding steps. PCR amplification typically involves repeated cycles of denaturation, hybridization and extension reactions to produce sufficient amounts of the target nucleic acid. The first step of each cycle of the PCR involves the separation of the nucleic acid duplex formed by the primer extension. Once the strands are separated, the next step in PCR involves hybridizing the separated strands with primers that flank the target sequence. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid.

In PCR methods, strand separation is normally achieved by heating the reaction to a sufficiently high temperature for a sufficient time to cause the denaturation of the duplex but not to cause an irreversible denaturation of the polymerase. Typical heat denaturation involves temperatures ranging from about 80.degree. C. to 105.degree. C. for times ranging from seconds to minutes. Strand separation, however, can be accomplished by any suitable denaturing method including physical, chemical, or enzymatic means. Strand separation may be induced by a helicase, for example, or an enzyme capable of exhibiting helicase activity.

In addition to PCR and IVT reactions, the methods and devices of the present invention are also applicable to a number of other reaction types, e.g., reverse transcription, nick translation, and the like.

The nucleic acids in a sample will generally be labeled to facilitate detection in subsequent steps. Labeling may be carried out during the amplification, in vitro transcription or nick translation processes. In particular, amplification, in vitro transcription or nick translation may incorporate a label into the amplified or transcribed sequence, either through the use of labeled primers or the incorporation of labeled dNTPs into the amplified sequence. Hybridization between the sample nucleic acid and the oligonucleotide probes upon the array is then detected, using, e.g., epifluorescence confocal microscopy. Typically, sample is mixed during hybridization to enhance hybridization of nucleic acids in the sample to nucleic acid probes on the array.

In some cases, hybridized oligonucleotides may be labeled following hybridization. For example, where biotin labeled dNTPs are used in, e.g. amplification or transcription, streptavidin linked reporter groups may be used to label hybridized complexes. Such operations are readily integratable into the systems of the present invention. Alternatively, the nucleic acids in the sample may be labeled following amplification. Post amplification labeling typically involves the covalent attachment of a particular detectable group upon the amplified sequences. Suitable labels or detectable groups include a variety of fluorescent or radioactive labeling groups well known in the art. These labels may also be coupled to the sequences using methods that are well known in the art.

Methods for detection depend upon the label selected. A fluorescent label is preferred because of its extreme sensitivity and simplicity. Standard labeling procedures are used to determine the positions where interactions between a sequence and a reagent take place. For example, if a target sequence is labeled and exposed to a matrix of different probes, only those locations where probes do interact with the target will exhibit any signal. Alternatively, other methods may be used to scan the matrix to determine where interaction takes place. Of course, the spectrum of interactions may be determined in a temporal manner by repeated scans of interactions which occur at each of a multiplicity of conditions. However, instead of testing each individual interaction separately, a multiplicity of sequence interactions may be simultaneously determined on a matrix.

Means of detecting labeled target (sample) nucleic acids hybridized to the probes of the high density array are known to those of skill in the art. Thus, for example, where a colorimetric label is used, simple visualization of the label is sufficient. Where a radioactive labeled probe is used, detection of the radiation (e.g with photographic film or a solid state detector) is sufficient.

In a preferred embodiment, however, the target nucleic acids are labeled with a fluorescent label and the localization of the label on the probe array is accomplished with fluorescent microscopy. The hybridized array is excited with a light source at the excitation wavelength of the particular fluorescent label and the resulting fluorescence at the emission wavelength is detected. In a particularly preferred embodiment, the excitation light source is a laser appropriate for the excitation of the fluorescent label.

The target polynucleotide may be labeled by any of a number of convenient detectable markers. A fluorescent label is preferred because it provides a very strong signal with low background. It is also optically detectable at high resolution and sensitivity through a quick scanning procedure. Other potential labeling moieties include, radioisotopes, chemiluminescent compounds, labeled binding proteins, heavy metal atoms, spectroscopic markers, magnetic labels, and linked enzymes.

Another method for labeling may bypass any label of the target sequence. The target may be exposed to the probes, and a double strand hybrid is formed at those positions only. Addition of a double strand specific reagent will detect where hybridization takes place. An intercalative dye such as ethidium bromide may be used as long as the probes themselves do not fold back on themselves to a significant extent forming hairpin loops. However, the length of the hairpin loops in short oligonucleotide probes would typically be insufficient to form a stable duplex.

Suitable chromogens will include molecules and compounds which absorb light in a distinctive range of wavelengths so that a color may be observed, or emit light when irradiated with radiation of a particular wave length or wave length range, e.g., fluorescers. Biliproteins, e.g., phycoerythrin, may also serve as labels.

A wide variety of suitable dyes are available, being primarily chosen to provide an intense color with minimal absorption by their surroundings. Illustrative dye types include quinoline dyes, triarylmethane dyes, acridine dyes, alizarine dyes, phthaleins, insect dyes, azo dyes, anthraquinoid dyes, cyanine dyes, phenazathionium dyes, and phenazoxonium dyes.

A wide variety of fluorescers may be employed either by themselves or in conjunction with quencher molecules. Fluorescers of interest fall into a variety of categories having certain primary functionalities. These primary functionalities include 1- and 2-aminonaphthalene, p,p′-diaminostilbenes, pyrenes, quaternary phenanthridine salts, 9-aminoacridines, p,p′-diaminobenzophenone imines, anthracenes, oxacarbocyanine, merocyanine, 3-aminoequilenin, perylene, bis-benzoxazole, bis-p-oxazolyl benzene, 1,2-benzophenazin, retinol, bis-3-aminopyridinium salts, hellebrigenin, tetracycline, sterophenol, benzimidzaolylphenylamine, 2-oxo-3-chromen, indole, xanthen, 7-hydroxycoumarin, phenoxazine, salicylate, strophanthidin, porphyrins, triarylmethanes and flavin. Individual fluorescent compounds which have functionalities for linking or which can be modified to incorporate such functionalities include, e.g., dansyl chloride; fluoresceins such as 3,6-dihydroxy-9-phenylxanthhydrol; rhodamineisothiocyanate; N-phenyl 1-amino-8-sulfonatonaphthalene; N-phenyl 2-amino-6-sulfonatonaphthalene; 4-acetamido-4-isothiocyanato-stilbene-2,2′-disulfonic acid; pyrene-3-sulfonic acid; 2-toluidinonaphthalene-6-sulfonate; N-phenyl, N-methyl 2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine; N,N′-dihexyl oxacarbocyanine; merocyanine, 4-(3′pyrenyl)butyrate; d-3-aminodesoxy-equilenin; 12-(9′-anthroyl)stearate; 2-methylanthracene; 9-vinylanthracene; 2,2′-(vinylene-p-phenylene)bisbenzoxazole; p-bis>2-(4-methyl-5-phenyl-oxazolyl)!benzene; 6-dimethylamino-1,2-benzophenazin; retinol; bis(3′-aminopyridinium) 1,10-decandiyl diiodide; sulfonaphthylhydrazone of hellibrienin; chlorotetracycline; N-(7-dimethylamino-4-methyl-2-oxo-3-chromenyl)maleimide; N->p-(2-benzimidazolyl)-phenyl!maleimide; N-(4-fluoranthyl)maleimide; bis(homovanillic acid); resazarin; 4-chloro-7-nitro-2,1,3-benzooxadiazole; merocyanine 540; resorufin; rose bengal; and 2,4-diphenyl-3(2H)-furanone.

Desirably, fluorescers should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye may differ from the unbound dye. Therefore, when referring to the various wavelength ranges and characteristics of the dyes, it is intended to indicate the dyes as employed and not the dye which is unconjugated and characterized in an arbitrary solvent.

Fluorescers are generally preferred because by irradiating a fluorescer with light, one can obtain a plurality of emissions. Thus, a single label can provide for a plurality of measurable events.

Detectable signal may also be provided by chemiluminescent and bioluminescent sources. Chemiluminescent sources include a compound which becomes electronically excited by a chemical reaction and may then emit light which serves as the detectable signal or donates energy to a fluorescent acceptor. A diverse number of families of compounds have been found to provide chemiluminescence under a variety of conditions. One family of compounds is 2,3-dihydro-1,-4-phthalazinedione. The most popular compound is luminol, which is the 5-amino compound. Other members of the family include the 5-amino-6,7,8-trimethoxy- and the dimethylamino>ca!benz analog. These compounds can be made to luminesce with alkaline hydrogen peroxide or calcium hypochlorite and base. Another family of compounds is the 2,4,5-triphenylimidazoles, with lophine as the common name for the parent product. Chemiluminescent analogs include para-dimethylamino and -methoxy substituents. Chemiluminescence may also be obtained with oxalates, usually oxalyl active esters, e.g., p-nitrophenyl and a peroxide, e.g., hydrogen peroxide, under basic conditions. Alternatively, luciferins may be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

In addition, amplified sequences may be subjected to other post amplification treatments. For example, in some cases, it may be desirable to fragment the sequence prior to hybridization with an oligonucleotide array, in order to provide segments which are more readily accessible to the probes, which avoid looping and/or hybridization to multiple probes. Fragmentation of the nucleic acids may generally be carried out by physical, chemical or enzymatic methods that are known in the art.

Following the various sample preparation operations, the sample will generally be subjected to one or more analysis operations. Particularly preferred analysis operations include, e.g. sequence based analyses using an oligonucleotide array and/or size based analyses using, e.g. microcapillary array electrophoresis.

In some embodiments it may be desirable to provide an additional, or alternative means for analyzing the nucleic acids from the sample

Microcapillary array electrophoresis generally involves the use of a thin capillary or channel which may or may not be filled with a particular separation medium. Electrophoresis of a sample through the capillary provides a size based separation profile for the sample. Microcapillary array electrophoresis generally provides a rapid method for size based sequencing, PCR product analysis and restriction fragment sizing. The high surface to volume ratio of these capillaries allows for the application of higher electric fields across the capillary without substantial thermal variation across the capillary, consequently allowing for more rapid separations. Furthermore, when combined with confocal imaging methods these methods provide sensitivity in the range of attomoles, which is comparable to the sensitivity of radioactive sequencing methods.

In many capillary electrophoresis methods, the capillaries e.g. fused silica capillaries or channels etched, machined or molded into planar substrates, are filled with an appropriate separation/sieving matrix. Typically, a variety of sieving matrices are known in the art may be used in the microcapillary arrays. Examples of such matrices include, e.g. hydroxyethyl cellulose, polyacrylamide and agarose. Gel matrices may be introduced and polymerized within the capillary channel. However, in some cases this may result in entrapment of bubbles within the channels which can interfere with sample separations. Accordingly, it is often desirable to place a preformed separation matrix within the capillary channel(s), prior to mating the planar elements of the capillary portion. Fixing the two parts, e.g. through sonic welding, permanently fixes the matrix within the channel. Polymerization outside of the channels helps to ensure that no bubbles are formed. Further, the pressure of the welding process helps to ensure a void-free system.

In addition to its use in nucleic acid “fingerprinting” and other sized based analyses the capillary arrays may also be used in sequencing applications. In particular, gel based sequencing techniques may be readily adapted for capillary array electrophoresis.

In addition to detection of mRNA or as the sole detection method expression products from the genes discussed above may be detected as indications of the biological condition of the tissue. Expression products may be detected in either the tissue sample as such, or in a body fluid sample, such as blood, serum, plasma, faeces, mucus, sputum, cerebrospinal fluid, and/or urine of the individual.

The expression products, peptides and proteins, may be detected by any suitable technique known to the person skilled in the art.

In a preferred embodiment the expression products are detected by means of specific antibodies directed to the various expression products, such as immunofluorescent and/or immunohistochemical staining of the tissue.

Immunohistochemical localization of expressed proteins may be carried out by immunostaining of tissue sections from the single tumors to determine which cells expressed the protein encoded by the transcript in question. The transcript levels may be used to select a group of proteins supposed to show variation from sample to sample making a rough correlation between the level of protein detected and the intensity of the transcript on the microarray possible.

For example sections may be cut from paraffin-embedded tissue blocks, mounted, and deparaffinized by incubation at 80° C. for 10 min. followed by immersion in heated oil at 60° C. for 10 min. (Estisol 312, Estichem A/S, Denmark) and rehydration. Antigen retrieval is achieved in TEG (TrisEDTA-Glycerol) buffer using microwaves at 900 W. The tissue sections may be cooled in the buffer for 15 min before a brief rinse in tap water. Endogenous peroxidase activity is blocked by incubating the sections with 1% H₂O₂ for 20 min. followed by three rinses in tap water, 1 min each. The sections may then be soaked in PBS buffer for 2 min. The next steps can be modified from the descriptions given by Oncogene Science Inc., in the Mouse Immunohistochemistry Detection System, XHCO1 (UniTect, Uniondale, N.Y., USA). Briefly, the tissue sections are incubated overnight at 4° C. with primary antibody (against beta-2 microglobulin (Dako), cytokeratin 8, cystatin-C (both from Europa, US), junB, CD59, E-cadherin, apo-E, cathepsin E, vimentin, IGFII (all from Santa Cruz), followed by three rinses in PBS buffer for 5 min each. Afterwards, the sections are incubated with biotinylated secondary antibody for 30 min, rinsed three times with PBS buffer and subsequently incubated with ABC (avidin-biotinlylated horseradish peroxidase complex) for 30 min. followed by three rinses in PBS buffer.

Staining may be performed by incubation with AEC (3-amino-ethylcarbazole) for 10 min. The tissue sections are counter stained with Mayers hematoxylin, washed in tap water for 5 min. and mounted with glycerol-gelatin. Positive and negative controls may be included in each staining round with all antibodies.

In yet another embodiment the expression products may be detected by means of conventional enzyme assays, such as ELISA methods.

Furthermore, the expression products may be detected by means of peptide/protein chips capable of specifically binding the peptides and/or proteins assessed. Thereby an expression pattern may be obtained.

### Assay

In a further aspect the invention relates to an assay for predicting the prognosis of a biological condition in animal tissue, comprising
at least one first marker capable of detecting an expression level of at least one gene selected from the group of genes consisting of gene No. 1 to gene No. 562.

Preferably the assay further comprises means for correlating the expression level to at least one standard expression level and/or at least one reference pattern.

The means for correlating preferably includes one or more standard expression levels and/or reference patterns for use in comparing or correlating the expression levels or patterns obtained from a tumor under examination to the standards.

Preferably the invention relates to an assay for determining an expression pattern of a bladder cell, comprising at least a first marker and/or a second marker, wherein the first marker is capable of detecting a gene from a first gene group as defined above, and/or the second marker is capable of detecting a gene from a second gene group as defined above, correlating the first expression level and/or the second expression level to a standard level of the assessed genes to predict the prognosis of a biological condition in the animal tissue. The marker(s) are preferably specifically detecting a gene as identified herein.

As described above, it is preferred to determine the expression level from more than one gene, and correspondingly, it is preferred to include more than one marker in the assay, such as at least two markers, such as at least three markers, such as at least four markers, such as at least five markers, such as at least six markers, such as at least seven markers, such as at least eight markers, such as at least nine markers, such as at least ten markers, such as at least 15 markers.

When using markers for at least two different groups, it is preferred that the above number of markers relate to markers in each group.

As discussed above the marker may be any nucleotide probe, such as a DNA, RNA, PNA, or LNA probe capable of hybridising to mRNA indicative of the expression level. The hybridisation conditions are preferably as described below for probes. In another embodiment the marker is an antibody capable of specifically binding the expression product in question.

Patterns can be compared manually by a person or by a computer or other machine. An algorithm can be used to detect similarities and differences. The algorithm may score and compare, for example, the genes which are expressed and the genes which are not expressed. Alternatively, the algorithm may look for changes in intensity of expression of a particular gene and score changes in intensity between two samples. Similarities may be determined on the basis of genes which are expressed in both samples and genes which are not expressed in both samples or on the basis of genes whose intensity of expression are numerically similar.

Generally, the detection operation will be performed using a reader device external to the diagnostic device. However, it may be desirable in some cases to incorporate the data gathering operation into the diagnostic device itself.

The detection apparatus may be a fluorescence detector, or a spectroscopic detector, or another detector.

Although hybridization is one type of specific interaction which is clearly useful for use in this mapping embodiment antibody reagents may also be very useful.

Gathering data from the various analysis operations, e.g. oligonucleotide and/or microcapillary arrays will typically be carried out using methods known in the art. For example, the arrays may be scanned using lasers to excite fluorescently labeled targets that have hybridized to regions of probe arrays mentioned above, which can then be imaged using charged coupled devices (“CCDs”) for a wide field scanning of the array. Alternatively, another particularly useful method for gathering data from the arrays is through the use of laser confocal microscopy which combines the ease and speed of a readily automated process with high resolution detection.

Following the data gathering operation, the data will typically be reported to a data analysis operation. To facilitate the sample analysis operation, the data obtained by the reader from the device will typically be analyzed using a digital computer. Typically, the computer will be appropriately programmed for receipt and storage of the data from the device, as well as for analysis and reporting of the data gathered, i.e., interpreting fluorescence data to determine the sequence of hybridizing probes, normalization of background and single base mismatch hybridizations, ordering of sequence data in SBH applications, and the like.

The invention also relates to a pharmaceutical composition for treating a biological condition, such as bladder tumors.

In one embodiment the pharmaceutical composition comprises one or more of the peptides being expression products as defined above. In a preferred embodiment, the peptides are bound to carriers. The peptides may suitably be coupled to a polymer carrier, for example a protein carrier, such as BSA. Such formulations are well-known to the person skilled in the art.

The peptides may be suppressor peptides normally lost or decreased in tumor tissue administered in order to stabilise tumors towards a less malignant stage. In another embodiment the peptides are onco-peptides capable of eliciting an immune response towards the tumor cells.

In another embodiment the pharmaceutical composition comprises genetic material, either genetic material for substitution therapy, or for suppressing therapy as discussed below.

In a third embodiment the pharmaceutical composition comprises at least one antibody produced as described above.

In the present context the term pharmaceutical composition is used synonymously with the term medicament. The medicament of the invention comprises an effective amount of one or more of the compounds as defined above, or a composition as defined above in combination with pharmaceutically acceptable additives. Such medicament may suitably be formulated for oral, percutaneous, intramuscular, intravenous, intracranial, intrathecal, intracerebroventricular, intranasal or pulmonal administration. For most indications a localised or substantially localised application is preferred.

Strategies in formulation development of medicaments and compositions based on the compounds of the present invention generally correspond to formulation strategies for any other protein-based drug product. Potential problems and the guidance required to overcome these problems are dealt with in several textbooks, e.g. “Therapeutic Peptides and Protein Formulation. Processing and Delivery Systems”, Ed. A. K. Banga, Technomic Publishing AG, Basel, 1995.

Injectables are usually prepared either as liquid solutions or suspensions, solid forms suitable for solution in, or suspension in, liquid prior to injection. The preparation may also be emulsified. The active ingredient is often mixed with excipients which are pharmaceutically acceptable and compatible with the active ingredient. Suitable excipients are, for example, water, saline, dextrose, glycerol, ethanol or the like, and combinations thereof. In addition, if desired, the preparation may contain minor amounts of auxiliary substances such as wetting or emulsifying agents, pH buffering agents, or which enhance the effectiveness or transportation of the preparation.

Formulations of the compounds of the invention can be prepared by techniques known to the person skilled in the art. The formulations may contain pharmaceutically acceptable carriers and excipients including microspheres, liposomes, microcapsules and nanoparticles.

The preparation may suitably be administered by injection, optionally at the site, where the active ingredient is to exert its effect. Additional formulations which are suitable for other modes of administration include suppositories, and in some cases, oral formulations. For suppositories, traditional binders and carriers include polyalkylene glycols or triglycerides. Such suppositories may be formed from mixtures containing the active ingredient(s) in the range of from 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and generally contain 10-95% of the active ingredient(s), preferably 25-70%.

The preparations are administered in a manner compatible with the dosage formulation, and in such amount as will be therapeutically effective. The quantity to be administered depends on the subject to be treated, including, e.g. the weight and age of the subject, the disease to be treated and the stage of disease. Suitable dosage ranges are of the order of several hundred μg active ingredient per administration with a preferred range of from about 0.1 μg to 1000 μg, such as in the range of from about 1 μg to 300 μg, and especially in the range of from about 10 μg to 50 μg. Administration may be performed once or may be followed by subsequent administrations. The dosage will also depend on the route of administration and will vary with the age and weight of the subject to be treated. A preferred dosis would be in the interval 30 mg to 70 mg per 70 kg body weight.

Some of the compounds of the present invention are sufficiently active, but for some of the others, the effect will be enhanced if the preparation further comprises pharmaceutically acceptable additives and/or carriers. Such additives and carriers will be known in the art. In some cases, it will be advantageous to include a compound, which promote delivery of the active substance to its target.

In many instances, it will be necessary to administrate the formulation multiple times. Administration may be a continuous infusion, such as intraventricular infusion or administration in more doses such as more times a day, daily, more times a week, weekly, etc.

### Vaccines

In a further embodiment the present invention relates to a vaccine for the prophylaxis or treatment of a biological condition comprising at least one expression product from at least one gene said gene being expressed as defined above.

The term vaccines is used with its normal meaning, i.e preparations of immunogenic material for administration to induce in the recipient an immunity to infection or intoxication by a given infecting agent. Vaccines may be administered by intravenous injection or through oral, nasal and/or mucosal administration. Vaccines may be either simple vaccines prepared from one species of expression products, such as proteins or peptides, or a variety of expression products, or they may be mixed vaccines containing two or more simple vaccines. They are prepared in such a manner as not to destroy the immunogenic material, although the methods of preparation vary, depending on the vaccine.

The enhanced immune response achieved according to the invention can be attributable to e.g. an enhanced increase in the level of immunoglobulins or in the level of T-cells including cytotoxic T-cells will result in immunisation of at least 50% of individuals exposed to said immunogenic composition or vaccine, such as at least 55%, for example at least 60%, such as at least 65%, for example at least 70%, for example at least 75%, such as at least 80%, for example at least 85%, such as at least 90%, for example at least 92%, such as at least 94%, for example at least 96%, such as at least 97%, for example at least 98%, such as at least 98.5%, for example at least 99%, for example at least 99.5% of the individuals exposed to said immunogenic composition or vaccine are immunised.

Compositions according to the invention may also comprise any carrier and/or adjuvant known in the art including functional equivalents thereof. Functionally equivalent carriers are capable of presenting the same immunogenic determinant in essentially the same steric conformation when used under similar conditions. Functionally equivalent adjuvants are capable of providing similar increases in the efficacy of the composition when used under similar conditions.

### Therapy

The invention further relates to a method of treating individuals suffering from the biological condition in question, in particular for treating a bladder tumor.

Accordingly, the invention relates to a method for reducing cell tumorigenicity or malignancy of a cell, said method comprising contacting a tumor cell with at least one peptide expressed by at least one gene selected from the group of genes consisting of gene No. 200-214, 233, 234, 235, 236, 244, 249, 251, 252, 255, 256, 259, 261, 262, 266, 268, 269, 273, 274, 275, 276, 277, 279, 280, 281, 282, 285, 286, 289, 293, 295, 296, 299, 301, 304, 306, 307, 308, 311, 312, 313, 314, 320, 322, 323, 325, 326, 327, 328, 330, 331, 332, 333, 334, 338, 341, 342, 343, 345, 348, 349, 350, 351, 352, 353, 355, 357, 360, 361, 363, 366, 367, 370, 373, 374, 375, 376, 385, 386, 387, 389, 390, 392, 394, 398, 400, 401, 405, 406, 407, 408, 410, 411, 412, 414, 415, 416, 418, 424, 426, 428, 433, 434, 435, 436, 438, 439, 440, 441, 442, 443, 445, 446, 453, 460, 461, 463, 464, 465, 466, 467, 469, 470, 471, 472, 473, 475, 476, 477, 479, 480, 481, 482, 483, 485, 486, 487, 488, 490, 492, 494, 496, 497, 498, 499, 503, 515, 516, 517, 521, 526, 527, 528, 530, 532, 533, 537, 539, 540, 541, 542, 543, 545, 554, 557, 560.

In order to increase the effect several different peptides may be used simultaneously, such as wherein the tumor cell is contacted with at least two different peptides.

In one embodiment the invention relates to a method of substitution therapy, ie. administration of genetic material generally expressed in normal cells, but lost or decreased in biological condition cells (tumor suppressors). Thus, the invention relates to a method for reducing cell tumorigenicity or malignancy of a cell, said method comprising

obtaining at least one gene selected from the group of genes consisting of gene No. 200-214, 233, 234, 235, 236, 244, 249, 251, 252, 255, 256, 259, 261, 262, 266, 268, 269, 273, 274, 275, 276, 277, 279, 280, 281, 282, 285, 286, 289, 293, 295, 296, 299, 301, 304, 306, 307, 308, 311, 312, 313, 314, 320, 322, 323, 325, 326, 327, 328, 330, 331, 332, 333, 334, 338, 341, 342, 343, 345, 348, 349, 350, 351, 352, 353, 355, 357, 360, 361, 363, 366, 367, 370, 373, 374, 375, 376, 385, 386, 387, 389, 390, 392, 394, 398, 400, 401, 405, 406, 407, 408, 410, 411, 412, 414, 415, 416, 418, 424, 426, 428, 433, 434, 435, 436, 438, 439, 440, 441, 442, 443, 445, 446, 453, 460, 461, 463, 464, 465, 466, 467, 469, 470, 471, 472, 473, 475, 476, 477, 479, 480, 481, 482, 483, 485, 486, 487, 488, 490, 492, 494, 496, 497, 498, 499, 503, 515, 516, 517, 521, 526, 527, 528, 530, 532, 533, 537, 539, 540, 541, 542, 543, 545, 554, 557, 560,
introducing said at least one gene into the tumor cell in a manner allowing expression of said gene(s).

In one embodiment at least one gene is introduced into the tumor cell. In another embodiment at least two genes are introduced into the tumor cell.

In one aspect of the invention small molecules that either inhibit increased gene expression or their effects or substitute decreased gene expression or their effects, are introduced to the cellular environment or the cells. Application of small molecules to tumor cells may be performed by e.g. local application or intravenous injection or by oral ingestion. Small molecules have the ability to restore function of reduced gene expression in tumor or cancer tissue.

In another aspect the invention relates to a therapy whereby genes (increase and/or decrease) generally are correlated to disease are inhibited by one or more of the following methods:

A method for reducing cell tumorigenicity or malignancy of a cell, said method comprising
obtaining at least one nucleotide probe capable of hybridising with at least one gene of a tumor cell, said at least one gene being selected from the group of genes consisting of gene Nos. 1-199, 215-232, 237, 238, 239, 240, 241, 242, 243, 245, 246, 247, 248, 250, 253, 254, 257, 258, 260, 263, 264, 265, 267, 270, 271, 272, 278, 283, 284, 287, 288, 290, 291, 292, 294, 297, 298, 300, 302, 303, 305, 309, 310, 315, 316, 317, 318, 319, 321, 324, 329, 335, 336, 337, 339, 340, 344, 346, 347, 354, 356, 358, 359, 362, 364, 365, 368, 369, 371, 372, 377, 378, 379, 380, 381, 382, 383, 384, 388, 391, 393, 395, 396, 397, 399, 402, 403, 404, 409, 413, 417, 419, 420, 421, 422, 423, 425, 427, 429, 430, 431, 432, 437, 444, 447, 448, 449, 450, 451, 452, 454, 455, 456, 457, 458, 459, 462, 468, 474, 478, 484, 489, 491, 493, 495, 500, 501, 502, 504, 505, 506, 507, 508, 509, 510, 511, 512, 513, 514, 518, 519, 520, 522, 523, 524, 525, 529, 531, 534, 535, 536, 538, 544, 546, 547, 548, 549, 550, 551, 552, 553, 555, 556, 558, 559, 561, 562,
introducing said at least one nucleotide probe into the tumor cell in a manner allowing the probe to hybridise to the at least one gene, thereby inhibiting expression of said at least one gene. This method is preferably based on anti-sense technology, whereby the hybridisation of said probe to the gene leads to a down-regulation of said gene.

In another preferred embodiment, the method for reducing cell tumorigenicity or malignancy of a cell is based on RNA interference, comprising small interfering RNAs (siRNAs) specifically directed against at least one gene being selected from the group of genes consisting of gene Nos. 1-199, 215-232, 237, 238, 239, 240, 241, 242, 243, 245, 246, 247, 248, 250, 253, 254, 257, 258, 260, 263, 264, 265, 267, 270, 271, 272, 278, 283, 284, 287, 288, 290, 291, 292, 294, 297, 298, 300, 302, 303, 305, 309, 310, 315, 316, 317, 318, 319, 321, 324, 329, 335, 336, 337, 339, 340, 344, 346, 347, 354, 356, 358, 359, 362, 364, 365, 368, 369, 371, 372, 377, 378, 379, 380, 381, 382, 383, 384, 388, 391, 393, 395, 396, 397, 399, 402, 403, 404, 409, 413, 417, 419, 420, 421, 422, 423, 425, 427, 429, 430, 431, 432, 437, 444, 447, 448, 449, 450, 451, 452, 454, 455, 456, 457, 458, 459, 462, 468, 474, 478, 484, 489, 491, 493, 495, 500, 501, 502, 504, 505, 506, 507, 508, 509, 510, 511, 512, 513, 514, 518, 519, 520, 522, 523, 524, 525, 529, 531, 534, 535, 536, 538, 544, 546, 547, 548, 549, 550, 551, 552, 553, 555, 556, 558, 559, 561, 562.

The down-regulation may of course also be based on a probe capable of hybridising to regulatory components of the genes in question, such as promoters.

The hybridization may be tested in vitro at conditions corresponding to in vivo conditions. Typically, hybridization conditions are of low to moderate stringency. These conditions favour specific interactions between completely complementary sequences, but allow some non-specific interaction between less than perfectly matched sequences to occur as well. After hybridization, the nucleic acids can be “washed” under moderate or high conditions of stringency to dissociate duplexes that are bound together by some non-specific interaction (the nucleic acids that form these duplexes are thus not completely complementary).

As is known in the art, the optimal conditions for washing are determined empirically, often by gradually increasing the stringency. The parameters that can be changed to affect stringency include, primarily, temperature and salt concentration. In general, the lower the salt concentration and the higher the temperature the higher the stringency. Washing can be initiated at a low temperature (for example, room temperature) using a solution containing a salt concentration that is equivalent to or lower than that of the hybridization solution. Sub-sequent washing can be carried out using progressively warmer solutions having the same salt concentration. As alternatives, the salt concentration can be lowered and the temperature maintained in the washing step, or the salt concentration can be lowered and the temperature increased. Additional parameters can also be altered. For example, use of a destabilizing agent, such as formamide, alters the stringency conditions.

In reactions where nucleic acids are hybridized, the conditions used to achieve a given level of stringency will vary. There is not one set of conditions, for example, that will allow duplexes to form between all nucleic acids that are 85% identical to one another; hybridization also depends on unique features of each nucleic acid. The length of the sequence, the composition of the sequence (for example, the content of purine-like nucleotides versus the content of pyrimidine-like nucleotides) and the type of nucleic acid (for example, DNA or RNA) affect hybridization. An additional consideration is whether one of the nucleic acids is immobilized (for example on a filter).

An example of a progression from lower to higher stringency conditions is the following, where the salt content is given as the relative abundance of SSC (a salt solution containing sodium chloride and sodium citrate; 2×SSC is 10-fold more concentrated than 0.2×SSC). Nucleic acids are hybridized at 42° C. in 2×SSC/0.1% SDS (sodium dodecylsulfate; a detergent) and then washed in 0.2×SSC/0.1% SDS at room temperature (for conditions of low stringency); 0.2×SSC/0.1% SDS at 42° C. (for conditions of moderate stringency); and 0.1×SSC at 68° C. (for conditions of high stringency). Washing can be carried out using only one of the conditions given, or each of the conditions can be used (for example, washing for 10-15 minutes each in the order listed above). Any or all of the washes can be repeated. As mentioned above, optimal conditions will vary and can be determined empirically.

In another aspect a method of reducing tumoregeneicity relates to the use of antibodies against an expression product of a cell from the biological tissue. The antibodies may be produced by any suitable method, such as a method comprising the steps of

obtaining expression product(s) from at least one gene said gene being expressed as defined above,
immunising a mammal with said expression product(s) obtaining antibodies against the expression product.

### Use

The methods described above may be used for producing an assay for diagnosing a biological condition in animal tissue, or for identification of the origin of a piece of tissue. Further, the methods of the invention may be used for prediction of a disease course and treatment response.

Furthermore, the invention relates to the use of a peptide as defined above for preparation of a pharmaceutical composition for the treatment of a biological condition in animal tissue.

Furthermore, the invention relates to the use of a gene as defined above for preparation of a pharmaceutical composition for the treatment of a biological condition in animal tissue.

Also, the invention relates to the use of a probe as defined above for preparation of a pharmaceutical composition for the treatment of a biological condition in animal tissue.

The genetic material discussed above for may be any of the described genes or functional parts thereof. The constructs may be introduced as a single DNA molecule encoding all of the genes, or different DNA molecules having one or more genes. The constructs may be introduced simultaneously or consecutively, each with the same or different markers.

The gene may be linked to the complex as such or protected by any suitable system normally used for transfection such as viral vectors or artificial viral envelope, liposomes or micellas, wherein the system is linked to the complex.

Numerous techniques for introducing DNA into eukaryotic cells are known to the skilled artisan. Often this is done by means of vectors, and often in the form of nucleic acid encapsidated by a (frequently virus-like) proteinaceous coat. Gene delivery systems may be applied to a wide range of clinical as well as experimental applications.

Vectors containing useful elements such as selectable and/or amplifiable markers, promoter/enhancer elements for expression in mammalian, particularly human, cells, and which may be used to prepare stocks of construct DNAs and for carrying out transfections are well known in the art. Many are commercially available.

Various techniques have been developed for modification of target tissue and cells in vivo. A number of virus vectors, discussed below, are known which allow transfection and random integration of the virus into the host. See, for example, Dubensky et al. (1984) Proc. Natl. Acad. Sci. USA 81:7529-7533; Kaneda et al., (1989) Science 243:375-378; Hiebert et al. (1989) Proc. Natl. Acad. Sci. USA 86:3594-3598; Hatzoglu et al., (1990) J. Biol. Chem. 265:17285-17293; Ferry et al. (1991) Proc. Natl. Acad. Sci. USA 88:8377-8381. Routes and modes of administering the vector include injection, e.g intravascularly or intramuscularly, inhalation, or other parenteral administration.

Advantages of adenovirus vectors for human gene therapy include the fact that recombination is rare, no human malignancies are known to be associated with such viruses, the adenovirus genome is double stranded DNA which can be manipulated to accept foreign genes of up to 7.5 kb in size, and live adenovirus is a safe human vaccine organisms.

Another vector which can express the DNA molecule of the present invention, and is useful in gene therapy, particularly in humans, is vaccinia virus, which can be rendered non-replicating (U.S. Pat. Nos. 5,225,336; 5,204,243; 5,155,020; 4,769,330).

Based on the concept of viral mimicry, artificial viral envelopes (AVE) are designed based on the structure and composition of a viral membrane, such as HIV-1 or RSV and used to deliver genes into cells in vitro and in vivo. See, for example, U.S. Pat. No. 5,252,348, Schreier H. et al., J. Mol. Recognit., 1995, 8:59-62; Schreier H et al., J. Biol. Chem., 1994, 269:9090-9098; Schreier, H., Pharm. Acta Helv. 1994, 68:145-159; Chander, R et al. Life Sci., 1992, 50:481-489, which references are hereby incorporated by reference in their entirety. The envelope is preferably produced in a two-step dialysis procedure where the “naked” envelope is formed initially, followed by unidirectional insertion of the viral surface glycoprotein of interest. This process and the physical characteristics of the resulting AVE are described in detail by Chander et al., (supra). Examples of AVE systems are (a) an AVE containing the HIV-1 surface glycoprotein gp160 (Chander et al., supra; Schreier et al., 1995, supra) or glycosyl phosphatidylinositol (GPI)-linked gp120 (Schreier et al., 1994, supra), respectively, and (b) an AVE containing the respiratory syncytial virus (RSV) attachment (G) and fusion (F) glycoproteins (Stecenko, A. A. et al., Pharm. Pharmacol. Lett. 1:127-129 (1992)). Thus, vesicles are constructed which mimic the natural membranes of enveloped viruses in their ability to bind to and deliver materials to cells bearing corresponding surface receptors.

AVEs are used to deliver genes both by intravenous injection and by instillation in the lungs. For example, AVEs are manufactured to mimic RSV, exhibiting the RSV F surface glycoprotein which provides selective entry into epithelial cells. F-AVE are loaded with a plasmid coding for the gene of interest, (or a reporter gene such as CAT not present in mammalian tissue).

The AVE system described herein in physically and chemically essentially identical to the natural virus yet is entirely “artificial”, as it is constructed from phospholipids, cholesterol, and recombinant viral surface glycoproteins. Hence, there is no carry-over of viral genetic information and no danger of inadvertant viral infection. Construction of the AVEs in two independent steps allows for bulk production of the plain lipid envelopes which, in a separate second step, can then be marked with the desired viral glycoprotein, also allowing for the preparation of protein cocktail formulations if desired.

Another delivery vehicle for use in the present invention are based on the recent description of attenuated Shigella as a DNA delivery system (Sizemore, D. R. et al., Science 270:299-302 (1995), which reference is incorporated by reference in its entirety). This approach exploits the ability of Shigellae to enter epithelial cells and escape the phagocytic vacuole as a method for delivering the gene construct into the cytoplasm of the target cell. Invasion with as few as one to five bacteria can result in expression of the foreign plasmid DNA delivered by these bacteria.

A preferred type of mediator of nonviral transfection in vitro and in vivo is cationic (ammonium derivatized) lipids. These positively charged lipids form complexes with negatively charged DNA, resulting in DNA charged neutralization and compaction. The complexes endocytosed upon association with the cell membrane, and the DNA somehow escapes the endosome, gaining access to the cytoplasm. Cationic lipid:DNA complexes appear highly stable under normal conditions. Studies of the cationic lipid DOTAP suggest the complex dissociates when the inner layer of the cell membrane is destabilized and anionic lipids from the inner layer displace DNA from the cationic lipid. Several cationic lipids are available commercially. Two of these, DMRI and DC-cholesterol, have been used in human clinical trials. First generation cationic lipids are less efficient than viral vectors. For delivery to lung, any inflammatory responses accompanying the liposome administration are reduced by changing the delivery mode to aerosol administration which distributes the dose more evenly.

### Drug Screening

Genes identified as changing in various stages of bladder cancer can be used as markers for drug screening. Thus by treating bladder cancer cells with test compounds or extracts, and monitoring the expression of genes identified as changing in the progression of bladder cancers, one can identify compounds or extracts which change expression of genes to a pattern which is of an earlier stage or even of normal bladder mucosa.

It is also within the scope of the invention to use small molecules in drug screening.

The following are non-limiting examples illustrating the present invention.

### EXAMPLES

### Example 1

### Identification of a Molecular Signature Defining Disease Progression in Patients with Superficial Bladder Carcinoma

### Patient Samples

Bladder tumor biopsies were obtained directly from surgery after removal of the necessary amount of tissue for routine pathology examination. The tumors were frozen at −80° C. in a guanidinium thiocyanate solution for preservation of the RNA. Informed consent was obtained in all cases, and the protocols were approved by the scientific ethical committee of Aarhus County. The samples for the no progression group were selected by the following criteria: a) Ta or T1 tumors with no prior higher stage tumors; b) a minimum follow up period of 12 months to the most recent routine cystoscopy examination of the bladder with no occurrence of tumors of higher stage. The samples for the progression group were selected by two criteria: a) Ta or T1 tumors with no prior higher stage tumors; b) subsequent progression to a higher stage tumor, see Table 1.

TABLE 1
Clinical data on all patients involved in the study

| | | | | | |
|---|---|---|---|---|---|
| | | | | | Follow- |
| | | | Progressed | Time to | up time |
| Group | Sample | Hist. | to: | progression | months |

Training set

| | | | | | |
|---|---|---|---|---|---|
| No prog. | 150-6 | Ta gr3 | — | — | 44 |
| No prog. | 997-1 | Ta gr2 | — | — | 24 |
| No prog. | 833-2 | Ta gr3 | — | — | 35 |
| No prog. | 1070-1 | Ta gr3 | — | — | 33 |
| No prog. | 968-1 | Ta gr2 | — | — | 26 |
| No prog. | 625-1 | T1 gr3 | — | — | 12 |
| No prog. | 880-1 | T1 gr3 | — | — | 47 |
| No prog. | 815-1 | Ta gr2 | — | — | 49 |
| No prog. | 861-1 | Ta gr2 | — | — | 45 |
| No prog. | 669-1 | Ta gr2 | — | — | 55 |
| No prog. | 368-4 | Ta gr2 | — | — | 16 |
| No prog. | 898-1 | Ta gr2 | — | — | 17 |
| No prog. | 576-6 | Ta gr2 | — | — | 36 |
| Prog. | 747-3 | Ta gr2 | T1 gr3 | 6 | |
| Prog. | 956-2 | Ta gr3 | T1 gr3 | 27 | — |
| Prog. | 1083-1 | Ta gr2 | T1 gr3 | 1 | — |
| Prog. | 686-3 | Ta gr2 | T1 gr2 | 6 | — |
| Prog. | 795-13 | Ta gr2 | T1 gr3 | 4 | — |
| Prog. | 865-1 | Ta gr2 | T1 gr2 | 5 | — |
| Prog. | 112-2 | Ta gr3 | T1 gr3 | 7 | — |
| Prog. | 825-3 | Ta gr3 | T1 gr3 | 6 | — |
| Prog. | 679-2 | Ta gr2 | T2+ gr3 | 31 | — |
| Prog. | 941-4 | Ta gr3 | T2+ gr3 | 10 | — |
| Prog. | 607-1 | T1 gr2 | T2+ gr3 | 3 | — |
| Prog. | 1017-1 | T1 gr3 | T2+ gr3 | 8 | — |
| Prog. | 1276-1 | T1 gr3 | T2+ gr3 | 7 | — |
| Prog. | 501-1 | T1 gr3 | T2+ gr3 | 26 | — |
| Prog. | 744-1 | T1 gr3 | T2+ gr3 | 14 | — |
| Prog. | 839-1 | T1 gr3 | T2+ gr3 | 12 | — |

Test set

| | | | | | |
|---|---|---|---|---|---|
| No prog. | 1008-1 | Ta gr2 | — | — | 55 |
| No prog. | 1060-1 | Ta gr2 | — | — | 48 |
| No prog. | 1086-1 | Ta gr2 | — | — | 34 |
| No prog. | 1105-1 | Ta gr2 | — | — | 31 |
| No prog. | 1145-1 | Ta gr2 | — | — | 39 |
| No prog. | 1352-1 | Ta gr2 | — | — | 26 |
| No prog. | 829-1 | Ta gr2 | — | — | 37 |
| No prog. | 942-1 | Ta gr2 | — | — | 37 |
| No prog. | 780-1 | Ta gr2 | — | — | 50 |
| Prog | 1327-1 | Ta gr2 | T1 gr3 | 8 | |
| Prog. | 1062-2 | Ta gr3 | T1 gr3 | 4 | — |
| Prog. | 1354-1 | Ta gr3 | T1 gr3 | 8 | — |
| Prog. | 1093-1 | Ta gr3 | T1 gr3 | 5 | — |
| Prog. | 925-7 | Ta gr2 | T1 gr3 | 4 | — |
| Prog. | 962-10 | Ta gr0 | T2+ gr3 | 1 | — |
| Prog. | 970-1 | Ta gr3 | T2+ gr3 | 1 | — |
| Prog. | 1027-1 | Ta gr3 | T2+ gr3 | 2 | — |
| Prog. | 1252-1 | T1 gr3 | T2+ gr3 | 5 | — |
| Prog. | 1191-1 | T1 gr4 | T2+ gr4 | 1 | — |

Delineation of Non-Progressing Tumors from Progressing Tumors

To delineate non-progressing tumors from progressing tumors we now profiled a total of 29 bladder tumor samples; 13 early stage bladder tumor samples without progression (median follow-up time 35 months) and 16 early stage bladder tumor samples with progression (median time to progression 7 months). See Table 1 for description of patient disease courses. We analyzed gene expression changes between the two groups of tumors by hybridizing the labeled RNA samples to customized Affymetrix GeneChips with 59,000 probe-sets to cover virtually the entire transcriptome (˜95% coverage). Low expressed and non-varying probesets were eliminated from the data set and the resulting 6,647 probe-sets that showed variation across the tumor samples were subjected to further analysis. These probe-sets represent 5,356 unique genes (Unigene clusters).

### Gene Expression Similarities Between Tumor Biopsies

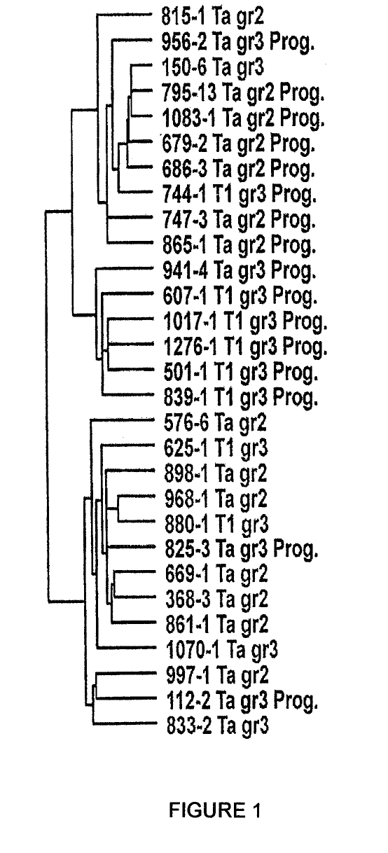

We analyzed gene expression similarities between the tumor biopsies using unsupervised hierarchical cluster analysis (FIG. 1). This showed a notable distinction between the non-progressing and the progressing tumors when using the 3,197 most varying probe-sets (s.d. ≧75) for clustering (4 errors; χ² test, P=0.0001). Using other gene-sets based on different gene variation criteria demonstrated the same distinction between the tumor groups. Two of the samples that show later progression (825-3 and 112-2) were found in the non-progression branch of the cluster dendrogram and two of the non-progressing samples (815-1 and 150-6) were found in the progression branch. This distinct separation of the samples indicated a considerable biological difference between the two groups of tumors. Notably, the T1 tumors did not cluster separately from Ta tumors; however, they did form a sub-cluster in the progressing branch of the dendrogram. Based on this we decided to look for a general signature of progression disregarding pathologic staging of the tumors.

### Selection of the 100 Most Significantly Up-Regulated Genes in Each Group Using T-Test Statistics

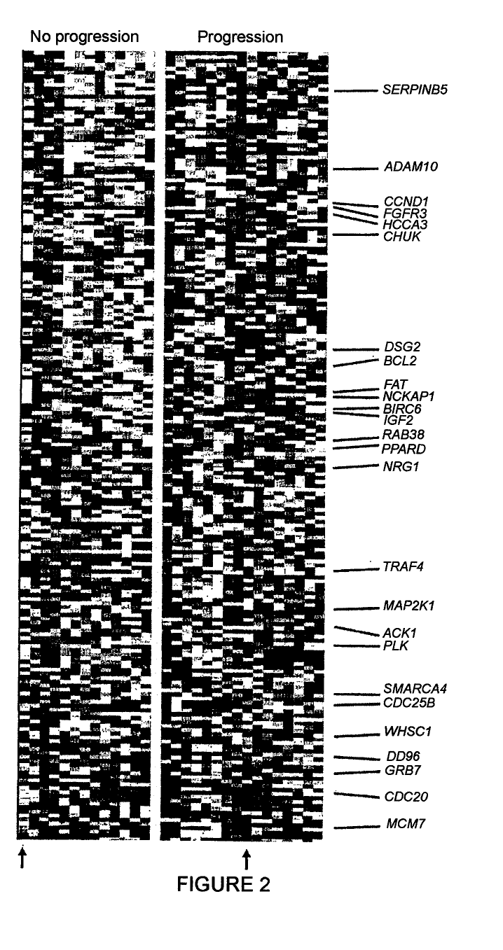

We delineated the non-progressing tumors from the progressing tumors by selecting the 100 most significantly up-regulated genes in each group using t-test statistics (FIG. 2 and Table 2). Among the genes up regulated in the non-progressing group we found the SERPINB5 and FAT tumor suppressor genes and the FGFR3 gene, which has been shown to be frequently mutated in superficial bladder tumors with low recurrence rates (van Rhijn et al. 2001). Among the genes up regulated in the progressing group we found the PLK (Yuan et al. 1997), CDC25B (Galaktionov et al. 1991), CDC20 (Weinstein et al. 1994) and MCM7 (Hiraiwa et al. 1997) genes, which are involved in regulating cell cycle and cell proliferation. Furthermore, in this group we identified the WHSC1, DD96 and GRB7 genes, which have been predicted/computed (Gene Ontology) to be involved in oncogenic transformation. Another interesting candidate in this group is the NRG1 gene, which through interaction with the HER2/HER3 receptors has been found to induce differentiation of lung epithelial cells (Liu & Kern 2002). The PPARD gene was also identified as up regulated in the tumors that show later progression. Disruption of this gene was found to decrease tumorigenicity in colon cancer cells (Park et al. 2001). Furthermore, PPARD regulates VEGF expression in bladder cancer cell lines (Fauconnet et al. 2002).

TABLE 2
The 200 best markers of progression

| Eos | Unigene | | | | Exemplar |
|---|---|---|---|---|---|
| Hu03 ID | Build 133 | Description | T-test | 5% perm | accession# |
| 416640 | Hs.79404 | neuron-specific protein | 6.03 | 5.62 | BE262478 |
| 442220 | Hs.8148 | selenoprotein T | 5.98 | 5.06 | AL037800 |
| 426982 | Hs.173091 | ubiquitin-like 3 | 5.9 | 4.88 | AA149707 |
| 416815 | Hs.80120 | UDP-N-acetyl-alpha-D-galactosamine: polypeptide N- | 5.52 | 4.67 | U41514 |
| | | acetylgalactosaminyltransferase 1 (GalNAc-T1) | | | |
| 435521 | Hs.6361 | mitogen-activated protein kinase kinase 1 interacting | 5.24 | 4.51 | W23814 |
| | | protein 1 | | | |
| 447343 | Hs.236894 | ESTs, Highly similar to S02392 alpha-2-macroglobulin | 5.23 | 4.44 | AA256641 |
| | | receptor precursor [H. sapiens] | | | |
| 452829 | Hs.63368 | ESTs, Weakly similar to TRHY_HUMAN TRICHOHYALI | 4.95 | 4.39 | AI955579 |
| | | [H. sapiens] | | | |
| 414895 | Hs.116278 | Homo sapiens cDNA FLJ13571 fis, clone | 4.94 | 4.31 | AW894856 |
| | | PLACE1008405 | | | |
| 426252 | Hs.28917 | ESTs | 4.9 | 4.26 | BE176980 |
| 444604 | Hs.11441 | chromosome 1 open reading frame 8 | 4.89 | 4.17 | AW327695 |
| 409632 | Hs.55279 | serine (or cysteine) proteinase inhibitor, clade B | 4.89 | 4.13 | W74001 |
| | | (ovalbumin), member 5 | | | |
| 446556 | Hs.15303 | KIAA0349 protein | 4.87 | 4.08 | AB002347 |
| 426799 | Hs.303154 | popeye protein 3 | 4.86 | 4.03 | H14843 |
| 428115 | Hs.300855 | KIAA0977 protein | 4.86 | 4.00 | AB023194 |
| 419847 | Hs.184544 | Homo sapiens, clone IMAGE: 3355383, mRNA, partial | 4.82 | 3.97 | AW390601 |
| | | cds | | | |
| 417839 | Hs.82712 | fragile X mental retardation, autosomal homolog 1 | 4.8 | 3.93 | AI815732 |
| 428284 | Hs.183435 | NM_004545: Homo sapiens NADH dehydrogenase | 4.78 | 3.92 | AA535762 |
| | | (ubiquinone) 1 beta subcomplex, 1 (7 kD, MNLL) | | | |
| | | (NDUFB1), mRNA. | | | |
| 422929 | Hs.94011 | ESTs, Weakly similar to MGB4_HUMAN MELANOMA- | 4.77 | 3.90 | AA356694 |
| | | ASSOCIATED ANTIGEN B4 [H. sapiens] | | | |
| 414762 | Hs.77257 | KIAA0068 protein | 4.72 | 3.86 | AW068349 |
| 453395 | Hs.377915 | mannosidase, alpha, class 2A, member 1 | 4.71 | 3.84 | D63998 |
| 421311 | Hs.283609 | hypothetical protein PRO2032 | 4.65 | 3.82 | N71848 |
| 446847 | Hs.82845 | Homo sapiens cDNA: FLJ21930 fis, clone HEP04301, | 4.65 | 3.82 | T51454 |
| | | highly similar to HSU90916 Human clone 23815 | | | |
| | | mRNA sequence | | | |
| 413840 | Hs.356228 | RNA binding motif protein, X chromosome | 4.62 | 3.79 | AI301558 |
| 418321 | Hs.84087 | KIAA0143 protein | 4.62 | 3.78 | D63477 |
| 430604 | Hs.247309 | succinate-CoA ligase, GDP-forming, beta subunit | 4.61 | 3.74 | AV650537 |
| 423185 | Hs.380062 | ornithine decarboxylase antizyme 1 | 4.61 | 3.74 | BE299590 |
| 417615 | Hs.82314 | hypoxanthine phosphoribosyltransferase 1 (Lesch- | 4.6 | 3.70 | BE548641 |
| | | Nyhan syndrome) | | | |
| 418504 | Hs.85335 | Homo sapiens mRNA; cDNA DKFZp564D1462 (from | 4.59 | 3.68 | BE159718 |
| | | clone DKFZp564D1462) | | | |
| 400846 | — | sortilin-related receptor, L(DLR class) A repeats- | 4.57 | 3.66 | — |
| | | containing (SORL1) | | | |
| 426028 | Hs.172028 | a disintegrin and metalloproteinase domain 10 | 4.53 | 3.65 | NM_001110 |
| | | (ADAM10) | | | |
| 425243 | Hs.155291 | KIAA0005 gene product | 4.47 | 3.63 | N89487 |
| 434978 | Hs.4310 | eukaryotic translation initiation factor 1A | 4.45 | 3.62 | AA321238 |
| 409513 | Hs.54642 | methionine adenosyltransferase II, beta | 4.43 | 3.59 | AW966728 |
| 433282 | Hs.49007 | hypothetical protein | 4.43 | 3.56 | BE539101 |
| 421628 | Hs.106210 | hypothetical protein FLJ10813 | 4.37 | 3.56 | AL121317 |
| 452170 | Hs.28285 | patched related protein translocated in renal cancer | 4.37 | 3.54 | AF064801 |
| 440014 | Hs.6856 | ash2 (absent, small, or homeotic, Drosophila, homolog)- | 4.37 | 3.52 | AW960782 |
| | | like | | | |
| 431857 | Hs.271742 | ADP-ribosyltransferase (NAD; poly (ADP-ribose) | 4.36 | 3.52 | W19144 |
| | | polymerase)-like 3 | | | |
| 417924 | Hs.82932 | cyclin D1 (PRAD1: parathyroid adenomatosis 1) | 4.35 | 3.51 | AU077231 |
| 421733 | Hs.1420 | fibroblast growth factor receptor 3 (achondroplasia, | 4.34 | 3.50 | AL119671 |
| | | thanatophoric dwarfism) | | | |
| 440197 | Hs.317714 | pallid (mouse) homolog, pallidin | 4.32 | 3.49 | AW340708 |
| 434055 | Hs.3726 | x 003 protein | 4.32 | 3.48 | AF168712 |
| 445831 | Hs.13351 | LanC (bacterial lantibiotic synthetase component C)- | 4.31 | 3.46 | NM_006055 |
| | | like 1 | | | |
| 439632 | Hs.334437 | hypothetical protein MGC4248 | 4.29 | 3.45 | AW410714 |
| 448813 | Hs.22142 | cytochrome b5 reductase b5R.2 | 4.28 | 3.44 | AF169802 |
| 449268 | Hs.23412 | hypothetical protein FLJ20160 | 4.28 | 3.43 | AW369278 |
| 429311 | Hs.198998 | conserved helix-loop-helix ubiquitous kinase | 4.28 | 3.42 | AF080157 |
| 423599 | Hs.31731 | peroxiredoxin 5 | 4.27 | 3.41 | AI805664 |
| 422913 | Hs.121599 | CGI-18 protein | 4.26 | 3.40 | NM_015947 |
| 418127 | Hs.83532 | membrane cofactor protein (CD46, trophoblast- | 4.26 | 3.39 | BE243982 |
| | | lymphocyte cross-reactive antigen) | | | |
| 425221 | Hs.155188 | TATA box binding protein (TBP)-associated factor, | 4.25 | 3.38 | AV649864 |
| | | RNA polymerase II, F, 55 kD | | | |
| 426682 | Hs.2056 | UDP glycosyltransferase 1 family, polypeptide A9 | 4.23 | 3.37 | AV660038 |
| 421101 | Hs.101840 | major histocompatibility complex, class I-like sequence | 4.23 | 3.37 | AF010446 |
| 444037 | Hs.380932 | CHMP1.5 protein | 4.22 | 3.35 | AV647686 |
| 443407 | Hs.348514 | ESTs, Moderately similar to 2109260A B cell growth | 4.21 | 3.35 | AA037683 |
| | | factor [H. sapiens] | | | |
| 448625 | Hs.178470 | hypothetical protein FLJ22662 | 4.21 | 3.34 | AW970786 |
| 450997 | Hs.35254 | hypothetical protein FLB6421 | 4.16 | 3.34 | AW580830 |
| 444336 | Hs.10882 | HMG-box containing protein 1 | 4.15 | 3.33 | AF019214 |
| 416977 | Hs.406103 | hypothetical protein FKSG44 | 4.14 | 3.32 | AW130242 |
| 420613 | Hs.406637 | ESTs, Weakly similar to A47582 B-cell growth factor | 4.13 | 3.31 | AI873871 |
| | | precursor [H. sapiens] | | | |
| 414843 | Hs.77492 | heterogeneous nuclear ribonucleoprotein A0 | 4.1 | 3.30 | BE386038 |
| 408288 | Hs.16886 | gb: zI73d06.r1 Stratagene colon (937204) Homo | 4.09 | 3.29 | AA053601 |
| | | sapiens cDNA clone 5′, mRNA sequence | | | |
| 422043 | Hs.110953 | retinoic acid induced 1 | 4.09 | 3.29 | AL133649 |
| 432864 | Hs.359682 | calpastatin | 4.08 | 3.28 | D16217 |
| 410047 | Hs.379753 | zinc finger protein 36 (KOX 18) | 4.06 | 3.28 | AI167810 |
| 400773 | — | NM_003105*: Homo sapiens sortilin-related receptor, | 4.06 | 3.27 | — |
| | | L(DLR class) A repeats-containing (SORL1), mRNA. | | | |
| 423960 | Hs.136309 | SH3-containing protein SH3GLB1 | 4.05 | 3.27 | AA164516 |
| 449626 | Hs.112860 | zinc finger protein 258 | 4.04 | 3.27 | AA774247 |
| 429953 | Hs.226581 | COX15 (yeast) homolog, cytochrome c oxidase assembly | 4.04 | 3.24 | NM_004376 |
| | | protein | | | |
| 428901 | Hs.146668 | KIAA1253 protein | 4.02 | 3.24 | AI929568 |
| 420079 | Hs.94896 | PTD011 protein | 3.99 | 3.22 | NM_014051 |
| 436576 | Hs.77542 | ESTs | 3.98 | 3.21 | AI458213 |
| 412841 | Hs.101395 | hypothetical protein MGC11352 | 3.97 | 3.21 | AI751157 |
| 431604 | Hs.264190 | vacuolar protein sorting 35 (yeast homolog) | 3.96 | 3.21 | AF175265 |
| 428318 | Hs.356190 | ubiquitin B | 3.96 | 3.19 | BE300110 |
| 430677 | Hs.359784 | desmoglein 2 | 3.95 | 3.19 | Z26317 |
| 407955 | Hs.9343 | ESTs | 3.94 | 3.18 | BE536739 |
| 426177 | Hs.167700 | Homo sapiens cDNA FLJ10174 fis, clone | 3.92 | 3.17 | AA373452 |
| | | HEMBA1003959 | | | |
| 429802 | Hs.5367 | ESTs, Weakly similar to I38022 hypothetical protein | 3.92 | 3.17 | H09548 |
| | | [H. sapiens] | | | |
| 423810 | Hs.132955 | BCL2/adenovirus E1B 19 kD-interacting protein 3-like | 3.92 | 3.16 | AL132665 |
| 421475 | Hs.104640 | HIV-1 inducer of short transcripts binding protein; | 3.91 | 3.15 | AF000561 |
| | | lymphoma related factor | | | |
| 436472 | Hs.46366 | KIAA0948 protein | 3.91 | 3.14 | AL045404 |
| 434263 | Hs.79187 | ESTs | 3.9 | 3.13 | N34895 |
| 400843 | — | NM_003105*: Homo sapiens sortilin-related receptor, | 3.9 | 3.13 | — |
| | | L(DLR class) A repeats-containing (SORL1), mRNA. | | | |
| 440357 | Hs.20950 | phospholysine phosphohistidine inorganic pyrophosphate | 3.89 | 3.12 | AA379353 |
| | | phosphatase | | | |
| 437223 | Hs.330716 | Homo sapiens cDNA FLJ14368 fis, clone | 3.88 | 3.12 | C15105 |
| | | HEMBA1001122 | | | |
| 426125 | Hs.166994 | FAT tumor suppressor (Drosophila) homolog | 3.86 | 3.11 | X87241 |
| 432554 | Hs.278411 | NCK-associated protein 1 | 3.86 | 3.10 | AI479813 |
| 422506 | Hs.300741 | sorcin | 3.85 | 3.10 | R20909 |
| 413786 | Hs.13500 | ESTs | 3.83 | 3.09 | AW613780 |
| 429561 | Hs.250646 | baculoviral IAP repeat-containing 6 | 3.83 | 3.08 | AF265555 |
| 404977 | — | Insulin-like growth factor 2 (somatomedin A) (IGF2) | 3.83 | 3.08 | — |
| 427722 | Hs.180479 | hypothetical protein FLJ20116 | 3.82 | 3.08 | AK000123 |
| 400844 | — | NM_003105*: Homo sapiens sortilin-related receptor, | 3.82 | 3.08 | — |
| | | L(DLR class) A repeats-containing (SORL1), mRNA. | | | |
| 426469 | Hs.363039 | methylmalonate-semialdehyde dehydrogenase | 3.81 | 3.07 | BE297886 |
| 439578 | Hs.350547 | nuclear receptor co-repressor/HDAC3 complex sub- | 3.81 | 3.06 | AW263124 |
| | | unit | | | |
| 426508 | Hs.170171 | glutamate-ammonia ligase (glutamine synthase) | 3.8 | 3.06 | W23184 |
| 448524 | Hs.21356 | hypothetical protein DKFZp762K2015 | 3.79 | 3.06 | AB032948 |
| 448357 | Hs.108923 | RAB38, member RAS oncogene family | 3.79 | 3.06 | N20169 |
| 425097 | Hs.154545 | PDZ domain containing guanine nucleotide exchange | 3.77 | 3.05 | NM_014247 |
| | | factor(GEF)1 | | | |
| 421649 | Hs.106415 | peroxisome proliferative activated receptor, delta | 5.76 | 5.50 | AA721217 |
| 427747 | Hs.180655 | serine/threonine kinase 12 | 5.41 | 5.03 | AW411425 |
| 439010 | Hs.75216 | Homo sapiens cDNA FLJ13713 fis, clone | 4.57 | 4.80 | AW170332 |
| | | PLACE2000398, moderately similar to LAR PROTEIN | | | |
| | | PRECURSOR (LEUKOCYTE ANTIGEN RELATED) | | | |
| | | (EC 3.1.3.48) | | | |
| 438818 | Hs.30738 | ESTs | 4.49 | 4.59 | AW979008 |
| 438013 | Hs.15670 | ESTs | 4.42 | 4.50 | AI002106 |
| 452929 | Hs.172816 | neuregulin 1 | 4.37 | 4.40 | AW954938 |
| 404826 | — | Target Exon | 4.22 | 4.32 | — |
| 429124 | Hs.196914 | minor histocompatibility antigen HA-1 | 4.2 | 4.26 | AW505086 |
| 421505 | Hs.285641 | KIAA1111 protein | 4.16 | 4.24 | AW249934 |
| 428712 | Hs.190452 | KIAA0365 gene product | 4.14 | 4.19 | AW085131 |
| 427239 | Hs.356512 | ubiquitin carrier protein | 4.11 | 4.10 | BE270447 |
| 421595 | Hs.301685 | KIAA0620 protein | 4.1 | 4.07 | AB014520 |
| 433844 | Hs.179647 | Homo sapiens cDNA FLJ12195 fis, clone | 4.04 | 4.02 | AA610175 |
| | | MAMMA1000865 | | | |
| 443679 | Hs.9670 | hypothetical protein FLJ10948 | 4.01 | 4.00 | AK001810 |
| 422959 | Hs.349256 | paired immunoglobulin-like receptor beta | 4.01 | 3.98 | AV647015 |
| 452012 | Hs.279766 | kinesin family member 4A | 3.98 | 3.96 | AA307703 |
| 435320 | Hs.117864 | ESTs | 3.97 | 3.91 | AA677934 |
| 456332 | Hs.399939 | gb: nc39d05.r1 NCI_CGAP_Pr2 Homo sapiens cDNA | 3.95 | 3.88 | AA228357 |
| | | clone, mRNA sequence | | | |
| 427999 | Hs.181369 | ubiquitin fusion degradation 1-like | 3.94 | 3.86 | AI435128 |
| 427681 | Hs.284232 | tumor necrosis factor receptor superfamily, member | 3.93 | 3.81 | AB018263 |
| | | 12 (translocating chain-association membrane protein) | | | |
| 413929 | Hs.75617 | collagen, type IV, alpha 2 | 3.93 | 3.79 | BE501689 |
| 420116 | Hs.95231 | FH1/FH2 domain-containing protein | 3.9 | 3.77 | NM_013241 |
| 433914 | Hs.112160 | Homo sapiens DNA helicase homolog (PIF1) mRNA, | 3.88 | 3.75 | AF108138 |
| | | partial cds | | | |
| 420732 | Hs.367762 | ESTs | 3.87 | 3.74 | AA789133 |
| 452517 | — | gb: RC-BT068-130399-068 BT068 Homo sapiens | 3.84 | 3.70 | AI904891 |
| | | cDNA, mRNA sequence | | | |
| 437524 | Hs.385719 | ESTs | 3.82 | 3.68 | AI627565 |
| 435158 | Hs.65588 | DAZ associated protein 1 | 3.8 | 3.66 | AW663317 |
| 448780 | Hs.267749 | Human DNA sequence from clone 366N23 on chromosome | 3.8 | 3.65 | W92071 |
| | | 6q27. Contains two genes similar to consecutive | | | |
| | | parts of the C. elegans UNC-93 (protein 1, | | | |
| | | C46F11.1) gene, a KIAA0173 and Tubulin-Tyrosine | | | |
| | | Ligase LIKE gene, a Mitotic Feedback Control Protein | | | |
| | | MADP2 H | | | |
| 445084 | Hs.250848 | hypothetical protein FLJ14761 | 3.79 | 3.64 | H38914 |
| 423138 | — | gb: EST385571 MAGE resequences, MAGM Homo | 3.75 | 3.60 | AW973426 |
| | | sapiens cDNA, mRNA sequence | | | |
| 419602 | Hs.91521 | hypothetical protein | 3.74 | 3.59 | AW248434 |
| 442549 | Hs.8375 | TNF receptor-associated factor 4 | 3.74 | 3.58 | AI751601 |
| 450893 | Hs.25625 | hypothetical protein FLJ11323 | 3.73 | 3.55 | AK002185 |
| 414223 | Hs.238246 | hypothetical protein FLJ22479 | 3.73 | 3.55 | AA954566 |
| 444312 | Hs.351142 | ESTs | 3.72 | 3.53 | R44007 |
| 425205 | Hs.155106 | receptor (calcitonin) activity modifying protein 2 | 3.71 | 3.51 | NM_005854 |
| 432327 | Hs.274363 | neuroglobin | 3.71 | 3.49 | R36571 |
| 451970 | Hs.211046 | ESTs | 3.67 | 3.48 | AI825732 |
| 408049 | Hs.345588 | desmoplakin (DPI, DPII) | 3.67 | 3.45 | AW076098 |
| 440100 | Hs.158549 | ESTs, Weakly similar to T2D3_HUMAN TRANSCRIPTION | 3.66 | 3.45 | BE382685 |
| | | INITIATION FACTOR TFIID 135 KDA | | | |
| | | SUBUNIT [H. sapiens] | | | |
| 426468 | Hs.117558 | ESTs | 3.65 | 3.43 | AA379306 |
| 402384 | — | NM_007181*: Homo sapiens mitogen-activated protein | 3.64 | 3.43 | — |
| | | kinase kinase kinase kinase 1 (MAP4K1), | | | |
| | | mRNA. | | | |
| 458132 | Hs.103267 | hypothetical protein FLJ22548 similar to gene trap | 3.64 | 3.42 | AW247012 |
| | | PAT 12 | | | |
| 447400 | Hs.18457 | hypothetical protein FLJ20315 | 3.64 | 3.42 | AK000322 |
| 443893 | Hs.115472 | ESTs, Weakly similar to 2004399A chromosomal | 3.63 | 3.41 | BE079602 |
| | | protein [H. sapiens] | | | |
| 424959 | Hs.153937 | activated p21cdc42Hs kinase | 3.62 | 3.40 | NM_005781 |
| 409586 | Hs.55044 | DKFZP586H2123 protein | 3.6 | 3.39 | AL050214 |
| 445692 | Hs.182099 | ESTs | 3.6 | 3.37 | AI248322 |
| 433052 | Hs.293003 | ESTs, Weakly similar to PC4259 ferritin associated | 3.6 | 3.36 | AW971983 |
| | | protein [H. sapiens] | | | |
| 421782 | Hs.108258 | actin binding protein; macrophin (microfilament and | 3.59 | 3.35 | AB029290 |
| | | actin filament cross-linker protein) | | | |
| 414907 | Hs.77597 | polo (Drosophia)-like kinase | 3.58 | 3.34 | X90725 |
| 454639 | — | gb: RC2-ST0158-091099-011-d05 ST0158 Homo | 3.57 | 3.33 | AW811633 |
| | | sapiens cDNA, mRNA sequence | | | |
| 434547 | Hs.106124 | ESTs | 3.56 | 3.32 | R26240 |
| 439130 | Hs.375195 | ESTs | 3.55 | 3.32 | AA306090 |
| 413564 | — | gb: 601146990F1 NIH_MGC_19 Homo sapiens cDNA | 3.54 | 3.31 | BE260120 |
| | | clone 5′, mRNA sequence | | | |
| 443471 | Hs.398102 | Homo sapiens clone FLB3442 PRO0872 mRNA, | 3.53 | 3.31 | AW236939 |
| | | complete cds | | | |
| 424415 | Hs.146580 | enolase 2, (gamma, neuronal) | 3.52 | 3.30 | NM_001975 |
| 405036 | — | NM_021628*: Homo sapiens arachidonate lipoxy- | 3.52 | 3.29 | — |
| | | genase 3 (ALOXE3), mRNA. VERSION | | | |
| | | NM_020229.1 GI | | | |
| 422068 | Hs.104520 | Homo sapiens cDNA FLJ13694 fis, clone | 3.52 | 3.29 | AI807519 |
| | | PLACE2000115 | | | |
| 424244 | Hs.143601 | hypothetical protein hCLA-iso | 3.52 | 3.28 | AV647184 |
| 451867 | Hs.27192 | hypothetical protein dJ1057B20.2 | 3.51 | 3.26 | W74157 |
| 429187 | Hs.163872 | ESTs, Weakly similar to S65657 alpha-1C-adrenergic | 3.49 | 3.26 | AA447648 |
| | | receptor splice form 2 [H. sapiens] | | | |
| 415200 | Hs.78202 | SWI/SNF related, matrix associated, actin dependent | 3.48 | 3.25 | AL040328 |
| | | regulator of chromatin, subfamily a, member 4 | | | |
| 405667 | — | Target Exon | 3.48 | 3.25 | — |
| 421075 | Hs.101474 | KIAA0807 protein | 3.47 | 3.23 | AB018350 |
| 424909 | Hs.153752 | cell division cycle 25B | 3.46 | 3.22 | S78187 |
| 451164 | Hs.60659 | ESTs, Weakly similar to T46471 hypothetical protein | 3.46 | 3.21 | AA015912 |
| | | DKFZp434L0130.1 [H. sapiens] | | | |
| 438644 | Hs.129037 | ESTs | 3.46 | 3.20 | AI126162 |
| 432258 | Hs.293039 | ESTs | 3.45 | 3.19 | AW973078 |
| 411817 | Hs.72241 | mitogen-activated protein kinase kinase 2 | 3.45 | 3.19 | BE302900 |
| 414918 | Hs.72222 | hypothetical protein FLJ13459 | 3.45 | 3.18 | AI219207 |
| 437256 | Hs.97871 | Homo sapiens, clone IMAGE: 3845253, mRNA, partial | 3.43 | 3.17 | AL137404 |
| | | cds | | | |
| 404208 | — | C6001282: gi\|4504223\|ref\|NP_000172.1\|glucuronidase, | 3.42 | 3.16 | — |
| | | beta [Homo sapiens] gi\|114963\|sp\|P082 | | | |
| 421989 | Hs.110457 | Wolf-Hirschhorn syndrome candidate 1 | 3.4 | 3.15 | AJ007042 |
| 438942 | Hs.6451 | PRO0659 protein | 3.39 | 3.14 | AW875398 |
| 412649 | Hs.74369 | integrin, alpha 7 | 3.38 | 3.14 | NM_002206 |
| 414840 | Hs.23823 | hairy/enhancer-of-split related with YRPW motif-like | 3.37 | 3.13 | R27319 |
| 434831 | Hs.273397 | KIAA0710 gene product | 3.35 | 3.12 | AA248060 |
| 431842 | Hs.271473 | epithelial protein up-regulated in carcinoma, membrane | 3.34 | 3.11 | NM_005764 |
| | | associated protein 17 | | | |
| 402328 | — | Target Exon | 3.34 | 3.10 | — |
| 405371 | — | NM_005569*: Homo sapiens LIM domain kinase 2 | 3.33 | 3.10 | — |
| | | (LIMK2), transcript variant 2a, mRNA. | | | |
| 441650 | Hs.132545 | ESTs | 3.32 | 3.09 | AI261960 |
| 418629 | Hs.86859 | growth factor receptor-bound protein 7 | 3.3 | 3.09 | BE247550 |
| 406002 | — | Target Exon | 3.3 | 3.08 | — |
| 420307 | Hs.66219 | ESTs | 3.29 | 3.08 | AW502869 |
| 425093 | Hs.154525 | KIAA1076 protein | 3.28 | 3.07 | AB028999 |
| 427351 | Hs.123253 | hypothetical protein FLJ22009 | 3.28 | 3.07 | AW402593 |
| 417900 | Hs.82906 | CDC20 (cell division cycle 20, S. cerevisiae, homolog) | 3.28 | 3.06 | BE250127 |
| 457228 | Hs.195471 | Human cosmid CRI-JC2015 at D10S289 in 10sp13 | 3.27 | 3.05 | U15177 |
| 421026 | Hs.101067 | GCN5 (general control of amino-acid synthesis, yeast, | 3.27 | 3.04 | AL047332 |
| | | homolog)-like 2 | | | |
| 430746 | Hs.406256 | ESTs | 3.27 | 3.03 | AW977370 |
| 409556 | Hs.54941 | phosphorylase kinase, alpha 2 (liver) | 3.27 | 3.03 | D38616 |
| 451225 | Hs.57655 | ESTs | 3.26 | 3.03 | AI433694 |
| 404913 | — | NM_024408*: Homo sapiens Notch (Drosophila) | 3.25 | 3.02 | — |
| | | homolog 2 (NOTCH2), mRNA. VERSION | | | |
| | | NM_024410.1 GI | | | |
| 404875 | — | NM_022819*: Homo sapiens phospholipase A2, group | 3.23 | 3.02 | — |
| | | IIF (PLA2G2F), mRNA. VERSION NM_020245.2 GI | | | |
| 404606 | — | Target Exon | 3.23 | 3.01 | — |
| 414732 | Hs.77152 | minichromosome maintenance deficient (S. cerevisiae) 7 | 3.22 | 3.01 | AW410976 |
| 425380 | Hs.32148 | AD-015 protein | 3.22 | 3.00 | AA356389 |
| 421186 | Hs.270563 | ESTs, Moderately similar to T12512 hypothetical | 3.21 | 2.98 | AI798039 |
| | | protein DKFZp434G232.1 [H. sapiens] | | | |
| 445462 | Hs.288649 | hypothetical protein MGC3077 | 3.2 | 2.97 | AA378776 |

Permutation analysis of 100 most significantly up-regulated genes in each group By permuting the sample labels 500 times we estimated the significance of the differentially expressed genes. The permutation analysis revealed that it was highly unlikely to find as good markers by chance, as similar godd markers were only found in 5% of the permutated data sets, see Table 2.

### Molecular Predictor of Progression

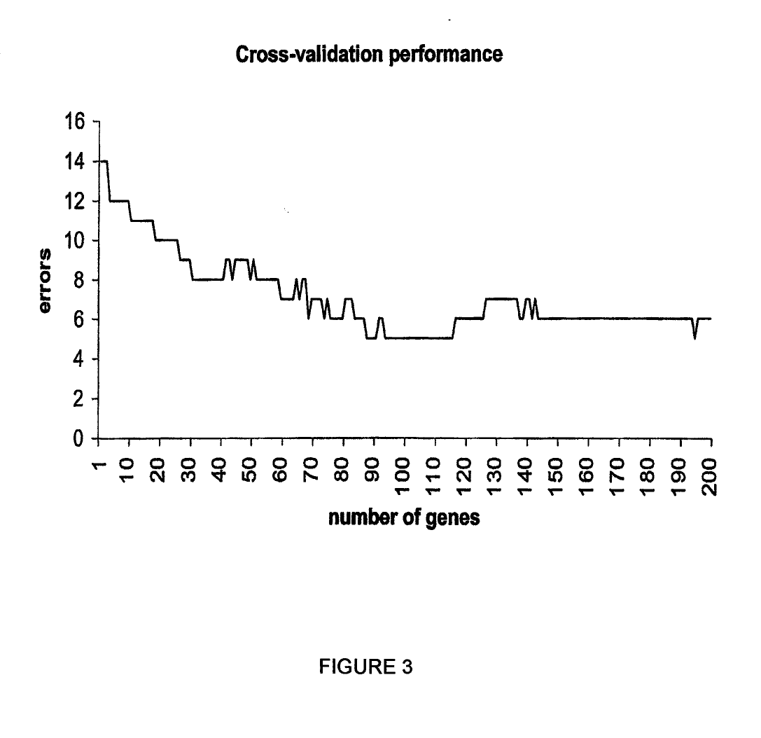

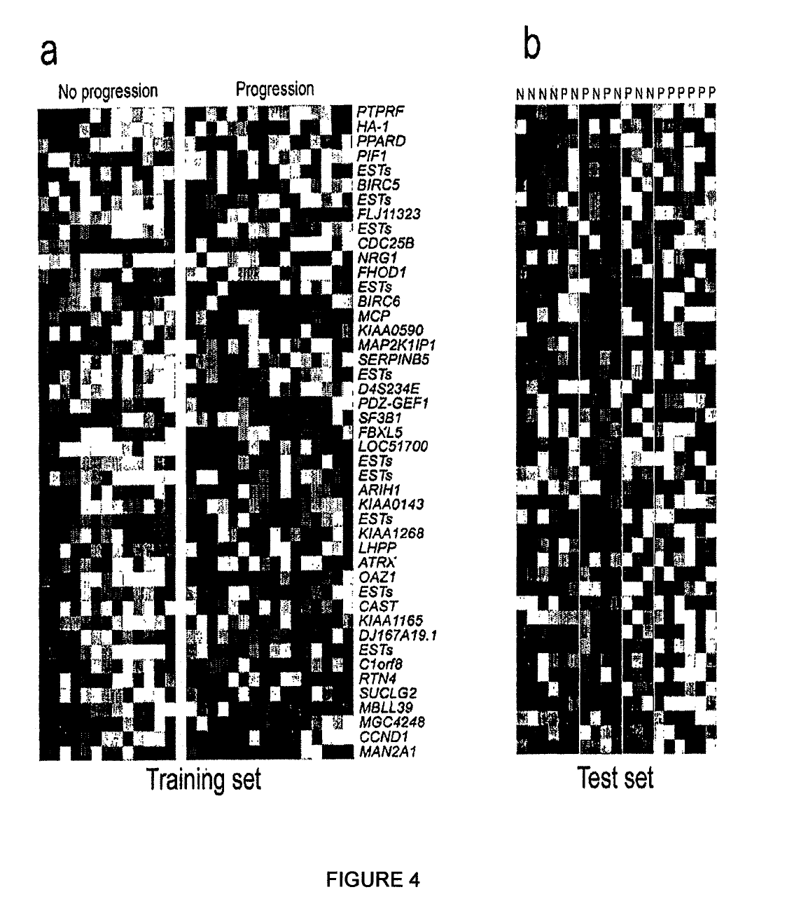

A molecular predictor of progression using a combination of genes may have higher prediction accuracy than when using single marker genes. Therefore, to identify the gene-set that gives the best prediction results using the lowest number of genes we built a predictor using the “leave one out” cross-validation approach, as previously described (Golub et al. 1999). Selecting the 100 best genes in each cross-validation loop gave the lowest number of pre-diction errors (5 errors, 83% correct classification) in our training set consisting of the 29 tumors (see FIG. 3). As in our previous study we used a maximum likelihood classification approach. We selected a gene-expression signature consisting of those 45 genes that were present in 75% of the cross-validation loops, and these represent our optimal gene-set for progression prediction (FIG. 4a and Table 3).

Many of these 45 genes were also found among the 200 best markers of progression, however, the cross-validation approach also identified other interesting markers of progression like BIRC5 (Survivin), an apoptosis inhibitor that is up regulated in the tumors that show later progression. BIRC5 has been reported to be expressed in most common cancers (Ambrosini et al. 1997). To validate the significance of the 45-gene expression signature we used a test set consisting of 19 early stage bladder tumors (9 tumors with no progression and 10 tumors with later progression). Total RNA from these samples were amplified, labeled and hybridized to customized 60mer-oligonucleotide microarray glass slides and the relative expressions of the 45 classifier genes were measured following appropriate normalization and background adjustments of the microarray data. The independent tumor samples were classified as non-progressing or progressing according to the degree of correlation to the average no progression profile from the training samples (FIG. 3b). When applying no cutoff limits to the predictions the predictor identified 74% of the samples correctly. However, as done recently in a breast cancer study (van't Veer et al. 2002), we applied correlation cutoff limits of 0.1 and −0.1 in order to disregard samples with really low correlation values and in this way we obtained 92% correct predictions of samples with correlation values above 0.1 or below −0.1. Although the test-set is limited in size the performance is notable and could be of clinical use.

TABLE 3
The 45 optimal genes for disease progression prediction.

| Eos | Unigene | | | | | Exemplar | |
|---|---|---|---|---|---|---|---|
| Hu03 ID | Build 133 | Description | T-Test | 5% perm | Gene Name | Accession | CV |
| 439010 | Hs.75216 | protein tyrosine phosphatase, receptor | 4.57 | 4.39 | PTPRF | AW170332 | 29 |
| | | type, F | | | | | |
| 429124 | Hs.196914 | minor histocompatibility antigen HA-1 | 4.20 | 4.09 | HA-1 | AW505086 | 29 |
| 421649 | Hs.106415 | peroxisome proliferative activated | 5.76 | 5.64 | PPARD | AA721217 | 29 |
| | | receptor, delta | | | | | |
| 433914 | Hs.112160 | DNA helicase homolog (PIF1) | 3.88 | 3.61 | PIF1 | AF108138 | 29 |
| 429187 | Hs.163872 | ESTs, Weakly similar to hypothetical | 3.49 | 3.17 | — | AA447648 | 28 |
| | | protein FLJ20489 | | | | | |
| 422765 | Hs.1578 | baculoviral IAP repeat-containing 5 | 2.68 | 2.56 | BIRC5 | AW409701 | 28 |
| | | (survivin) | | | | | |
| 433844 | Hs.179647 | ESTs | 4.04 | 3.80 | | AA610175 | 26 |
| 450893 | Hs.25625 | Hypothetical protein FLJ11323 | 3.73 | 3.46 | FLJ11323 | AK002185 | 25 |
| 452866 | Hs.268016 | ESTs | 3.10 | 3.02 | | R26969 | 24 |
| 424909 | Hs.153752 | cell division cycle 25B | 3.46 | 3.16 | CDC25B | S78187 | 24 |
| 452929 | Hs.172816 | neuregulin 1 | 4.37 | 4.23 | NRG1 | AW954938 | 23 |
| 420116 | Hs.95231 | formin homology 2 domain containing 1 | 3.90 | 3.63 | FHOD1 | NM_013241 | 22 |
| 453963 | Hs.28959 | cDNA FLJ36513 fis, clone | 3.44 | 2.88 | — | AA040311 | 29 |
| | | TRACH2001523 | | | | | |
| 429561 | Hs.250646 | baculoviral IAP repeat-containing 6 | 3.83 | 3.03 | BIRC6 | AF265555 | 29 |
| | | (apollon) | | | | | |
| 418127 | Hs.83532 | membrane cofactor protein (CD46, | 4.26 | 3.37 | MCP | BE243982 | 29 |
| | | trophoblast-lymphocyte cross-reactive | | | | | |
| | | antigen) | | | | | |
| 422119 | Hs.111862 | KIAA0590 gene product | 2.33 | 1.95 | KIAA0590 | AI277829 | 29 |
| 435521 | Hs.6361 | mitogen-activated protein kinase | 5.24 | 4.53 | MAP2K1IP1 | W23814 | 29 |
| | | kinase 1 interacting protein 1 | | | | | |
| 409632 | Hs.55279 | serine (or cysteine) proteinase inhibitor, | 4.89 | 4.11 | SERPINB5 | W74001 | 29 |
| | | clade B (ovalbumin), member 5 | | | | | |
| 452829 | Hs.63368 | ESTs | 4.95 | 4.31 | — | AI955579 | 29 |
| 416640 | Hs.79404 | DNA segment on chromosome 4 | 6.03 | 5.51 | D4S234E | BE262478 | 29 |
| | | (unique) 234 expressed sequence | | | | | |
| 425097 | Hs.154545 | PDZ domain containing guanine nucleotide | 3.77 | 3.18 | PDZ-GEF1 | NM_014247 | 28 |
| | | exchange factor(GEF)1 | | | | | |
| 445926 | Hs.334826 | splicing factor 3b, subunit 1, 155 kDa | 2.40 | 2.03 | SF3B1 | AF054284 | 28 |
| 437325 | Hs.5548 | F-box and leucine-rich repeat protein 5 | 2.48 | 2.09 | FBXL5 | AF142481 | 28 |
| 448813 | Hs.22142 | cytochrome b5 reductase b5R.2 | 4.28 | 3.41 | LOC51700 | AF169802 | 28 |
| 426799 | Hs.303154 | ESTs | 4.86 | 4.04 | — | H14843 | 28 |
| 446847 | Hs.82845 | ESTs | 4.65 | 3.79 | — | T51454 | 28 |
| 428016 | Hs.181461 | ariadne homolog, ubiquitin-conjugating | 3.77 | 3.15 | ARIH1 | AJ243190 | 27 |
| | | enzyme E2 binding protein, 1 (Drosophila) | | | | | |
| 418321 | Hs.84087 | KIAA0143 protein | 4.62 | 3.76 | KIAA0143 | D63477 | 27 |
| 422984 | Hs.351597 | ESTs | 3.50 | 2.93 | | W28614 | 26 |
| 408688 | Hs.152925 | KIAA1268 protein | 3.52 | 2.95 | KIAA1268 | AI634522 | 26 |
| 440357 | Hs.20950 | phospholysine phosphohistidine inorganic | 3.89 | 3.07 | LHPP | AA379353 | 26 |
| | | pyrophosphate phosphatase | | | | | |
| 420269 | Hs.96264 | alpha thalassemia/mental retardation | 3.39 | 2.85 | ATRX | U72937 | 26 |
| | | syndrome X-linked (RAD54 (S. cerevisiae) | | | | | |
| | | homolog) | | | | | |
| 423185 | ? | ornithine decarboxylase antizyme 1 | 4.61 | 3.71 | OAZ1 | BE299590 | 26 |
| 443407 | Hs.348514 | clone IMAGE: 4052238, mRNA, partial cds | 4.21 | 3.32 | — | AA037683 | 25 |
| 457329 | Hs.359682 | calpastatin | 3.59 | 2.99 | CAST | AI634860 | 25 |
| 452714 | Hs.30340 | KIAA1165: likely ortholog of mouse | 3.62 | 3.01 | KIAA1165 | AW770994 | 25 |
| | | Nedd4 WW domain-binding protein 5A | | | | | |
| 444773 | Hs.11923 | hypothetical protein DJ167A19.1 | 3.71 | 3.11 | DJ167A19.1 | BE156256 | 24 |
| 418504 | Hs.85335 | ESTs | 4.59 | 3.67 | — | BE159718 | 24 |
| 444604 | Hs.11441 | Chromosome 1 open reading frame 8 | 4.89 | 4.17 | C1orf8 | AW327695 | 23 |
| 410691 | Hs.65450 | reticulon 4 | | | RTN4 | AW239226 | 23 |
| 430604 | Hs.247309 | succinate-CoA ligase, GDP-forming, | 4.61 | 3.72 | SUCLG2 | AV650537 | 23 |
| | | beta subunit | | | | | |
| 421311 | Hs.283609 | muscleblind-like protein MBLL39 | 4.65 | 3.82 | MBLL39 | N71848 | 23 |
| 439632 | Hs.334437 | hypothetical protein MGC4248 | 4.29 | 3.42 | MGC4248 | AW410714 | 22 |
| 417924 | Hs.82932 | cyclin D1 (PRAD1: parathyroid adenomatosis 1) | 4.35 | 3.49 | CCND1 | AU077231 | 22 |
| 453395 | Hs.377915 | mannosidase, alpha, class 2A, member 1 | 4.71 | 3.84 | MAN2A1 | D63998 | 22 |

### Permutation Analysis of 45 Genes

Again permutation analysis revealed that for all of the 45 genes similar good markers were only found in 5% of the 500 permuted datasets (see Table 3).

### Expression Profiling of Metachrone Higher Stage Tumors

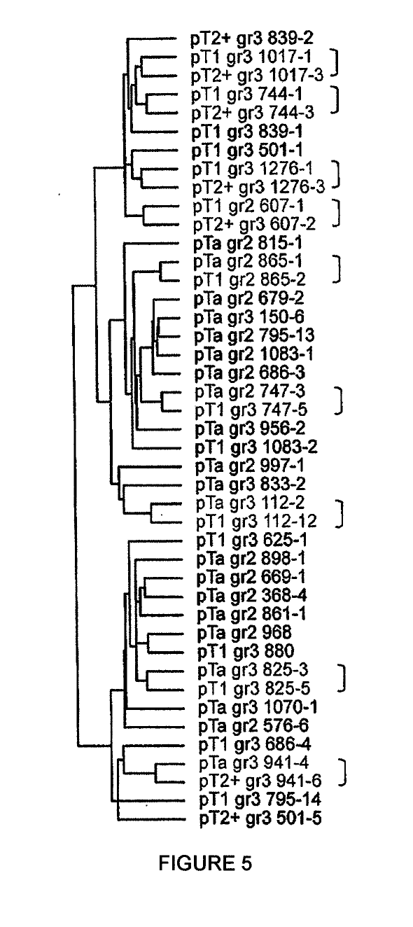

Expression profiling of the metachrone higher stage tumors could provide important information on the degree of expression similarities between the primary and the secondary tumors. Tissues from secondary tumors were available from 14 of the patients with disease progression and these were also hybridized to the customized Affymetrix GeneChips. Hierarchical cluster analysis of all tumor samples based on the 3,213 most varying probesets showed that tumors originating from the same patient in 9 of the cases clustered tightly together indicating a high degree of intra individual similarity in expression profiles (FIG. 5). Notable, one tight clustering pair of tumors was a Ta and a T2+ tumor (patient 941). It was remarkable that Ta and T1 tumors and T1 or T2+ tumors from a single individual were more similar than e.g. Ta tumors from two individuals. There was no correlation between presence and absence of the tight clustering of samples from the same patient and time interval to tumor progression. The tight clustering of the 9 tumor pairs probably reflects the monoclonal nature of many bladder tumors (Sidransky et al. 1997). A set of genomic abnormalities like chromosomal gains and losses characterize bladder tumors of different stages from single individuals (Primdahl et al. 2002), and such physical abnormalities could be one of the causes of the strong similarity of metachronous tumors. The fact that 5 of the tumor pairs clustered apart may be explained by an oligoclonal origin of these tumors.

### Customized GeneChip Design, Normalization and Expression Measures

We used a customized Affymetrix GeneChip (Eos Hu03) designed by Eos Biotech Inc., as described (Eaves et al. 2002). Approximately 45,000 mRNA/EST clusters and 6,200 predicted exons are represented by the 59,000 probesets on Eos Hu03 array. Data were normalized using protocols and software developed at Eos Biotechnology, Inc. (WO0079465). An “average intensity” (AI) for each probeset was calculated by taking the trimean of probe intensities following background subtraction and normalization to a gamma distribution (Turkey 1977).

cRNA Preparation, Array Hybridization and Scanning

Preparation of cRNA from total RNA and subsequent hybridization and scanning of the customized GeneChip microarrays (Eos Hu03) were performed as described previously (Dyrskjot et al. 2003).

### Custom Oligonucleotide Microarray Procedures

Three 60mer oligonucleotides were designed for each of the 45 genes using Array Designer 2.0. All steps in the customized oligonucleotide microarray analysis were performed essentially as described (Kruhoffer et al.) Each of the probes was spotted in duplicates and all hybridisations were carried out twice. The samples were labelled with Cy3 and a common reference pool was labelled with Cy5. The reference pool was made by pooling of cRNA generated from investigated samples and from universal human RNA. Following scanning of the glass slides the fluorescent intensities were quantified and background adjusted using SPOT 2.0 (Jain et al. 2002). Data were subsequently normalized using a LOWESS normalisation procedure implemented in the SMA package to R. To select the best oligonucleotide probe for each of the 45 genes, 13 of the samples from the training set were re-analysed on the custom oligonucleotide microarray platform and the obtained expression ratios were compared to the expression levels from the Affymetrix GeneChips. The oligonucleotide probes with the highest correlation to the Affymetrix GeneChip probes were selected.

### Expression Data Analysis

Before analysing the expression data from the Eos Hu03 GeneChips control probes were removed and only probes with AI levels above 100 in at least 8 experiments and with max/min equal to or above 1.6 were selected. This filtering generated a gene-set consisting of 6,647 probes for further analysis. Average linkage hierarchical cluster analysis of the tumour samples was carried out using a modified Pearson correlation as similarity metric (Eisen et al. 1998). Genes and arrays were median centered and normalised to the magnitude of 1 before clustering. We used the GeneCluster 2.0 software for the supervised selection of markers and for performing permutation tests. The 45 genes for predicting progression were selected by t-test statistics and cross-validation performance as previously described (Dyrskjot et al. 2003) and independent samples were classified according to the correlation to the average no progression signature profile of the 45 genes.

### Example 2

### Identifying Distinct Classes of Bladder Carcinoma Using Microarrays

### Patient Disease Course Information—Class Discovery

We selected tumours from the entire spectrum of bladder carcinoma for expression profiling in order to discover the molecular classes of the disease. The tumours analysed are listed in Table 4 below together with the available patient disease course information.

TABLE 4
Disease course information of all patients involved-class discovery.

| | | | | | Reviewed | | Carcinoma in |
|---|---|---|---|---|---|---|---|
| Group | Patient | Previous tumours | Tumour examined on array | Pattern | histology | Subsequent tumours | situ* |
| A | 709-1 | | Ta gr 2 (200297) | Papillary | Ta gr3 | | no |
| | 968-1 | | Ta gr 2 (011098) | Papillary | + | Ta gr 2 (150101) | no |
| | 934-1 | | Ta gr 2 (220798) | Papillary | + | | no |
| | 928-1 | | Ta gr 2 (240698) | Papillary | + | | no |
| | 930-1 | | Ta gr 2 (300698) | Papillary | + | | no |
| B | 989-1 | | Ta gr 3 (281098) | Papillary | + | | no |
| | 1264-1 | | Ta gr 3 (130600) | Papillary | + | Ta gr 2 (231000) | no |
| | | | | | | Ta gr 2 (220101) | |
| | | | | | | Ta gr 2 (300401) | |
| | 876-5 | Ta gr 2 (230398) | Ta gr 3 (170400) | Papillary | + | | no |
| | | Ta gr 2 (271098) | | | | | |
| | | Ta gr 2 (090699) | | | | | |
| | | Ta gr 2 (011199) | | | | | |
| | 669-7 | Ta gr 2 (101296) | Ta gr 3 (230899) | Papillary | Ta gr2 | Ta gr 2 (120100) | no |
| | | Ta gr 2 (150897) | | | | Ta gr 2 (250500) | |
| | | Ta gr 1 (161297) | | | | Ta gr 2 (250900) | |
| | | Ta gr 3 (270498) | | | | Ta gr 2 (050201) | |
| | | Ta gr 2 (220299) | | | | | |
| | 716-2 | Ta gr 2 (070397) | Ta gr 3 (230497) | Papillary | + | Ta gr 2 (040697) | no |
| | | | | | | Ta gr 1 (170698) | |
| C | 1070-1 | | Ta gr 3 (150399) | Papillary | + | Ta gr 3 (291099) | Subsequent |
| | | | | | | | visit |
| | 956-2 | | Ta gr 3 (061299) | Papillary | + | Ta gr 3 (061200) | Sampling visit |
| | 1062-2 | | Ta gr 3 (120799) | Papillary | + | T1 gr 3 (161199) | Sampling visit |
| | 1166-1 | | Ta gr 3 (271099) | Papillary | + | | Sampling visit |
| | 1330-1 | | Ta gr 3 (311000) | Papillary | + | | Sampling visit |
| D | 112-10 | Ta gr 2 (070794) | Ta gr 3 (060198) | Papillary | + | Ta gr 3 (110698) | Previous visit |
| | | Ta gr 3 (011294) | | | | T1 gr 3 (191098) | |
| | | T1 gr 3 (150695) | | | | Ta gr 3 (240299) | |
| | | Ta gr 3 (121095) | | | | T1 gr 3 (050799) | |
| | | T1 gr 3 (040396) | | | | T1 gr 3 (081199) | |
| | | Ta gr 2 (200896) | | | | T1 gr 3 (180400) | |
| | | Ta gr 2 (111296) | | | | | |
| | | Ta gr 2 (230497) | | | | | |
| | | Ta gr 2 (030997) | | | | | |
| | 320-7 | T1 gr 3 (011194) | Ta gr 3 (290997) | Papillary | + | Ta gr 3 (290198) | Sampling visit |
| | | T1 gr 3 (150896) | | | | Ta gr 3 (290698) | |
| | | Ta gr 3 (100897) | | | | | |
| | 747-7 | Ta gr 2 (010597) | Ta gr 3 (161298) | Papillary | + | Ta gr 2 (050599) | Sampling visit |
| | | Ta gr 2 (220597) | | | | Ta gr 2 (280999) | |
| | | Ta gr 2 (230997) | | | | Ta gr 2 (141299) | |
| | | Ta gr 2 (260198) | | | | | |
| | | T1 gr 3 (270498) | | | | | |
| | | Ta gr 2 (170898) | | | | | |
| | 967-3 | T1 gr 3 (280998) | Ta gr 3 (140699) | Papillary | + | T1 gr 3 (080999) | Sampling visit |
| | | T1 gr 3 (250199) | | | | | |
| E | 625-1 | | T1 gr 3 (200996) | Papillary | + | | No |
| | 847-1 | | T1 gr 3 (210198) | Papillary | + | | No |
| | 1257-1 | | T1 gr 3 (240500) | Solid | + | | Sampling visit |
| | 919-1 | | T1 gr 3 (220698) | Papillary | + | | No |
| | 880-1 | | T1 gr 3 (300398) | Papillary | + | Ta gr 2 (091198) | No |
| | | | | | | Ta gr 1 (090399) | |
| | | | | | | Ta gr 2 (050900) | |
| | | | | | | Ta gr 2 (190301) | |
| | 812-1 | | T1 gr 3 (061098) | Papillary | + | | No |
| | 1269-1 | | T1 gr 3 (230600) | Papillary | − | | No |
| | 1083-2 | Ta gr 2 (280499) | T1 gr 3 (120599) | Papillary | − | | No |
| | 1238-1 | | T1 gr 3 (020500) | Papillary | + | T2 gr 3 (211100) | No |
| | | | | | | Ta gr 2 (211100) | |
| | 1065-1 | | T1 gr 3 (160399) | Papillary | − | | Subsequent |
| | | | | | | | visit |
| | 1134-1 | | T1 gr 3 (181099) | Papillary | T2 gr3 | T1 gr 3 (280200) | Sampling visit |
| | | | | | | T1 gr 3 (020500) | |
| | | | | | | T1 gr 3 (131100) | |
| F | 1164-1 | | T2+ gr 4 (101299) | Solid | gr 3 | | No |
| | 1032-1 | | T2+ gr ? (050199) | Mixed | − | | Not measured |
| | 1117-1 | | T2+ gr 3 (010999) | Solid | + | | Sampling visit |
| | 1178-1 | | T2+ gr 3 (200100) | Solid | + | | Not measured |
| | 1078-1 | | T2+ gr 3 (120499) | Solid | + | | Not measured |
| | 875-1 | | T2+ gr 3 (180398) | Solid | + | | No |
| | 1044-1 | | T2+ gr 3 (010299) | Solid | + | T2+ gr 3 (060999) | Not measured |
| | 1133-1 | | T2+ gr 3 (081099) | Solid | + | | Not measured |
| | 1068-1 | | T2+ gr 3 (220399) | Solid | + | | No |
| | 937-1 | | T2+ gr 3 (280798) | Solid | − | | Not measured |

Group A: Ta gr2 tumours - no recurrence within 2 years.
Group B: Ta gr3 tumours - no prior T1 tumour and no carcinoma in situ in random biopsies.
Group C: Ta gr3 tumours - no prior T1 tumour but carcinoma in situ in random biopsies.
Group D: Ta gr3 tumours - a prior T1 tumour and carcinoma in situ in random biopsies.
Group E: T1 gr3 tumours - no prior T2+ tumour. Group F: T2+ tumours gr3/4 - only primary tumours.
*Carcinoma in situ detected in selected site biopsies at previous, sampling or subsequent visits.

### Two-Way Hierarchical Cluster Analysis of Tumor Samples

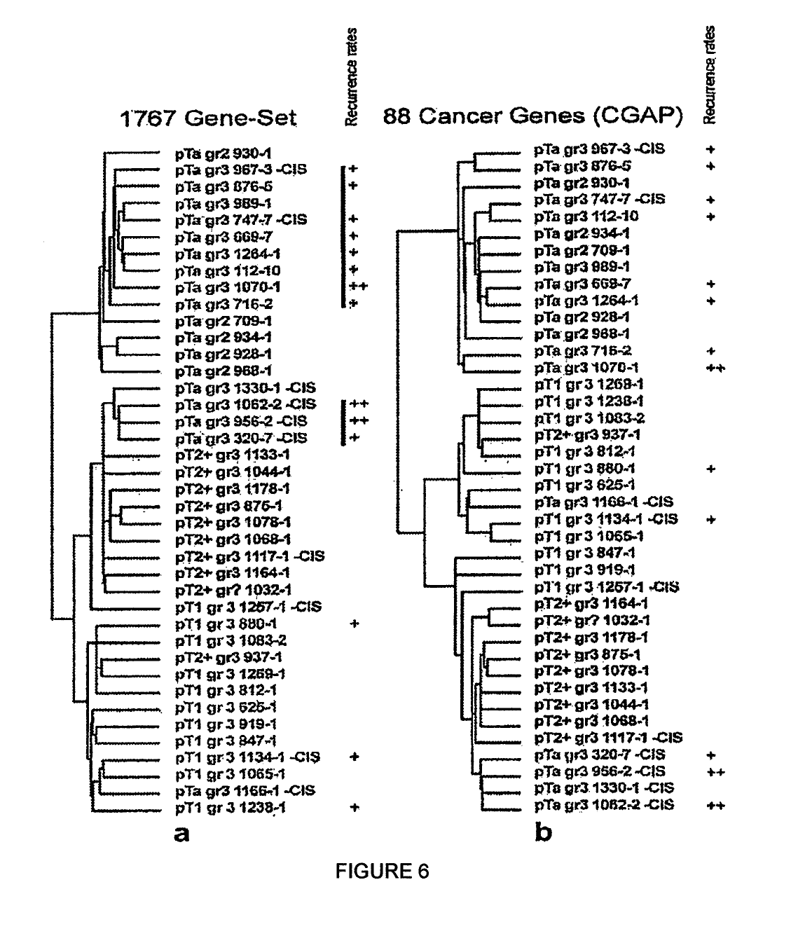

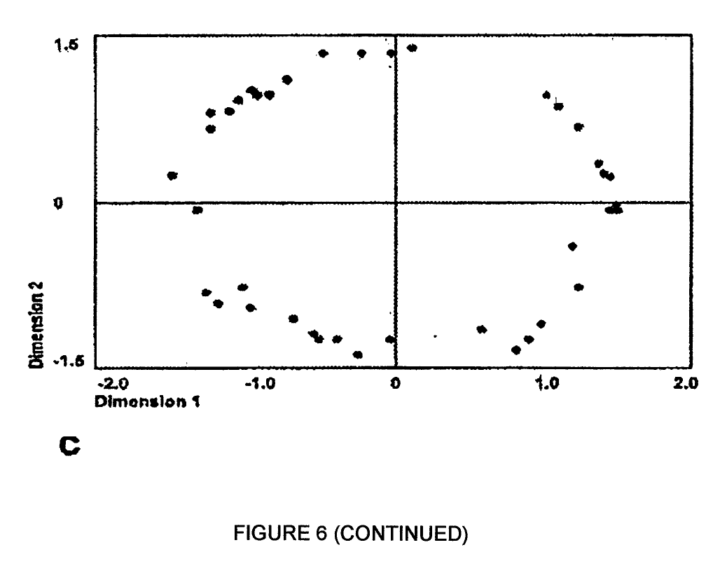

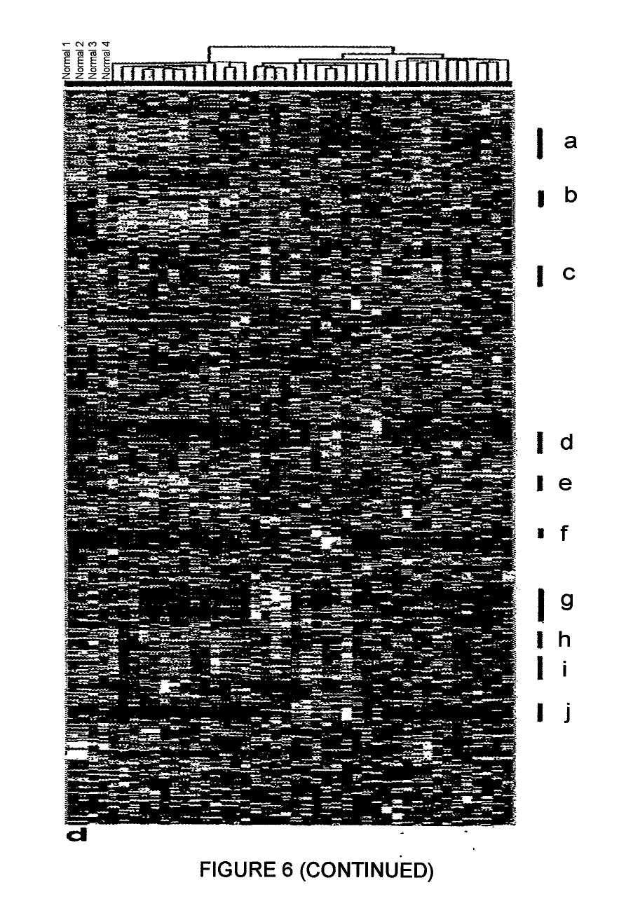

A two-way hierarchical cluster analysis of the tumour samples based on the 1767 gene-set (see class discovery using hierarchical clustering) remarkably separated all 40 tumours according to conventional pathological stages and grades with only few exceptions (FIG. 6a). We identified two main branches containing the superficial Ta tumours, and the invasive T1 and T2+ tumours. In the superficial branch two sub-clusters of tumours could be identified, one holding 8 tumours that had frequent recurrences and one holding 3 out of the five Ta grade 2 tumours with no recurrences. In the invasive branch, it was notable that four Ta grade 3 tumours clustered tightly with the muscle invasive T2+ tumours. These four Ta tumours, from patients with no previous tumour history, showed concomitant CIS in the surrounding mucosa, indicating that this sub-fraction of Ta tumours has some of the more aggressive features found in muscle invasive tumours. The stage T1 cluster could be separated into three sub-clusters with no clear clinical difference. The one stage T1 grade 3 tumour that clustered with the stage T2+ muscle invasive tumours was the only T1 tumour that showed a solid growth pattern, all others showing papillary growth. Nine out of ten T2+ tumours were found in one single cluster. The remarkable distinct separation of the tumour groups according to stage, with practically no overlap between groups, was also demonstrated by multidimensional scaling analysis (FIG. 6c).

In an attempt to reduce the number of genes needed for class prediction we identified those genes that were scored by the Cancer Genome Anatomy Project (at NCI) as belonging to cancer-related groups such as tumour suppressors, oncogenes, cell cycle, etc. These genes were then selected from the initial 1767 gene-set, and those 88 which showed largest variation (SD of the gene vector >=4), were used for hierarchical clustering of the tumour samples. The obtained clusters was almost identical to the 1767 gene-set cluster dendrogram (FIG. 6b), indicating that the tumour clustering does not simply reflect larger amounts of stromal components in the invasive tumour biopsies.

The clustering of the 1767 genes revealed several characteristic profiles in which there was a distinct difference between the tumour groups (FIG. 6d; black lines identifying clusters a to j).

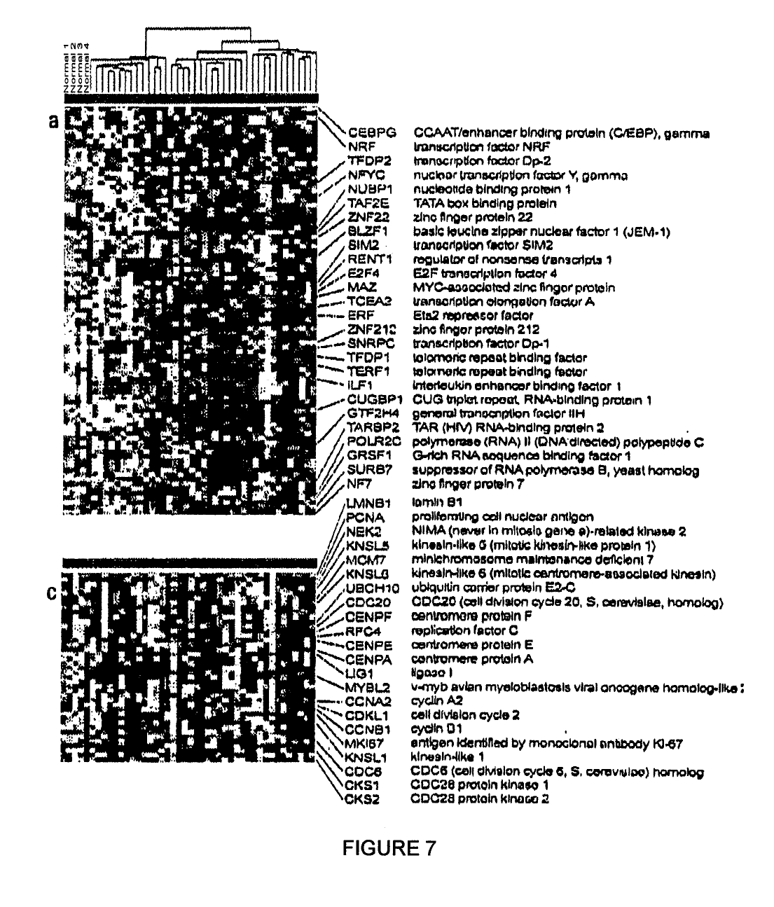

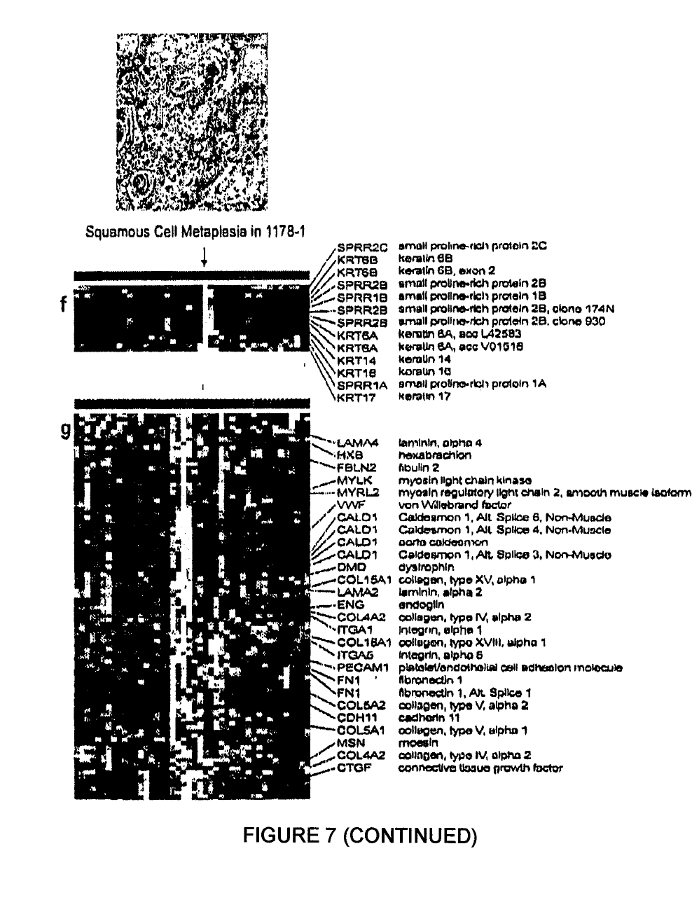

Cluster a, shows a high expression level in all the Ta grade 3 tumours (FIG. 7a) and, as a novel finding, contains genes encoding 8 transcription factors as well as other nuclear genes related to transcriptional activity. Cluster c contains genes that are up-regulated in both Ta grade 3 with high recurrence rate and CIS, in T2+ and some T1 tumours. This cluster shows a remarkable tight co-regulation of genes related to cell cycle control and mitosis (FIG. 7c). Genes encoding cyclins, PCNA as well as a number of centromere related proteins are present in this cluster. They indicate increased cellular proliferation and may form new targets for small molecule therapy (Seymour 1999). Cluster f shows a tight cluster of genes related to keratinisation (FIG. 7f). Two tumours (875-1 and 1178-1) had a very high expression of these genes and a re-evaluation of the pathology slides revealed that these were the only two samples to show squamous metaplasia. Thus, activation of this cluster of genes promotes the squamous metaplasia not infrequently seen by light microscopy in invasive bladder tumours. The genes in this cluster is listed in Table 5.

TABLE 5
Genes for classifying samples with squamous metaplasia

| Chip acc. # | UniGene Build 162 | description |
|---|---|---|
| D83657_at | Hs.19413 | NM_005621; S100 calcium-binding protein A12 |
| HG3945-HT4215_at | | |
| J00124_at | | |
| L05187_at | | |
| L05188_f_at | Hs.505327 | |
| L10343_at | Hs.112341 | NM_002638; skin-derived protease inhibitor 3 preproprotein |
| L42583_f_at | Hs.367762 | NM_005554; keratin 6A |
| L42601_f_at | Hs.367762 | NM_005554; keratin 6A |
| L42611_f_at | Hs.446417 | NM_173086; keratin 6 isoform K6e |
| M19888_at | Hs.1076 | NM_003125; small proline-rich protein 1B (cornifin) |
| M20030_f_at | Hs.505352 | |
| M21005_at | | |
| M21302_at | Hs.505327 | |
| M21539_at | Hs.2421 | NM_006518; small proline-rich protein 2C |
| M86757_s_at | Hs.112408 | NM_002963; S100 calcium-binding protein A7 |
| S72493_s_at | Hs.432448 | NM_005557; keratin 16 |
| U70981_at | Hs.336046 | NM_000640; interleukin 13 receptor, alpha 2 precursor |
| V01516_f_at | Hs.367762 | NM_005554; keratin 6A |
| X53065_f_at | | |
| X57766_at | Hs.143751 | NM_005940; matrix metalloproteinase 11 preproprotein |
| Z19574_rna1_at | | |

Cluster g contains genes that are up-regulated in T2+ tumours and in the Ta grade 3 tumours with CIS that cluster in the invasive branch (FIG. 7g). This cluster contains genes related to angiogenesis and connective tissue such as laminin, myosin, caldesmon, collagen, dystrophin, fibronectin, and endoglin. The increased transcription of these genes may indicate a profound remodelling of the stroma that could reflect signalling from the tumour cells, from infiltrating lymphocytes, or both. Some of these may also form new drug targets (Fox et al. 2001). It is remarkable that these genes are those that most clearly separate the Ta grade 3 tumours surrounded by CIS from all other Ta grade 3 tumours. The presence of adjacent CIS is usually diagnosed by taking a set of eight biopsies from different places in the bladder mucosa. However, the present data clearly indicate that analysis of stroma remodelling genes in the Ta tumours could eliminate this invasive procedure.

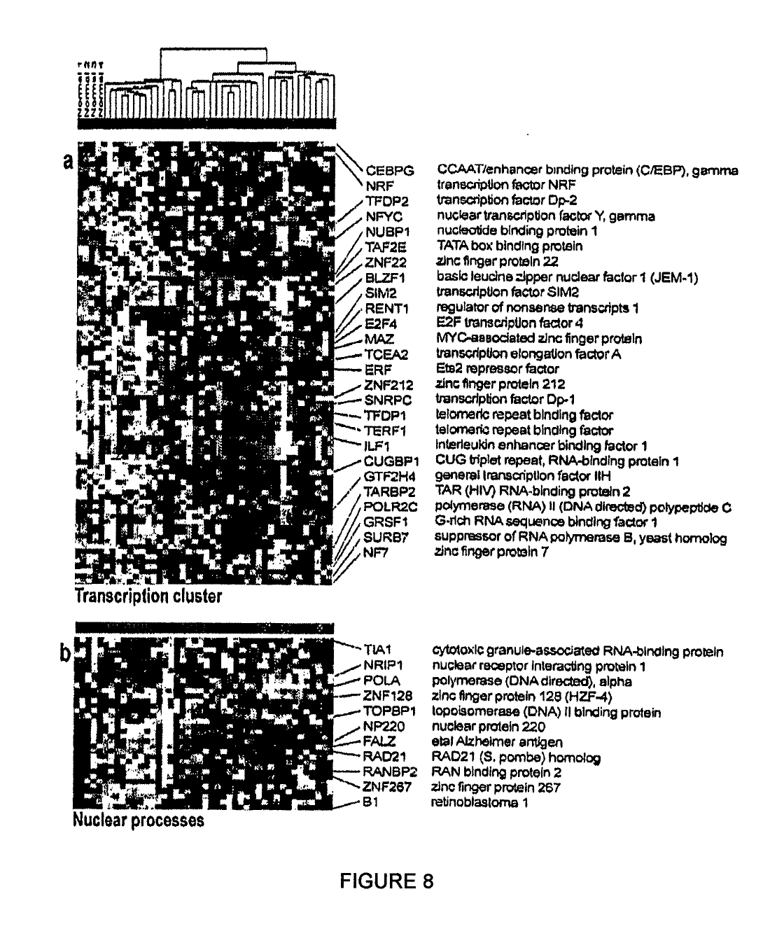

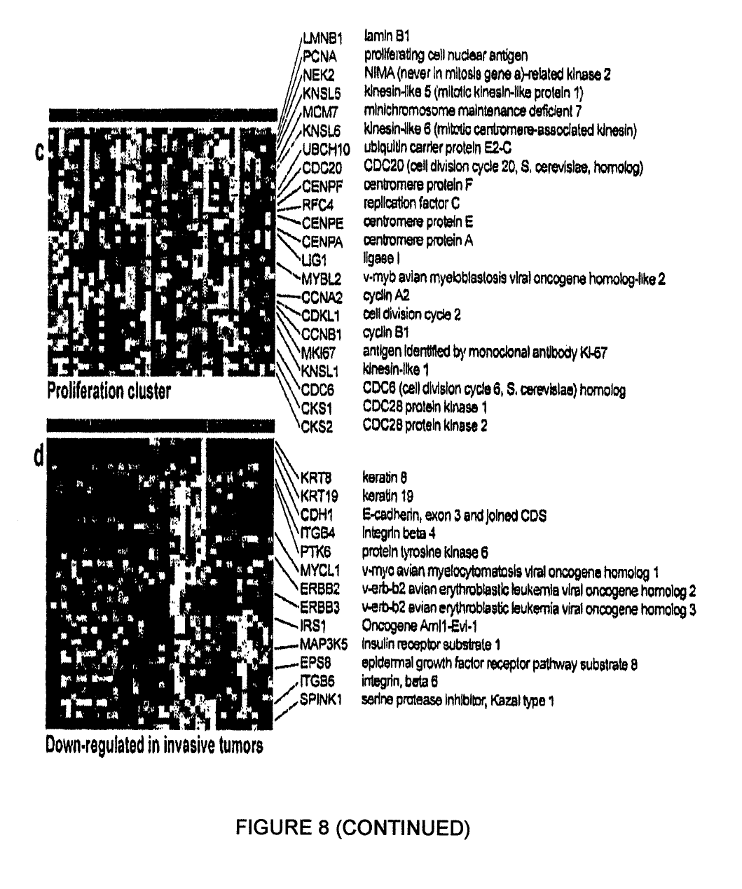

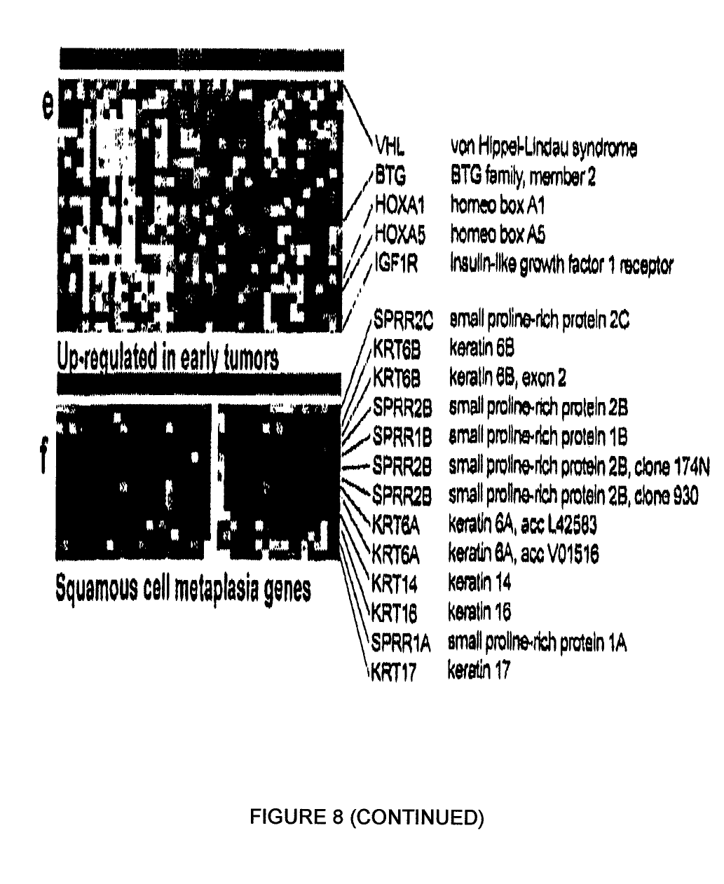

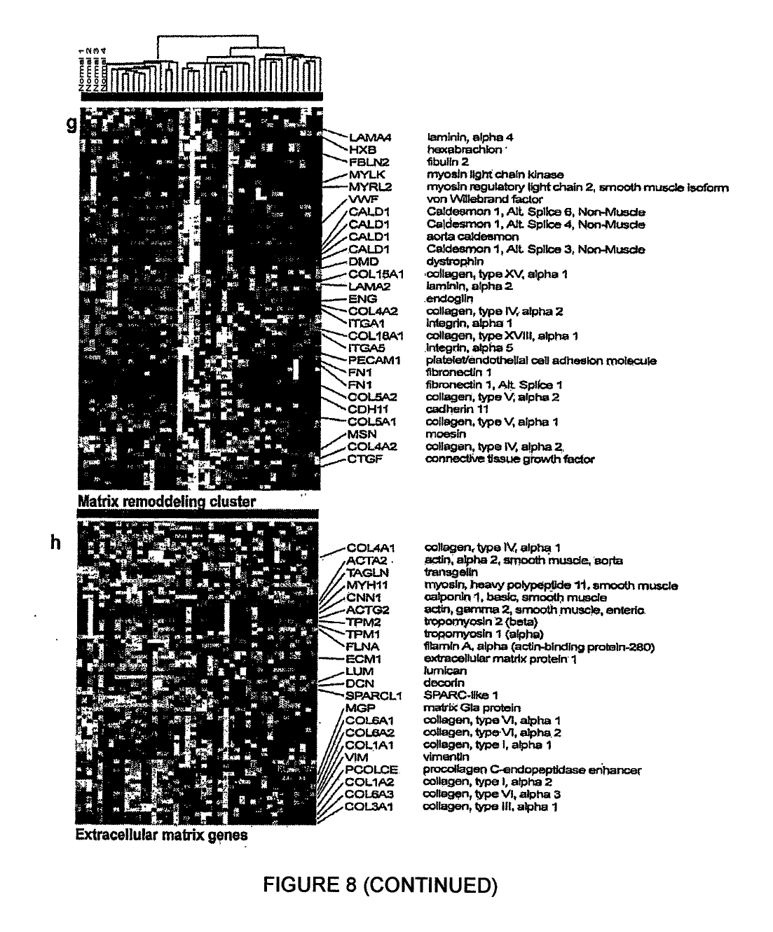

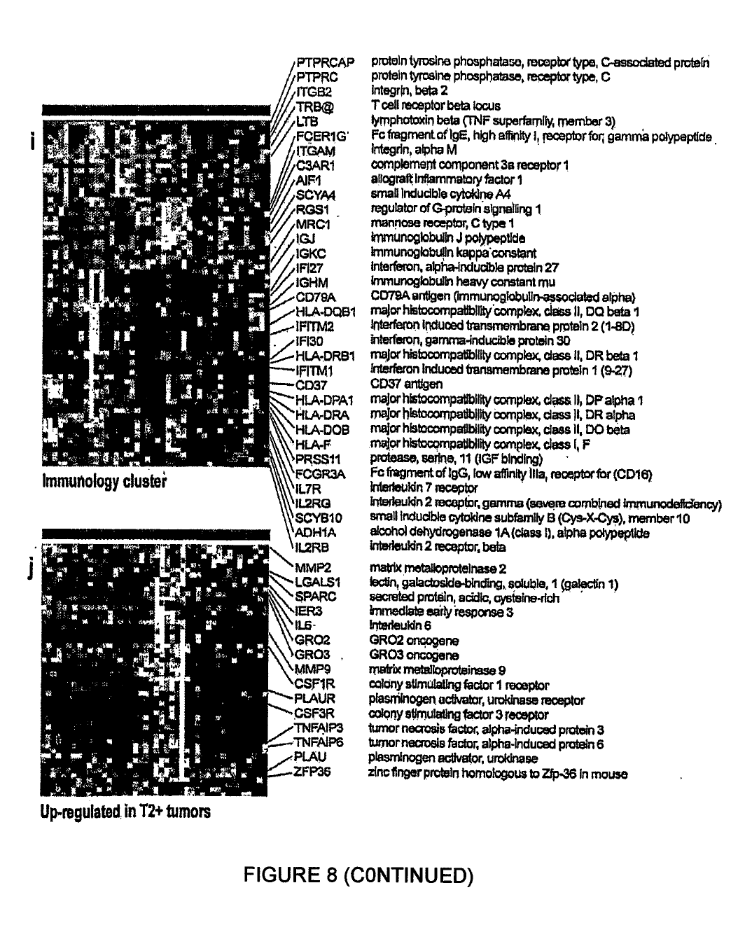

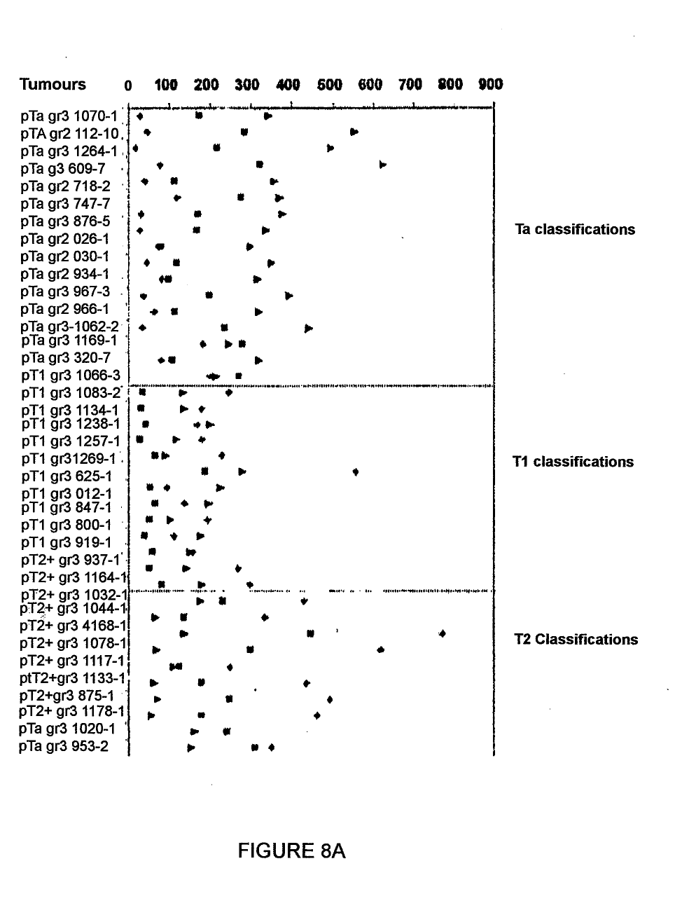

The clusters b, d, e, h, i, and j contain genes related to nuclear proteins, cell adhesion, growth factors, stromal proteins, immune system, and proteases, respectively (see FIG. 8). A summary of the stage related gene expression is shown in Table 6.

TABLE 6
Table 6. Summary of stage related gene expression
Functional gene clusters^{a}

| | | Nuclear | | Matrix re- | Extracellular | Immune |
|---|---|---|---|---|---|---|
| Tumour stage | Transcription | processes | Proliferation | modelling | matrix | system |
| Ta gr2 | ↑ | — | — | — | ↓↓ | ↓ |
| Ta gr3 | ↑↑↑ | ↑↑ | ↑↑ | — | ↓↓ | ↓ |
| T1 gr3 | ↑^{b} | — | ↑↑^{b} | — | ↓ | ↑^{b} |
| T2 gr3 | ↑ | — | ↑↑↑ | ↑↑↑ | ↑ | ↑ |
| Ta gr3 + CIS | ↑↑↑ | ↑↑ | ↑↑↑ | ↑↑↑ | ↑ | ↑ |

^{a}For a detailed description of gene clusters see FIG. 8.
^{b}An increase in gene expression was only found in about half of the samples analysed.

### Class Prediction of Bladder Tumours

An objective class prediction of bladder tumours based on a limited gene-set is clinically useful. We therefore built a classifier using tumours correctly separated in the three main groups as identified in the cluster dendrogram (FIG. 6a). We used a maximum likelihood classification method with a “leave one out” cross-validation scheme (Shipp et al. 2002; van't Veer et al. 2002) in which one test tumour was removed from the set, and a set of predictive genes was selected from the remaining tumour samples for classifying the test tumour. This process was repeated for all tumours. Predictive genes that showed the largest possible separation of the three groups were selected for classification, and each tumour was classified according to how close it was to the mean of the three groups (FIG. 8a).

### Classification of Samples

From the hierarchical cluster analysis of the samples (class discovery) we identified three major “molecular classes” of bladder carcinoma highly associated with the pathologic staging of the samples. Based on this finding we decided to build a molecular classifier that assigns tumours to these three “molecular classes”. To build the classifier, we only used the tumours in which there was a correlation between the “molecular class” and the associated pathologic stage. Consequently, a T1 tumour clustering in the “molecular class” of T2 tumours was not used to build the classifier.

The genes used in the classifier were those genes with the highest values of the ratio (B/W) of the variation between the groups to the variation within the groups. High values of the ratio (B/W) signify genes with good group separation performance. We calculated the sum over the genes of the squared distance from the sample value to the group mean and classified the sample as belonging to the group where the distance to the group mean was smallest. If the relative difference between the distance to the closest and the second closest group compared to the distance to the closest group were below 5%, the classification failed and the sample was classified as belonging to both groups. The relative difference is referred to as the classifier strength.

### Classifier Performance

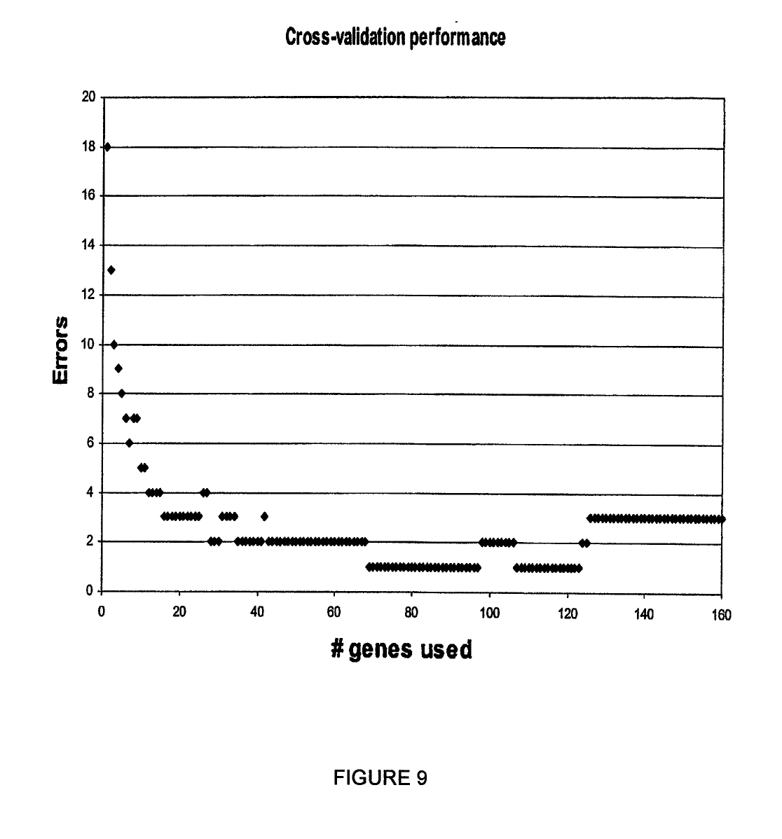

The classifier performance was tested using from 1-160 genes in cross-validation loops. FIG. 9 shows that the closest correlation to histopathology is obtained in the cross-validation model using from 69-97 genes. Based on this we chose the model using 80 genes for cross-validation as our final classifier model.

### Classifier Model Using 71 Genes

We selected those genes for our final classifier model that were used in at least 75% (25 times) of the cross-validation loops. These 71 genes are listed in table 7.

TABLE 7
Feature: Accession number on HuGene fl array. Number: Number of times used in
the 80 genes cross validation loops. Test (B/W): see below.

| | Unigene | | | Test |
|---|---|---|---|---|
| Feature | Build 162 | Description | Number | (B/W) |
| AF000231_at | Hs.75618 | NM_004663; Ras-related protein Rab-11A | 33 | 26.77 |
| D13666_s_at | Hs.136348 | NM_006475; osteoblast specific factor 2 (fasciclin I-like) | 33 | 27.71 |
| D49372_s_at | Hs.54460 | NM_002986; small inducible cytokine A11 precursor | 31 | 25.78 |
| D83920_at | Hs.440898 | NM_002003; ficolin 1 precursor | 33 | 31.18 |
| D86479_at | Hs.439463 | NM_001129; adipocyte enhancer binding protein 1 precursor | 33 | 28.29 |
| D89077_at | Hs.75367 | NM_006748; Src-like-adaptor | 33 | 30.03 |
| D89377_at | Hs.89404 | NM_002449; msh homeo box homolog 2 | 33 | 51.50 |
| HG4069-HT4339_s_at | | | 27 | 25.06 |
| HG67-HT67_f_at | | | 33 | 27.81 |
| HG907-HT907_at | | | 33 | 25.76 |
| J02871_s_at | Hs.436317 | NM_000779; cytochrome P450, family 4, subfamily B, | 33 | 32.61 |
| | | polypeptide 1 | | |
| J03278_at | Hs.307783 | NM_002609; platelet-derived growth factor receptor beta | 33 | 28.02 |
| | | precursor | | |
| J04058_at | Hs.169919 | NM_000126; electron transfer flavoprotein, alpha poly- | 33 | 29.46 |
| | | peptide | | |
| J05032_at | Hs.32393 | NM_001349; aspartyl-tRNA synthetase | 33 | 38.21 |
| J05070_at | Hs.151738 | NM_004994; matrix metalloproteinase 9 preproprotein | 33 | 35.34 |
| J05448_at | Hs.79402 | NM_002694; DNA directed RNA polymerase II polypeptide | 32 | 26.51 |
| | | C NM_032940; DNA directed RNA polymerase II | | |
| | | polypeptide C | | |
| K01396_at | Hs.297681 | NM_000295; serine (or cysteine) proteinase inhibitor, | 33 | 28.66 |
| | | clade A (alpha-1 antiproteinase, antitrypsin), member 1 | | |
| L13720_at | Hs.437710 | NM_000820; growth arrest-specific 6 | 33 | 29.69 |
| M12125_at | Hs.300772 | NM_003289; tropomyosin 2 (beta) | 28 | 24.89 |
| M15395_at | Hs.375957 | NM_000211; integrin beta chain, beta 2 precursor | 33 | 29.40 |
| M16591_s_at | Hs.89555 | NM_002110; hemopoietic cell kinase isoform p61HCK | 33 | 32.34 |
| M20530_at | | | 33 | 30.28 |
| M23178_s_at | Hs.73817 | NM_002983; chemokine (C-C motif) ligand 3 | 33 | 35.36 |
| M32011_at | Hs.949 | NM_000433; neutrophil cytosolic factor 2 | 33 | 41.88 |
| M33195_at | Hs.433300 | NM_004106; Fc fragment of IgE, high affinity I, receptor | 33 | 30.40 |
| | | for, gamma polypeptide precursor | | |
| M55998_s_at | Hs.172928 | NM_000088; alpha 1 type I collagen preproprotein | 33 | 26.83 |
| M57731_s_at | Hs.75765 | NM_002089; chemokine (C—X—C motif) ligand 2 | 33 | 31.84 |
| M68840_at | Hs.183109 | NM_000240; monoamine oxidase A | 33 | 32.39 |
| M69203_s_at | Hs.75703 | NM_002984; chemokine (C-C motif) ligand 4 precursor | 33 | 36.21 |
| M72885_rna1_s_at | | | 33 | 27.94 |
| M83822_at | Hs.209846 | NM_006726; LPS-responsive vesicle trafficking, beach | 33 | 26.44 |
| | | and anchor containing | | |
| S77393_at | Hs.145754 | NM_016531; Kruppel-like factor 3 (basic) | 33 | 49.85 |
| U01833_at | Hs.81469 | NM_002484; nucleotide binding protein 1 (MinD homolog, | 33 | 30.62 |
| | | E. coli) | | |
| U07231_at | Hs.309763 | NM_002092; G-rich RNA sequence binding factor 1 | 33 | 39.10 |
| U09937_rna1_s_at | | | 33 | 30.88 |
| U10550_at | Hs.79022 | NM_005261; GTP-binding mitogen-induced T-cell protein | 28 | 25.26 |
| | | NM_181702; GTP-binding mitogen-induced T-cell protein | | |
| U20158_at | Hs.2488 | NM_005565; lymphocyte cytosolic protein 2 | 33 | 32.41 |
| U41315_rna1_s_at | | | 33 | 43.56 |
| U47414_at | Hs.13291 | NM_004354; cyclin G2 | 33 | 44.42 |
| U49352_at | Hs.414754 | NM_001359; 2,4-dienoyl CoA reductase 1 precursor | 33 | 37.04 |
| U50708_at | Hs.1265 | NM_000056; branched chain keto acid dehydrogenase | 33 | 42.89 |
| | | E1, beta polypeptide precursor NM_183050; branched | | |
| | | chain keto acid dehydrogenase E1, beta polypeptide | | |
| | | precursor | | |
| U52101_at | Hs.9999 | NM_001425; epithelial membrane protein 3 | 33 | 29.86 |
| U64520_at | Hs.66708 | NM_004781; vesicle-associated membrane protein 3 | 33 | 30.17 |
| | | (cellubrevin) | | |
| U65093_at | Hs.82071 | NM_006079; Cbp/p300-interacting transactivator, with | 33 | 32.07 |
| | | Glu/Asp-rich carboxy-terminal domain, 2 | | |
| U68019_at | Hs.288261 | NM_005902; MAD, mothers against decapentaplegic | 31 | 26.70 |
| | | homolog 3 | | |
| U68385_at | Hs.380923 | | 33 | 31.56 |
| U74324_at | Hs.90875 | NM_002871; RAB-interacting factor | 33 | 30.26 |
| U77970_at | Hs.321164 | NM_002518; neuronal PAS domain protein 2 | 33 | 50.37 |
| | | NM_032235; | | |
| U90549_at | Hs.236774 | NM_006353; high mobility group nucleosomal binding | 33 | 32.16 |
| | | domain 4 | | |
| X04085_rna1_at | | | 28 | 25.13 |
| X07743_at | Hs.77436 | NM_002664; pleckstrin | 33 | 28.13 |
| X13334_at | Hs.75627 | NM_000591; CD14 antigen precursor | 33 | 35.79 |
| X14046_at | Hs.153053 | NM_001774; CD37 antigen | 30 | 24.70 |
| X15880_at | Hs.415997 | NM_001848; collagen, type VI, alpha 1 precursor | 33 | 31.51 |
| X15882_at | Hs.420269 | NM_001849; alpha 2 type VI collagen isoform 2C2 precursor | 33 | 32.32 |
| | | NM_058174; alpha 2 type VI collagen isoform | | |
| | | 2C2a precursor NM_058175; alpha 2 type VI collagen | | |
| | | isoform 2C2a precursor | | |
| X51408_at | Hs.380138 | NM_001822; chimerin (chimaerin) 1 | 33 | 30.51 |
| X53800_s_at | Hs.89690 | NM_002090; chemokine (C—X—C motif) ligand 3 | 33 | 33.63 |
| X54489_rna1_at | | | 33 | 33.57 |
| X57579_s_at | | | 33 | 41.43 |
| X64072_s_at | Hs.375957 | NM_000211; integrin beta chain, beta 2 precursor | 33 | 43.21 |
| X67491_f_at | Hs.355697 | NM_005271; glutamate dehydrogenase 1 | 33 | 30.97 |
| X68194_at | Hs.80919 | NM_006754; synaptophysin-like protein isoform a | 33 | 46.53 |
| | | NM_182715; synaptophysin-like protein isoform b | | |
| X73882_at | Hs.254605 | NM_003980; microtubule-associated protein 7 | 33 | 53.16 |
| X78520_at | Hs.372528 | NM_001829; chloride channel 3 | 33 | 47.38 |
| Y00787_s_at | Hs.624 | NM_000584; interleukin 8 precursor | 32 | 27.54 |
| Z12173_at | Hs.334534 | NM_002076; glucosamine (N-acetyl)-6-sulfatase precursor | 30 | 25.44 |
| Z19554_s_at | Hs.435800 | NM_003380; vimentin | 27 | 24.59 |
| Z26491_s_at | Hs.240013 | NM_000754; catechol-O-methyltransferase isoform MB- | 32 | 26.92 |
| | | COMT NM_007310; catechol-O-methyltransferase isoform | | |
| | | S-COMT | | |
| Z29331_at | Hs.372758 | NM_003344; ubiquitin-conjugating enzyme E2H isoform 1 | 33 | 33.49 |
| | | NM_182697; ubiquitin-conjugating enzyme E2H isoform 2 | | |
| Z48605_at | Hs.421825 | NM_006903; inorganic pyrophosphatase 2 isoform 2 | 33 | 44.45 |
| | | NM_176865; NM_176866; inorganic pyrophosphatase 2 | | |
| | | isoform 3 NM_176867; inorganic pyrophosphatase 2 | | |
| | | isoform 4 NM_176869; inorganic pyrophosphatase 2 | | |
| | | isoform 1 | | |
| Z74615_at | Hs.172928 | NM_000088; alpha 1 type I collagen preproprotein | 33 | 55.18 |

### Test for Significance of Classifier

To test the class separation performance of the 71 selected genes we compared the B/W ratios with the similar ratios of all the genes calculated from permutations of the arrays. For each permutation we construct three pseudogroups, pseudo-Ta, pseudo-T1, and pseudo-T2, so that the proportion of samples from the three original groups is approximately the same in the three pseudogroups. We then calculate the ratio of the variation between the pseudogroups to the variation within the pseudogroups for all the genes. For 500 permutations we only two times had one gene for which the B/W value was higher than the lowest value for the original B/W values of the 71 selected genes (the two values being 25.28 and 25.93).

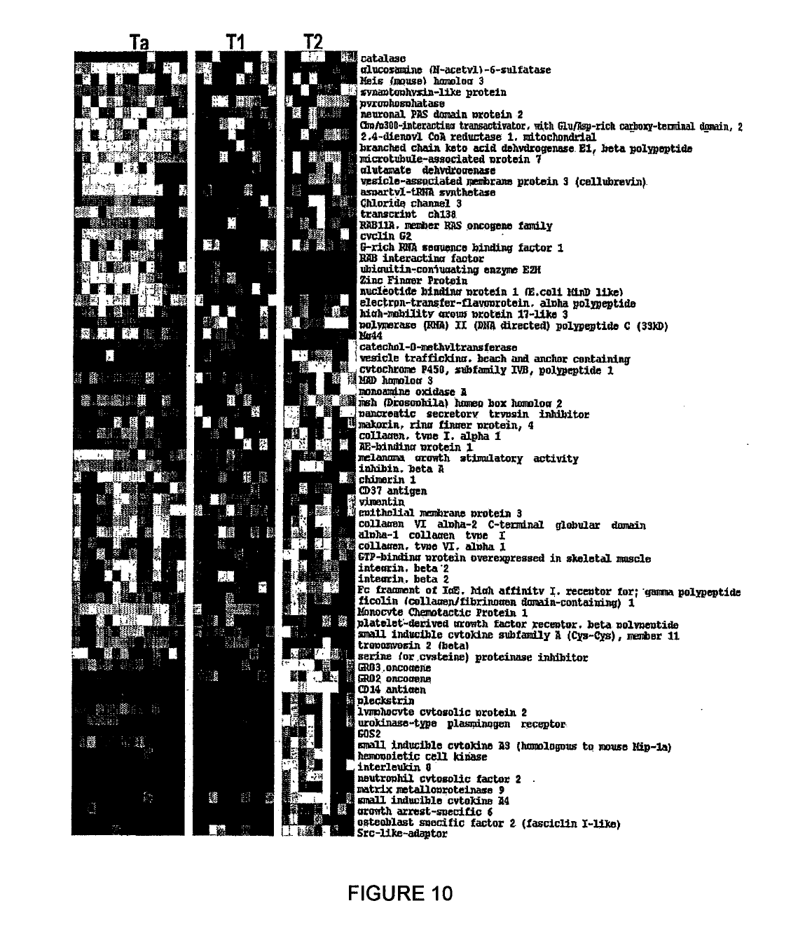

The classifier performance was tested using from 1-160 genes in cross-validation loops, and a model using an 80 gene cross-validation scheme showed the best correlation to pathologic staging (p<10⁻⁹). The 71 genes that were used in at least 75% of the cross validation loops were selected to constitute our final classifier model. See the expression profiles of the 71 genes in FIG. 10. The genes are clustered to obtain a better overview of similar expression patterns. From this it is obvious that the T1 stage is characterised by having expression patterns in common with either Ta or T2 tumours. There are no single genes that can be used as a T1 marker.

### Permutation Analysis

To test the class separation performance of the 71 selected genes we compared their performance to those of a permutated set of pseudo-Ta, T1 and T2 tumours. In 500 permutations we only detected two genes with a performance equal to the poorest performing classifying genes.

### Classification Using 80 Predictive Genes and Other Gene-Sets

The classification using 80 predictive genes in cross-validation loops identified the Ta group with no surrounding CIS and no previous tumor or no previous tumor of a higher stage (Table 8). Interestingly, the Ta tumours surrounded by CIS that were classified as T2 or T1 clearly demonstrate the potential of the classification method for identifying surrounding CIS in a non-invasive way, thereby supplementing clinical and pathologic information.

TABLE 8
Table 8 Clinical data on disease courses and results of molecular classification

| | | | | | Carci- | | | | |
|---|---|---|---|---|---|---|---|---|---|
| | | Previous | Tumour | Subsequent | noma | Reviewed | Molecular classifier | | |
| Tumours^{a} | Patient | tumours | analysed | tumours | in situ^{b} | histology^{c} | 320 | 80 | 20 |

Ta grade II tumours - no progression

| | | | | | | | |
|---|---|---|---|---|---|---|---|
| 709-1 | Ta gr2 | | No | Ta gr3 | Ta | Ta | Ta |
| 968-1 | Ta gr2 | 1 Ta | No | | Ta/T1 | Ta | Ta |
| 934-1 | Ta gr2 | | No | | T1 | Ta | Ta |
| 928-1 | Ta gr2 | | No | | Ta | Ta | T1 |
| 930-1 | Ta gr2 | | No | | Ta | Ta | Ta |

Ta grade III tumours - no prior T1 tumour or CIS

| | | | | | | | | |
|---|---|---|---|---|---|---|---|---|
| 989-1 | | Ta gr3 | | No | | Ta | Ta | Ta |
| 1264-1 | | Ta gr3 | 3 Ta | No | | Ta | Ta | Ta |
| 876-5 | 4 Ta | Ta gr3 | | No | | Ta | Ta | Ta |
| 669-7 | 5 Ta | Ta gr3 | 4 Ta | No | Ta gr2 | Ta | Ta | Ta |
| 716-2 | 1 Ta | Ta gr3 | 2 Ta | No | | Ta | Ta | Ta |

Ta grade III tumours - no prior T1 tumour but CIS in selected site biopsies

| | | | | | | |
|---|---|---|---|---|---|---|
| 1070-1 | Ta gr3 | 1 Ta | Subsequent | Ta | Ta | Ta |
| | | | visit | | | |
| 956-2 | Ta gr3 | 1 Ta | Sampling | T2 | T2 | T2/T1 |
| | | | visit | | | |
| 1062-2 | Ta gr3 | 1 T1 | Sampling | T2/Ta | T1/Ta | Ta |
| | | | visit | | | |
| 1166-1 | Ta gr3 | | Sampling | Ta/T1 | Ta | Ta |
| | | | visit | | | |
| 1330-1 | Ta gr3 | | Sampling | T2 | T2 | Ta |
| | | | visit | | | |

Ta grade III tumours - a prior T1 tumour and CIS in selected site biopsies

| | | | | | | | |
|---|---|---|---|---|---|---|---|
| 747-7 | 5 Ta, 1 T1 | Ta gr3 | 3 Ta | Sampling | Ta | Ta | Ta |
| | | | | visit | | | |
| 112-10 | 7 Ta, 2 T1 | Ta gr3 | 2 Ta, 4 T1 | Previous | Ta | Ta | Ta |
| | | | | visit | | | |
| 320-7 | 1 Ta, 2 T1 | Ta gr3 | 2 Ta | Sampling | T2 | T2 | Ta |
| | | | | visit | | | |
| 967-3 | 2 T1 | Ta gr3 | 1 T1 | Sampling | Ta | Ta | Ta |
| | | | | visit | | | |

T1 grade III tumours - no prior muscle invasive tumour

| | | | | | | | | |
|---|---|---|---|---|---|---|---|---|
| 625-1 | | T1 gr3 | | No | | T1 | T1 | T1 |
| 847-1 | | T1 gr3 | | No | | T1 | T1 | T1 |
| 1257-1 | | T1 gr3 | | Sampling | | T1 | T1 | T1 |
| | | | | visit | | | | |
| 919-1 | | T1 gr3 | | No | | T1 | T1 | T1 |
| 880-1 | | T1 gr3 | 4 Ta | No | | T1 | T1 | T1 |
| 812-1 | | T1 gr3 | | No | | T1 | T1 | T1 |
| 1269-1 | | T1 gr3 | | No | No review | T1 | T1 | T1 |
| 1083-2 | 1 Ta | T1 gr3 | | No | No review | T1 | T1 | T1 |
| 1238-1 | | T1 gr3 | 1 Ta, 1 T2+ | No | | T1 | T1 | T1 |
| 1065-1 | | T1 gr3 | | Subsequent | No review | T1 | T1 | T1 |
| | | | | visit | | | | |
| 1134-1 | | T1 gr3 | 3 T1 | Sampling | T2 gr3 | T1 | T1 | T1 |
| | | | | visit | | | | |

T2+ grade III/IV tumours - only primary tumours

| | | | | | | | |
|---|---|---|---|---|---|---|---|
| 1164-1 | T2+ gr4 | | No | T2+ gr3 | T2/T1 | T1 | T1 |
| 1032-1 | T2+ gr? | | ND | No review | T2 | T2 | T2 |
| 1117-1 | T2+ gr3 | | ND | | T2 | T2 | T1 |
| 1178-1 | T2+ gr3 | | ND | | T2 | T2 | T2 |
| 1078-1 | T2+ gr3 | | ND | | T2 | T2 | T2 |
| 875-1 | T2+ gr3 | | No | | T2 | T2 | T2 |
| 1044-1 | T2+ gr3 | 1 T2+ | ND | | T2 | T2 | T2 |
| 1133-1 | T2+ gr3 | | ND | | T2 | T2 | T2 |
| 1068-1 | T2+ gr3 | | No | | T2 | T2 | T2 |
| 937-1 | T2+ gr3 | | ND | No review | T1 | T1 | T1 |

^{a}Examples of tumour histology.
^{b}Carcinoma in situ detected in selected site biopsies at the time of sampling tumour tissue for the arrays or at previous or subsequent visits.
^{c}All tumours were reviewed by a single uro-pathologist and any change compared to the routine classification is listed.
^{d}Molecular classification based on 320, 80, and 20 genes cross-validation loops.

### Classification Using Other Gene-Sets

Classification was also carried out using other gene-sets (10, 20, 32, 40, 80, 160, and 320 genes). These gene-sets demonstrated the same classification tendency as the 71 genes. See Tables 9-15 for gene-sets.

TABLE 9
320 genes for classifier

| Chip acc. # | UniGene Build 162 | description |
|---|---|---|
| AB000220_at | Hs.171921 | NM_006379; semaphorin 3C |
| AB000220_at | Hs.171921 | NM_006379; semaphorin 3C |
| AC002073_cds1_at | | |
| AF000231_at | Hs.75618 | NM_004663; Ras- |
| | | related protein Rab-11A |
| D10922_s_at | Hs.99855 | NM_001462; formyl |
| | | peptide receptor-like 1 |
| D10925_at | Hs.301921 | NM_001295; |
| | | chemokine (C-C motif) |
| | | receptor 1 |
| D11086_at | Hs.84 | NM_000206; interleukin |
| | | 2 receptor, gamma |
| | | chain, precursor |
| D11151_at | Hs.211202 | NM_001957; endothelin |
| | | receptor type A |
| D13435_at | Hs.426142 | NM_002643; phosphatidylinositol |
| | | glycan, class F isoform 1 |
| | | NM_173074; phosphatidylinositol |
| | | glycan, class F isoform 2 |
| D13666_s_at | Hs.136348 | NM_006475; osteoblast |
| | | specific factor 2 (fasciclin I-like) |
| D14520_at | Hs.84728 | NM_001730; Kruppel-like factor 5 |
| D21878_at | Hs.169998 | NM_004334; bone marrow stromal cell |
| | | antigen 1 precursor |
| D26443_at | Hs.371369 | NM_004172; solute |
| | | carrier family 1 (glial |
| | | high affinity glutamate transporter), |
| | | member 3 |
| D28589_at | Hs.17719 | |
| D42046_at | Hs.194665 | |
| D45370_at | Hs.74120 | NM_006829; adipose |
| | | specific 2 |
| D49372_s_at | Hs.54460 | NM_002986; small |
| | | inducible cytokine A11 |
| | | precursor |
| D50495_at | Hs.224397 | NM_003195; transcription |
| | | elongation factor A |
| | | (SII), 2 |
| D63135_at | Hs.27935 | NM_032646; tweety |
| | | homolog 2 |
| D64053_at | Hs.198288 | NM_002849; protein |
| | | tyrosine phosphatase, |
| | | receptor type, R isoform |
| | | 1 precursor |
| | | NM_130846; protein |
| | | tyrosine phosphatase, |
| | | receptor type, R isoform 2 |
| D83920_at | Hs.440898 | NM_002003; ficolin 1 |
| | | precursor |
| D85131_s_at | Hs.433881 | NM_002383; MYC- |
| | | associated zinc finger |
| | | protein |
| D86062_s_at | Hs.413482 | NM_004649; chromosome |
| | | 21 open reading |
| | | frame 33 |
| D86479_at | Hs.439463 | NM_001129; adipocyte |
| | | enhancer binding protein |
| | | 1 precursor |
| D86957_at | Hs.307944 | |
| D86959_at | Hs.105751 | NM_014720; Ste20- |
| | | related serine/threonine |
| | | kinase |
| D86976_at | Hs.196914 | |
| D87433_at | Hs.301989 | NM_015136; stabilin 1 |
| D87443_at | Hs.409862 | NM_014758; sorting |
| | | nexin 19 |
| D87682_at | Hs.134792 | |
| D89077_at | Hs.75367 | NM_006748; Src-like- |
| | | adaptor |
| D89377_at | Hs.89404 | NM_002449; msh |
| | | homeo box homolog 2 |
| D90279_s_at | Hs.433695 | NM_000093; alpha 1 |
| | | type V collagen prepro- |
| | | protein |
| HG1996-HT2044_at | | |
| HG2090-HT2152_s_at | | |
| HG2463-HT2559_at | | |
| HG2994-HT4850_s_at | | |
| HG3044-HT3742_s_at | | |
| HG3187-HT3366_s_at | | |
| HG3342-HT3519_s_at | | |
| HG371-HT26388_s_at | | |
| HG4069-HT4339_s_at | | |
| HG67-HT67_f_at | | |
| HG907-HT907_at | | |
| J02871_s_at | Hs.436317 | NM_000779; cytochrome |
| | | P450, family 4, |
| | | subfamily B, polypeptide 1 |
| J03040_at | Hs.111779 | NM_003118; secreted |
| | | protein, acidic, cysteine- |
| | | rich (osteonectin) |
| J03060_at | | |
| J03068_at | | |
| J03241_s_at | Hs.2025 | NM_003239; transforming |
| | | growth factor, beta 3 |
| J03278_at | Hs.307783 | NM_002609; platelet- |
| | | derived growth factor |
| | | receptor beta precursor |
| J03909_at | | |
| J03925_at | Hs.172631 | NM_000632; integrin |
| | | alpha M precursor |
| J04056_at | Hs.88778 | NM_001757; carbonyl |
| | | reductase 1 |
| J04058_at | Hs.169919 | NM_000126; electron |
| | | transfer flavoprotein, |
| | | alpha polypeptide |
| J04093_s_at | Hs.278896 | NM_019075; UDP |
| | | glycosyltransferase 1 |
| | | family, polypeptide A10 |
| J04130_s_at | Hs.75703 | NM_002984; |
| | | chemokine (C-C motif) |
| | | ligand 4 precursor |
| J04152_rna1_s_at | | |
| J04162_at | Hs.372679 | NM_000569; Fc fragment |
| | | of IgG, low affinity |
| | | IIIa, receptor for (CD16) |
| J04456_at | Hs.407909 | NM_002305; beta- |
| | | galactosidase binding |
| | | lectin precursor |
| J05032_at | Hs.32393 | NM_001349; aspartyl- |
| | | tRNA synthetase |
| J05036_s_at | Hs.1355 | NM_001910; cathepsin |
| | | E isoform a preproprotein |
| | | NM_148964; |
| | | cathepsin E isoform b |
| | | preproprotein |
| J05070_at | Hs.151738 | NM_004994; matrix |
| | | metalloproteinase 9 |
| | | preproprotein |
| J05448_at | Hs.79402 | NM_002694; DNA |
| | | directed RNA polymerase |
| | | II polypeptide C |
| | | NM_032940; DNA |
| | | directed RNA polymerase |
| | | II polypeptide C |
| K01396_at | Hs.297681 | NM_000295; serine (or |
| | | cysteine) proteinase |
| | | inhibitor, clade A (alpha- |
| | | 1 antiproteinase, |
| | | antitrypsin), member 1 |
| K03430_at | | |
| L06797_s_at | Hs.421986 | NM_003467; |
| | | chemokine (C—X—C |
| | | motif) receptor 4 |
| L10343_at | Hs.112341 | NM_002638; skin- |
| | | derived protease inhibitor |
| | | 3 preproprotein |
| L11708_at | Hs.155109 | NM_002153; hydroxysteroid |
| | | (17-beta) dehydrogenase 2 |
| L13391_at | Hs.78944 | NM_002923; regulator |
| | | of G-protein signalling |
| | | 2, 24 kDa |
| L13698_at | Hs.65029 | NM_002048; growth |
| | | arrest-specific 1 |
| L13720_at | Hs.437710 | NM_000820; growth |
| | | arrest-specific 6 |
| L13923_at | Hs.750 | NM_000138; fibrillin 1 |
| AB000220_at | Hs.171921 | NM_006379; semaphorin |
| | | 3C |
| AC002073_cds1_at | | |
| AF000231_at | Hs.75618 | NM_004663; Ras- |
| | | related protein Rab-11A |
| D10922_s_at | Hs.99855 | NM_001462; formyl |
| | | peptide receptor-like 1 |
| D10925_at | Hs.301921 | NM_001295; |
| | | chemokine (C-C motif) |
| | | receptor 1 |
| D11086_at | Hs.84 | NM_000206; interleukin |
| | | 2 receptor, gamma |
| | | chain, precursor |
| D11151_at | Hs.211202 | NM_001957; endothelin |
| | | receptor type A |
| D13435_at | Hs.426142 | NM_002643; phosphatidylinositol |
| | | glycan, |
| | | class F isoform 1 |
| | | NM_173074; phosphatidylinositol |
| | | glycan, |
| | | class F isoform 2 |
| D13666_s_at | Hs.136348 | NM_006475; osteoblast |
| | | specific factor 2 (fasciclin |
| | | I-like) |
| D14520_at | Hs.84728 | NM_001730; Kruppel- |
| | | like factor 5 |
| D21878_at | Hs.169998 | NM_004334; bone |
| | | marrow stromal cell |
| | | antigen 1 precursor |
| D26443_at | Hs.371369 | NM_004172; solute |
| | | carrier family 1 (glial |
| | | high affinity glutamate |
| | | transporter), member 3 |
| D28589_at | Hs.17719 | |
| D42046_at | Hs.194665 | |
| D45370_at | Hs.74120 | NM_006829; adipose |
| | | specific 2 |
| D49372_s_at | Hs.54460 | NM_002986; small |
| | | inducible cytokine A11 |
| | | precursor |
| D50495_at | Hs.224397 | NM_003195; transcription |
| | | elongation factor A |
| | | (SII), 2 |
| D63135_at | Hs.27935 | NM_032646; tweety |
| | | homolog 2 |
| D64053_at | Hs.198288 | NM_002849; protein |
| | | tyrosine phosphatase, |
| | | receptor type, R isoform |
| | | 1 precursor |
| | | NM_130846; protein |
| | | tyrosine phosphatase, |
| | | receptor type, R isoform 2 |
| D83920_at | Hs.440898 | NM_002003; ficolin 1 |
| | | precursor |
| D85131_s_at | Hs.433881 | NM_002383; MYC- |
| | | associated zinc finger |
| | | protein |
| D86062_s_at | Hs.413482 | NM_004649; chromosome |
| | | 21 open reading |
| | | frame 33 |
| D86479_at | Hs.439463 | NM_001129; adipocyte |
| | | enhancer binding protein |
| | | 1 precursor |
| D86957_at | Hs.307944 | |
| D86959_at | Hs.105751 | NM_014720; Ste20- |
| | | related serine/threonine |
| | | kinase |
| D86976_at | Hs.196914 | |
| D87433_at | Hs.301989 | NM_015136; stabilin 1 |
| D87443_at | Hs.409862 | NM_014758; sorting |
| | | nexin 19 |
| D87682_at | Hs.134792 | |
| D89077_at | Hs.75367 | NM_006748; Src-like- |
| | | adaptor |
| D89377_at | Hs.89404 | NM_002449; msh |
| | | homeo box homolog 2 |
| D90279_s_at | Hs.433695 | NM_000093; alpha 1 |
| | | type V collagen prepro- |
| | | protein |
| HG1996-HT2044_at | | |
| HG2090-HT2152_s_at | | |
| HG2463-HT2559_at | | |
| HG2994-HT4850_s_at | | |
| HG3044-HT3742_s_at | | |
| HG3187-HT3366_s_at | | |
| HG3342-HT3519_s_at | | |
| HG371-HT26388_s_at | | |
| HG4069-HT4339_s_at | | |
| HG67-HT67_f_at | | |
| HG907-HT907_at | | |
| J02871_s_at | Hs.436317 | NM_000779; cytochrome |
| | | P450, family 4, |
| | | subfamily B, polypeptide 1 |
| J03040_at | Hs.111779 | NM_003118; secreted |
| | | protein, acidic, cysteine- |
| | | rich (osteonectin) |
| J03060_at | | |
| J03068_at | | |
| J03241_s_at | Hs.2025 | NM_003239; transforming |
| | | growth factor, beta 3 |
| J03278_at | Hs.307783 | NM_002609; platelet- |
| | | derived growth factor |
| | | receptor beta precursor |
| J03909_at | | |
| J03925_at | Hs.172631 | NM_000632; integrin |
| | | alpha M precursor |
| J04056_at | Hs.88778 | NM_001757; carbonyl |
| | | reductase 1 |
| J04058_at | Hs.169919 | NM_000126; electron |
| | | transfer flavoprotein, |
| | | alpha polypeptide |
| J04093_s_at | Hs.278896 | NM_019075; UDP |
| | | glycosyltransferase 1 |
| | | family, polypeptide A10 |
| J04130_s_at | Hs.75703 | NM_002984; |
| | | chemokine (C-C motif) |
| | | ligand 4 precursor |
| J04152_rna1_s_at | | |
| J04162_at | Hs.372679 | NM_000569; Fc fragment |
| | | of IgG, low affinity |
| | | IIIa, receptor for (CD16) |
| J04456_at | Hs.407909 | NM_002305; beta- |
| | | galactosidase binding |
| | | lectin precursor |
| J05032_at | Hs.32393 | NM_001349; aspartyl- |
| | | tRNA synthetase |
| J05036_s_at | Hs.1355 | NM_001910; cathepsin |
| | | E isoform a preproprotein |
| | | NM_148964; |
| | | cathepsin E isoform b |
| | | preproprotein |
| J05070_at | Hs.151738 | NM_004994; matrix |
| | | metalloproteinase 9 |
| | | preproprotein |
| J05448_at | Hs.79402 | NM_002694; DNA |
| | | directed RNA polymerase |
| | | II polypeptide C |
| | | NM_032940; DNA |
| | | directed RNA polymerase |
| | | II polypeptide C |
| K01396_at | Hs.297681 | NM_000295; serine (or |
| | | cysteine) proteinase |
| | | inhibitor, clade A (alpha- |
| | | 1 antiproteinase, |
| | | antitrypsin), member 1 |
| K03430_at | | |
| L06797_s_at | Hs.421986 | NM_003467; |
| | | chemokine (C—X—C |
| | | motif) receptor 4 |
| L10343_at | Hs.112341 | NM_002638; skin- |
| | | derived protease inhibitor |
| | | 3 preproprotein |
| L11708_at | Hs.155109 | NM_002153; hydroxysteroid |
| | | (17-beta) dehydrogenase 2 |
| L13391_at | Hs.78944 | NM_002923; regulator |
| | | of G-protein signalling |
| | | 2, 24 kDa |
| L13698_at | Hs.65029 | NM_002048; growth |
| | | arrest-specific 1 |
| L13720_at | Hs.437710 | NM_000820; growth |
| | | arrest-specific 6 |
| L13923_at | Hs.750 | NM_000138; fibrillin 1 |
| AB000220_at | Hs.171921 | NM_006379; semaphorin |
| | | 3C |
| AC002073_cds1_at | | |
| AF000231_at | Hs.75618 | NM_004663; Ras- |
| | | related protein Rab-11A |
| D10922_s_at | Hs.99855 | NM_001462; formyl |
| | | peptide receptor-like 1 |
| D10925_at | Hs.301921 | NM_001295; |
| | | chemokine (C-C motif) |
| | | receptor 1 |
| D11086_at | Hs.84 | NM_000206; interleukin |
| | | 2 receptor, gamma |
| | | chain, precursor |
| D11151_at | Hs.211202 | NM_001957; endothelin |
| | | receptor type A |
| D13435_at | Hs.426142 | NM_002643; phosphatidylinositol |
| | | glycan, |
| | | class F isoform 1 |
| | | NM_173074; phosphatidylinositol |
| | | glycan, |
| | | class F isoform 2 |
| D13666_s_at | Hs.136348 | NM_006475; osteoblast |
| | | specific factor 2 (fasciclin |
| | | I-like) |
| D14520_at | Hs.84728 | NM_001730; Kruppel- |
| | | like factor 5 |
| D21878_at | Hs.169998 | NM_004334; bone |
| | | marrow stromal cell |
| | | antigen 1 precursor |
| D26443_at | Hs.371369 | NM_004172; solute |
| | | carrier family 1 (glial |
| | | high affinity glutamate |
| | | transporter), member 3 |
| D28589_at | Hs.17719 | |
| D42046_at | Hs.194665 | |
| D45370_at | Hs.74120 | NM_006829; adipose |
| | | specific 2 |
| D49372_s_at | Hs.54460 | NM_002986; small |
| | | inducible cytokine A11 |
| | | precursor |
| D50495_at | Hs.224397 | NM_003195; transcription |
| | | elongation factor A |
| | | (SII), 2 |
| D63135_at | Hs.27935 | NM_032646; tweety |
| | | homolog 2 |
| D64053_at | Hs.198288 | NM_002849; protein |
| | | tyrosine phosphatase, |
| | | receptor type, R isoform |
| | | 1 precursor |
| | | NM_130846; protein |
| | | tyrosine phosphatase, |
| | | receptor type, R isoform 2 |
| D83920_at | Hs.440898 | NM_002003; ficolin 1 |
| | | precursor |
| D85131_s_at | Hs.433881 | NM_002383; MYC- |
| | | associated zinc finger |
| | | protein |
| D86062_s_at | Hs.413482 | NM_004649; chromosome |
| | | 21 open reading |
| | | frame 33 |
| D86479_at | Hs.439463 | NM_001129; adipocyte |
| | | enhancer binding protein |
| | | 1 precursor |
| D86957_at | Hs.307944 | |
| D86959_at | Hs.105751 | NM_014720; Ste20- |
| | | related serine/threonine |
| | | kinase |
| D86976_at | Hs.196914 | |
| D87433_at | Hs.301989 | NM_015136; stabilin 1 |
| D87443_at | Hs.409862 | NM_014758; sorting |
| | | nexin 19 |
| D87682_at | Hs.134792 | |
| D89077_at | Hs.75367 | NM_006748; Src-like- |
| | | adaptor |
| D89377_at | Hs.89404 | NM_002449; msh |
| | | homeo box homolog 2 |
| D90279_s_at | Hs.433695 | NM_000093; alpha 1 |
| | | type V collagen prepro- |
| | | protein |
| HG1996-HT2044_at | | |
| HG2090-HT2152_s_at | | |
| HG2463-HT2559_at | | |
| HG2994-HT4850_s_at | | |
| HG3044-HT3742_s_at | | |
| HG3187-HT3366_s_at | | |
| HG3342-HT3519_s_at | | |
| HG371-HT26388_s_at | | |
| HG4069-HT4339_s_at | | |
| HG67-HT67_f_at | | |
| HG907-HT907_at | | |
| J02871_s_at | Hs.436317 | NM_000779; cytochrome |
| | | P450, family 4, |
| | | subfamily B, polypeptide 1 |
| J03040_at | Hs.111779 | NM_003118; secreted |
| | | protein, acidic, cysteine- |
| | | rich (osteonectin) |
| J03060_at | | |
| J03068_at | | |
| J03241_s_at | Hs.2025 | NM_003239; transforming |
| | | growth factor, beta 3 |
| J03278_at | Hs.307783 | NM_002609; platelet- |
| | | derived growth factor |
| | | receptor beta precursor |
| J03909_at | | |
| J03925_at | Hs.172631 | NM_000632; integrin |
| | | alpha M precursor |
| J04056_at | Hs.88778 | NM_001757; carbonyl |
| | | reductase 1 |
| J04058_at | Hs.169919 | NM_000126; electron |
| | | transfer flavoprotein, |
| | | alpha polypeptide |
| J04093_s_at | Hs.278896 | NM_019075; UDP |
| | | glycosyltransferase 1 |
| | | family, polypeptide A10 |
| J04130_s_at | Hs.75703 | NM_002984; |
| | | chemokine (C-C motif) |
| | | ligand 4 precursor |
| J04152_rna1_s_at | | |
| J04162_at | Hs.372679 | NM_000569; Fc fragment |
| | | of IgG, low affinity |
| | | IIIa, receptor for (CD16) |
| J04456_at | Hs.407909 | NM_002305; beta- |
| | | galactosidase binding |
| | | lectin precursor |
| J05032_at | Hs.32393 | NM_001349; aspartyl- |
| | | tRNA synthetase |
| J05036_s_at | Hs.1355 | NM_001910; cathepsin |
| | | E isoform a preproprotein |
| | | NM_148964; |
| | | cathepsin E isoform b |
| | | preproprotein |
| J05070_at | Hs.151738 | NM_004994; matrix |
| | | metalloproteinase 9 |
| | | preproprotein |
| J05448_at | Hs.79402 | NM_002694; DNA |
| | | directed RNA polymerase |
| | | II polypeptide C |
| | | NM_032940; DNA |
| | | directed RNA polymerase |
| | | II polypeptide C |
| K01396_at | Hs.297681 | NM_000295; serine (or |
| | | cysteine) proteinase |
| | | inhibitor, clade A (alpha- |
| | | 1 antiproteinase, |
| | | antitrypsin), member 1 |
| K03430_at | | |
| L06797_s_at | Hs.421986 | NM_003467; |
| | | chemokine (C—X—C |
| | | motif) receptor 4 |
| L10343_at | Hs.112341 | NM_002638; skin- |
| | | derived protease inhibitor |
| | | 3 preproprotein |
| L11708_at | Hs.155109 | NM_002153; hydroxysteroid |
| | | (17-beta) dehydrogenase 2 |
| L13391_at | Hs.78944 | NM_002923; regulator |
| | | of G-protein signalling |
| | | 2, 24 kDa |
| L13698_at | Hs.65029 | NM_002048; growth |
| | | arrest-specific 1 |
| L13720_at | Hs.437710 | NM_000820; growth |
| | | arrest-specific 6 |
| L13923_at | Hs.750 | NM_000138; fibrillin 1 |
| AB000220_at | Hs.171921 | NM_006379; semaphorin |
| | | 3C |
| AC002073_cds1_at | | |
| AF000231_at | Hs.75618 | NM_004663; Ras- |
| | | related protein Rab-11A |
| D10922_s_at | Hs.99855 | NM_001462; formyl |
| | | peptide receptor-like 1 |
| D10925_at | Hs.301921 | NM_001295; |
| | | chemokine (C-C motif) |
| | | receptor 1 |
| D11086_at | Hs.84 | NM_000206; interleukin |
| | | 2 receptor, gamma |
| | | chain, precursor |
| D11151_at | Hs.211202 | NM_001957; endothelin |
| | | receptor type A |
| D13435_at | Hs.426142 | NM_002643; phosphatidylinositol |
| | | glycan, |
| | | class F isoform 1 |
| | | NM_173074; phosphatidylinositol |
| | | glycan, |
| | | class F isoform 2 |
| D13666_s_at | Hs.136348 | NM_006475; osteoblast |
| | | specific factor 2 (fasciclin |
| | | I-like) |
| D14520_at | Hs.84728 | NM_001730; Kruppel- |
| | | like factor 5 |
| D21878_at | Hs.169998 | NM_004334; bone |
| | | marrow stromal cell |
| | | antigen 1 precursor |
| D26443_at | Hs.371369 | NM_004172; solute |
| | | carrier family 1 (glial |
| | | high affinity glutamate |
| | | transporter), member 3 |
| D28589_at | Hs.17719 | |
| D42046_at | Hs.194665 | |
| D45370_at | Hs.74120 | NM_006829; adipose |
| | | specific 2 |
| D49372_s_at | Hs.54460 | NM_002986; small |
| | | inducible cytokine A11 |
| | | precursor |
| D50495_at | Hs.224397 | NM_003195; transcription |
| | | elongation factor A |
| | | (SII), 2 |
| D63135_at | Hs.27935 | NM_032646; tweety |
| | | homolog 2 |
| D64053_at | Hs.198288 | NM_002849; protein |
| | | tyrosine phosphatase, |
| | | receptor type, R isoform |
| | | 1 precursor |
| | | NM_130846; protein |
| | | tyrosine phosphatase, |
| | | receptor type, R isoform 2 |
| D83920_at | Hs.440898 | NM_002003; ficolin 1 |
| | | precursor |
| D85131_s_at | Hs.433881 | NM_002383; MYC- |
| | | associated zinc finger |
| | | protein |
| D86062_s_at | Hs.413482 | NM_004649; chromosome |
| | | 21 open reading |
| | | frame 33 |
| D86479_at | Hs.439463 | NM_001129; adipocyte |
| | | enhancer binding protein |
| | | 1 precursor |
| D86957_at | Hs.307944 | |
| D86959_at | Hs.105751 | NM_014720; Ste20- |
| | | related serine/threonine |
| | | kinase |
| D86976_at | Hs.196914 | |
| D87433_at | Hs.301989 | NM_015136; stabilin 1 |
| D87443_at | Hs.409862 | NM_014758; sorting |
| | | nexin 19 |
| D87682_at | Hs.134792 | |
| D89077_at | Hs.75367 | NM_006748; Src-like- |
| | | adaptor |
| D89377_at | Hs.89404 | NM_002449; msh |
| | | homeo box homolog 2 |
| D90279_s_at | Hs.433695 | NM_000093; alpha 1 |
| | | type V collagen prepro- |
| | | protein |
| HG1996-HT2044_at | | |
| HG2090-HT2152_s_at | | |
| HG2463-HT2559_at | | |
| HG2994-HT4850_s_at | | |
| HG3044-HT3742_s_at | | |
| HG3187-HT3366_s_at | | |
| HG3342-HT3519_s_at | | |
| HG371-HT26388_s_at | | |
| HG4069-HT4339_s_at | | |
| HG67-HT67_f_at | | |
| HG907-HT907_at | | |
| J02871_s_at | Hs.436317 | NM_000779; cytochrome |
| | | P450, family 4, |
| | | subfamily B, polypeptide 1 |
| J03040_at | Hs.111779 | NM_003118; secreted |
| | | protein, acidic, cysteine- |
| | | rich (osteonectin) |
| J03060_at | | |
| J03068_at | | |
| J03241_s_at | Hs.2025 | NM_003239; transforming |
| | | growth factor, beta 3 |
| J03278_at | Hs.307783 | NM_002609; platelet- |
| | | derived growth factor |
| | | receptor beta precursor |
| J03909_at | | |
| J03925_at | Hs.172631 | NM_000632; integrin |
| | | alpha M precursor |
| J04056_at | Hs.88778 | NM_001757; carbonyl |
| | | reductase 1 |
| J04058_at | Hs.169919 | NM_000126; electron |
| | | transfer flavoprotein, |
| | | alpha polypeptide |
| J04093_s_at | Hs.278896 | NM_019075; UDP |
| | | glycosyltransferase 1 |
| | | family, polypeptide A10 |
| J04130_s_at | Hs.75703 | NM_002984; |
| | | chemokine (C-C motif) |
| | | ligand 4 precursor |
| J04152_rna1_s_at | | |
| J04162_at | Hs.372679 | NM_000569; Fc fragment |
| | | of IgG, low affinity |
| | | IIIa, receptor for (CD16) |
| J04456_at | Hs.407909 | NM_002305; beta- |
| | | galactosidase binding |
| | | lectin precursor |
| J05032_at | Hs.32393 | NM_001349; aspartyl- |
| | | tRNA synthetase |
| J05036_s_at | Hs.1355 | NM_001910; cathepsin |
| | | E isoform a preproprotein |
| | | NM_148964; |
| | | cathepsin E isoform b |
| | | preproprotein |
| J05070_at | Hs.151738 | NM_004994; matrix |
| | | metalloproteinase 9 |
| | | preproprotein |
| J05448_at | Hs.79402 | NM_002694; DNA |
| | | directed RNA polymerase |
| | | II polypeptide C |
| | | NM_032940; DNA |
| | | directed RNA polymerase |
| | | II polypeptide C |
| K01396_at | Hs.297681 | NM_000295; serine (or |
| | | cysteine) proteinase |
| | | inhibitor, clade A (alpha- |
| | | 1 antiproteinase, |
| | | antitrypsin), member 1 |
| K03430_at | | |
| L06797_s_at | Hs.421986 | NM_003467; |
| | | chemokine (C—X—C |
| | | motif) receptor 4 |
| L10343_at | Hs.112341 | NM_002638; skin- |
| | | derived protease inhibitor |
| | | 3 preproprotein |
| L11708_at | Hs.155109 | NM_002153; hydroxysteroid |
| | | (17-beta) dehydrogenase 2 |
| L13391_at | Hs.78944 | NM_002923; regulator |
| | | of G-protein signalling |
| | | 2, 24 kDa |
| L13698_at | Hs.65029 | NM_002048; growth |
| | | arrest-specific 1 |
| L13720_at | Hs.437710 | NM_000820; growth |
| | | arrest-specific 6 |
| L13923_at | Hs.750 | NM_000138; fibrillin 1 |
| AB000220_at | Hs.171921 | NM_006379; semaphorin |
| | | 3C |
| AC002073_cds1_at | | |
| AF000231_at | Hs.75618 | NM_004663; Ras- |
| | | related protein Rab-11A |
| D10922_s_at | Hs.99855 | NM_001462; formyl |
| | | peptide receptor-like 1 |
| D10925_at | Hs.301921 | NM_001295; |
| | | chemokine (C-C motif) |
| | | receptor 1 |
| D11086_at | Hs.84 | NM_000206; interleukin |
| | | 2 receptor, gamma |
| | | chain, precursor |
| D11151_at | Hs.211202 | NM_001957; endothelin |
| | | receptor type A |
| D13435_at | Hs.426142 | NM_002643; phosphatidylinositol |
| | | glycan, |
| | | class F isoform 1 |
| | | NM_173074; phosphatidylinositol |
| | | glycan, |
| | | class F isoform 2 |
| D13666_s_at | Hs.136348 | NM_006475; osteoblast |
| | | specific factor 2 (fasciclin |
| | | I-like) |
| D14520_at | Hs.84728 | NM_001730; Kruppel- |
| | | like factor 5 |
| D21878_at | Hs.169998 | NM_004334; bone |
| | | marrow stromal cell |
| | | antigen 1 precursor |
| D26443_at | Hs.371369 | NM_004172; solute |
| | | carrier family 1 (glial |
| | | high affinity glutamate |
| | | transporter), member 3 |
| D28589_at | Hs.17719 | |
| D42046_at | Hs.194665 | |
| D45370_at | Hs.74120 | NM_006829; adipose |
| | | specific 2 |
| D49372_s_at | Hs.54460 | NM_002986; small |
| | | inducible cytokine A11 |
| | | precursor |
| D50495_at | Hs.224397 | NM_003195; transcription |
| | | elongation factor A |
| | | (SII), 2 |
| D63135_at | Hs.27935 | NM_032646; tweety |
| | | homolog 2 |
| D64053_at | Hs.198288 | NM_002849; protein |
| | | tyrosine phosphatase, |
| | | receptor type, R isoform |
| | | 1 precursor |
| | | NM_130846; protein |
| | | tyrosine phosphatase, |
| | | receptor type, R isoform 2 |
| D83920_at | Hs.440898 | NM_002003; ficolin 1 |
| | | precursor |
| D85131_s_at | Hs.433881 | NM_002383; MYC- |
| | | associated zinc finger |
| | | protein |
| D86062_s_at | Hs.413482 | NM_004649; chromosome |
| | | 21 open reading |
| | | frame 33 |
| D86479_at | Hs.439463 | NM_001129; adipocyte |
| | | enhancer binding protein |
| | | 1 precursor |
| D86957_at | Hs.307944 | |
| D86959_at | Hs.105751 | NM_014720; Ste20- |
| | | related serine/threonine |
| | | kinase |
| D86976_at | Hs.196914 | |
| D87433_at | Hs.301989 | NM_015136; stabilin 1 |
| D87443_at | Hs.409862 | NM_014758; sorting |
| | | nexin 19 |
| D87682_at | Hs.134792 | |
| D89077_at | Hs.75367 | NM_006748; Src-like- |
| | | adaptor |
| D89377_at | Hs.89404 | NM_002449; msh |
| | | homeo box homolog 2 |
| D90279_s_at | Hs.433695 | NM_000093; alpha 1 |
| | | type V collagen prepro- |
| | | protein |
| HG1996-HT2044_at | | |
| HG2090-HT2152_s_at | | |
| HG2463-HT2559_at | | |
| HG2994-HT4850_s_at | | |

TABLE 10
160 Genes for classifier

| Chip acc. # | UniGene Build 162 | Description |
|---|---|---|
| AF000231_at | Hs.75618 | NM_004663; Ras-related protein Rab-11A |
| D13666_s_at | Hs.136348 | NM_006475; osteoblast specific factor 2 (fasciclin I-like) |
| D21878_at | Hs.169998 | NM_004334; bone marrow stromal cell antigen 1 precursor |
| D45370_at | Hs.74120 | NM_006829; adipose specific 2 |
| D49372_s_at | Hs.54460 | NM_002986; small inducible cytokine A11 precursor |
| D83920_at | Hs.440898 | NM_002003; ficolin 1 precursor |
| D85131_s_at | Hs.433881 | NM_002383; MYC-associated zinc finger protein |
| D86062_s_at | Hs.413482 | NM_004649; chromosome 21 open reading frame 33 |
| D86479_at | Hs.439463 | NM_001129; adipocyte enhancer binding protein 1 precursor |
| D86957_at | Hs.307944 | |
| D86976_at | Hs.196914 | |
| D87433_at | Hs.301989 | NM_015136; stabilin 1 |
| D89077_at | Hs.75367 | NM_006748; Src-like-adaptor |
| D89377_at | Hs.89404 | NM_002449; msh homeo box homolog 2 |
| HG3044-HT3742_s_at | | |
| HG371-HT26388_s_at | | |
| HG4069-HT4339_s_at | | |
| HG67-HT67_f_at | | |
| HG907-HT907_at | | |
| J02871_s_at | Hs.436317 | NM_000779; cytochrome P450, family 4, subfamily B, poly- |
| | | peptide 1 |
| J03040_at | Hs.111779 | NM_003118; secreted protein, acidic, cysteine-rich (osteonectin) |
| J03068_at | | |
| J03241_s_at | Hs.2025 | NM_003239; transforming growth factor, beta 3 |
| J03278_at | Hs.307783 | NM_002609; platelet-derived growth factor receptor beta |
| | | precursor |
| J03909_at | | |
| J04058_at | Hs.169919 | NM_000126; electron transfer flavoprotein, alpha polypeptide |
| J04130_s_at | Hs.75703 | NM_002984; chemokine (C-C motif) ligand 4 precursor |
| J04162_at | Hs.372679 | NM_000569; Fc fragment of IgG, low affinity IIIa, receptor for |
| | | (CD16) |
| J04456_at | Hs.407909 | NM_002305; beta-galactosidase binding lectin precursor |
| J05032_at | Hs.32393 | NM_001349; aspartyl-tRNA synthetase |
| J05070_at | Hs.151738 | NM_004994; matrix metalloproteinase 9 preproprotein |
| J05448_at | Hs.79402 | NM_002694; DNA directed RNA polymerase II polypeptide C |
| | | NM_032940; DNA directed RNA polymerase II polypeptide C |
| K01396_at | Hs.297681 | NM_000295; serine (or cysteine) proteinase inhibitor, clade A |
| | | (alpha-1 antiproteinase, antitrypsin), member 1 |
| K03430_at | | |
| L13698_at | Hs.65029 | NM_002048; growth arrest-specific 1 |
| L13720_at | Hs.437710 | NM_000820; growth arrest-specific 6 |
| L13923_at | Hs.750 | NM_000138; fibrillin 1 |
| L15409_at | Hs.421597 | NM_000551; elogin binding protein |
| L17325_at | Hs.195825 | NM_006867; RNA-binding protein with multiple splicing |
| L19872_at | Hs.170087 | NM_001621; aryl hydrocarbon receptor |
| L27476_at | Hs.75608 | NM_004817; tight junction protein 2 (zona occludens 2) |
| L33799_at | Hs.202097 | NM_002593; procollagen C-endopeptidase enhancer |
| L40388_at | Hs.30212 | NM_004236; thyroid receptor interacting protein 15 |
| L40904_at | Hs.387667 | NM_005037; peroxisome proliferative activated receptor |
| | | gamma isoform 1 NM_015869; peroxisome proliferative activated |
| | | receptor gamma isoform 2 NM_138711; peroxisome |
| | | proliferative activated receptor gamma isoform 1 NM_138712; |
| | | peroxisome proliferative activated receptor gamma isoform 1 |
| L41919_rna1_at | | |
| M11433_at | Hs.101850 | NM_002899; retinol binding protein 1, cellular |
| M11718_at | Hs.283393 | NM_000393; alpha 2 type V collagen preproprotein |
| M12125_at | Hs.300772 | NM_003289; tropomyosin 2 (beta) |
| M14218_at | Hs.442047 | NM_000048; argininosuccinate lyase |
| M15395_at | Hs.375957 | NM_000211; integrin beta chain, beta 2 precursor |
| M16591_s_at | Hs.89555 | NM_002110; hemopoietic cell kinase isoform p61HCK |
| M17219_at | Hs.203862 | NM_002069; guanine nucleotide binding protein (G protein), |
| | | alpha inhibiting activity polypeptide 1 |
| M20530_at | | |
| M23178_s_at | Hs.73817 | NM_002983; chemokine (C-C motif) ligand 3 |
| M28130_rna1_s_at | | |
| M29550_at | Hs.187543 | NM_021132; protein phosphatase 3 (formerly 2B), catalytic |
| | | subunit, beta isoform (calcineurin A beta) |
| M31165_at | Hs.407546 | NM_007115; tumor necrosis factor, alpha-induced protein 6 |
| | | precursor |
| M32011_at | Hs.949 | NM_000433; neutrophil cytosolic factor 2 |
| M33195_at | Hs.433300 | NM_004106; Fc fragment of IgE, high affinity I, receptor for, |
| | | gamma polypeptide precursor |
| M37033_at | Hs.443057 | NM_000560; CD53 antigen |
| M37766_at | Hs.901 | NM_001778; CD48 antigen (B-cell membrane protein) |
| M55998_s_at | Hs.172928 | NM_000088; alpha 1 type I collagen preproprotein |
| M57731_s_at | Hs.75765 | NM_002089; chemokine (C—X—C motif) ligand 2 |
| M62840_at | Hs.82542 | NM_001637; acyloxyacyl hydrolase precursor |
| M63262_at | | |
| M68840_at | Hs.183109 | NM_000240; monoamine oxidase A |
| M69203_s_at | Hs.75703 | NM_002984; chemokine (C-C motif) ligand 4 precursor |
| M72885_rna1_s_at | | |
| M77349_at | Hs.421496 | NM_000358; transforming growth factor, beta-induced, 68 kDa |
| M82882_at | Hs.124030 | NM_172373; E74-like factor 1 (ets domain transcription factor) |
| M83822_at | Hs.209846 | NM_006726; LPS-responsive vesicle trafficking, beach and |
| | | anchor containing |
| M92934_at | Hs.410037 | NM_001901; connective tissue growth factor |
| M95178_at | Hs.119000 | NM_001102; actinin, alpha 1 |
| S69115_at | Hs.10306 | NM_005601; natural killer cell group 7 sequence |
| S77393_at | Hs.145754 | NM_016531; Kruppel-like factor 3 (basic) |
| S78187_at | Hs.153752 | NM_004358; cell division cycle 25B isoform 1 NM_021872; |
| | | cell division cycle 25B isoform 2 NM_021873; cell division |
| | | cycle 25B isoform 3 NM_021874; cell division cycle 25B isoform 4 |
| U01833_at | Hs.81469 | NM_002484; nucleotide binding protein 1 (MinD homolog, E. coli) |
| U07231_at | Hs.309763 | NM_002092; G-rich RNA sequence binding factor 1 |
| U09278_at | Hs.436852 | NM_004460; fibroblast activation protein, alpha subunit |
| U09937_rna1_s_at | | |
| U10550_at | Hs.79022 | NM_005261; GTP-binding mitogen-induced T-cell protein |
| | | NM_181702; GTP-binding mitogen-induced T-cell protein |
| U12424_s_at | Hs.108646 | NM_000408; glycerol-3-phosphate dehydrogenase 2 (mitochondrial) |
| U16306_at | Hs.434488 | NM_004385; chondroitin sulfate proteoglycan 2 (versican) |
| U20158_at | Hs.2488 | NM_005565; lymphocyte cytosolic protein 2 |
| U20536_s_at | Hs.3280 | NM_001226; caspase 6 isoform alpha preproprotein |
| | | NM_032992; caspase 6 isoform beta |
| U24266_at | Hs.77448 | NM_003748; aldehyde dehydrogenase 4A1 precursor |
| | | NM_170726; aldehyde dehydrogenase 4A1 precursor |
| U28249_at | Hs.301350 | NM_005971; FXYD domain containing ion transport regulator |
| | | 3 isoform 1 precursor NM_021910; FXYD domain containing |
| | | ion transport regulator 3 isoform 2 precursor |
| U28488_s_at | Hs.155935 | NM_004054; complement component 3a receptor 1 |
| U29680_at | Hs.227817 | NM_004049; BCL2-related protein A1 |
| U37143_at | Hs.152096 | NM_000775; cytochrome P450, family 2, subfamily J, polypeptide 2 |
| U38864_at | Hs.108139 | NM_012256; zinc finger protein 212 |
| U39840_at | Hs.163484 | NM_004496; forkhead box A1 |
| U41315_rna1_s_at | | |
| U44111_at | Hs.42151 | NM_006895; histamine N-methyltransferase |
| U47414_at | Hs.13291 | NM_004354; cyclin G2 |
| U49352_at | Hs.414754 | NM_001359; 2,4-dienoyl CoA reductase 1 precursor |
| U50708_at | Hs.1265 | NM_000056; branched chain keto acid dehydrogenase E1, |
| | | beta polypeptide precursor NM_183050; branched chain keto |
| | | acid dehydrogenase E1, beta polypeptide precursor |
| U52101_at | Hs.9999 | NM_001425; epithelial membrane protein 3 |
| U59914_at | Hs.153863 | NM_005585; MAD, mothers against decapentaplegic homolog 6 |
| U60205_at | Hs.393239 | NM_006745; sterol-C4-methyl oxidase-like |
| U61981_at | Hs.42674 | NM_002439; mutS homolog 3 |
| U64520_at | Hs.66708 | NM_004781; vesicle-associated membrane protein 3 (cellubrevin) |
| U65093_at | Hs.82071 | NM_006079; Cbp/p300-interacting transactivator, with |
| | | Glu/Asp-rich carboxy-terminal domain, 2 |
| U66619_at | Hs.444445 | NM_003078; SWI/SNF-related matrix-associated actin- |
| | | dependent regulator of chromatin d3 |
| U68019_at | Hs.288261 | NM_005902; MAD, mothers against decapentaplegic homolog 3 |
| U68385_at | Hs.380923 | |
| U68485_at | Hs.193163 | NM_004305; bridging integrator 1 isoform 8 NM_139343; |
| | | bridging integrator 1 isoform 1 NM_139344; bridging integrator |
| | | 1 isoform 2 NM_139345; bridging integrator 1 isoform 3 |
| | | NM_139346; bridging integrator 1 isoform 4 NM_139347; |
| | | bridging integrator 1 isoform 5 NM_139348; bridging integrator |
| | | 1 isoform 6 NM_139349; bridging integrator 1 isoform 7 |
| | | NM_139350; bridging integrator 1 isoform 9 NM_139351; |
| | | bridging integrator 1 isoform 10 |
| U74324_at | Hs.90875 | NM_002871; RAB-interacting factor |
| U77970_at | Hs.321164 | NM_002518; neuronal PAS domain protein 2 NM_032235; |
| U83303_cds2_at | Hs.164021 | NM_002993; chemokine (C—X—C motif) ligand 6 (granulocyte |
| | | chemotactic protein 2) |
| U88871_at | Hs.79993 | NM_000288; peroxisomal biogenesis factor 7 |
| U90549_at | Hs.236774 | NM_006353; high mobility group nucleosomal binding domain 4 |
| U90716_at | Hs.79187 | NM_001338; coxsackie virus and adenovirus receptor |
| V00594_at | Hs.118786 | NM_005953; metallothionein 2A |
| V00594_s_at | Hs.118786 | NM_005953; metallothionein 2A |
| X02761_s_at | Hs.418138 | NM_002026; fibronectin 1 isoform 1 preproprotein |
| | | NM_054034; fibronectin 1 isoform 2 preproprotein |
| X04011_at | Hs.88974 | NM_000397; cytochrome b-245, beta polypeptide (chronic |
| | | granulomatous disease) |
| X04085_rna1_at | | |
| X07438_s_at | | |
| X07743_at | Hs.77436 | NM_002664; pleckstrin |
| X13334_at | Hs.75627 | NM_000591; CD14 antigen precursor |
| X14046_at | Hs.153053 | NM_001774; CD37 antigen |
| X14813_at | Hs.166160 | NM_001607; acetyl-Coenzyme A acyltransferase 1 |
| X15880_at | Hs.415997 | NM_001848; collagen, type VI, alpha 1 precursor |
| X15882_at | Hs.420269 | NM_001849; alpha 2 type VI collagen isoform 2C2 precursor |
| | | NM_058174; alpha 2 type VI collagen isoform 2C2a precursor |
| | | NM_058175; alpha 2 type VI collagen isoform 2C2a precursor |
| X51408_at | Hs.380138 | NM_001822; chimerin (chimaerin) 1 |
| X53800_s_at | Hs.89690 | NM_002090; chemokine (C—X—C motif) ligand 3 |
| X54489_rna1_at | | |
| X57351_s_at | Hs.174195 | NM_006435; interferon induced transmembrane protein 2 (1- |
| | | 8D) |
| X57579_s_at | | |
| X58072_at | Hs.169946 | NM_002051; GATA binding protein 3 NM_032742; |
| X62048_at | Hs.249441 | NM_003390; wee1 tyrosine kinase |
| X64072_s_at | Hs.375957 | NM_000211; integrin beta chain, beta 2 precursor |
| X65614_at | Hs.2962 | NM_005980; S100 calcium binding protein P |
| X66945_at | Hs.748 | NM_000604; fibroblast growth factor receptor 1 isoform 1 |
| | | precursor NM_015850; fibroblast growth factor receptor 1 |
| | | isoform 2 precursor NM_023105; fibroblast growth factor |
| | | receptor 1 isoform 3 precursor NM_023106; fibroblast growth |
| | | factor receptor 1 isoform 4 precursor NM_023107; fibroblast |
| | | growth factor receptor 1 isoform 5 precursor NM_023108; |
| | | fibroblast growth factor receptor 1 isoform 6 precursor |
| | | NM_023109; fibroblast growth factor receptor 1 isoform 7 |
| | | precursor NM_023110; fibroblast growth factor receptor 1 |
| | | isoform 8 precursor NM_023111; fibroblast growth factor |
| | | receptor 1 isoform 9 precursor |
| X67491_f_at | Hs.355697 | NM_005271; glutamate dehydrogenase 1 |
| X68194_at | Hs.80919 | NM_006754; synaptophysin-like protein isoform a |
| | | NM_182715; synaptophysin-like protein isoform b |
| X73882_at | Hs.254605 | NM_003980; microtubule-associated protein 7 |
| X78520_at | Hs.372528 | NM_001829; chloride channel 3 |
| X78549_at | Hs.51133 | NM_005975; PTK6 protein tyrosine kinase 6 |
| X78565_at | Hs.98998 | NM_002160; tenascin C (hexabrachion) |
| X78669_at | Hs.79088 | NM_002902; reticulocalbin 2, EF-hand calcium binding domain |
| X83618_at | Hs.59889 | NM_005518; 3-hydroxy-3-methylglutaryl-Coenzyme A synthase |
| | | 2 (mitochondrial) |
| X84908_at | Hs.78060 | NM_000293; phosphorylase kinase, beta |
| X90908_at | Hs.147391 | NM_001445; gastrotropin |
| X91504_at | Hs.389277 | NM_003224; ADP-ribosylation factor related protein 1 |
| X95632_s_at | Hs.387906 | NM_005759; abl-interactor 2 |
| X97267_rna1_s_at | | |
| Y00705_at | Hs.407856 | NM_003122; serine protease inhibitor, Kazal type 1 |
| Y00787_s_at | Hs.624 | NM_000584; interleukin 8 precursor |
| Y00815_at | Hs.75216 | NM_002840; protein tyrosine phosphatase, receptor type, F |
| | | isoform 1 precursor NM_130440; protein tyrosine phosphatase, |
| | | receptor type, F isoform 2 precursor |
| Y08374_rna1_at | | |
| Z12173_at | Hs.334534 | NM_002076; glucosamine (N-acetyl)-6-sulfatase precursor |
| Z19554_s_at | Hs.435800 | NM_003380; vimentin |
| Z26491_s_at | Hs.240013 | NM_000754; catechol-O-methyltransferase isoform MB-COMT |
| | | NM_007310; catechol-O-methyltransferase isoform S-COMT |
| Z29331_at | Hs.372758 | NM_003344; ubiquitin-conjugating enzyme E2H isoform 1 |
| | | NM_182697; ubiquitin-conjugating enzyme E2H isoform 2 |
| Z35491_at | Hs.377484 | NM_004323; BCL2-associated athanogene isoform 1L |
| Z48199_at | Hs.82109 | NM_002997; syndecan 1 |
| Z48605_at | Hs.421825 | NM_006903; inorganic pyrophosphatase 2 isoform 2 |
| | | NM_176865; NM_176866; inorganic pyrophosphatase 2 |
| | | isoform 3 NM_176867; inorganic pyrophosphatase 2 isoform 4 |
| | | NM_176869; inorganic pyrophosphatase 2 isoform 1 |
| Z74615_at | Hs.172928 | NM_000088; alpha 1 type I collagen preproprotein |

TABLE 11
80 genes for classifier

| Chip acc. # | UniGene Build 162 | Description |
|---|---|---|
| AF000231_at | Hs.75618 | NM_004663; Ras-related protein Rab-11A |
| D13666_s_at | Hs.136348 | NM_006475; osteoblast specific factor 2 (fasciclin I-like) |
| D49372_s_at | Hs.54460 | NM_002986; small inducible cytokine A11 precursor |
| D83920_at | Hs.440898 | NM_002003; ficolin 1 precursor |
| D86479_at | Hs.439463 | NM_001129; adipocyte enhancer binding protein 1 precursor |
| D87433_at | Hs.301989 | NM_015136; stabilin 1 |
| D89077_at | Hs.75367 | NM_006748; Src-like-adaptor |
| D89377_at | Hs.89404 | NM_002449; msh homeo box homolog 2 |
| HG4069-HT4339_s_at | | |
| HG67-HT67_f_at | | |
| HG907-HT907_at | | |
| J02871_s_at | Hs.436317 | NM_000779; cytochrome P450, family 4, subfamily B, poly- |
| | | peptide 1 |
| J03278_at | Hs.307783 | NM_002609; platelet-derived growth factor receptor beta |
| | | precursor |
| J04058_at | Hs.169919 | NM_000126; electron transfer flavoprotein, alpha polypeptide |
| J05032_at | Hs.32393 | NM_001349; aspartyl-tRNA synthetase |
| J05070_at | Hs.151738 | NM_004994; matrix metalloproteinase 9 preproprotein |
| J05448_at | Hs.79402 | NM_002694; DNA directed RNA polymerase II polypeptide C |
| | | NM_032940; DNA directed RNA polymerase II polypeptide C |
| K01396_at | Hs.297681 | NM_000295; serine (or cysteine) proteinase inhibitor, clade A |
| | | (alpha-1 antiproteinase, antitrypsin), member 1 |
| L13720_at | Hs.437710 | NM_000820; growth arrest-specific 6 |
| L40904_at | Hs.387667 | NM_005037; peroxisome proliferative activated receptor |
| | | gamma isoform 1 NM_015869; peroxisome proliferative activated |
| | | receptor gamma isoform 2 NM_138711; peroxisome |
| | | proliferative activated receptor gamma isoform 1 NM_138712; |
| | | peroxisome proliferative activated receptor gamma isoform 1 |
| M12125_at | Hs.300772 | NM_003289; tropomyosin 2 (beta) |
| M15395_at | Hs.375957 | NM_000211; integrin beta chain, beta 2 precursor |
| M16591_s_at | Hs.89555 | NM_002110; hemopoietic cell kinase isoform p61HCK |
| M20530_at | | |
| M23178_s_at | Hs.73817 | NM_002983; chemokine (C-C motif) ligand 3 |
| M32011_at | Hs.949 | NM_000433; neutrophil cytosolic factor 2 |
| M33195_at | Hs.433300 | NM_004106; Fc fragment of IgE, high affinity I, receptor for, |
| | | gamma polypeptide precursor |
| M55998_s_at | Hs.172928 | NM_000088; alpha 1 type I collagen preproprotein |
| M57731_s_at | Hs.75765 | NM_002089; chemokine (C—X—C motif) ligand 2 |
| M63262_at | | |
| M68840_at | Hs.183109 | NM_000240; monoamine oxidase A |
| M69203_s_at | Hs.75703 | NM_002984; chemokine (C-C motif) ligand 4 precursor |
| M72885_rna1_s_at | | |
| M83822_at | Hs.209846 | NM_006726; LPS-responsive vesicle trafficking, beach and |
| | | anchor containing |
| S77393_at | Hs.145754 | NM_016531; Kruppel-like factor 3 (basic) |
| U01833_at | Hs.81469 | NM_002484; nucleotide binding protein 1 (MinD homolog, E. coli) |
| U07231_at | Hs.309763 | NM_002092; G-rich RNA sequence binding factor 1 |
| U09937_rna1_s_at | | |
| U10550_at | Hs.79022 | NM_005261; GTP-binding mitogen-induced T-cell protein |
| | | NM_181702; GTP-binding mitogen-induced T-cell protein |
| U20158_at | Hs.2488 | NM_005565; lymphocyte cytosolic protein 2 |
| U28488_s_at | Hs.155935 | NM_004054; complement component 3a receptor 1 |
| U29680_at | Hs.227817 | NM_004049; BCL2-related protein A1 |
| U41315_rna1_s_at | | |
| U47414_at | Hs.13291 | NM_004354; cyclin G2 |
| U49352_at | Hs.414754 | NM_001359; 2,4-dienoyl CoA reductase 1 precursor |
| U50708_at | Hs.1265 | NM_000056; branched chain keto acid dehydrogenase E1, |
| | | beta polypeptide precursor NM_183050; branched chain keto |
| | | acid dehydrogenase E1, beta polypeptide precursor |
| U52101_at | Hs.9999 | NM_001425; epithelial membrane protein 3 |
| U59914_at | Hs.153863 | NM_005585; MAD, mothers against decapentaplegic homolog 6 |
| U64520_at | Hs.66708 | NM_004781; vesicle-associated membrane protein 3 (cellubrevin) |
| U65093_at | Hs.82071 | NM_006079; Cbp/p300-interacting transactivator, with |
| | | Glu/Asp-rich carboxy-terminal domain, 2 |
| U68019_at | Hs.288261 | NM_005902; MAD, mothers against decapentaplegic homolog 3 |
| U68385_at | Hs.380923 | |
| U74324_at | Hs.90875 | NM_002871; RAB-interacting factor |
| U77970_at | Hs.321164 | NM_002518; neuronal PAS domain protein 2 NM_032235; |
| U90549_at | Hs.236774 | NM_006353; high mobility group nucleosomal binding domain 4 |
| X04085_rna1_at | | |
| X07438_s_at | | |
| X07743_at | Hs.77436 | NM_002664; pleckstrin |
| X13334_at | Hs.75627 | NM_000591; CD14 antigen precursor |
| X14046_at | Hs.153053 | NM_001774; CD37 antigen |
| X15880_at | Hs.415997 | NM_001848; collagen, type VI, alpha 1 precursor |
| X15882_at | Hs.420269 | NM_001849; alpha 2 type VI collagen isoform 2C2 precursor |
| | | NM_058174; alpha 2 type VI collagen isoform 2C2a precursor |
| | | NM_058175; alpha 2 type VI collagen isoform 2C2a precursor |
| X51408_at | Hs.380138 | NM_001822; chimerin (chimaerin) 1 |
| X53800_s_at | Hs.89690 | NM_002090; chemokine (C—X—C motif) ligand 3 |
| X54489_rna1_at | | |
| X57579_s_at | | |
| X62048_at | Hs.249441 | NM_003390; wee1 tyrosine kinase |
| X64072_s_at | Hs.375957 | NM_000211; integrin beta chain, beta 2 precursor |
| X67491_f_at | Hs.355697 | NM_005271; glutamate dehydrogenase 1 |
| X68194_at | Hs.80919 | NM_006754; synaptophysin-like protein isoform a |
| | | NM_182715; synaptophysin-like protein isoform b |
| X73882_at | Hs.254605 | NM_003980; microtubule-associated protein 7 |
| X78520_at | Hs.372528 | NM_001829; chloride channel 3 |
| X97267_rna1_s_at | | |
| Y00787_s_at | Hs.624 | NM_000584; interleukin 8 precursor |
| Z12173_at | Hs.334534 | NM_002076; glucosamine (N-acetyl)-6-sulfatase precursor |
| Z19554_s_at | Hs.435800 | NM_003380; vimentin |
| Z26491_s_at | Hs.240013 | NM_000754; catechol-O-methyltransferase isoform MB-COMT |
| | | NM_007310; catechol-O-methyltransferase isoform S-COMT |
| Z29331_at | Hs.372758 | NM_003344; ubiquitin-conjugating enzyme E2H isoform 1 |
| | | NM_182697; ubiquitin-conjugating enzyme E2H isoform 2 |
| Z48605_at | Hs.421825 | NM_006903; inorganic pyrophosphatase 2 isoform 2 |
| | | NM_176865; NM_176866; inorganic pyrophosphatase 2 |
| | | isoform 3 NM_176867; inorganic pyrophosphatase 2 isoform 4 |
| | | NM_176869; inorganic pyrophosphatase 2 isoform 1 |
| Z74615_at | Hs.172928 | NM_000088; alpha 1 type I collagen preproprotein |

TABLE 12
40 genes for classifier

| Chip acc. # | UniGene Build 162 | description |
|---|---|---|
| D83920_at | Hs.440898 | NM_002003; ficolin 1 precursor |
| D89377_at | Hs.89404 | NM_002449; msh homeo box homolog 2 |
| J02871_s_at | Hs.436317 | NM_000779; cytochrome P450, family 4, subfamily B, poly- |
| | | peptide 1 |
| J05032_at | Hs.32393 | NM_001349; aspartyl-tRNA synthetase |
| J05070_at | Hs.151738 | NM_004994; matrix metalloproteinase 9 preproprotein |
| M16591_s_at | Hs.89555 | NM_002110; hemopoietic cell kinase isoform p61HCK |
| M23178_s_at | Hs.73817 | NM_002983; chemokine (C-C motif) ligand 3 |
| M32011_at | Hs.949 | NM_000433; neutrophil cytosolic factor 2 |
| M33195_at | Hs.433300 | NM_004106; Fc fragment of IgE, high affinity I, receptor for, |
| | | gamma polypeptide precursor |
| M57731_s_at | Hs.75765 | NM_002089; chemokine (C—X—C motif) ligand 2 |
| M68840_at | Hs.183109 | NM_000240; monoamine oxidase A |
| M69203_s_at | Hs.75703 | NM_002984; chemokine (C-C motif) ligand 4 precursor |
| S77393_at | Hs.145754 | NM_016531; Kruppel-like factor 3 (basic) |
| U01833_at | Hs.81469 | NM_002484; nucleotide binding protein 1 (MinD homolog, E. coli) |
| U07231_at | Hs.309763 | NM_002092; G-rich RNA sequence binding factor 1 |
| U09937_rna1_s_at | | |
| U20158_at | Hs.2488 | NM_005565; lymphocyte cytosolic protein 2 |
| U41315_rna1_s_at | | |
| U47414_at | Hs.13291 | NM_004354; cyclin G2 |
| U49352_at | Hs.414754 | NM_001359; 2,4-dienoyl CoA reductase 1 precursor |
| U50708_at | Hs.1265 | NM_000056; branched chain keto acid dehydrogenase E1, |
| | | beta polypeptide precursor NM_183050; branched chain keto |
| | | acid dehydrogenase E1, beta polypeptide precursor |
| U65093_at | Hs.82071 | NM_006079; Cbp/p300-interacting transactivator, with |
| | | Glu/Asp-rich carboxy-terminal domain, 2 |
| U68385_at | Hs.380923 | |
| U77970_at | Hs.321164 | NM_002518; neuronal PAS domain protein 2 NM_032235; |
| U90549_at | Hs.236774 | NM_006353; high mobility group nucleosomal binding domain 4 |
| X13334_at | Hs.75627 | NM_000591; CD14 antigen precursor |
| X15880_at | Hs.415997 | NM_001848; collagen, type VI, alpha 1 precursor |
| X15882_at | Hs.420269 | NM_001849; alpha 2 type VI collagen isoform 2C2 precursor |
| | | NM_058174; alpha 2 type VI collagen isoform 2C2a precursor |
| | | NM_058175; alpha 2 type VI collagen isoform 2C2a precursor |
| X51408_at | Hs.380138 | NM_001822; chimerin (chimaerin) 1 |
| X53800_s_at | Hs.89690 | NM_002090; chemokine (C—X—C motif) ligand 3 |
| X54489_rna1_at | | |
| X57579_s_at | | |
| X64072_s_at | Hs.375957 | NM_000211; integrin beta chain, beta 2 precursor |
| X67491_f_at | Hs.355697 | NM_005271; glutamate dehydrogenase 1 |
| X68194_at | Hs.80919 | NM_006754; synaptophysin-like protein isoform a |
| | | NM_182715; synaptophysin-like protein isoform b |
| X73882_at | Hs.254605 | NM_003980; microtubule-associated protein 7 |
| X78520_at | Hs.372528 | NM_001829; chloride channel 3 |
| Z29331_at | Hs.372758 | NM_003344; ubiquitin-conjugating enzyme E2H isoform 1 |
| | | NM_182697; ubiquitin-conjugating enzyme E2H isoform 2 |
| Z48605_at | Hs.421825 | NM_006903; inorganic pyrophosphatase 2 isoform 2 |
| | | NM_176865; NM_176866; inorganic pyrophosphatase 2 |
| | | isoform 3 NM_176867; inorganic pyrophosphatase 2 isoform 4 |
| | | NM_176869; inorganic pyrophosphatase 2 isoform 1 |
| Z74615_at | Hs.172928 | NM_000088; alpha 1 type I collagen preproprotein |

TABLE 13
20 genes for classifier

| Chip acc. # | UniGene Build 162 | description |
|---|---|---|
| D89377_at | Hs.89404 | NM_002449; msh homeo box homolog 2 |
| J05032_at | Hs.32393 | NM_001349; aspartyl-tRNA synthetase |
| M23178_s_at | Hs.73817 | NM_002983; chemokine (C-C motif) ligand 3 |
| M32011_at | Hs.949 | NM_000433; neutrophil cytosolic factor 2 |
| M69203_s_at | Hs.75703 | NM_002984; chemokine (C-C motif) ligand 4 precursor |
| S77393_at | Hs.145754 | NM_016531; Kruppel-like factor 3 (basic) |
| U07231_at | Hs.309763 | NM_002092; G-rich RNA sequence binding factor 1 |
| U41315_rna1_s_at | | |
| U47414_at | Hs.13291 | NM_004354; cyclin G2 |
| U49352_at | Hs.414754 | NM_001359; 2,4-dienoyl CoA reductase 1 precursor |
| U50708_at | Hs.1265 | NM_000056; branched chain keto acid dehydrogenase E1, |
| | | beta polypeptide precursor NM_183050; branched chain keto |
| | | acid dehydrogenase E1, beta polypeptide precursor |
| U77970_at | Hs.321164 | NM_002518; neuronal PAS domain protein 2 NM_032235; |
| X13334_at | Hs.75627 | NM_000591; CD14 antigen precursor |
| X57579_s_at | | |
| X64072_s_at | Hs.375957 | NM_000211; integrin beta chain, beta 2 precursor |
| X68194_at | Hs.80919 | NM_006754; synaptophysin-like protein isoform a |
| | | NM_182715; synaptophysin-like protein isoform b |
| X73882_at | Hs.254605 | NM_003980; microtubule-associated protein 7 |
| X78520_at | Hs.372528 | NM_001829; chloride channel 3 |
| Z48605_at | Hs.421825 | NM_006903; inorganic pyrophosphatase 2 isoform 2 |
| | | NM_176865; NM_176866; inorganic pyrophosphatase 2 |
| | | isoform 3 NM_176867; inorganic pyrophosphatase 2 isoform 4 |
| | | NM_176869; inorganic pyrophosphatase 2 isoform 1 |
| Z74615_at | Hs.172928 | NM_000088; alpha 1 type I collagen preproprotein |

TABLE 14
10 genes for classifier

| Chip acc. # | UniGene Build 162 | description |
|---|---|---|
| D89377_at | Hs.89404 | NM_002449; msh homeo box homolog 2 |
| S77393_at | Hs.145754 | NM_016531; Kruppel-like factor 3 (basic) |
| U41315_rna1_s_at | | |
| U47414_at | Hs.13291 | NM_004354; cyclin G2 |
| U77970_at | Hs.321164 | NM_002518; neuronal PAS domain protein 2 NM_032235; |
| X68194_at | Hs.80919 | NM_006754; synaptophysin-like protein isoform a |
| | | NM_182715; synaptophysin-like protein isoform b |
| X73882_at | Hs.254605 | NM_003980; microtubule-associated protein 7 |
| X78520_at | Hs.372528 | NM_001829; chloride channel 3 |
| Z48605_at | Hs.421825 | NM_006903; inorganic pyrophosphatase 2 isoform 2 |
| | | NM_176865; NM_176866; inorganic pyrophosphatase 2 |
| | | isoform 3 NM_176867; inorganic pyrophosphatase 2 isoform 4 |
| | | NM_176869; inorganic pyrophosphatase 2 isoform 1 |
| Z74615_at | Hs.172928 | NM_000088; alpha 1 type I collagen preproprotein |

TABLE 15
32 genes for classifier

| Chip acc. # | UniGene Build 162 | description |
|---|---|---|
| D83920_at | Hs.440898 | NM_002003; ficolin 1 precursor |
| HG67-HT67_f_at | | |
| HG907-HT907_at | | |
| J05032_at | Hs.32393 | NM_001349; aspartyl-tRNA synthetase |
| K01396_at | Hs.297681 | NM_000295; serine (or cysteine) proteinase inhibitor, clade A |
| | | (alpha-1 antiproteinase, antitrypsin), member 1 |
| M16591_s_at | Hs.89555 | NM_002110; hemopoietic cell kinase isoform p61HCK |
| M32011_at | Hs.949 | NM_000433; neutrophil cytosolic factor 2 |
| M33195_at | Hs.433300 | NM_004106; Fc fragment of IgE, high affinity I, receptor for, |
| | | gamma polypeptide precursor |
| M37033_at | Hs.443057 | NM_000560; CD53 antigen |
| M57731_s_at | Hs.75765 | NM_002089; chemokine (C—X—C motif) ligand 2 |
| M63262_at | | |
| S77393_at | Hs.145754 | NM_016531; Kruppel-like factor 3 (basic) |
| U01833_at | Hs.81469 | NM_002484; nucleotide binding protein 1 (MinD homolog, E. coli) |
| U07231_at | Hs.309763 | NM_002092; G-rich RNA sequence binding factor 1 |
| U41315_rna1_s_at | | |
| U47414_at | Hs.13291 | NM_004354; cyclin G2 |
| U50708_at | Hs.1265 | NM_000056; branched chain keto acid dehydrogenase E1, |
| | | beta polypeptide precursor NM_183050; branched chain keto |
| | | acid dehydrogenase E1, beta polypeptide precursor |
| U52101_at | Hs.9999 | NM_001425; epithelial membrane protein 3 |
| U74324_at | Hs.90875 | NM_002871; RAB-interacting factor |
| U77970_at | Hs.321164 | NM_002518; neuronal PAS domain protein 2 NM_032235; |
| U90549_at | Hs.236774 | NM_006353; high mobility group nucleosomal binding domain 4 |
| X13334_at | Hs.75627 | NM_000591; CD14 antigen precursor |
| X54489_rna1_at | | |
| X57579_s_at | | |
| X64072_s_at | Hs.375957 | NM_000211; integrin beta chain, beta 2 precursor |
| X68194_at | Hs.80919 | NM_006754; synaptophysin-like protein isoform a |
| | | NM_182715; synaptophysin-like protein isoform b |
| X73882_at | Hs.254605 | NM_003980; microtubule-associated protein 7 |
| X78520_at | Hs.372528 | NM_001829; chloride channel 3 |
| X95632_s_at | Hs.387906 | NM_005759; abl-interactor 2 |
| Z29331_at | Hs.372758 | NM_003344; ubiquitin-conjugating enzyme E2H isoform 1 |
| | | NM_182697; ubiquitin-conjugating enzyme E2H isoform 2 |
| Z48605_at | Hs.421825 | NM_006903; inorganic pyrophosphatase 2 isoform 2 |
| | | NM_176865; NM_176866; inorganic pyrophosphatase 2 |
| | | isoform 3 NM_176867; inorganic pyrophosphatase 2 isoform 4 |
| | | NM_176869; inorganic pyrophosphatase 2 isoform 1 |
| Z74615_at | Hs.172928 | NM_000088; alpha 1 type I collagen preproprotein |

### Recurrence Predictor

We furthermore tested an outcome predictor able to identify the likely presence or absence of recurrence in patients with superficial Ta tumours (see Table 16).

Table 16. Patient Disease Course Information—Recurrence Vs. No Recurrence

From the hierarchical cluster analysis of the tumour samples we found that the tumours with a high recurrence frequency were separated from the tumours with low recurrence frequency. To study this further we profiled two groups of Ta tumours-15 tumours with low recurrence frequency and 16 tumours with high recurrence frequency. To avoid influence from other tumour characteristics we only used tumours that showed the same growth pattern and tumours that showed no sign of concomitant carcinoma in situ. Furthermore, the tumours were all primary tumours. The tumours used for identifying genes differentially expressed in recurrent and non-recurrent tumours are listed in Table 16 below.

TABLE 16
Disease course information of all patients involved.

| Group | Patient | Tumour (date) | Pattern | Carcinoma in situ | Time to recurrence |
|---|---|---|---|---|---|
| A | 968-1 | Ta gr2 | Papillary | no | 27 month |
| A | 928-1 | Ta gr2 | Papillary | no | 38 month. |
| A | 934-1 | Ta gr2 (220798) | Papillary | no | — |
| A | 709-1 | Ta gr2 (210798) | Papillary | no | — |
| A | 930-1 | Ta gr2 (300698) | Papillary | no | — |
| A | 524-1 | Ta gr2 (201095) | Papillary | no | — |
| A | 455-1 | Ta gr2 (060695) | Papillary | no | — |
| A | 370-1 | Ta gr2 (100195) | Papillary | no | — |
| A | 810-1 | Ta gr2 (031097) | Papillary | no | — |
| A | 1146-1 | Ta gr2 (231199) | Papillary | no | — |
| A | 1161-1 | Ta gr2 (101299) | Mixed | no | — |
| A | 1006-1 | Ta gr2 (231198) | Papillary | no | — |
| A | 942-1 | Ta gr2 | Papillary | no | 24 month. |
| A | 1060-1 | Ta gr2 | Papillary | no | 36 month. |
| A | 1255-1 | Ta gr2 | Papillary | no | 24 month. |
| B | 441-1 | Ta gr2 | Papillary | no | 6 month. |
| B | 780-1 | Ta gr2 | Papillary | no | 2 month. |
| B | 815-2 | Ta gr2 | Papillary | no | 6 month. |
| B | 829-1 | Ta gr2 | Papillary | no | 4 month. |
| B | 861-1 | Ta gr2 | Papillary | no | 4 month. |
| B | 925-1 | Ta gr2 | Papillary | no | 5 month. |
| B | 1008-1 | Ta gr2 | Papillary | no | 5 month. |
| B | 1086-1 | Ta gr2 | Papillary | no | 6 month. |
| B | 1105-1 | Ta gr2 | Papillary | no | 8 month. |
| B | 1145-1 | Ta gr2 | Papillary | no | 4 month. |
| B | 1327-1 | Ta gr2 | Papillary | no | 5 month. |
| B | 1352-1 | Ta gr2 | Papillary | no | 6 month. |
| B | 1379-1 | Ta gr2 | Papillary | no | 5 month. |
| B | 533-1 | Ta gr2 | Papillary | no | 4 month. |
| B | 679-1 | Ta gr2 | Papillary | no | 4 month. |
| B | 692-1 | Ta gr2 | Papillary | no | 5 month. |

Group A: Primary tumours from patients with no recurrence of the disease for 2 years.
Group B: Primary tumours from patients with recurrence of the disease within 8 months.

### Supervised Learning Prediction of Recurrence

In this part of the work we identified genes differentially expressed between non-recurring and recurring tumours. Cross-validation and prediction was performed as previously described, except that genes are selected based on the value of the Wilcoxon statistic for difference between the two groups.

### Prediction Performance

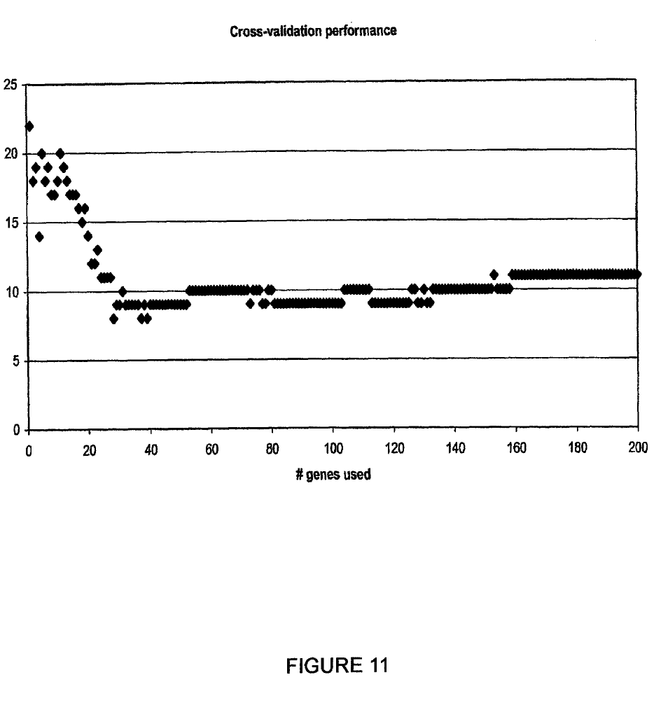

The prediction performance was tested using from 1-200 genes in the cross-validation loops. FIG. 11 shows that the lowest error rate (8 errors) is obtained in e.g. the cross-validation model using from 39 genes. Based on this we selected this cross-validation model as our final predictor. The results of the predictions from the 39 gene cross-validation loops are listed in Table 17. The predictor misclassified four of the samples in each group and in one of the predictions the difference in the distances between the two group means is below the 5% difference limit as described above.

The probability of misclassifying 8 or less arrays by a random classification is 0.0053.

TABLE 17
Recurrence prediction results of 39 gene cross-validation loops.

| | | | | | Prediction |
|---|---|---|---|---|---|
| Group | Patient | Tumour (date) | Prediction | Error | strength |
| A | 968-1 | Ta gr2 | 0 | | 0.19 |
| A | 928-1 | Ta gr2 | 0 | | 0.49 |
| A | 934-1 | Ta gr2 (220798) | 0 | | 1.73 |
| A | 709-1 | Ta gr2 (210798) | 0 | | 0.45 |
| A | 930-1 | Ta gr2 (300698) | 0 | | 0.82 |
| A | 524-1 | Ta gr2 (201095) | 0 | | 0.14 |
| A | 455-1 | Ta gr2 (060695) | 1 | * | 0.68 |
| A | 370-1 | Ta gr2 (100195) | 0 | | 0.32 |
| A | 810-1 | Ta gr2 (031097) | 0 | | 0.45 |
| A | 1146-1 | Ta gr2 (231199) | 0 | | 0.98 |
| A | 1161-1 | Ta gr2 (101299) | 0 | | 0.03 |
| A | 1006-1 | Ta gr2 (231198) | 1 | * | 1.57 |
| A | 942-1 | Ta gr2 | 0 | | 0.31 |
| A | 1060-1 | Ta gr2 | 1 | * | 0.81 |
| A | 1255-1 | Ta gr2 | 1 | * | 0.71 |
| B | 441-1 | Ta gr2 | 1 | | 1.03 |
| B | 780-1 | Ta gr2 | 1 | | 0.37 |
| B | 815-2 | Ta gr2 | 1 | | 0.35 |
| B | 829-1 | Ta gr2 | 1 | | 0.75 |
| B | 861-1 | Ta gr2 | 0 | * | 2.55 |
| B | 925-1 | Ta gr2 | 1 | | 0.78 |
| B | 1008-1 | Ta gr2 | 0 | * | 0.12 |
| B | 1086-1 | Ta gr2 | 0 | * | 0.51 |
| B | 1105-1 | Ta gr2 | 1 | | 0.37 |
| B | 1145-1 | Ta gr2 | 1 | | 0.44 |
| B | 1327-1 | Ta gr2 | 1 | | 1.96 |
| B | 1352-1 | Ta gr2 | 0 | * | 0.97 |
| B | 1379-1 | Ta gr2 | 1 | | 0.67 |
| B | 533-1 | Ta gr2 | 1 | | 0.31 |
| B | 679-1 | Ta gr2 | 1 | | 0.82 |
| B | 692-1 | Ta gr2 | 1 | | 0.45 |

Group A: Primary tumours from patients with no recurrence of the disease for 2 years.
Group B: Primary tumours from patients with recurrence of the disease within 8 months.
Prediction, 0 = no recurrence, 1 = recurrence.

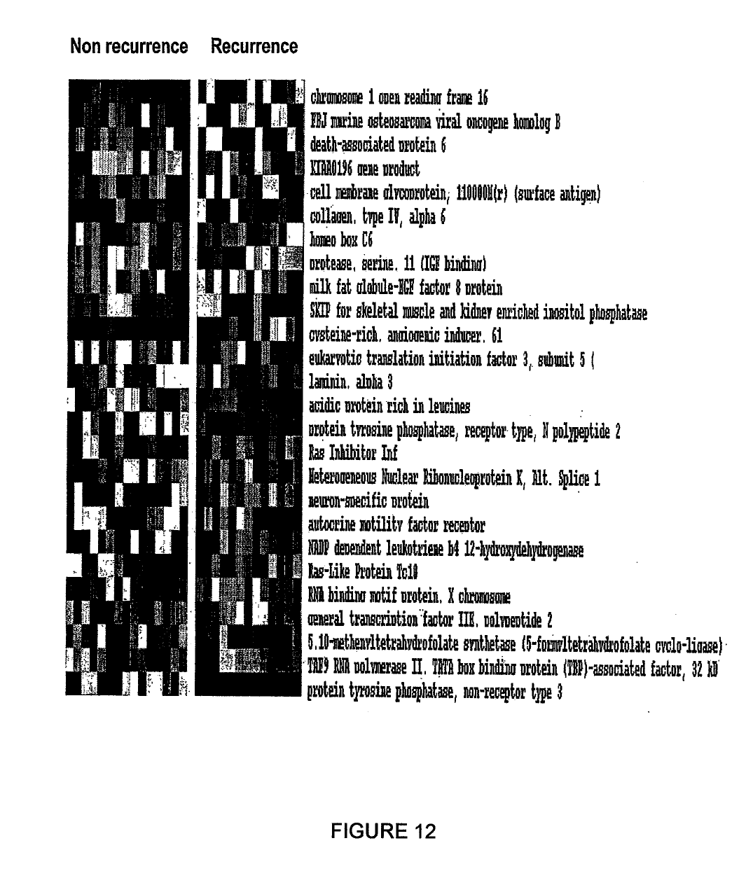

The optimal number of genes in cross-validation loops was found to be 39 (75% of the samples were correct classified, p<0.006) and from this we selected those 26 genes that were used in at least 75% of the cross-validation loops to constitute our final recurrence predictor. Consequently, this set of genes is to be used for predicting recurrence in independent samples. We tested the strength of the predictive genes by permutation analysis, see Table 18. We selected the genes used in at least 29 of the 31 cross-validation loops to constitute our final recurrence prediction model. The expression pattern of those 26 genes is shown in FIG. 12.

TABLE 18
The 26 genes that we find optimal for recurrence prediction.

| | Unigene | | | |
|---|---|---|---|---|
| Feature | build 168 | Description | Number | Test (W-N) |
| AF006041_at | Hs.336916 | NM_001350; death-associated protein 6 | 31 | 0.054 (161-7) |
| D21337_at | Hs.408 | NM_001847; type IV alpha 6 collagen | 31 | 0.058 (160-6) |
| | | isoform A precursor NM_033641; type IV | | |
| | | alpha 6 collagen isoform B precursor | | |
| D49387_at | Hs.294584 | NM_012212; NADP-dependent leukotriene | 31 | 0.118 (313-8) |
| | | B4 12-hydroxydehydrogenase | | |
| D64154_at | Hs.90107 | NM_007002; adhesion regulating molecule | 31 | 0.078 (165-9) |
| | | 1 precursor NM_175573; adhesion regulating | | |
| | | molecule 1 precursor | | |
| D83780_at | Hs.437991 | NM_014846; KIAA0196 gene product | 31 | 0.094 (159-4) |
| D87258_at | Hs.75111 | NM_002775; protease, serine, 11 | 30 | 0.112 (168-11) |
| D87437_at | Hs.43660 | NM_014837; chromosome 1 open reading | 31 | 0.058 (160-6) |
| | | frame 16 | | |
| HG1879-HT1919_at | | | 31 | 0.122 (314-7) |
| HG3076-HT3238_s_at | | | 31 | 0.080 (309-17) |
| HG511-HT511_at | | | 31 | 0.348 (319-2) |
| L34155_at | Hs.83450 | NM_000227; laminin alpha 3 subunit precursor | 31 | 0.122 (314-7) |
| L38928_at | Hs.118131 | NM_006441; 5,10-methenyltetrahydrofolate | 29 | 0.348 (319-2) |
| | | synthetase (5-formyltetrahydrofolate cyclo- | | |
| | | ligase) | | |
| L49169_at | Hs.75678 | NM_006732; FBJ murine osteosarcoma | 31 | 0.108 (155-2) |
| | | viral oncogene homolog B | | |
| M16938_s_at | Hs.820 | NM_004503; homeo box C6 isoform 1 | 29 | 0.09 (170-16) |
| | | NM_153693; homeo box C6 isoform 2 | | |
| M63175_at | Hs.295137 | NM_001144; autocrine motility factor receptor | 29 | 0.098 (308-18) |
| | | isoform a NM_138958; autocrine motility | | |
| | | factor receptor isoform b | | |
| M64572_at | Hs.405666 | NM_002829; protein tyrosine phosphatase, | 31 | 0.064 (305-31) |
| | | non-receptor type 3 | | |
| M98528_at | Hs.79404 | NM_014392; DNA segment on chromosome | 31 | 0.122 (314-7) |
| | | 4 (unique) 234 expressed sequence | | |
| U21858_at | Hs.60679 | NM_003187; TBP-associated factor 9 | 31 | 0.122 (314-7) |
| | | NM_016283; adrenal gland protein AD-004 | | |
| U45973_at | Hs.178347 | NM_016532; skeletal muscle and kidney | 31 | 0.094 (310-14) |
| | | enriched inositol phosphatase isoform 1 | | |
| | | NM_130766; skeletal muscle and kidney | | |
| | | enriched inositol phosphatase isoform 2 | | |
| U58516_at | Hs.3745 | NM_005928; milk fat globule-EGF factor 8 | 29 | 0.100 (175-28) |
| | | protein | | |
| U62015_at | Hs.8867 | NM_001554; cysteine-rich, angiogenic | 31 | 0.106 (169-13) |
| | | inducer, 61 | | |
| U66702_at | Hs.74624 | NM_002847; protein tyrosine phosphatase, | 31 | 0.146 (149-1) |
| | | receptor type, N polypeptide 2 isoform 1 | | |
| | | precursor NM_130842; protein tyrosine | | |
| | | phosphatase, receptor type, N polypeptide | | |
| | | 2 isoform 2 precursor NM_130843; protein | | |
| | | tyrosine phosphatase, receptor type, N | | |
| | | polypeptide 2 isoform 3 precursor | | |
| U70439_s_at | Hs.84264 | NM_006401; acidic (leucine-rich) nuclear | 30 | 0.08 (309-17) |
| | | phosphoprotein 32 family, member B | | |
| U94855_at | Hs.381255 | NM_003754; eukaryotic translation initiation | 30 | 0.092 (311-12) |
| | | factor 3, subunit 5 epsilon, 47 kDa | | |
| X63469_at | Hs.77100 | NM_002095; general transcription factor | 31 | 0.092 (311-12) |
| | | IIE, polypeptide 2, beta 34 kDa | | |
| Z23064_at | Hs.380118 | NM_002139; RNA binding motif protein, X | 30 | 0.066 (307-24) |
| | | chromosome | | |

Number: Number of times the gene has been used in a cross-validation loop. Test: The numbers in parenthesis are the value W of the Wilcoxon test statistic for no difference between the two groups together with the number N of genes for which the Wilcoxon test statistic is bigger than or equal to the value W. The test value is obtained from 500 permutations of the arrays. In each permutation we form new pseudogroups where both of the pseudogroups have the same proportion of arrays from the two original groups. For each permutation we count the number of genes for which the Wilcoxon test statistic based on the pseudogroups is bigger than or equal to W, and the test value is the proportion of the permutations for which this number is bigger than or equal to N. Thus the test value measures the significance of the observed value W. Consequently, for most of our selected genes we only find as least as good predictive genes in about 10% of the formed pseudogroups.

We present data on expression patterns that classify the benign and muscle-invasive bladder carcinomas. Furthermore, we can identify subgroups of bladder cancer such as Ta tumours with surrounding CIS, Ta tumours with a high probability of progression as well as recurrence, and T2 tumours with squamous metaplasia. As a novel finding, the matrix remodelling gene cluster was specifically expressed in the tumours having the worst prognosis, namely the T2 tumours and tumours surrounded by CIS. For some of these genes new small molecule inhibitors already exist (Kerr et al. 2002), and thus they form drug targets. At present it is not possible clinically to identify patients who will experience recurrence and not recurrence, but it would be a great benefit to both the patients and the health system by reducing the number of unnecessary control examinations in bladder tumour patients. To determine the optimal gene-set for separating non-recurrent and recurrent tumours, we again applied a cross-validation scheme using from 1-200 genes. We determined the optimal number of genes in cross-validation loops to be 39 (75% of the samples were correct classified, p<0.01, FIG. 11) and from this we selected those 26 genes (FIG. 12) that were used in at least 75% of the cross-validation loops to constitute our final recurrence predictor. Consequently, this set of genes is to be used for predicting recurrence in independent samples. We tested the strength of the predictive genes by performing 500 permutations of the arrays. This revealed that for most of our predictive genes we would only in a small number of the new pseudo-groups obtain at least as good predictors as in the real groups.

### Biological Material

66 bladder tumour biopsies were sampled from patients following removal of the necessary amount of tissue for routine pathology examination. The tumours were frozen immediately after surgery and stored at −80° C. in a guanidinium thiocyanate solution. All tumours were graded according to Bergkvist et al. 1965 and re-evaluated by a single pathologist. As normal urothelial reference samples we used a pool of biopsies (from 37 patients) as well as three single bladder biopsies from patients with prostatic hyperplasia or urinary incontinence. Informed consent was obtained in all cases and protocols were approved by the local scientific ethical committee.

RNA Purification and cRNA Preparation

Total RNA was isolated from crude tumour biopsies using a Polytron homogenisator and the RNAzol B RNA isolation method (WAK-Chemie Medical GmbH). 10 μg total RNA was used as starting material for the cDNA preparation. The first and second strand cDNA synthesis was performed using the SuperScript Choice System (Life Technologies) according to the manufacturers instructions except using an oligo-dT primer containing a T7 RNA polymerase promoter site. Labelled cRNA was prepared using the BioArray High Yield RNA Transcript Labelling Kit (Enzo). Biotin labelled CTP and UTP (Enzo) were used in the reaction together with unlabeled NTP's. Following the IVT reaction, the unincorporated nucleotides were removed using RNeasy columns (Qiagen).

### Array Hybridisation and Scanning

15 μg of cRNA was fragmented at 94° C. for 35 min in a fragmentation buffer containing 40 mM Tris-acetate pH 8.1, 100 mM KOAc, 30 mM MgOAc. Prior to hybridisation, the fragmented cRNA in a 6×SSPE-T hybridisation buffer (1 M NaCl, 10 mM Tris pH 7.6, 0.005% Triton), was heated to 95° C. for 5 min and subsequently to 45° C. for 5 min before loading onto the Affymetrix probe array cartridge (HuGeneFL). The probe array was then incubated for 16 h at 45° C. at constant rotation (60 rpm). The washing and staining procedure was performed in the Affymetrix Fluidics Station. The probe array was exposed to 10 washes in 6×SSPE-T at 25° C. followed by 4 washes in 0.5×SSPE-T at 50° C. The biotinylated cRNA was stained with a streptavidin-phycoerythrin conjugate, final concentration 2 μg/μl (Molecular Probes, Eugene, Oreg.) in 6×SSPE-T for 30 min at 25° C. followed by 10 washes in 6×SSPE-T at 25° C. The probe arrays were scanned at 560 nm using a confocal laser-scanning microscope (Hewlett Packard GeneArray Scanner G2500A). The readings from the quantitative scanning were analysed by the Affymetrix Gene Expression Analysis Software. An antibody amplification step followed using normal goat IgG as blocking reagent, final concentration 0.1 mg/ml (Sigma) and biotinylated anti-streptavidin antibody (goat), final concentration 3 μg/ml (Vector Laboratories). This was followed by a staining step with a streptavidin-phycoerythrin conjugate, final concentration 2 μg/μl (Molecular Probes, Eugene, Oreg.) in 6×SSPE-T for 30 min at 25° C. and 10 washes in 6×SSPE-T at 25° C. The arrays were then subjected to a second scan under similar conditions as described above.

### Class Discovery Using Hierarchical Clustering

All microarray results were scaled to a global intensity of 150 units using the Affymetrix GeneChip software. Other ways of array normalisation exist (Li and Hung 2001), however, using the dCHIP approach did not change the expression profiles of the obtained classifier genes in this study (results not shown). For hierarchical cluster analysis and molecular classification procedures we used expression level ratios between tumours and the normal urothelium reference pool calculated using the comparison analysis implemented in the Affymetrix GeneChip software. In order to avoid expression ratios based on saturated gene-probes, we used the antibody amplified expression-data for genes with a mean Average Difference value across all samples below 1000 and the non-amplified expression-data for genes with values equal to or above 1000 in mean Average Difference value across all samples. Consequently, gene expression levels across all samples were either from the amplified or the non-amplified expression-data. We applied different filtering criteria to the expression data in order to avoid including non-varying and very low expressed genes in the data analysis. Firstly, we selected only genes that showed significant changes in expression levels compared to the normal reference pool in at least three samples. Secondly, only genes with at least three “Present” calls across all samples were selected. Thirdly, we eliminated genes varying less than 2 standard deviations across all samples. The final gene-set contained 1767 genes following filtering. Two-way hierarchical agglomerative cluster analysis was performed using the Cluster software²⁵. We used average linkage clustering with a modified Pearson correlation as similarity metric. Genes and arrays were median centred and normalised to the magnitude of 1 prior to cluster analysis. The TreeView software was used for visualisation of the cluster analysis results (Eisen et al. 1998). Multidimensional scaling was performed on median centred and normalised data using an implementation in the SPSS statistical software package.

### Tumour Stage Classifier

We based the classifier on the log-transformed expression level ratios. For these trans-formed values we used a normal distribution with the mean dependent on the gene and the group (Ta, T1, and T2, respectively) and the variance dependent on the gene only. For each gene we calculated the variation within the groups (W) and the three variations between two groups (B(Ta/T1), B(Ta/T2), B(T1/T2)) and used the three ratios B/W to select genes. We selected those genes having a high value of B(Ta/T1)/W, those genes having a high value of B(Ta/T2)/W, and those genes with a high value of B(T1/T2)/W. To classify a sample, we calculated the sum over the genes of the squared distance from the sample value to the group mean, standardised by the variance. Thus, we got a distance to each of the three groups and the sample was classified as belonging to the group in which the distance was smallest. When calculating these distances the group means and the variances were estimated from all the samples in the training set excluding the sample being classified.

### Recurrence Prediction Using a Supervised Learning Method

Average Difference values were generated using the Affymetrix GeneChip software and all values below 20 were set to 20 to avoid very low and negative numbers. We only included genes that had a “Present” call in at least 7 samples and genes that showed intensity variation (Max−Min>100, Max/Min>2). The values were log transformed and rescaled. We used a supervised learning method essentially as described (Shipp et al. 2002). Genes were selected using t-test statistics and cross-validation and sample classification was performed as described above.

### Immunohistochemistry

Tumour tissue microarrays were prepared essentially as described (Kononen et al. 1998), with four representative 0.6 mm paraffin cores from each study case. Immunohistochemical staining was performed using standard highly sensitive techniques after appropriate heat-induced antigen retrieval. Primary polyclonal goat antibodies against Smad 6 (S-20) and cyclin G2 (N-19) were from Santa Cruz Biotechnology. Antibodies to p53 (monoclonal DO-7) and Her-2 (polyclonal anti-c-erbB-2) were from Dako A/S. Ki-67 monoclonal antibody (MIBI) was from Novocastra Laboratories Ltd. Staining intensity was scored at four levels, Negative, Weak, Moderate and Strong by an experienced pathologist who considered both colour intensity and number of stained cells, and who was unaware of array results.

### Example 3

A molecular classifier detects carcinoma in situ expression signatures in tumors and normal urothelium of the bladder.

### Clinical Samples

Bladder tumour samples were obtained directly from surgery following removal of tissue for routine pathological examination. The samples were immediately submerged in a guadinium thiocyanate solution for RNA preservation and stored at −80° C. Informed consent was obtained in all cases, and the protocols were approved by the scientific ethical committee of Aarhus County. Samples in the No-CIS group were selected based on the following criteria: a) Ta tumours with no CIS in selected site biopsies in all visits; b) no previous muscle invasive tumour. Samples in the CIS group were selected based on the criteria: a) Ta or T1 tumours with CIS in selected site biopsies in any visit (preferable Ta tumours with CIS in the sampling visit); b) no previous muscle invasive tumours. Normal biopsies were obtained from individuals with prostatic hyperplasia or urinary incontinence. CIS and “normal” biopsies were obtained from cystectomy specimens directly following removal of the bladder. A grid was placed in the bladder for orientation and biopsies were taken from 8 positions covering the bladder surface. At each position, three biopsies were taken—two for pathologic examination and one in between these for RNA extraction for microarray expression profiling. The samples for RNA extraction were immediately transferred to the guadinium thiocyanate solution and stored at −80° C. until use. Samples used for RNA extraction were assumed to have CIS if CIS was detected in both adjacent biopsies. The “normal” samples were assumed to be normal if both adjacent biopsies were normal.

cRNA Preparation, Array Hybridisation and Scanning

Purification of total RNA, preparation of cRNA from cDNA and hybridisation and scanning were performed as previously described (Dyrskjot et al. 2003). The labelled samples were hybridised to Affymetrix U133A GeneChips.

### Expression Data Analysis

Following scanning all data were normalised using the RMA normalisation approach in the Bioconductor Affy package to R. Variation filters were applied to the data to eliminate non-varying and presumably non-expressed genes. For gene-set 1 this was done by only including genes with a minimum expression above 200 in at least 5 samples and genes with max/min expression intensities above or equal to 3. The filtering for gene-set 2 including only genes with a minimum expression of 200 in at least 3 samples and genes with max/min expression intensities above or equal to 3. Average linkage hierarchical cluster analysis was carried out using the Cluster software with a modified Pearson correlation as similarity metric (Eisen et al. 1998). We used the TreeView software for visualisation of the cluster analysis results (Eisen et al. 1998). Genes were log-transformed, median centred and normalised to the magnitude of 1 before clustering. We used GeneCluster 2.0 (http://www-genome.wi.mit.edu/cancer/software/genecluster2/qc2.html) for the supervised selection of markers and for permutation testing. The algorithms used in the software are based on (Golub et al. 1999, Tamayo et al. 1999). Classifiers for CIS detection were built using the same methods as described previously (Dyrskjot et al. 2003).

### Gene Expression Profiling

We used high-density oligonucleotide microarrays for gene expression profiling of approximately 22,000 genes in 28 superficial bladder tumour biopsies (13 tumours with surrounding CIS and 15 without surrounding CIS) and in 13 invasive carcinomas. See table 19 for patient disease course descriptions. Furthermore, expression profiles were obtained from 9 normal biopsies and from 10 biopsies from cystectomy specimens (5 histologically normal biopsies and 5 biopsies with CIS).

TABLE 19
Clinical data on patient disease courses and results of molecular CIS classification

| Sample | | Previous | Tumour | Subsequent | | |
|---|---|---|---|---|---|---|
| group^{a} | Patient^{b} | tumours | analysed | tumours | CIS^{c} | CIS classifier^{d} |
| 1 | 1060-1 | | Ta gr2 | 2 Ta | No | No CIS |
| 1 | 1146-1 | | Ta gr2 | | No | No CIS |
| 1 | 1216-1 | | Ta gr2 | | No | No CIS |
| 1 | 1303-1 | | Ta gr2 | | No | No CIS |
| 1 | 524-1 | | Ta gr2 | | No | No CIS |
| 1 | 692-1 | | Ta gr2 | 2 Ta | No | No CIS |
| 1 | 1264-1 | | Ta gr3 | 20 Ta | No | No CIS |
| 1 | 1350-1 | | Ta gr3 | 1 Ta | No | No CIS |
| 1 | 1354-1 | | Ta gr3 | 11 T1 | No | No CIS |
| 1 | 775-1 | | Ta gr3 | 1 Ta | No | No CIS |
| 1 | 1066-1 | | Ta gr3 | 1 Ta | No | No CIS |
| 1 | 1276-1 | | Ta gr3 | 2 T1 | No | No CIS |
| 1 | 1070-1 | | Ta gr3 | 1 Ta | No | No CIS |
| 1 | 989-1 | | Ta gr3 | | No | No CIS |
| 1 | 1482-1 | | Ta gr3 | 20 Ta | No | CIS |
| 2 | 1345-2 | 1 T1 | Ta gr3 | | Sampling visit | CIS |
| 2 | 1062-2 | | Ta gr3 | 1 T1 | Sampling visit | CIS |
| 2 | 956-2 | | Ta gr3 | 1 Ta | Sampling visit | CIS |
| 2 | 320-7 | 1 Ta, 2 T1 | Ta gr3 | 2 Ta | Sampling visit | CIS |
| 2 | 1330-1 | | Ta gr3 | | Sampling visit | CIS |
| 2 | 602-8 | 5 Ta | Ta gr3 | 3 Ta | Sampling visit | CIS |
| 2 | 763-1 | | Ta gr2 | 14 Ta | Sampling visit | CIS |
| 2 | 1024-1 | | T1 gr3 | 2 Ta, 1 T1 | Sampling visit | CIS |
| 2 | 1182-1 | | Ta gr3 | 7 Ta | Subsequent visit | CIS |
| 2 | 1093-1 | | Ta gr3 | 4 Ta, 1 T1 | Subsequent visit | CIS |
| 2 | 979-1 | | Ta gr3 | | Sampling visit | CIS |
| 2 | 1337-1 | | T1 gr3 | | Sampling visit | CIS |
| 2 | 1625-1 | | Ta gr2 | | Sampling visit | CIS |
| 3 | 1015-1 | | T3b gr4 | | No | — |
| 3 | 1337-1 | | T4a gr3 | | Sampling visit | — |
| 3 | 1041-1 | | T4b gr3 | | No | — |
| 3 | 1044-1 | | T4b gr3 | | ND | — |
| 3 | 1055-1 | 1 Ta gr2 | T3a gr3 | | No | — |
| 3 | 1109-1 | | T2 gr3 | 1 T2-4 | No | — |
| 3 | 1124-1 | | T4a gr3 | 2 T2-4 | No | — |
| 3 | 1154-1 | | T3a gr3 | 1 Ta, 1 T2-4 | No | — |
| 3 | 1167-1 | 1 T2-4 | T3b gr4 | 2 T2-4 | ND | — |
| 3 | 1178-1 | | T4b gr3 | | ND | — |
| 3 | 1215-1 | | T4b gr3 | | ND | — |
| 3 | 1271-1 | | T3b gr4 | | No | — |
| 3 | 1321-1 | 1 T1 | T3b gr? | | ND | — |

^{a}The tumour groups involved were TCC without CIS (1), TCC with CIS (2) and invasive TCC (3).
^{b}The numbers indicate the patient number followed by the clinic visit number.
^{c}CIS in selected site biopsies in previous, present or subsequent visits to the clinic. ND: not determined.
^{d}Molecular classification of the samples using 25 genes in cross-validation loops.

### Hierarchical Cluster Analysis

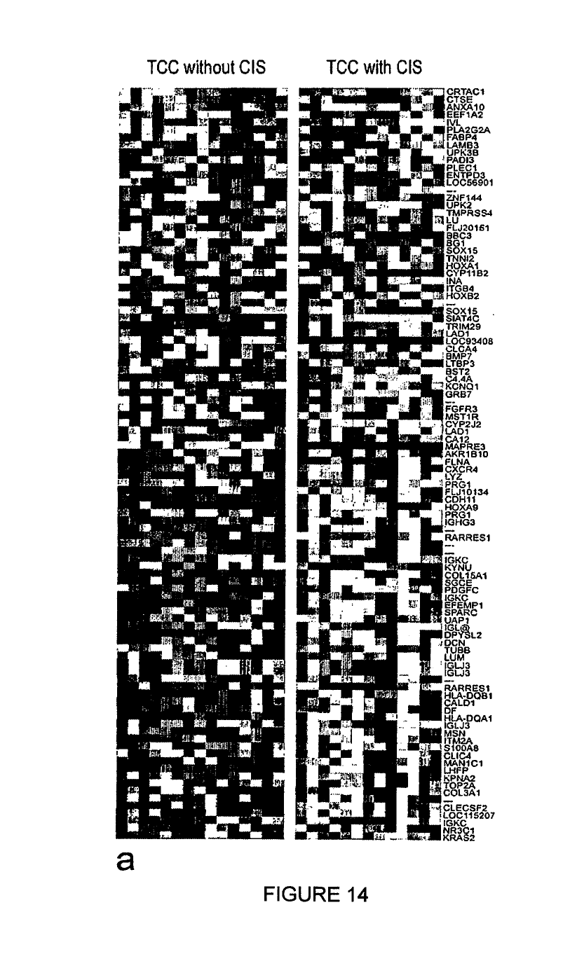

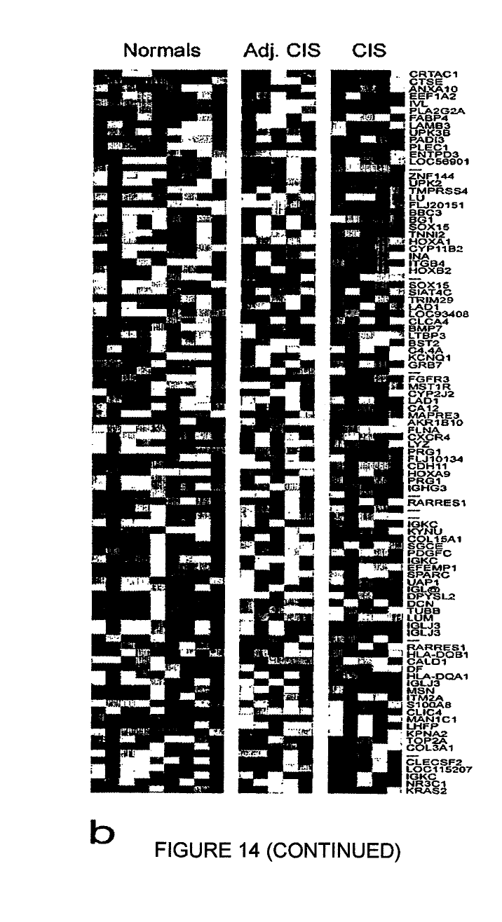

Following appropriate normalisation and expression intensity calculations we selected those genes that showed high variation across the 41 TCC samples for further analysis. The filtering produced a gene-set consisting of 5,491 genes (gene-set 1) and two-way hierarchical cluster analysis was performed based on this gene-set. The sample clustering showed a separation of the three groups of samples with only few exceptions (FIG. 14a). Superficial TCC with surrounding CIS clustered in the one main branch of the dendrogram, while the superficial TCC without CIS and the invasive TCC clustered in two separate sub-branches in the other main branch of the dendrogram. The only exceptions were that the invasive TCC samples 1044-1 and 1124-1 clustered in the CIS group and two TCC with CIS clustered in the invasive group (samples 1330-1 and 956-2). The only TCC without CIS that clustered in the CIS group was sample 1482-1. The distinct clustering of the tumour groups indicated a large difference in gene expression patterns.

Hierarchical clustering of the genes (FIG. 14c) identified large clusters of genes characteristic for the each tumour phenotype. Cluster 1 showed a cluster of genes down-regulated in cystectomy biopsies, TCC with adjacent CIS and in some invasive carcinomas (FIG. 14c). There is no obvious functional relationship between the genes in this cluster. Cluster 2 showed a tight cluster of genes related to immunology and cluster 3 contained mostly genes expressed in muscle and connective tissue. Expression of genes in this cluster was observed in the normal and cystectomy samples, in a fraction of the TCC with CIS and in the invasive tumours. Cluster 4 contained genes up-regulated in the cystectomy biopsies, TCC with adjacent CIS and in invasive carcinomas (FIG. 14c). This cluster includes genes involved in cell cycle regulation, cell proliferation and apoptosis. However, for most of the genes in this cluster there is not apparent functional relationship either. Comparisons of chromosomal location of the genes in the clusters revealed no correlation between the observed gene clusters and chromosomal position of the identified genes. A positive correlation could have indicated chromosomal loss or gain or chromosomal inactivation by e.g. methylation of common promoter regions.

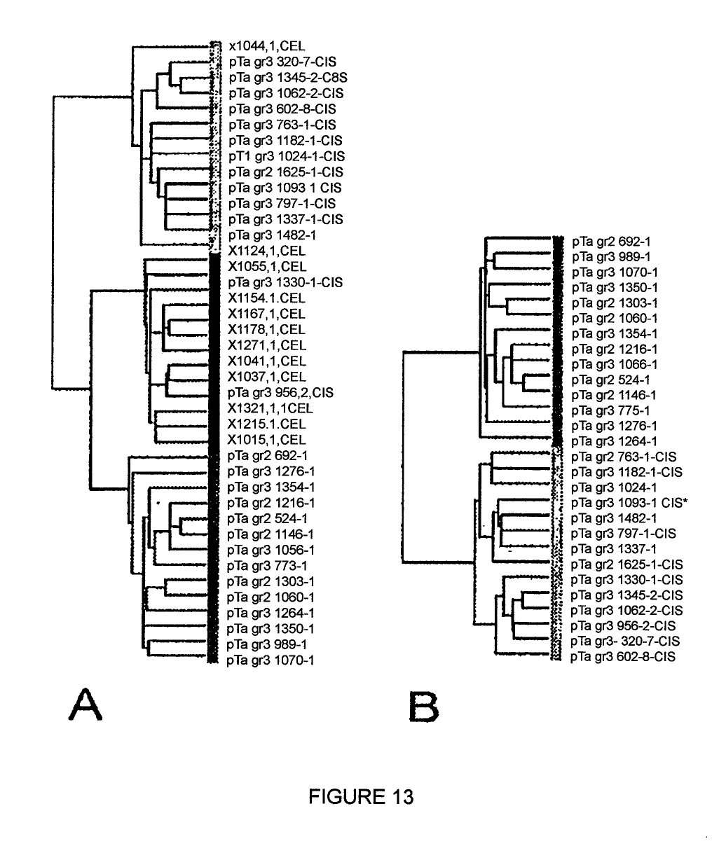

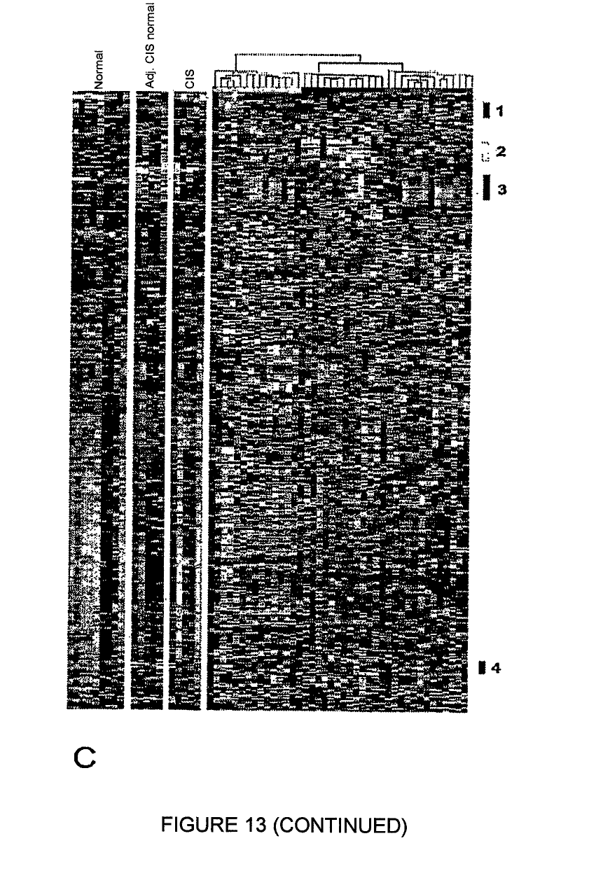

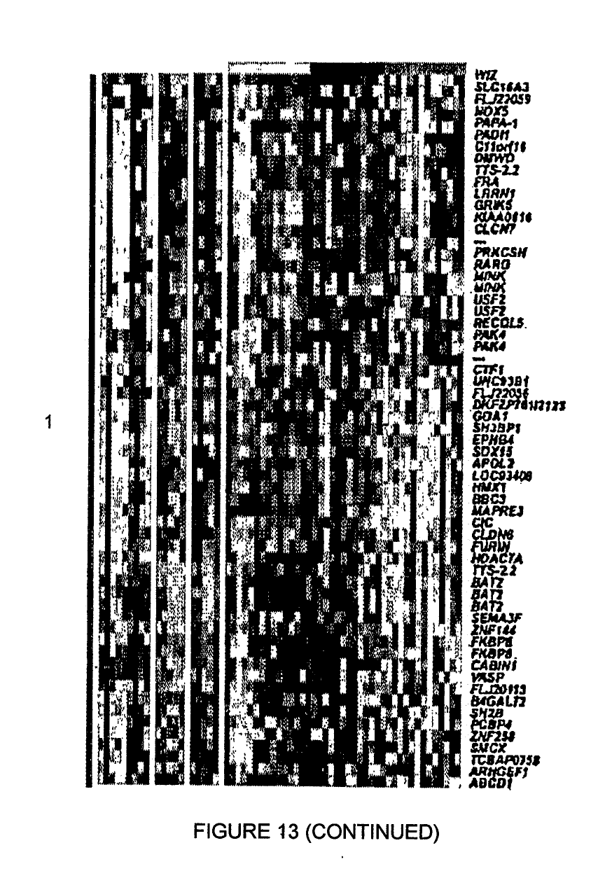

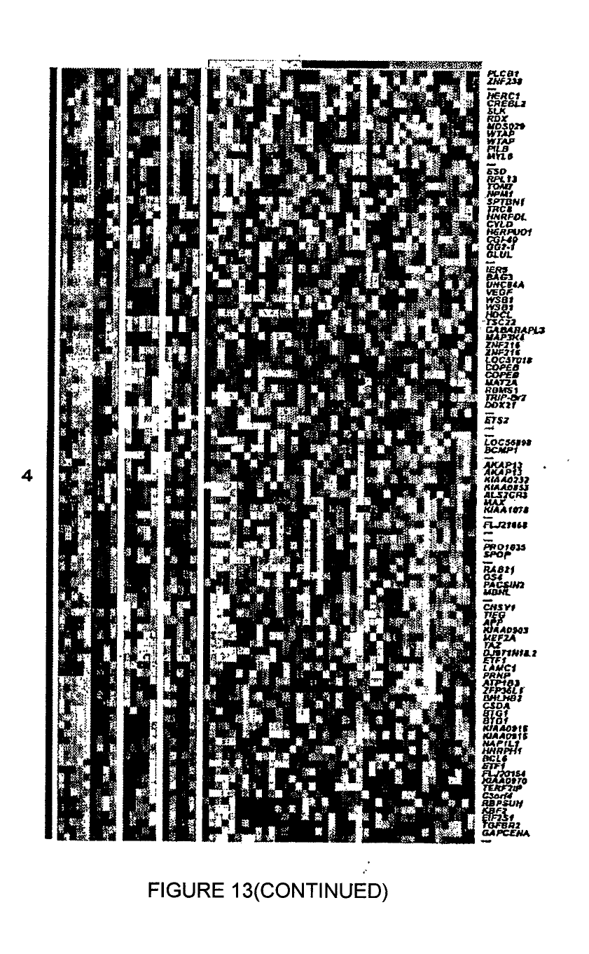

To analyse the impact of surrounding CIS lesions further we used the 28 superficial tumours only, and created a new gene set consisting of 5,252 varying genes (gene-set 2). Hierarchical cluster analysis of the tumour samples (FIG. 13b) based on the new gene-set separated the samples according to the presence of CIS in the surrounding urothelium with only 1 exception (P<0.000001, χ²-test). Sample 1482-1 clustered in the TCC with CIS group, however, no CIS has been detected in selected site biopsies during routine examinations of this patient. Tumour samples 1182-1 and 1093-1 did not have CIS in selected site biopsies in the same visit as the profiled tumour but showed this in later visits. However, the profile of these two superficial tumour samples already showed the adjacent CIS profile.

### Marker Selection

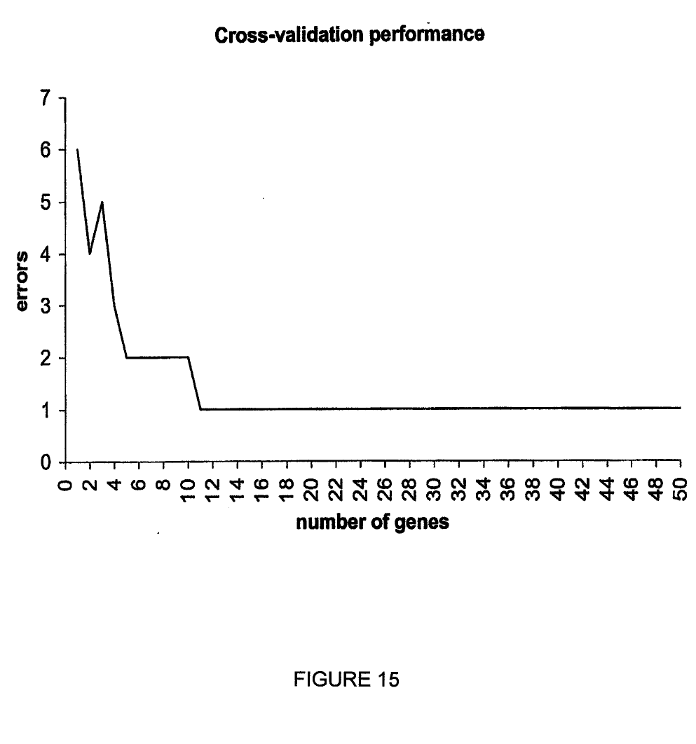

To delineate the tumours with surrounding CIS from the tumours without CIS we used t-test statistics to select the 50 most up-regulated genes in each group (FIG. 15a). Permutation of the sample labels 500 times revealed that the 50 genes up-regulated in the CIS-group are highly significant differentially expressed and unlikely to find by chance, as all markers were significant on a 5% confidence level. Consequently, in 500 random datasets it was only possible to select as good genes in less than 5% of the datasets. The 50 genes up-regulated in the no-CIS group showed a poorer performance in the permutation tests, as these were not significant on a 5% confidence level. See Table 20 for details. The relative expression of these 100 genes is 9 normal and 10 biopsies from cystectomies with CIS are shown in FIG. 15b. The no-CIS profile was found in all of the normal samples. However, all histologically normal samples adjacent to the CIS lesions as well as the CIS biopsies showed the CIS profile.

TABLE 20
The best 100 markers

| Feature | | | Perm | Perm | Perm | | |
|---|---|---|---|---|---|---|---|
| (U133 array) | Class | T-test | 1% | 5% | 10% | UniGene Build 162 | RefSeq; description |
| 221204_s_at | no_CIS | 3.74 | 5.12 | 4.61 | 4.33 | Hs.326444 | NM_018058; cartilage |
| | | | | | | | acidic protein 1 |
| 205927_s_at | no_CIS | 3.67 | 4.53 | 3.98 | 3.73 | Hs.1355 | NM_001910; cathepsin E |
| | | | | | | | isoform a preproprotein |
| | | | | | | | NM_148964; cathepsin E |
| | | | | | | | isoform b preproprotein |
| 210143_at | no_CIS | 3.35 | 4.03 | 3.73 | 3.45 | Hs.188401 | NM_007193; annexin A10 |
| 204540_at | no_CIS | 3.15 | 3.87 | 3.51 | 3.32 | Hs.433839 | NM_001958; eukaryotic |
| | | | | | | | translation elongation |
| | | | | | | | factor 1 alpha 2 |
| 214599_at | no_CIS | 3.02 | 3.75 | 3.37 | 3.14 | Hs.157091 | NM_005547; involucrin |
| 203649_s_at | no_CIS | 2.84 | 3.63 | 3.20 | 3.00 | Hs.76422 | NM_000300; phospholipase |
| | | | | | | | A2, group IIA (platelets, |
| | | | | | | | synovial fluid) |
| 203980_at | no_CIS | 2.74 | 3.47 | 3.12 | 2.89 | Hs.391561 | NM_001442; fatty acid |
| | | | | | | | binding protein 4, adipocyte |
| 209270_at | no_CIS | 2.39 | 3.38 | 3.10 | 2.85 | Hs.436983 | NM_000228; laminin |
| | | | | | | | subunit beta 3 precursor |
| 206658_at | no_CIS | 2.35 | 3.37 | 3.05 | 2.78 | Hs.284211 | NM_030570; uroplakin 3B |
| | | | | | | | isoform a NM_182683; |
| | | | | | | | uroplakin 3B isoform c |
| | | | | | | | NM_182684; uroplakin 3B |
| | | | | | | | isoform b |
| 220779_at | no_CIS | 2.35 | 3.33 | 2.97 | 2.73 | Hs.149195 | NM_016233; peptidylarginine |
| | | | | | | | deiminase type |
| | | | | | | | III |
| 216971_s_at | no_CIS | 2.28 | 3.29 | 2.91 | 2.71 | Hs.79706 | NM_000445; plectin 1, |
| | | | | | | | intermediate filament |
| | | | | | | | binding protein 500 kDa |
| 206191_at | no_CIS | 2.25 | 3.24 | 2.86 | 2.68 | Hs.47042 | NM_001248; ectonucleoside |
| | | | | | | | triphosphate diphosphohydrolase 3 |
| 218484_at | no_CIS | 2.18 | 3.20 | 2.81 | 2.62 | Hs.221447 | NM_020142; |
| | | | | | | | NADH: ubiquinone oxidoreductase |
| | | | | | | | MLRQ |
| | | | | | | | subunit homolog |
| 221854_at | no_CIS | 2.1 | 3.19 | 2.80 | 2.60 | Hs.313068 | NM_000299; plakophilin 1 |
| 203792_x_at | no_CIS | 2.02 | 3.16 | 2.74 | 2.55 | Hs.371617 | NM_007144; ring finger |
| | | | | | | | protein 110 |
| 207862_at | no_CIS | 2.01 | 3.16 | 2.72 | 2.52 | Hs.379613 | NM_006760; uroplakin 2 |
| 218960_at | no_CIS | 1.93 | 3.14 | 2.65 | 2.47 | Hs.414005 | NM_019894; transmembrane |
| | | | | | | | protease, serine 4 |
| | | | | | | | isoform 1 NM_183247; |
| | | | | | | | transmembrane protease, |
| | | | | | | | serine 4 isoform 2 |
| 203009_at | no_CIS | 1.93 | 3.12 | 2.62 | 2.45 | Hs.155048 | NM_005581; Lutheran |
| | | | | | | | blood group (Auberger b |
| | | | | | | | antigen included) |
| 204508_s_at | no_CIS | 1.88 | 3.10 | 2.60 | 2.42 | Hs.279916 | NM_017689; hypothetical |
| | | | | | | | protein FLJ20151 |
| 211692_s_at | no_CIS | 1.87 | 3.06 | 2.58 | 2.39 | Hs.87246 | NM_014417; BCL2 binding |
| | | | | | | | component 3 |
| 206465_at | no_CIS | 1.86 | 3.04 | 2.54 | 2.38 | Hs.277543 | NM_015162; lipidosin |
| 206122_at | no_CIS | 1.85 | 2.92 | 2.52 | 2.36 | Hs.95582 | NM_006942; SRY-box 15 |
| 206393_at | no_CIS | 1.83 | 2.89 | 2.49 | 2.33 | Hs.83760 | NM_003282; troponin I, |
| | | | | | | | skeletal, fast |
| 214639_s_at | no_CIS | 1.79 | 2.87 | 2.49 | 2.30 | Hs.67397 | NM_005522; homeobox |
| | | | | | | | A1 protein isoform a |
| | | | | | | | NM_153620; homeobox |
| | | | | | | | A1 protein isoform b |
| 214630_at | no_CIS | 1.79 | 2.84 | 2.44 | 2.28 | Hs.184927 | NM_000497; cytochrome |
| | | | | | | | P450, subfamily XIB |
| | | | | | | | (steroid 11-beta- |
| | | | | | | | hydroxylase), polypeptide |
| | | | | | | | 1 precursor |
| 204465_s_at | no_CIS | 1.77 | 2.81 | 2.42 | 2.27 | Hs.76888 | NM_004692; |
| | | | | | | | NM_032727; internexin |
| | | | | | | | neuronal intermediate |
| | | | | | | | filament protein, alpha |
| 204990_s_at | no_CIS | 1.76 | 2.79 | 2.41 | 2.24 | Hs.85266 | NM_000213; integrin, |
| | | | | | | | beta 4 |
| 205453_at | no_CIS | 1.75 | 2.77 | 2.39 | 2.22 | Hs.290432 | NM_002145; homeo box |
| | | | | | | | B2 |
| 215812_s_at | no_CIS | 1.74 | 2.77 | 2.37 | 2.20 | Hs.499113 | NM_018058; cartilage |
| | | | | | | | acidic protein 1 |
| 217040_x_at | no_CIS | 1.74 | 2.75 | 2.36 | 2.18 | Hs.95582 | NM_001910; cathepsin E |
| | | | | | | | isoform a preproprotein |
| | | | | | | | NM_148964; cathepsin E |
| | | | | | | | isoform b preproprotein |
| 203759_at | no_CIS | 1.73 | 2.75 | 2.34 | 2.17 | Hs.75268 | NM_007193; annexin A10 |
| 211002_s_at | no_CIS | 1.73 | 2.74 | 2.33 | 2.17 | Hs.82237 | NM_001958; eukaryotic |
| | | | | | | | translation elongation |
| | | | | | | | factor 1 alpha 2 |
| 216641_s_at | no_CIS | 1.73 | 2.73 | 2.31 | 2.15 | Hs.18141 | NM_005547; involucrin |
| 221660_at | no_CIS | 1.71 | 2.67 | 2.30 | 2.13 | Hs.247831 | NM_000300; phospholipase |
| | | | | | | | A2, group IIA (platelets, |
| | | | | | | | synovial fluid) |
| 220026_at | no_CIS | 1.71 | 2.66 | 2.28 | 2.13 | Hs.227059 | NM_001442; fatty acid |
| | | | | | | | binding protein 4, adipocyte |
| 209591_s_at | no_CIS | 1.69 | 2.63 | 2.28 | 2.11 | Hs.170195 | NM_000228; laminin |
| | | | | | | | subunit beta 3 precursor |
| 219922_s_at | no_CIS | 1.68 | 2.61 | 2.26 | 2.08 | Hs.289019 | NM_030570; uroplakin 3B |
| | | | | | | | isoform a NM_182683; |
| | | | | | | | uroplakin 3B isoform c |
| | | | | | | | NM_182684; uroplakin 3B |
| | | | | | | | isoform b |
| 201641_at | no_CIS | 1.67 | 2.61 | 2.26 | 2.07 | Hs.118110 | NM_016233; peptidylarginine |
| | | | | | | | deiminase type |
| | | | | | | | III |
| 204952_at | no_CIS | 1.66 | 2.59 | 2.24 | 2.07 | Hs.377028 | NM_000445; plectin 1, |
| | | | | | | | intermediate filament |
| | | | | | | | binding protein 500 kDa |
| 204487_s_at | no_CIS | 1.65 | 2.59 | 2.23 | 2.06 | Hs.367809 | NM_001248; ectonucleoside |
| | | | | | | | triphosphate diphosphohydrolase 3 |
| 210761_s_at | no_CIS | 1.64 | 2.59 | 2.23 | 2.05 | Hs.86859 | NM_020142; |
| | | | | | | | NADH: ubiquinone oxidoreductase |
| | | | | | | | MLRQ |
| | | | | | | | subunit homolog |
| 217626_at | no_CIS | 1.63 | 2.58 | 2.21 | 2.04 | Hs.201967 | NM_000299; plakophilin 1 |
| 204380_s_at | no_CIS | 1.62 | 2.58 | 2.19 | 2.03 | Hs.1420 | NM_007144; ring finger |
| | | | | | | | protein 110 |
| 205455_at | no_CIS | 1.61 | 2.58 | 2.17 | 2.02 | Hs.2942 | NM_006760; uroplakin 2 |
| 205073_at | no_CIS | 1.61 | 2.58 | 2.17 | 2.01 | Hs.152096 | NM_019894; transmembrane |
| | | | | | | | protease, serine 4 |
| | | | | | | | isoform 1 NM_183247; |
| | | | | | | | transmembrane protease, |
| | | | | | | | serine 4 isoform 2 |
| 203287_at | no_CIS | 1.61 | 2.58 | 2.16 | 2.00 | Hs.18141 | NM_005581; Lutheran |
| | | | | | | | blood group (Auberger b |
| | | | | | | | antigen included) |
| 210735_s_at | no_CIS | 1.58 | 2.55 | 2.15 | 1.99 | Hs.5338 | NM_017689; hypothetical |
| | | | | | | | protein FLJ20151 |
| 203842_s_at | no_CIS | 1.57 | 2.54 | 2.15 | 1.97 | Hs.172740 | NM_014417; BCL2 binding |
| | | | | | | | component 3 |
| 206561_s_at | no_CIS | 1.57 | 2.53 | 2.14 | 1.96 | Hs.116724 | NM_015162; lipidosin |
| 214752_x_at | no_CIS | 1.56 | 2.52 | 2.13 | 1.95 | Hs.195464 | NM_006942; SRY-box 15 |
| 217028_at | CIS | 4.87 | 5.17 | 4.67 | 4.40 | Hs.421986 | NM_003282; troponin I, |
| | | | | | | | skeletal, fast |
| 213975_s_at | CIS | 4.65 | 4.43 | 4.01 | 3.76 | Hs.234734 | NM_005522; homeobox |
| | | | | | | | A1 protein isoform a |
| | | | | | | | NM_153620; homeobox |
| | | | | | | | A1 protein isoform b |
| 201859_at | CIS | 4.59 | 4.15 | 3.70 | 3.45 | Hs.1908 | NM_000497; cytochrome |
| | | | | | | | P450, subfamily XIB |
| | | | | | | | (steroid 11-beta- |
| | | | | | | | hydroxylase), polypeptide |
| | | | | | | | 1 precursor |
| 219410_at | CIS | 4.49 | 3.98 | 3.49 | 3.29 | Hs.104800 | NM_004692; |
| | | | | | | | NM_032727; internexin |
| | | | | | | | neuronal intermediate |
| | | | | | | | filament protein, alpha |
| 207173_x_at | CIS | 4.37 | 3.88 | 3.33 | 3.11 | Hs.443435 | NM_000213; integrin, |
| | | | | | | | beta 4 |
| 214651_s_at | CIS | 4.14 | 3.83 | 3.22 | 2.99 | Hs.127428 | NM_002145; homeo box |
| | | | | | | | B2 |
| 201858_s_at | CIS | 4.06 | 3.78 | 3.09 | 2.91 | Hs.1908 | NM_018058; cartilage |
| | | | | | | | acidic protein 1 |
| 211430_s_at | CIS | 4.03 | 3.63 | 3.05 | 2.83 | Hs.413826 | NM_001910; cathepsin E |
| | | | | | | | isoform a preproprotein |
| | | | | | | | NM_148964; cathepsin E |
| | | | | | | | isoform b preproprotein |
| 213891_s_at | CIS | 3.86 | 3.63 | 3.02 | 2.77 | Hs.359289 | NM_007193; annexin A10 |
| 221872_at | CIS | 3.82 | 3.52 | 2.89 | 2.73 | Hs.82547 | NM_001958; eukaryotic |
| | | | | | | | translation elongation |
| | | | | | | | factor 1 alpha 2 |
| 212386_at | CIS | 3.77 | 3.50 | 2.87 | 2.69 | Hs.359289 | NM_005547; involucrin |
| 211161_s_at | CIS | 3.76 | 3.42 | 2.84 | 2.65 | | NM_000300; phospholipase |
| | | | | | | | A2, group IIA (platelets, |
| | | | | | | | synovial fluid) |
| 214669_x_at | CIS | 3.55 | 3.36 | 2.80 | 2.62 | Hs.377975 | NM_001442; fatty acid |
| | | | | | | | binding protein 4, adipocyte |
| 217388_s_at | CIS | 3.44 | 3.31 | 2.79 | 2.58 | Hs.444471 | NM_000228; laminin |
| | | | | | | | subunit beta 3 precursor |
| 203477_at | CIS | 3.36 | 3.28 | 2.75 | 2.56 | Hs.409034 | NM_030570; uroplakin 3B |
| | | | | | | | isoform a NM_182683; |
| | | | | | | | uroplakin 3B isoform c |
| | | | | | | | NM_182684; uroplakin 3B |
| | | | | | | | isoform b |
| 204688_at | CIS | 3.35 | 3.26 | 2.74 | 2.52 | Hs.409798 | NM_016233; peptidylarginine |
| | | | | | | | deiminase type |
| | | | | | | | III |
| 218718_at | CIS | 3.35 | 3.22 | 2.70 | 2.48 | Hs.43080 | NM_000445; plectin 1, |
| | | | | | | | intermediate filament |
| | | | | | | | binding protein 500 kDa |
| 215176_x_at | CIS | 3.32 | 3.14 | 2.67 | 2.45 | Hs.503443 | NM_001248; ectonucleoside |
| | | | | | | | triphosphate diphosphohydrolase 3 |
| 201842_s_at | CIS | 3.31 | 3.11 | 2.65 | 2.44 | Hs.76224 | NM_020142; |
| | | | | | | | NADH: ubiquinone oxidoreductase |
| | | | | | | | MLRQ |
| | | | | | | | subunit homolog |
| 212667_at | CIS | 3.3 | 3.11 | 2.63 | 2.42 | Hs.111779 | NM_000299; plakophilin 1 |
| 209340_at | CIS | 3.27 | 3.10 | 2.61 | 2.39 | Hs.21293 | NM_007144; ring finger |
| | | | | | | | protein 110 |
| 215379_x_at | CIS | 3.26 | 3.10 | 2.59 | 2.39 | Hs.449601 | NM_006760; uroplakin 2 |
| 200762_at | CIS | 3.25 | 3.05 | 2.56 | 2.34 | Hs.173381 | NM_019894; transmembrane |
| | | | | | | | protease, serine 4 |
| | | | | | | | isoform 1 NM_183247; |
| | | | | | | | transmembrane protease, |
| | | | | | | | serine 4 isoform 2 |
| 211896_s_at | CIS | 3.21 | 3.05 | 2.53 | 2.32 | Hs.156316 | NM_005581; Lutheran |
| | | | | | | | blood group (Auberger b |
| | | | | | | | antigen included) |
| 204141_at | CIS | 3.19 | 3.05 | 2.53 | 2.28 | Hs.300701 | NM_017689; hypothetical |
| | | | | | | | protein FLJ20151 |
| 201744_s_at | CIS | 3.18 | 3.03 | 2.50 | 2.27 | Hs.406475 | NM_014417; BCL2 binding |
| | | | | | | | component 3 |
| 209138_x_at | CIS | 3.17 | 3.03 | 2.47 | 2.24 | Hs.505407 | NM_015162; lipidosin |
| 214677_x_at | CIS | 3.14 | 3.02 | 2.47 | 2.23 | Hs.449601 | NM_006942; SRY-box 15 |
| 212077_at | CIS | 3.11 | 2.99 | 2.46 | 2.21 | Hs.443811 | NM_003282; troponin I, |
| | | | | | | | skeletal, fast |
| 206392_s_at | CIS | 3.11 | 2.98 | 2.43 | 2.20 | Hs.82547 | NM_005522; homeobox |
| | | | | | | | A1 protein isoform a |
| | | | | | | | NM_153620; homeobox |
| | | | | | | | A1 protein isoform b |
| 212998_x_at | CIS | 3.09 | 2.94 | 2.40 | 2.19 | Hs.375115 | NM_000497; cytochrome |
| | | | | | | | P450, subfamily XIB |
| | | | | | | | (steroid 11-beta- |
| | | | | | | | hydroxylase), polypeptide |
| | | | | | | | 1 precursor |
| 201616_s_at | CIS | 3.08 | 2.93 | 2.38 | 2.18 | Hs.443811 | NM_004692; |
| | | | | | | | NM_032727; internexin |
| | | | | | | | neuronal intermediate |
| | | | | | | | filament protein, alpha |
| 205382_s_at | CIS | 3.07 | 2.88 | 2.37 | 2.15 | Hs.155597 | NM_000213; integrin, |
| | | | | | | | beta 4 |
| 212671_s_at | CIS | 3.07 | 2.85 | 2.35 | 2.14 | Hs.387679 | NM_002145; homeo box |
| | | | | | | | B2 |
| 215121_x_at | CIS | 3.06 | 2.84 | 2.34 | 2.13 | Hs.356861 | NM_018058; cartilage |
| | | | | | | | acidic protein 1 |
| 200600_at | CIS | 3.05 | 2.83 | 2.33 | 2.11 | Hs.170328 | NM_001910; cathepsin E |
| | | | | | | | isoform a preproprotein |
| | | | | | | | NM_148964; cathepsin E |
| | | | | | | | isoform b preproprotein |
| 202746_at | CIS | 3.03 | 2.80 | 2.32 | 2.10 | Hs.17109 | NM_007193; annexin A10 |
| 202917_s_at | CIS | 3 | 2.79 | 2.31 | 2.08 | Hs.416073 | NM_001958; eukaryotic |
| | | | | | | | translation elongation |
| | | | | | | | factor 1 alpha 2 |
| 201560_at | CIS | 3 | 2.79 | 2.30 | 2.08 | Hs.25035 | NM_005547; involucrin |
| 218918_at | CIS | 2.99 | 2.77 | 2.29 | 2.06 | Hs.8910 | NM_000300; phospholipase |
| | | | | | | | A2, group IIA (platelets, |
| | | | | | | | synovial fluid) |
| 218656_s_at | CIS | 2.99 | 2.76 | 2.27 | 2.06 | Hs.93765 | NM_001442; fatty acid |
| | | | | | | | binding protein 4, adipocyte |
| 201088_at | CIS | 2.99 | 2.76 | 2.26 | 2.04 | Hs.159557 | NM_000228; laminin |
| | | | | | | | subunit beta 3 precursor |
| 201291_s_at | CIS | 2.97 | 2.75 | 2.25 | 2.04 | Hs.156346 | NM_030570; uroplakin 3B |
| | | | | | | | isoform a NM_182683; |
| | | | | | | | uroplakin 3B isoform c |
| | | | | | | | NM_182684; uroplakin 3B |
| | | | | | | | isoform b |
| 215076_s_at | CIS | 2.95 | 2.72 | 2.24 | 2.03 | Hs.443625 | NM_016233; peptidylarginine |
| | | | | | | | deiminase type |
| | | | | | | | III |
| 212195_at | CIS | 2.94 | 2.71 | 2.22 | 2.02 | Hs.71968 | NM_000445; plectin 1, |
| | | | | | | | intermediate filament |
| | | | | | | | binding protein 500 kDa |
| 209732_at | CIS | 2.94 | 2.68 | 2.22 | 2.00 | Hs.85201 | NM_001248; ectonucleoside |
| | | | | | | | triphosphate diphosphohydrolase 3 |
| 212192_at | CIS | 2.94 | 2.67 | 2.22 | 1.99 | Hs.109438 | NM_020142; |
| | | | | | | | NADH: ubiquinone oxidoreductase |
| | | | | | | | MLRQ |
| | | | | | | | subunit homolog |
| 221671_x_at | CIS | 2.92 | 2.67 | 2.20 | 1.98 | Hs.377975 | NM_000299; plakophilin 1 |
| 211671_s_at | CIS | 2.91 | 2.66 | 2.20 | 1.98 | Hs.126608 | NM_007144; ring finger |
| | | | | | | | protein 110 |
| 214352_s_at | CIS | 2.88 | 2.66 | 2.19 | 1.97 | Hs.412107 | NM_006760; uroplakin 2 |

Feature: Probe-set on U133A GeneChip
Class: The group in which the marker is up-regulated
T-test: The t-test value
Perm 1%: The 1% permutation level
Perm 5%: The 5% permutation level
Perm 10%: The 10% permutation level

### Construction of a Molecular CIS Classifier

A classifier able to diagnose CIS from gene expressions in TCC or in bladder biopsies may increase the detection rate of CIS. Our first approach was to be able to classify superficial TCC with or without CIS in the surrounding mucosa. This could have the diverse effect that the number of random biopsies to be taken could be reduced.

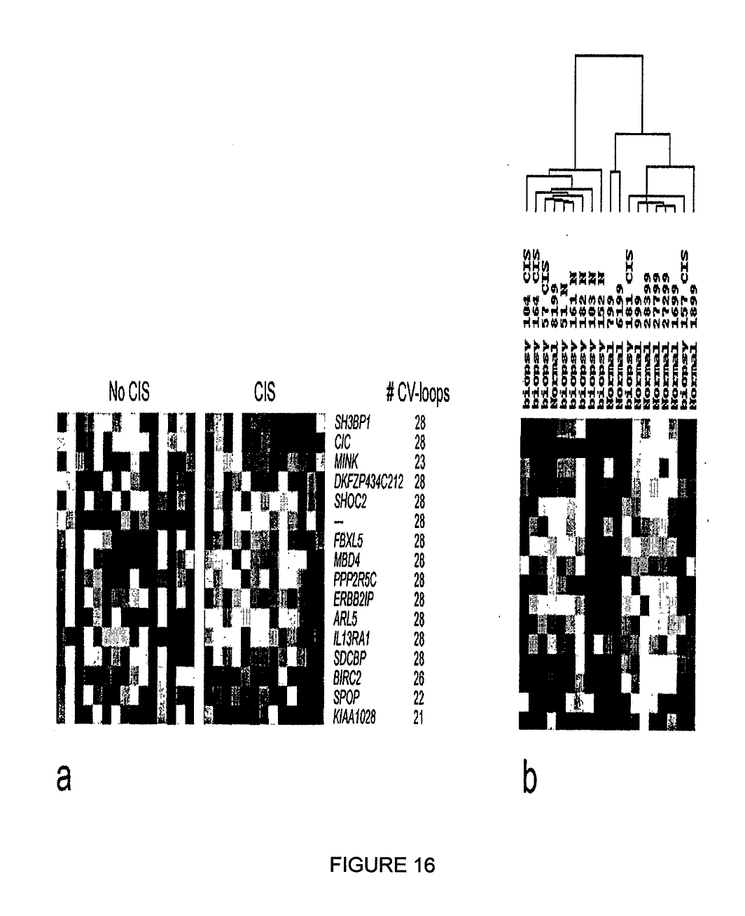

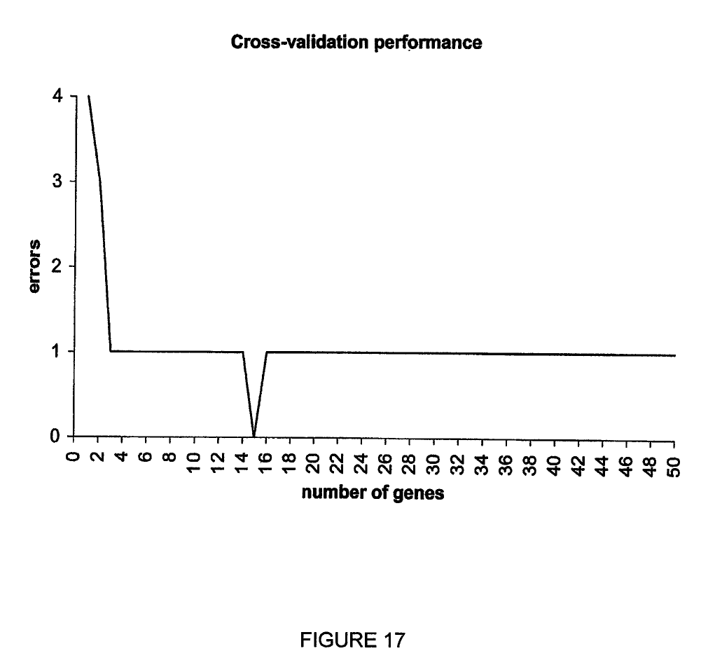

We build a CIS-classifier as previously described (Dyrskjot et al. 2003) using cross-validation for determining the optimal number of genes for classifying CIS with fewest errors. The best classifier performance (1 error) was obtained in cross-validation loops using 25 genes (see FIG. 16); 16 of these were included in 70% of the cross-validation loops and these were selected to represent our final classifier for CIS diagnosis (FIG. 17a and table 21). Permutation analysis shoved that 13 of these were significant at a 1% confidence level—the remaining three genes were above a 10% confidence level.

TABLE 21
The 16 gene molecular classifier of CIS

| Feature | | | Perm | Perm | Perm | UniGene Build | |
|---|---|---|---|---|---|---|---|
| (U133a array) | Class | t-test | 1% | 5% | 10% | 162 | RefSeq; description |
| 213633_at | no_CIS | 1.51 | 2.46 | 2.04 | 1.85 | Hs.97858 | NM_018957; SH3- |
| | | | | | | | domain binding protein 1 |
| 212784_at | no_CIS | 1.36 | 2.27 | 1.86 | 1.70 | Hs.388236 | NM_015125; capicua |
| | | | | | | | homolog |
| 209241_x_at | no_CIS | 1.13 | 1.78 | 1.48 | 1.33 | Hs.112028 | NM_015716; mis- |
| | | | | | | | shapen/NIK-related |
| | | | | | | | kinase isoform 1 |
| | | | | | | | NM_153827; mis- |
| | | | | | | | shapen/NIK-related |
| | | | | | | | kinase isoform 3 |
| | | | | | | | NM_170663; mis- |
| | | | | | | | shapen/NIK-related |
| | | | | | | | kinase isoform 2 |
| 217941_s_at | CIS | 2.3 | 1.96 | 1.66 | 1.47 | Hs.8117 | NM_018695; erbb2 |
| | | | | | | | interacting protein |
| 201877_s_at | CIS | 2.27 | 1.90 | 1.62 | 1.45 | Hs.249955 | NM_002719; gamma |
| | | | | | | | isoform of regulatory |
| | | | | | | | subunit B56, protein |
| | | | | | | | phosphatase 2A isoform |
| | | | | | | | a NM_178586; gamma |
| | | | | | | | isoform of regulatory |
| | | | | | | | subunit B56, protein |
| | | | | | | | phosphatase 2A isoform |
| | | | | | | | b NM_178587; gamma |
| | | | | | | | isoform of regulatory |
| | | | | | | | subunit B56, protein |
| | | | | | | | phosphatase 2A isoform |
| | | | | | | | c NM_178588; gamma |
| | | | | | | | isoform of regulatory |
| | | | | | | | subunit B56, protein |
| | | | | | | | phosphatase 2A isoform d |
| 209630_s_at | CIS | 1.97 | 1.54 | 1.31 | 1.15 | Hs.444354 | NM_012164; F-box and |
| | | | | | | | WD-40 domain protein 2 |
| 202777_at | CIS | 1.93 | 1.51 | 1.29 | 1.12 | Hs.104315 | NM_007373; soc-2 |
| | | | | | | | suppressor of clear |
| | | | | | | | homolog |
| 200958_s_at | CIS | 1.92 | 1.49 | 1.28 | 1.11 | Hs.164067 | NM_005625; syndecan |
| | | | | | | | binding protein |
| | | | | | | | (syntenin) |
| 209579_s_at | CIS | 1.79 | 1.36 | 1.16 | 1.01 | Hs.35947 | NM_003925; methyl- |
| | | | | | | | CpG binding domain |
| | | | | | | | protein 4 |
| 209004_s_at | CIS | 1.63 | 1.21 | 1.00 | 0.89 | Hs.5548 | NM_012161; F-box and |
| | | | | | | | leucine-rich repeat |
| | | | | | | | protein 5 isoform 1 |
| | | | | | | | NM_033535; F-box and |
| | | | | | | | leucine-rich repeat |
| | | | | | | | protein 5 isoform 2 |
| 218150_at | CIS | 1.6 | 1.18 | 0.98 | 0.86 | Hs.342849 | NM_012097; ADP- |
| | | | | | | | ribosylation factor-like 5 |
| | | | | | | | isoform 1 NM_177985; |
| | | | | | | | ADP-ribosylation factor- |
| | | | | | | | like 5 isoform 2 |
| 202076_at | CIS | 1.53 | 1.12 | 0.92 | 0.82 | Hs.289107 | NM_001166; baculoviral |
| | | | | | | | IAP repeat-containing |
| | | | | | | | protein 2 |
| 204640_s_at | CIS | 1.45 | 1.03 | 0.83 | 0.75 | Hs.129951 | NM_003563; speckle- |
| | | | | | | | type POZ protein |
| 201887_at | CIS | 1.32 | 0.92 | 0.74 | 0.66 | Hs.285115 | NM_001560; interleukin |
| | | | | | | | 13 receptor, alpha 1 |
| | | | | | | | precursor |
| 212802_s_at | CIS | 1.31 | 0.91 | 0.72 | 0.65 | Hs.287266 | |
| 212899_at | CIS | 1.29 | 0.89 | 0.71 | 0.64 | Hs.129836 | NM_015076; cyclin- |
| | | | | | | | dependent kinase |
| | | | | | | | (CDC2-like) 11 |

Feature: Probe-set on U133A GeneChip
Class: The group in which the marker is up-regulated
T-test: The t-test value
Perm 1%: The 1% permutation level
Perm 5%: The 5% permutation level
Perm 10%: The 10% permutation level

### Exploration of Strength of CIS Classifier

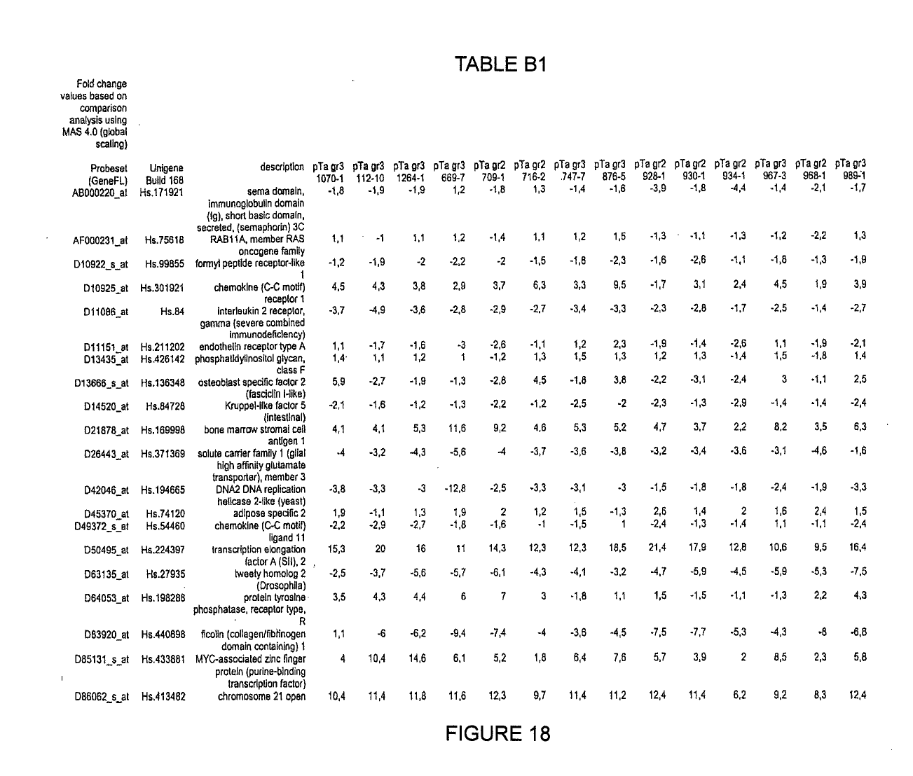

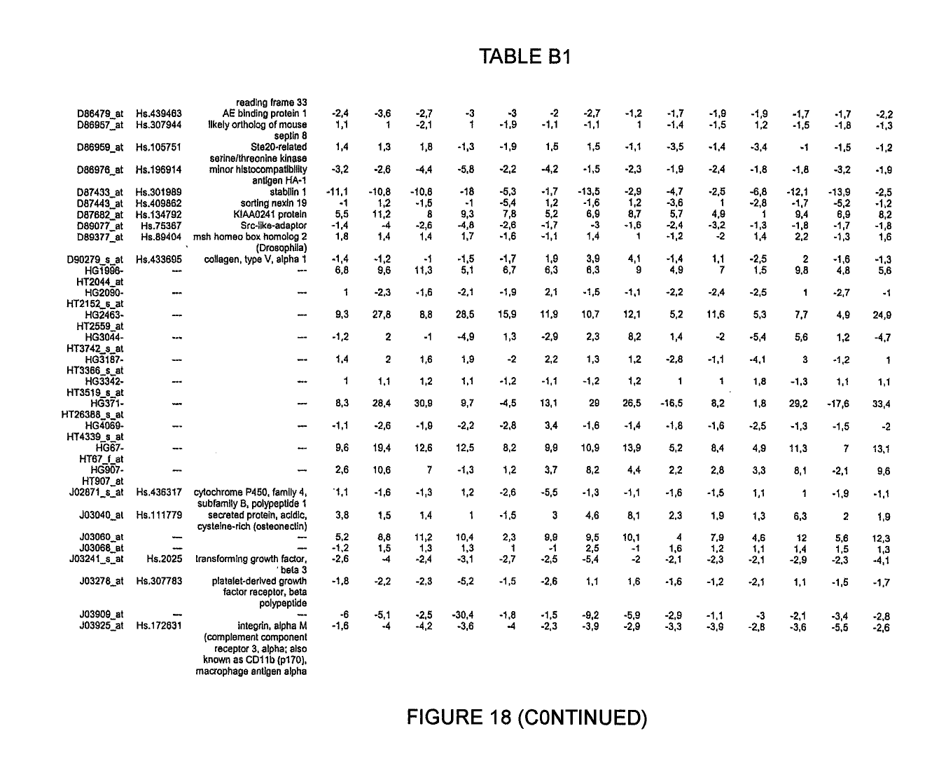

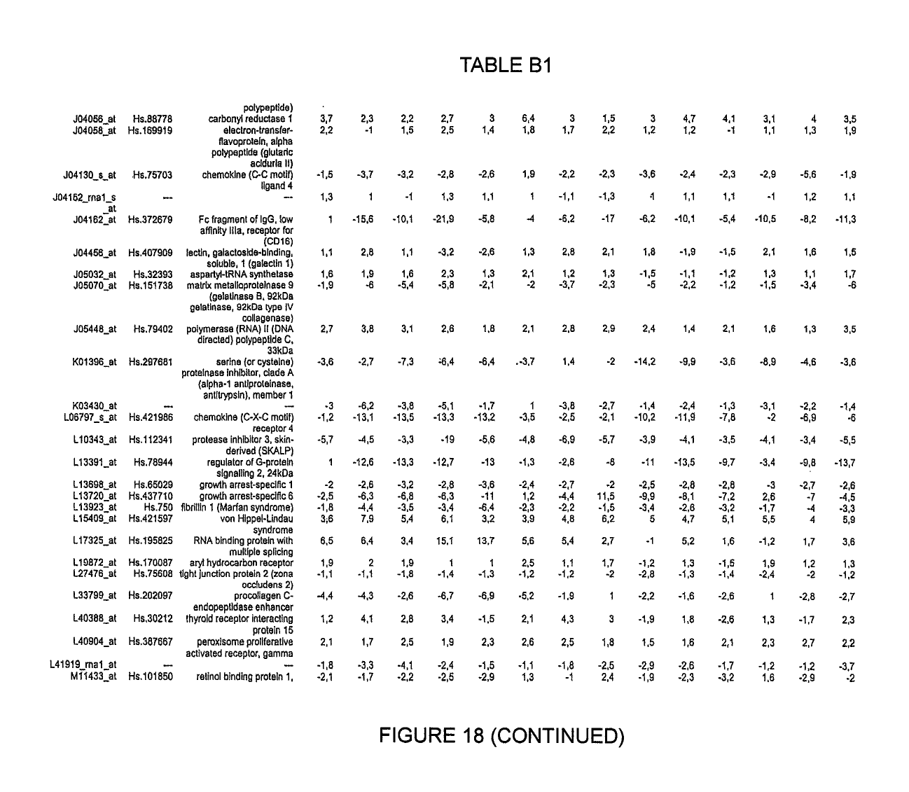

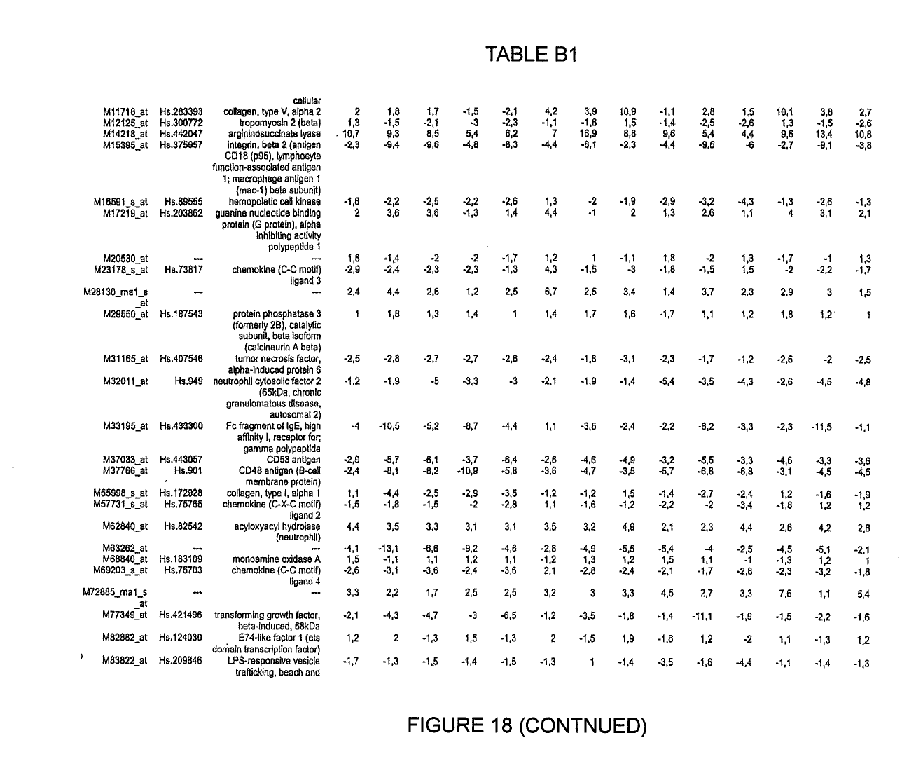

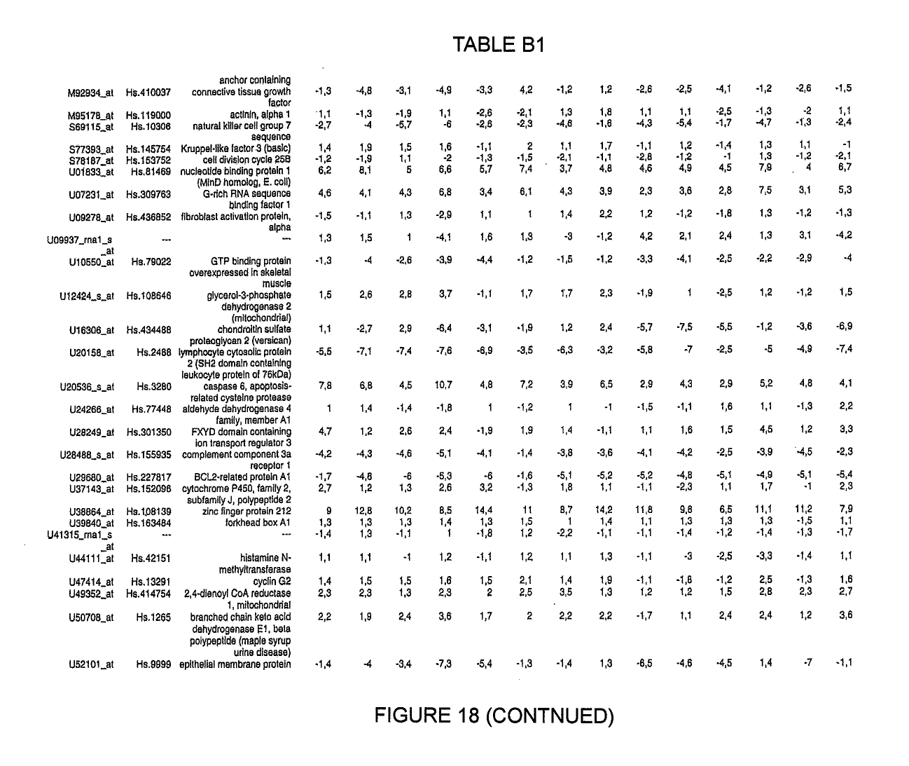

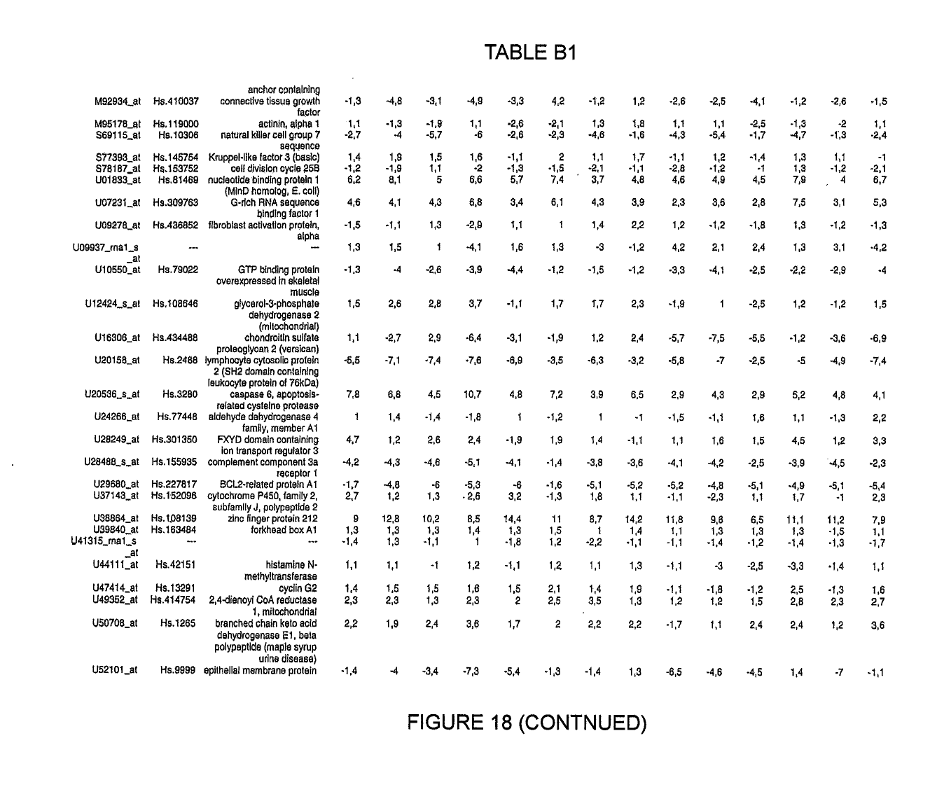

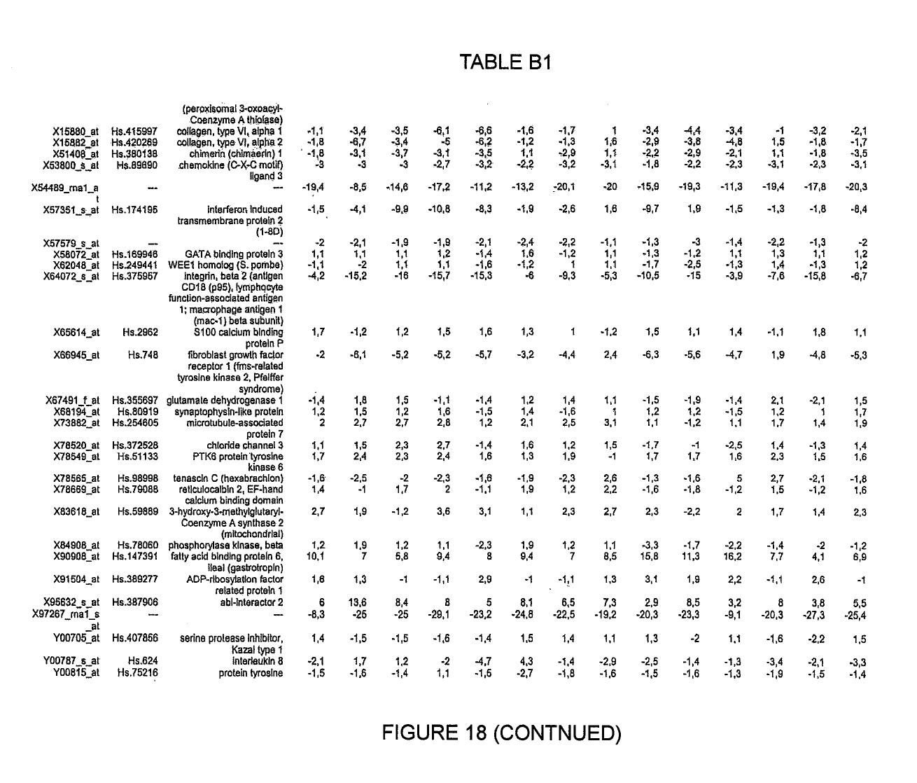

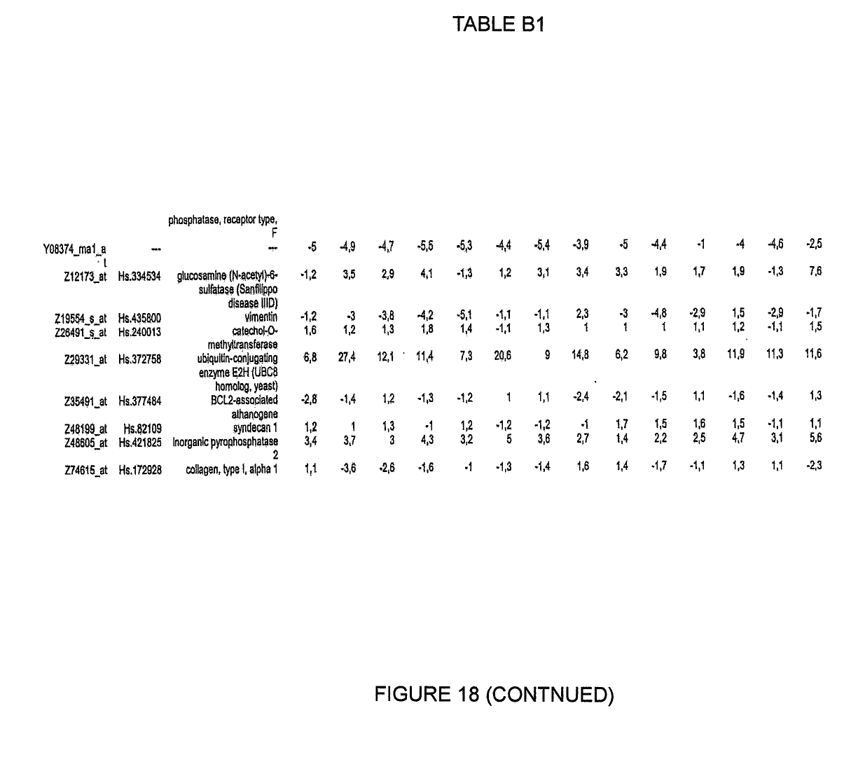

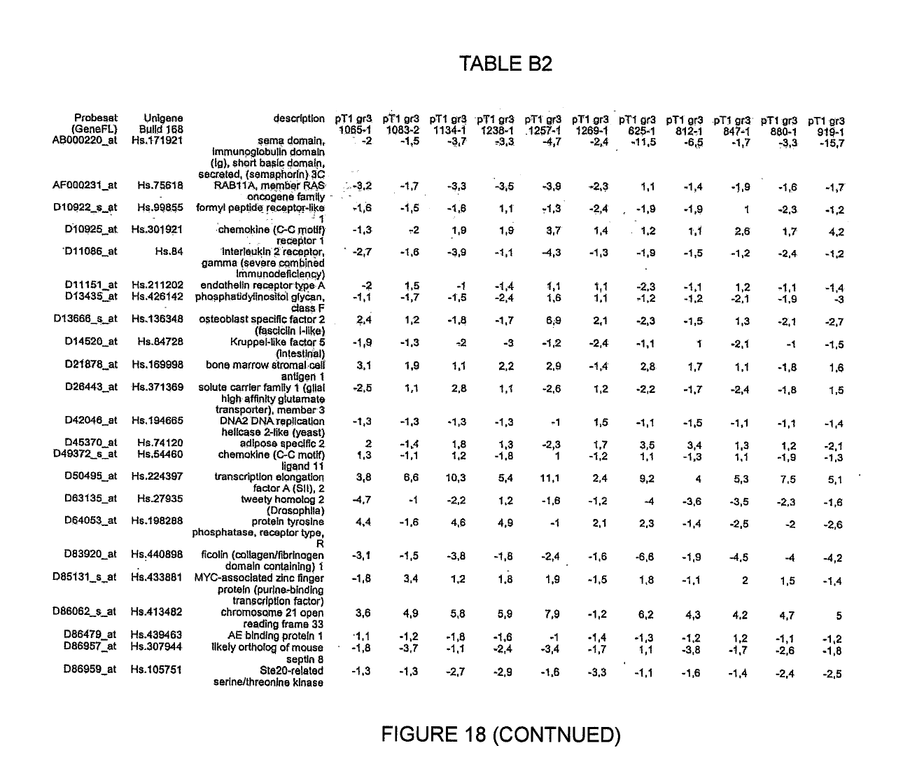

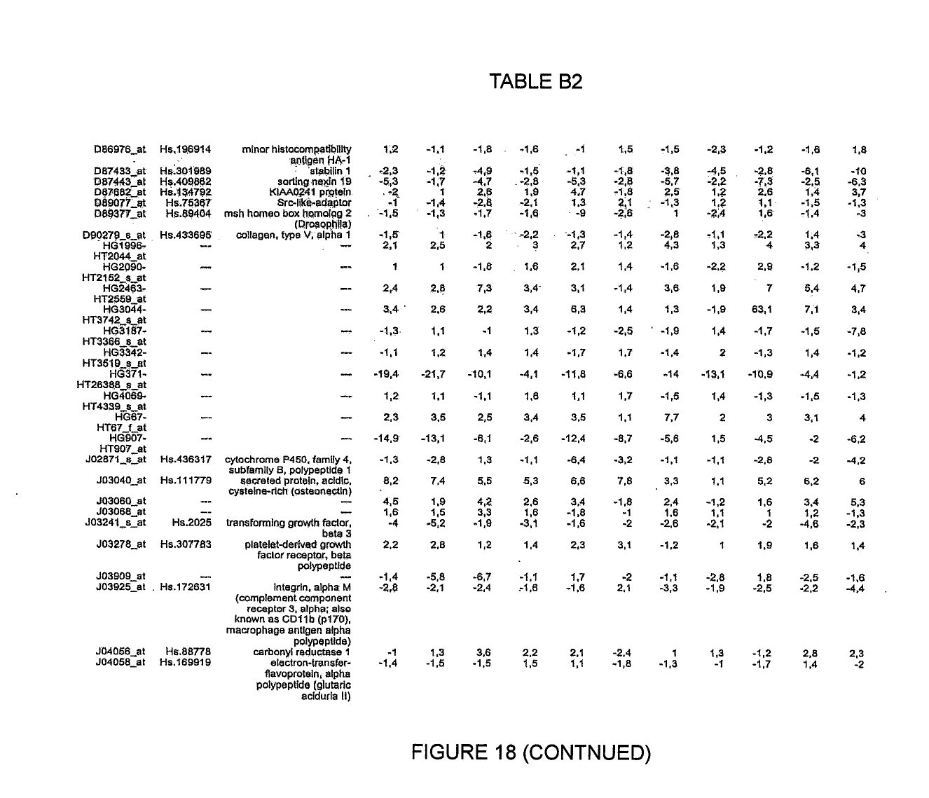

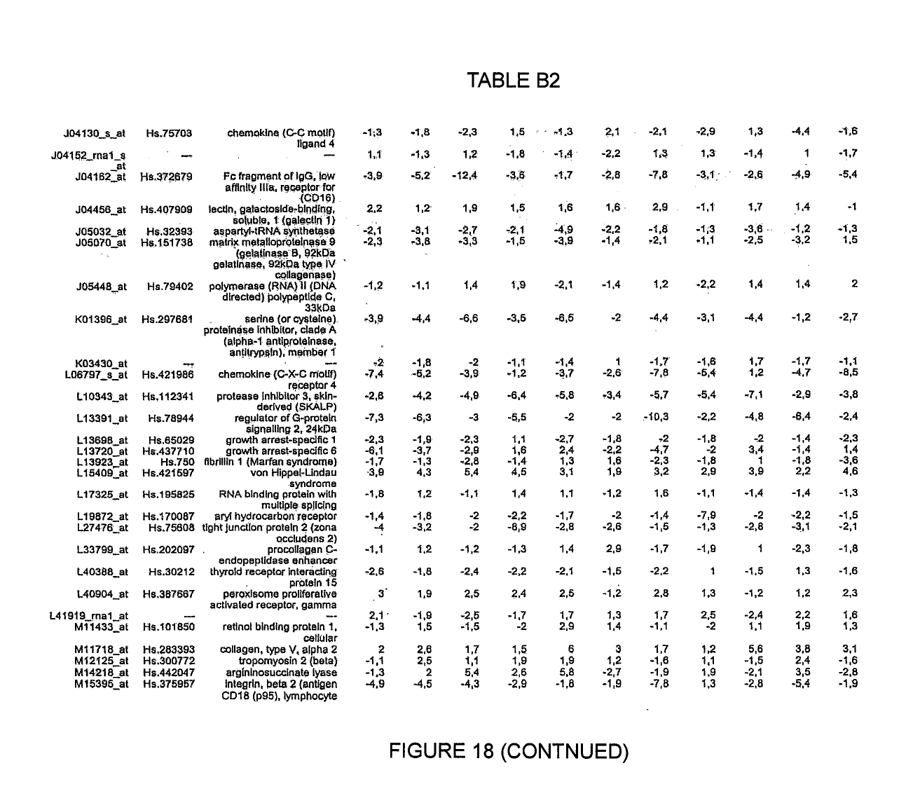

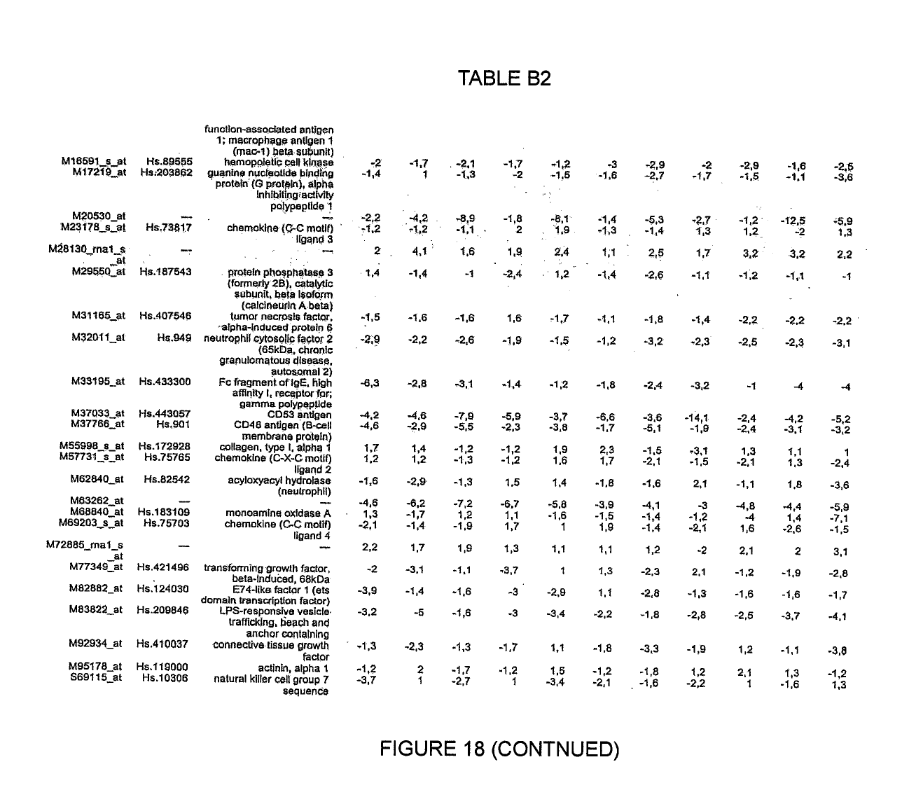

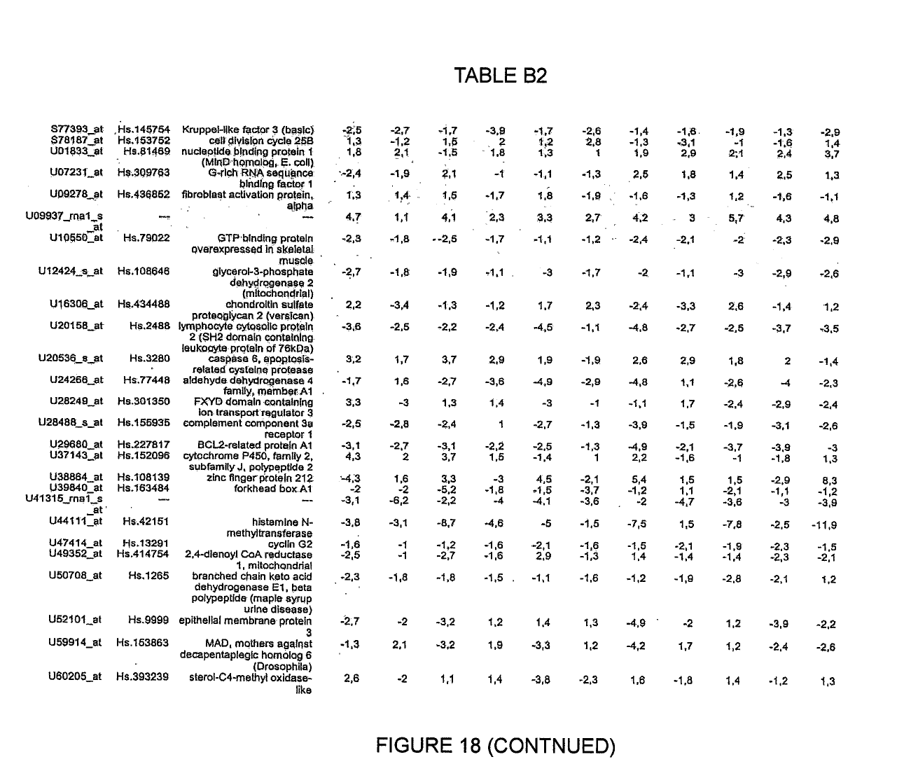

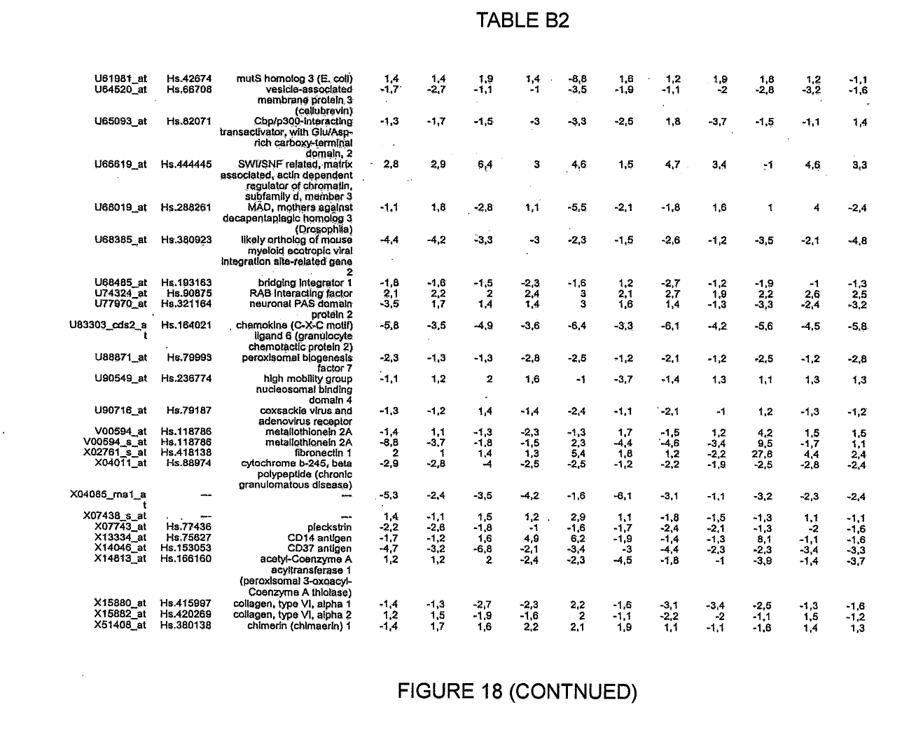

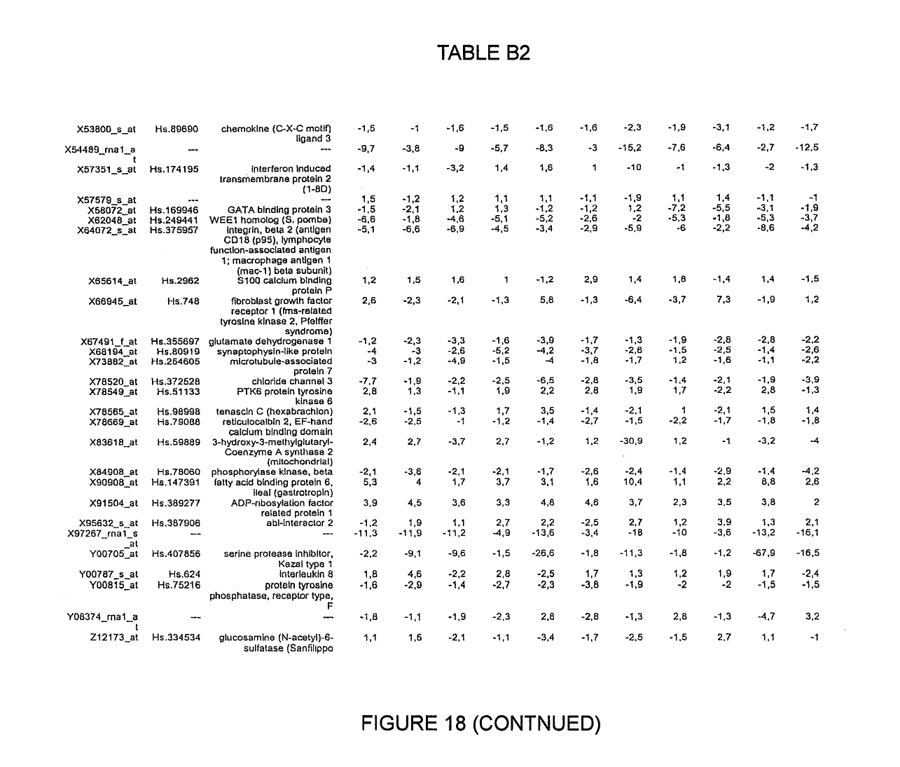

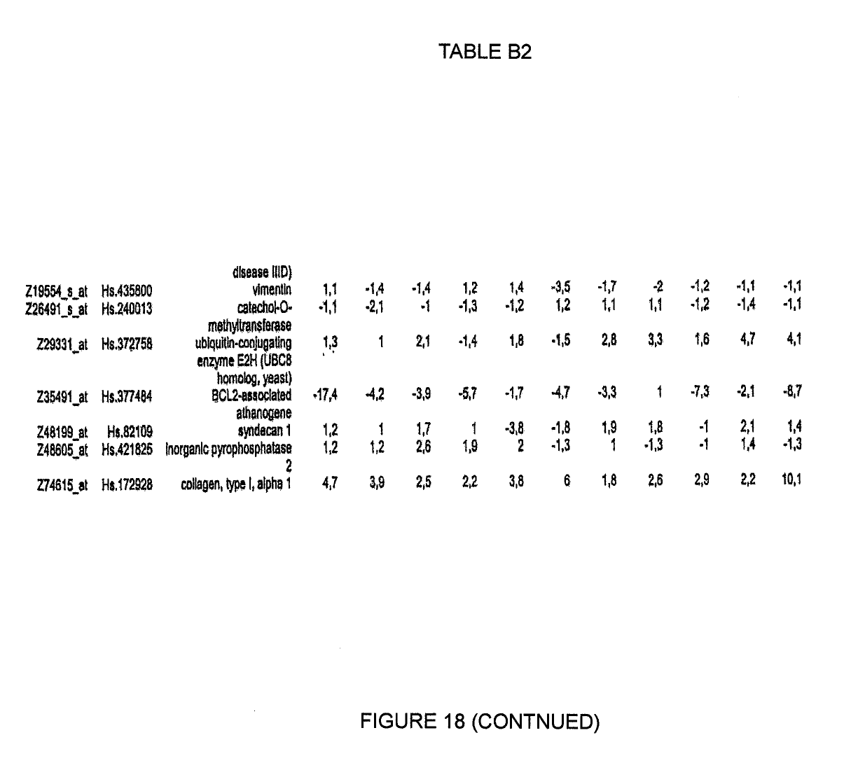

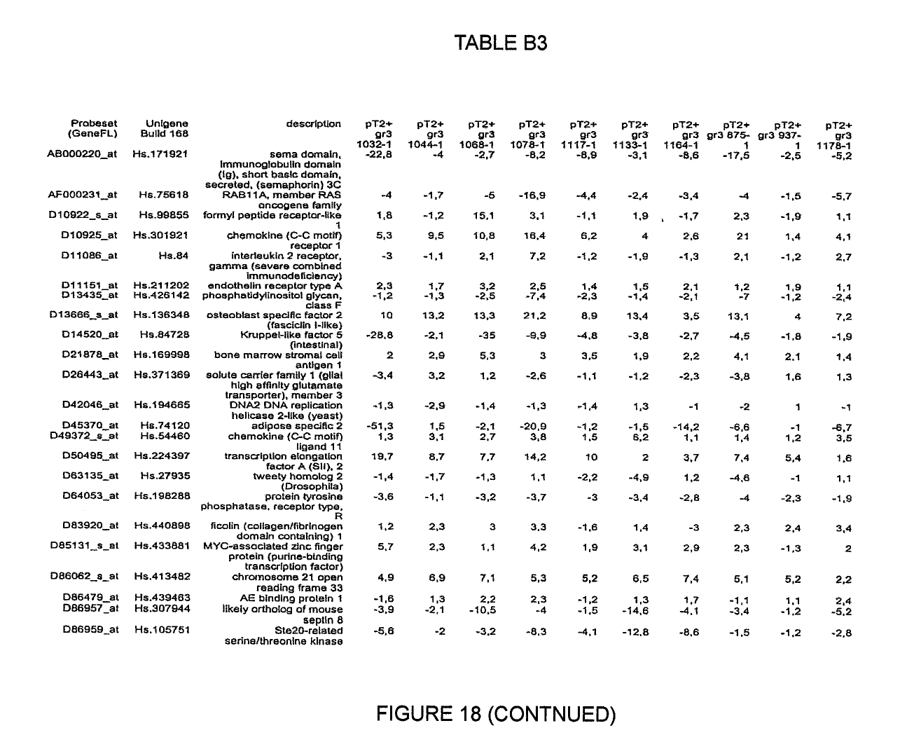

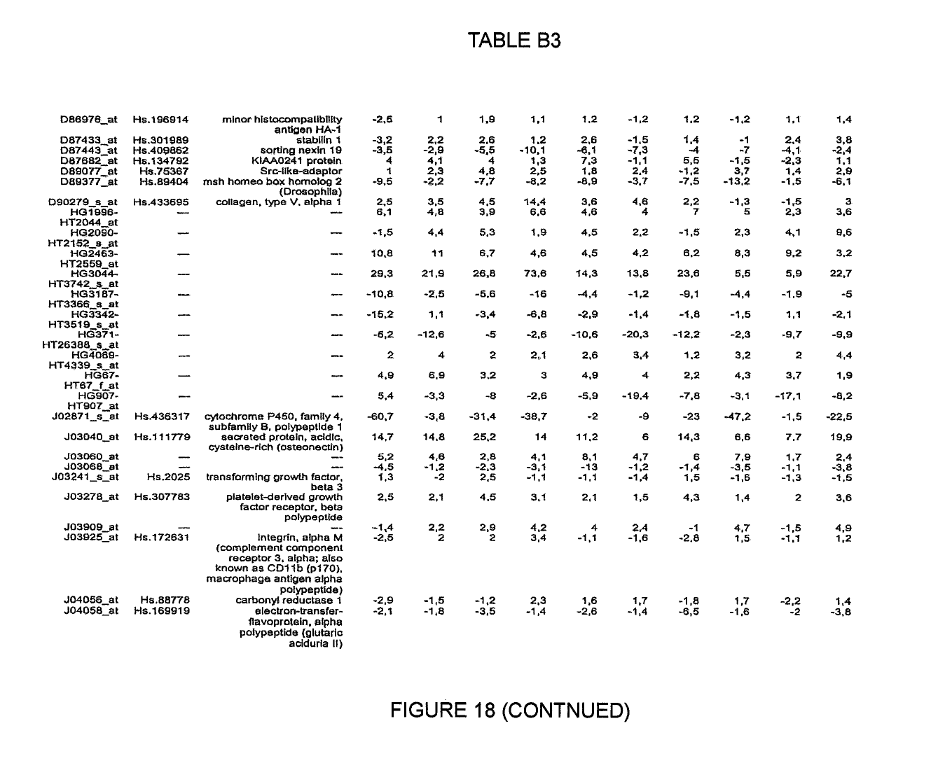

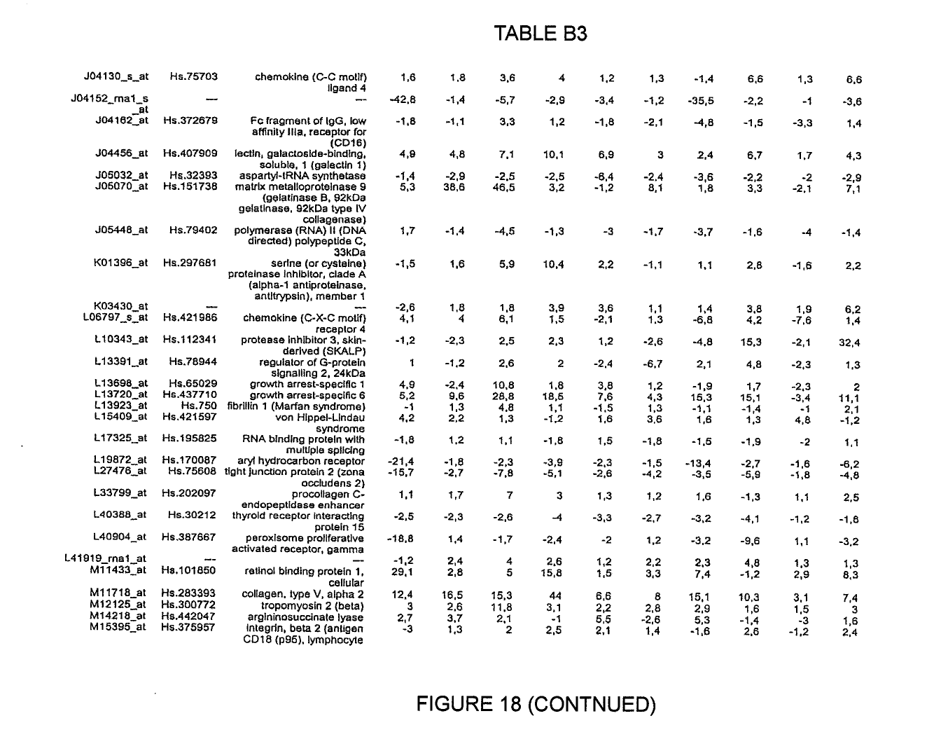

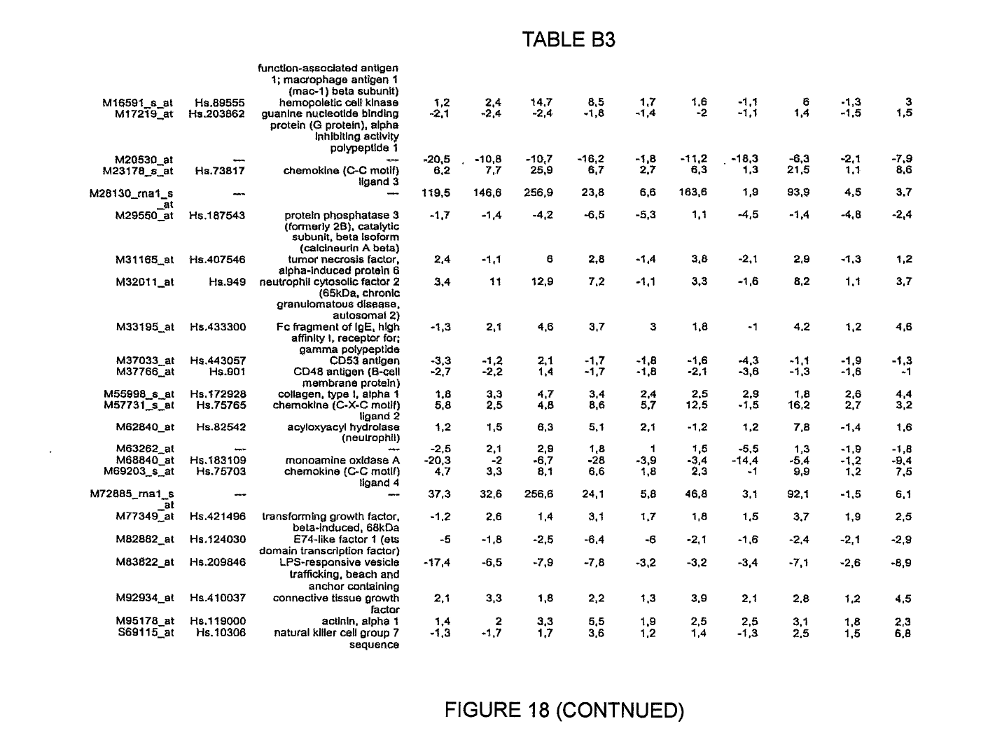

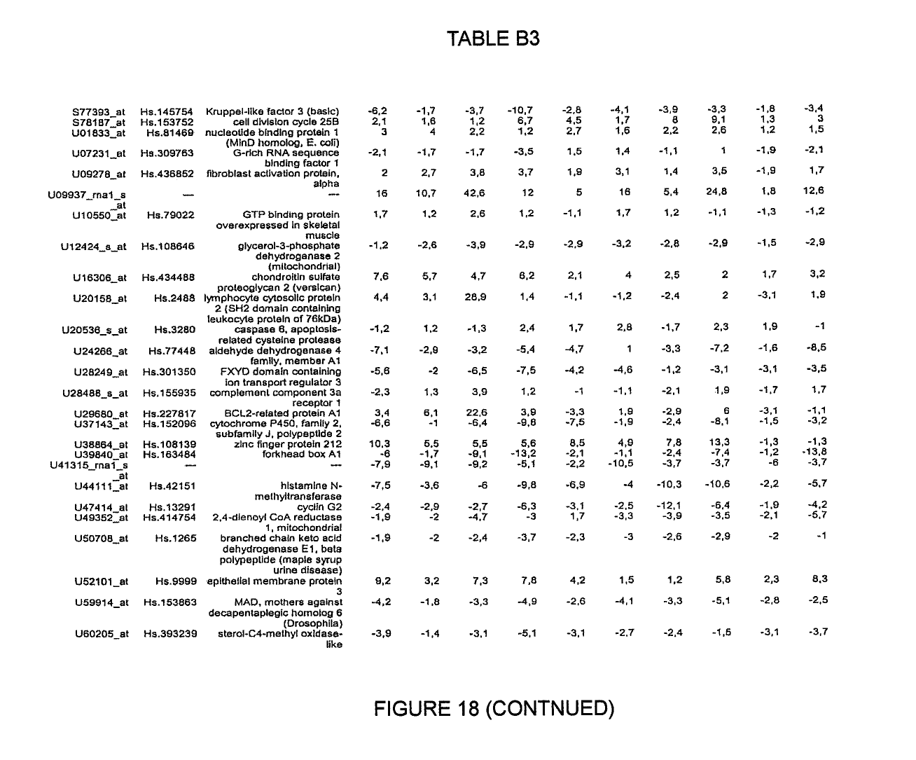

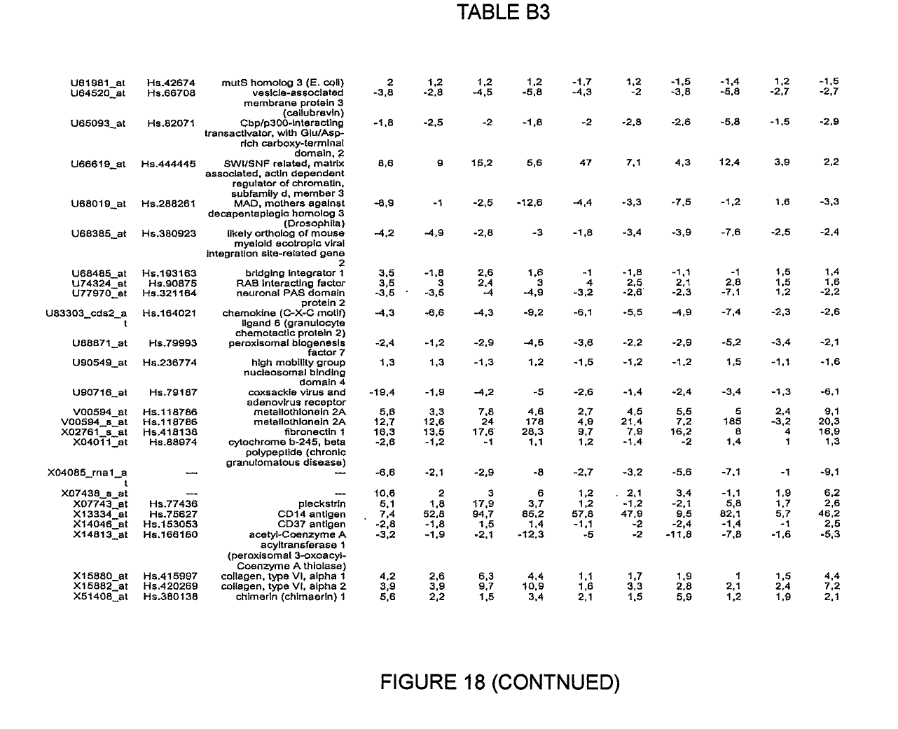

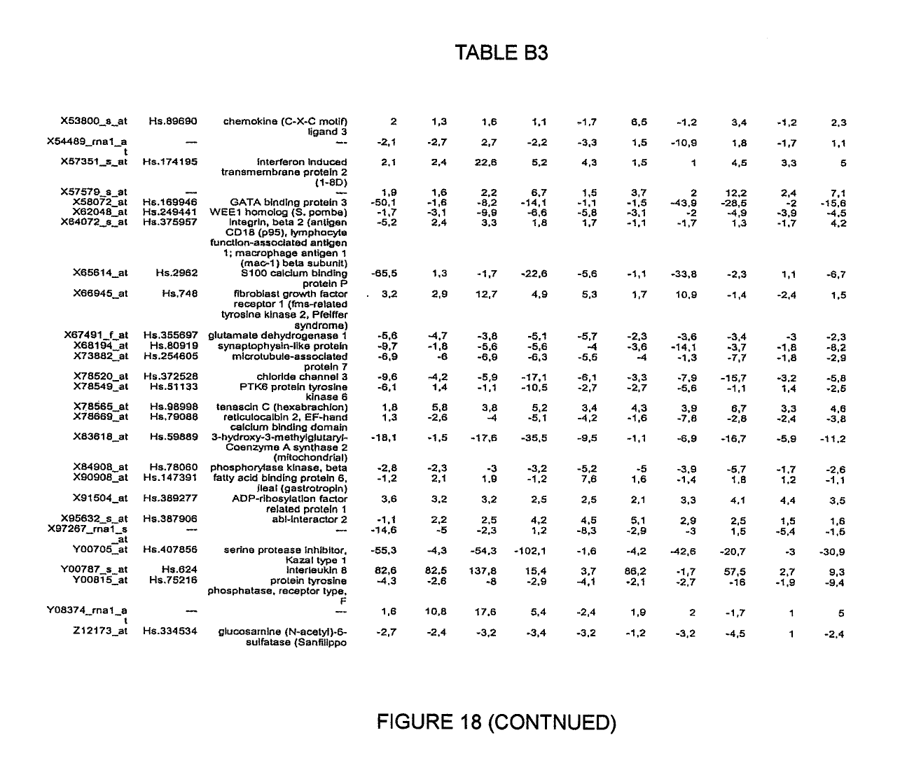

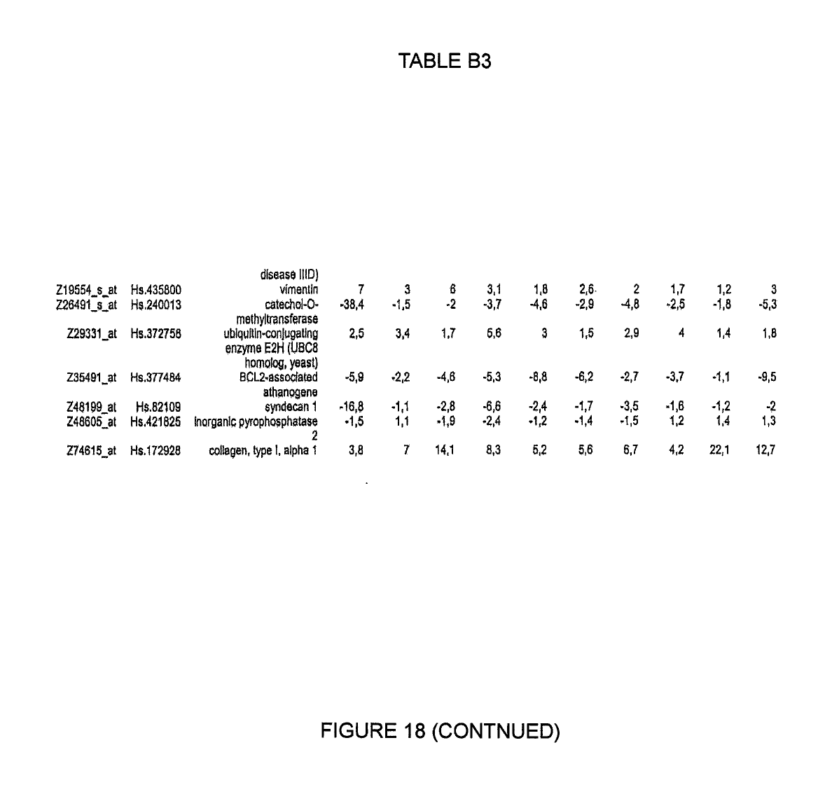

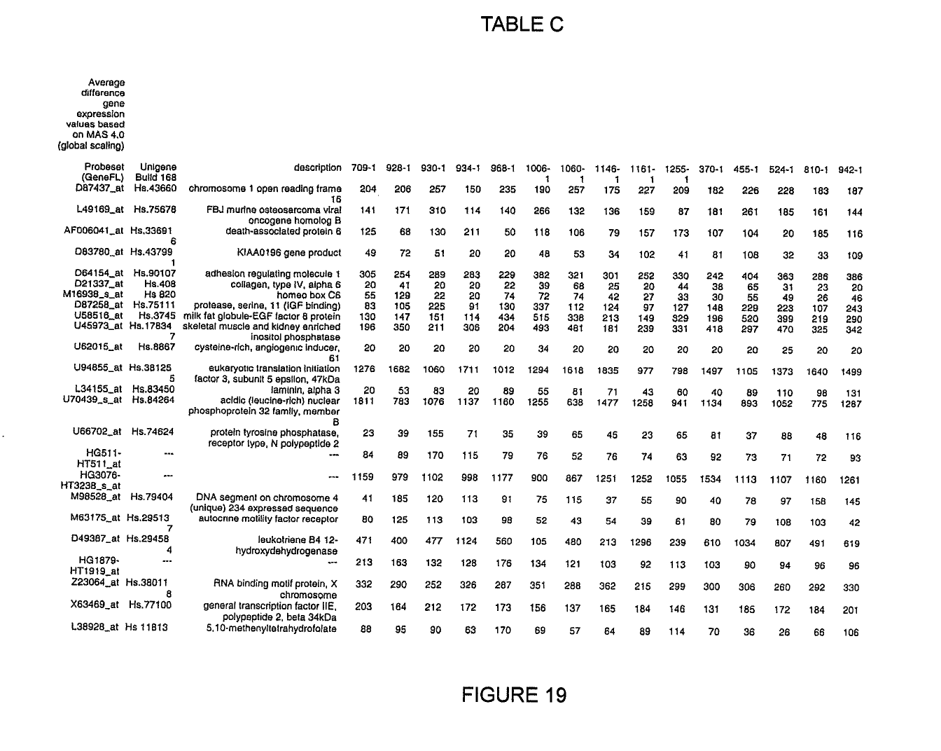

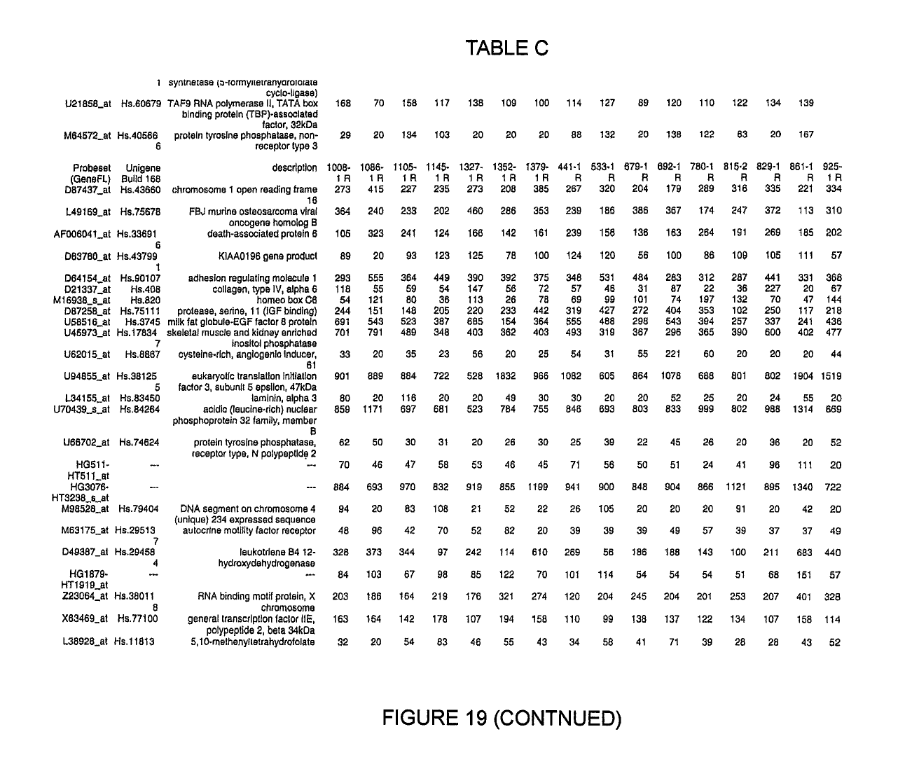

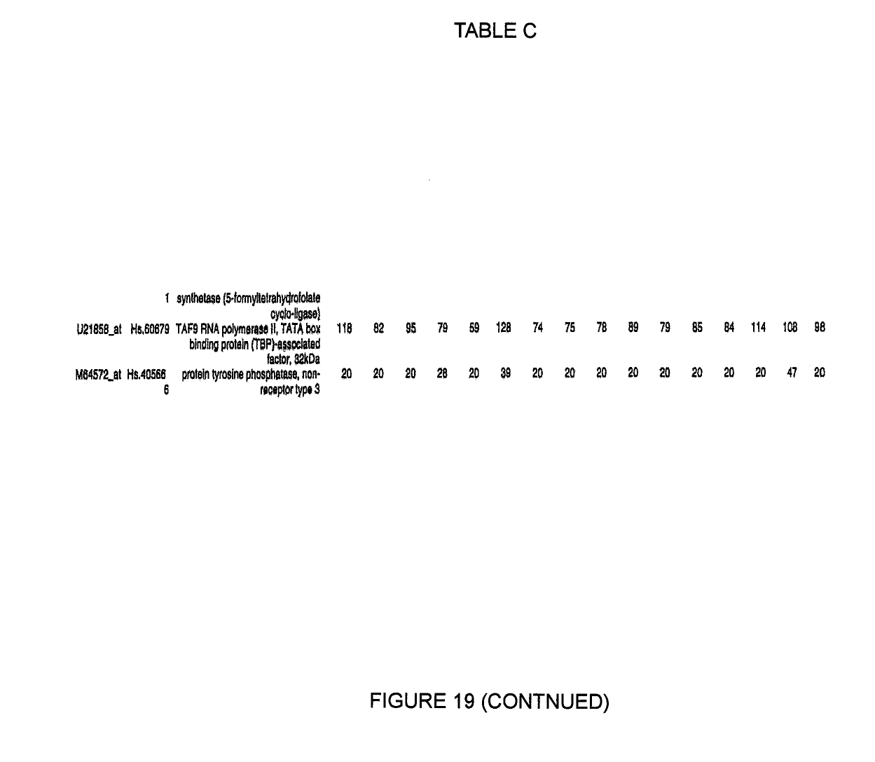

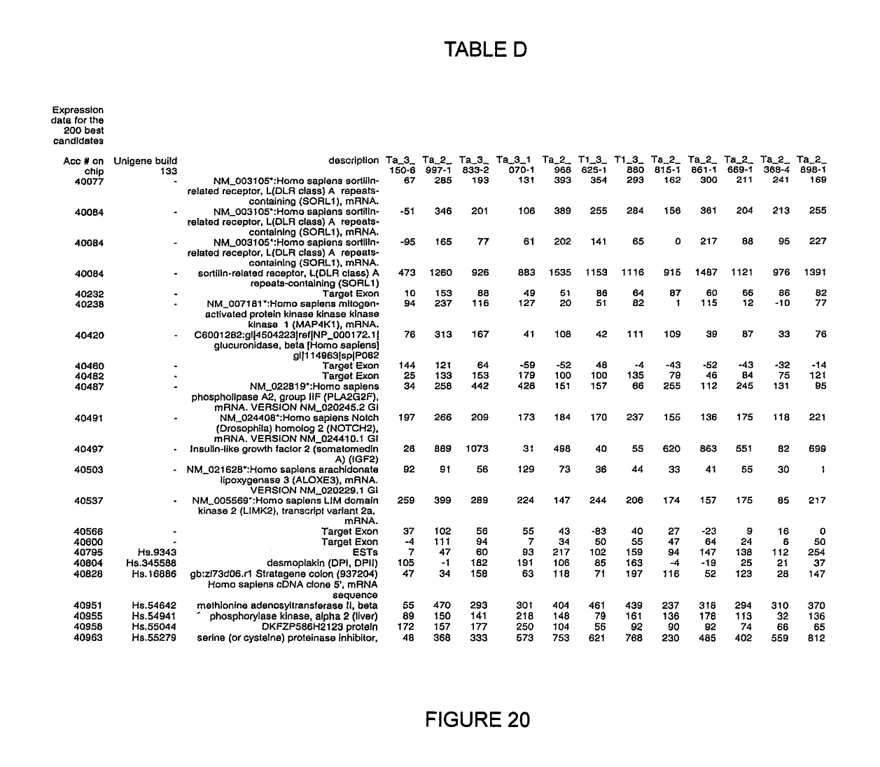

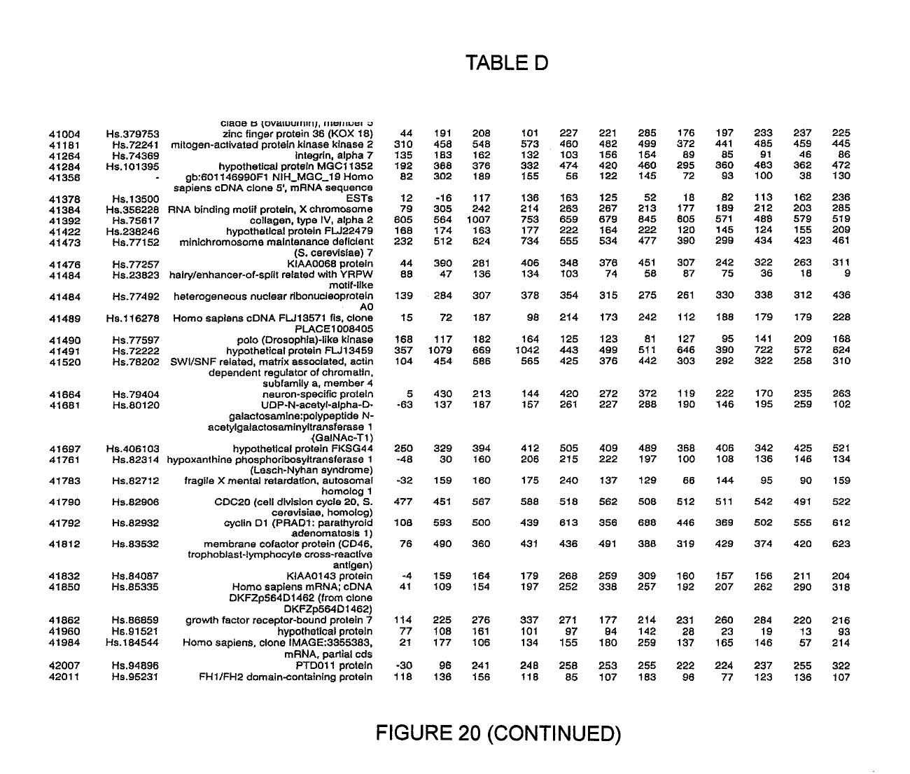

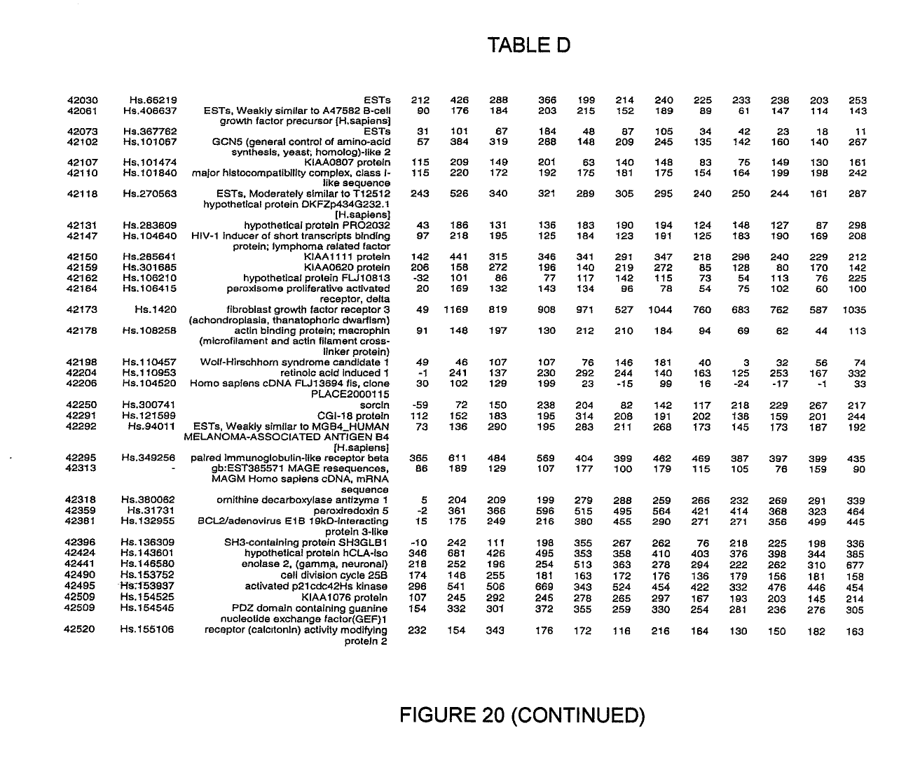

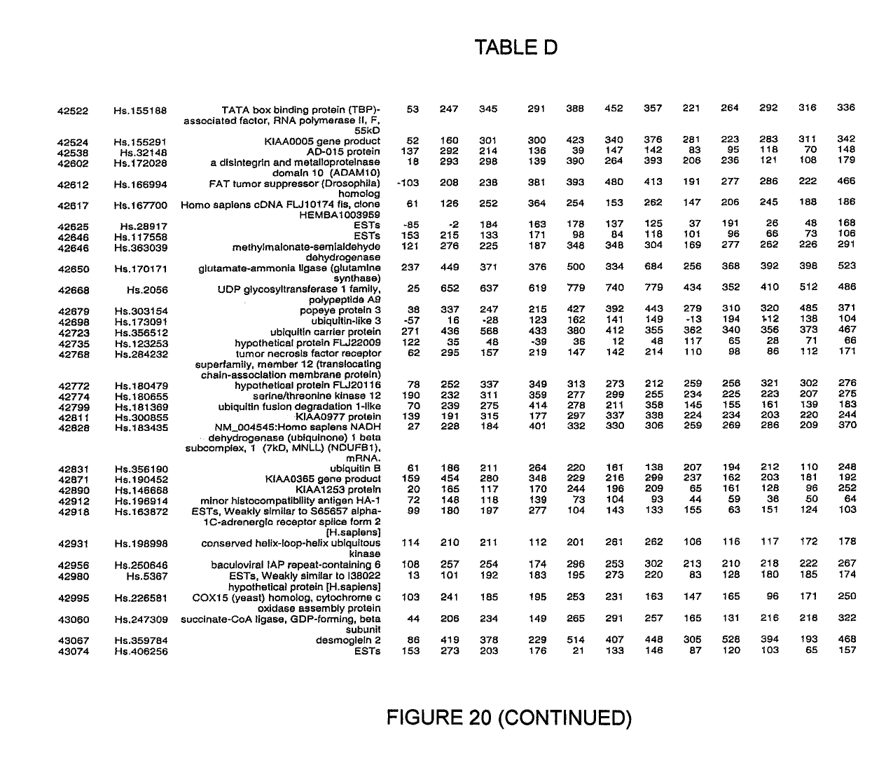

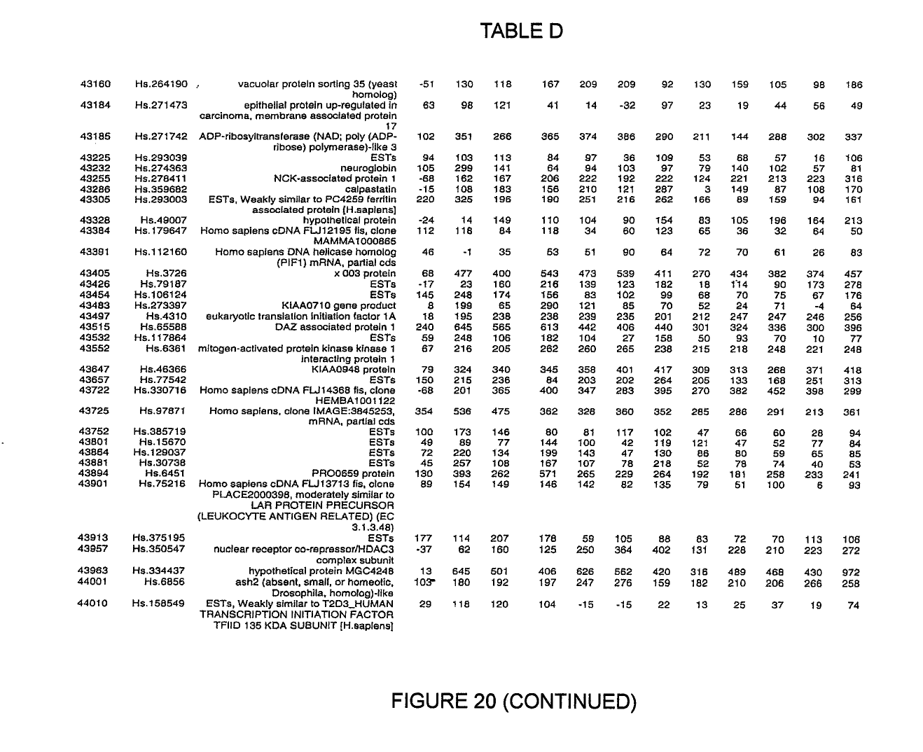

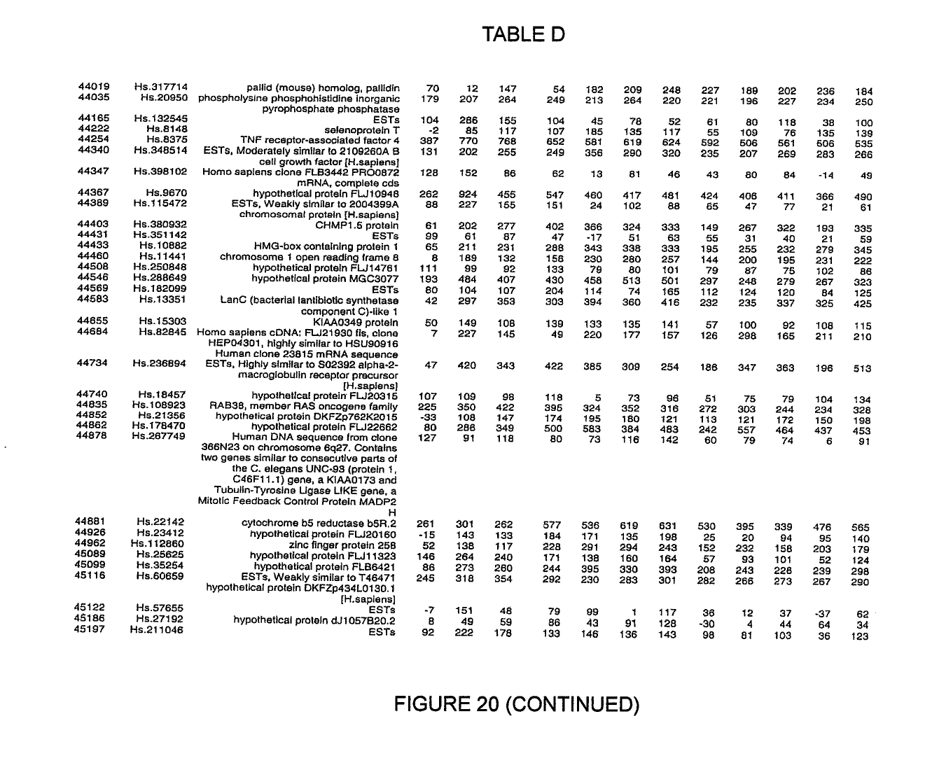

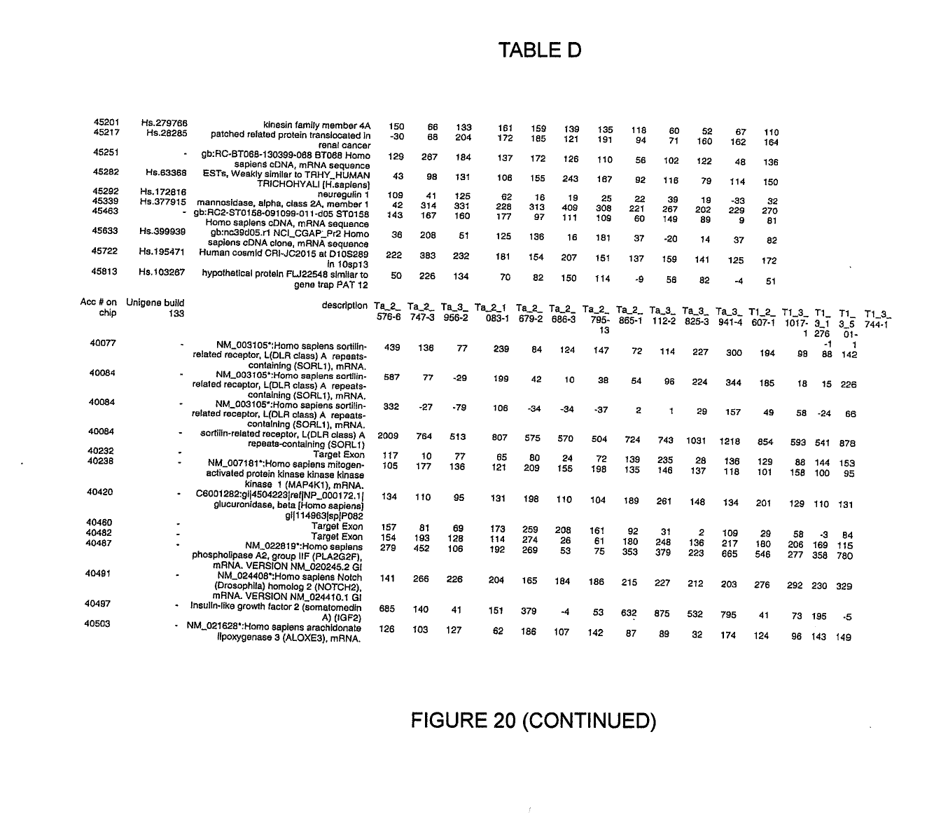

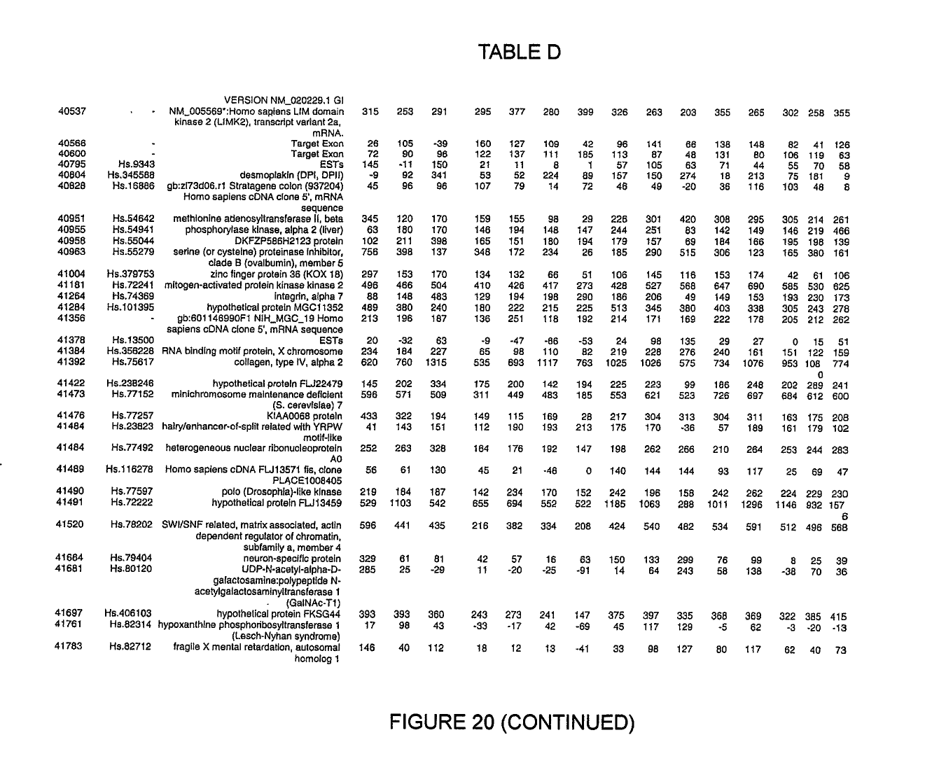

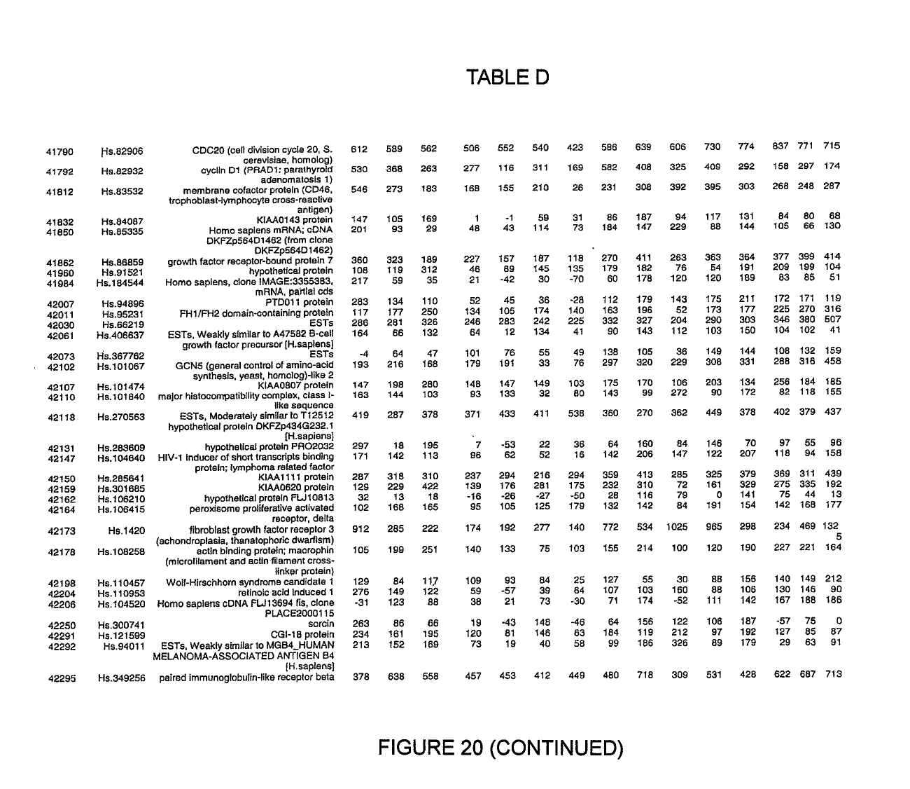

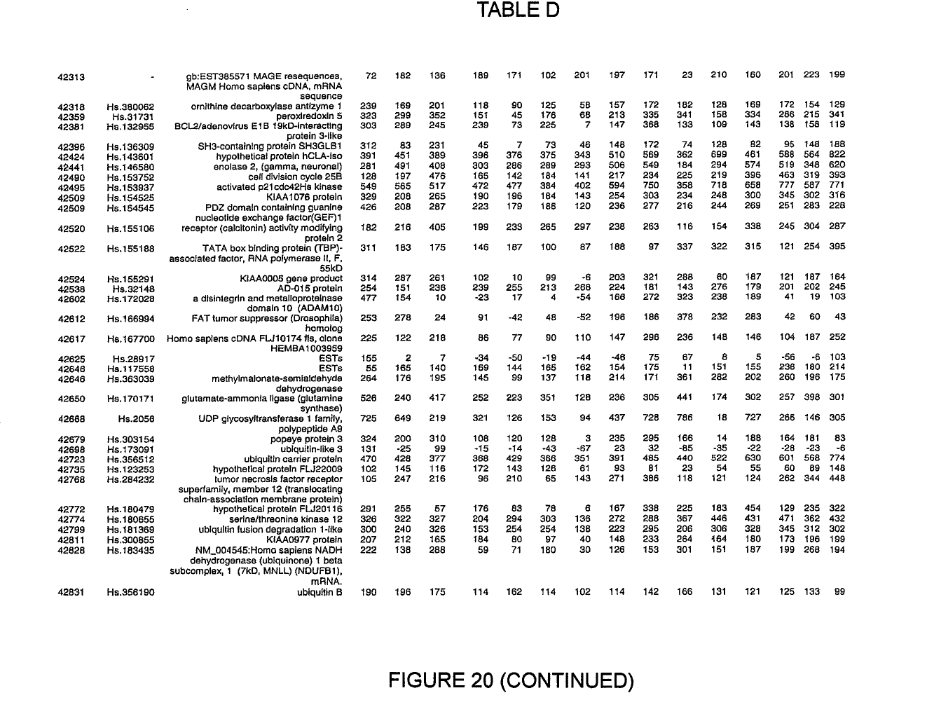

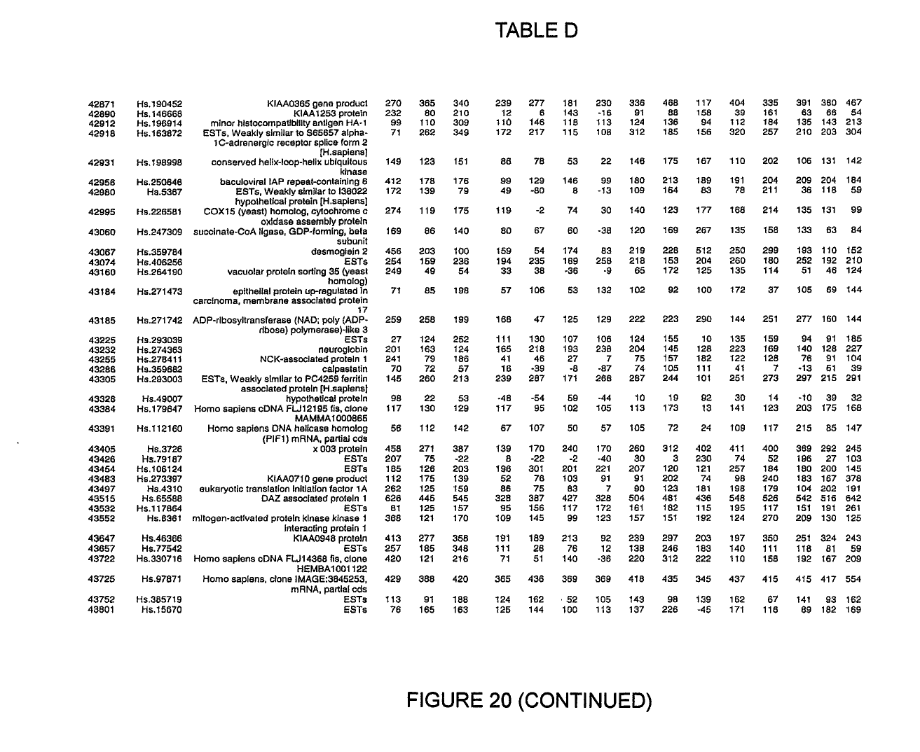

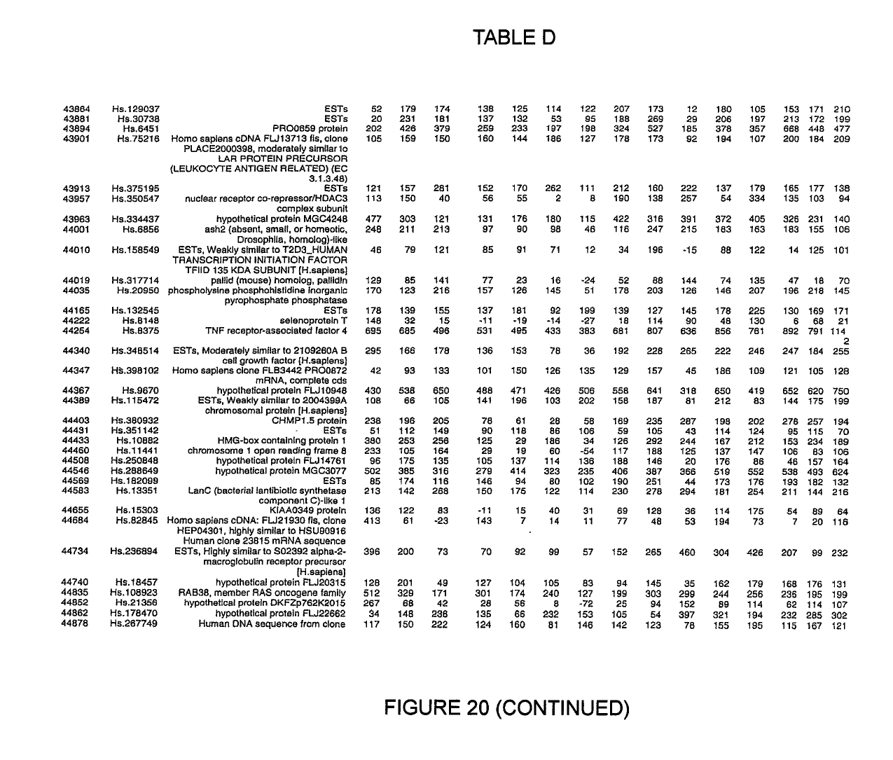

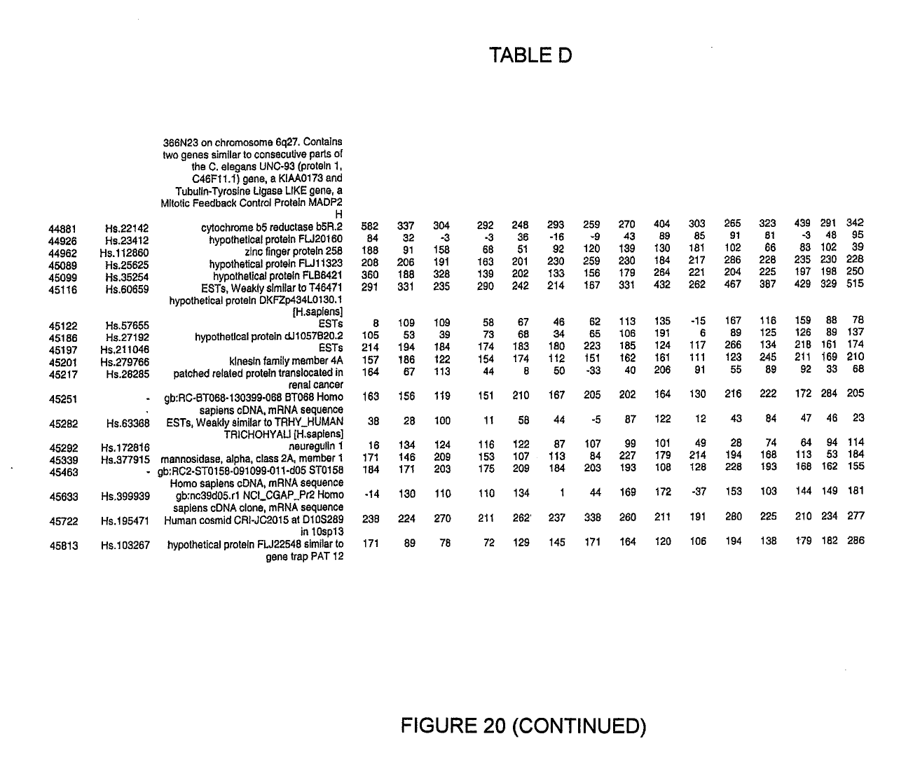

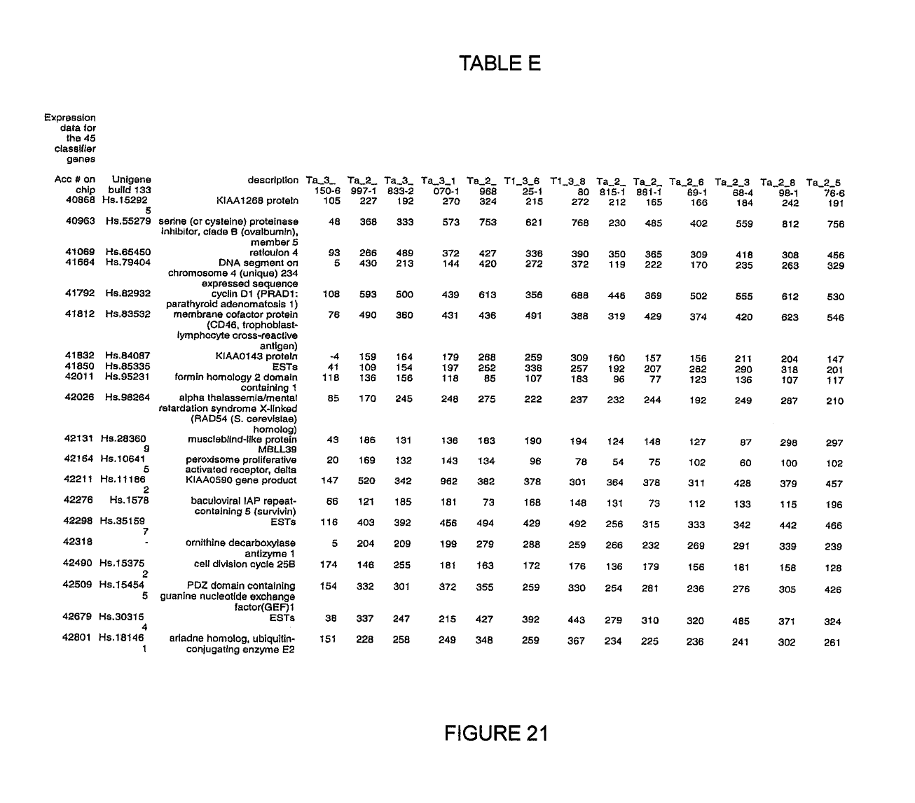

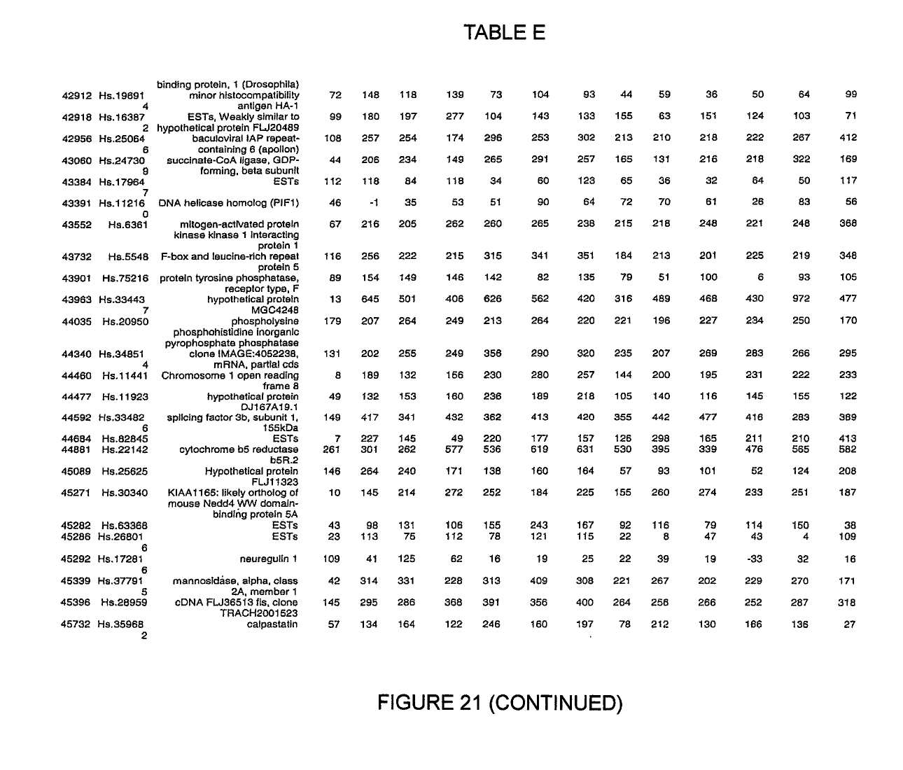

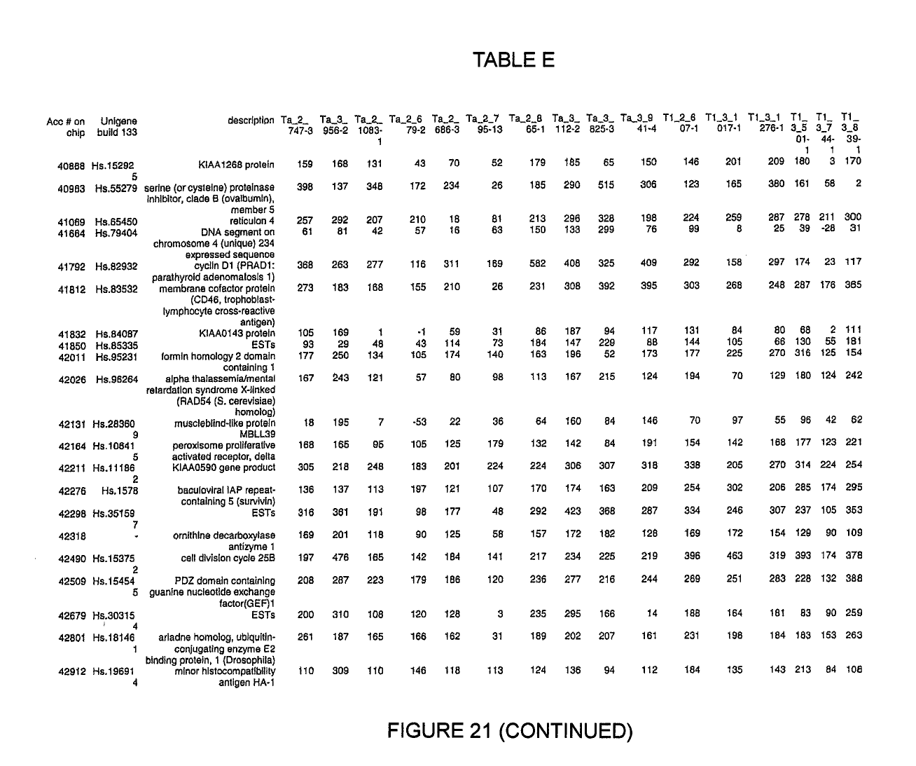

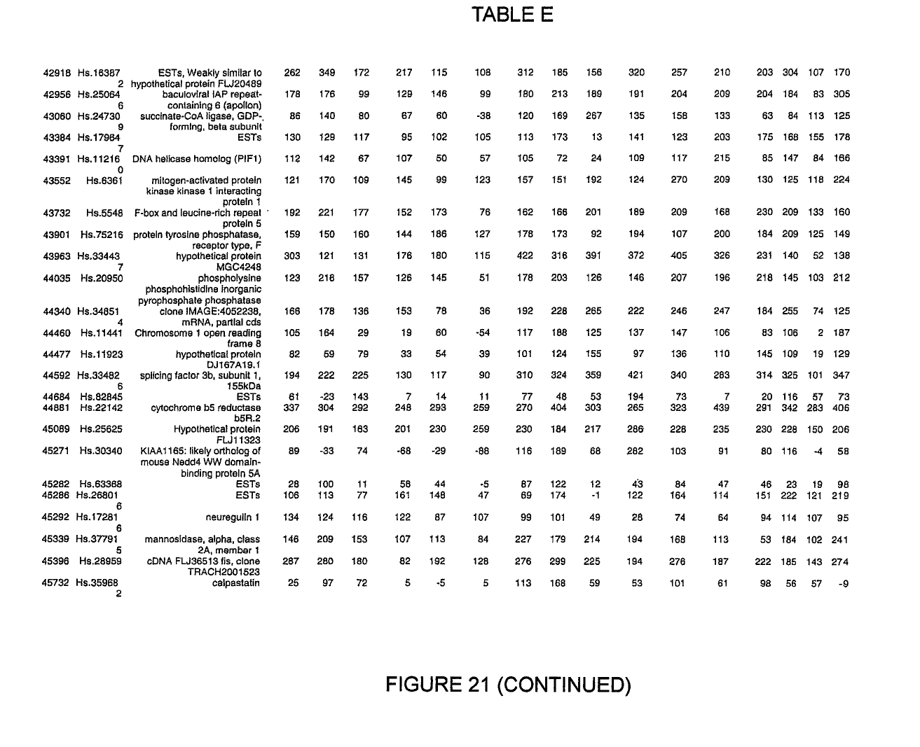

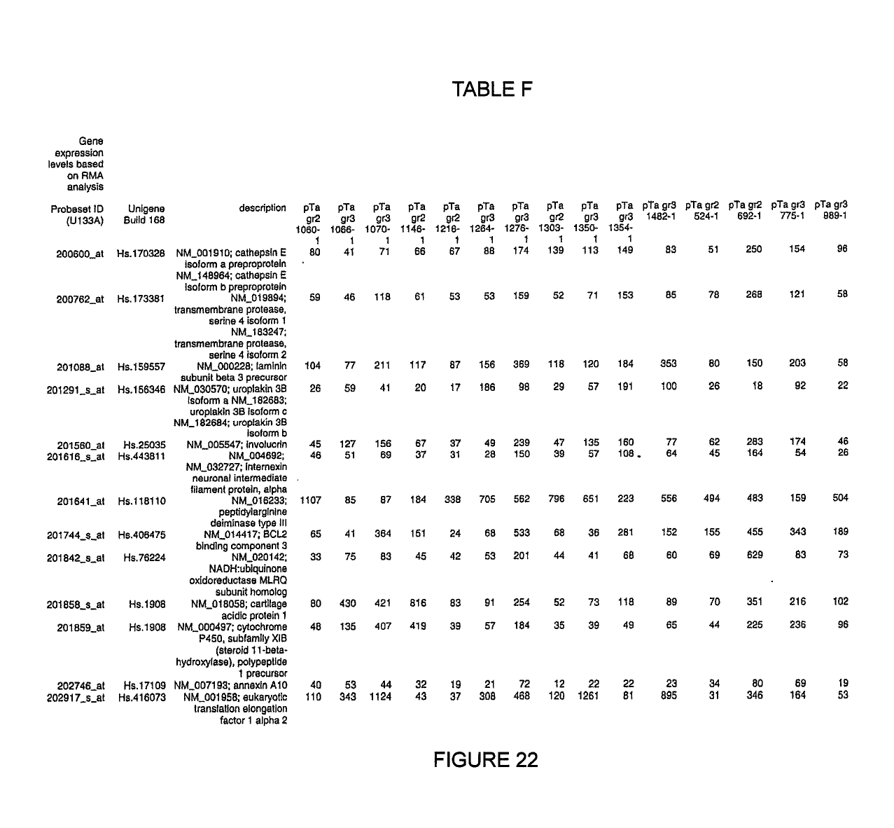

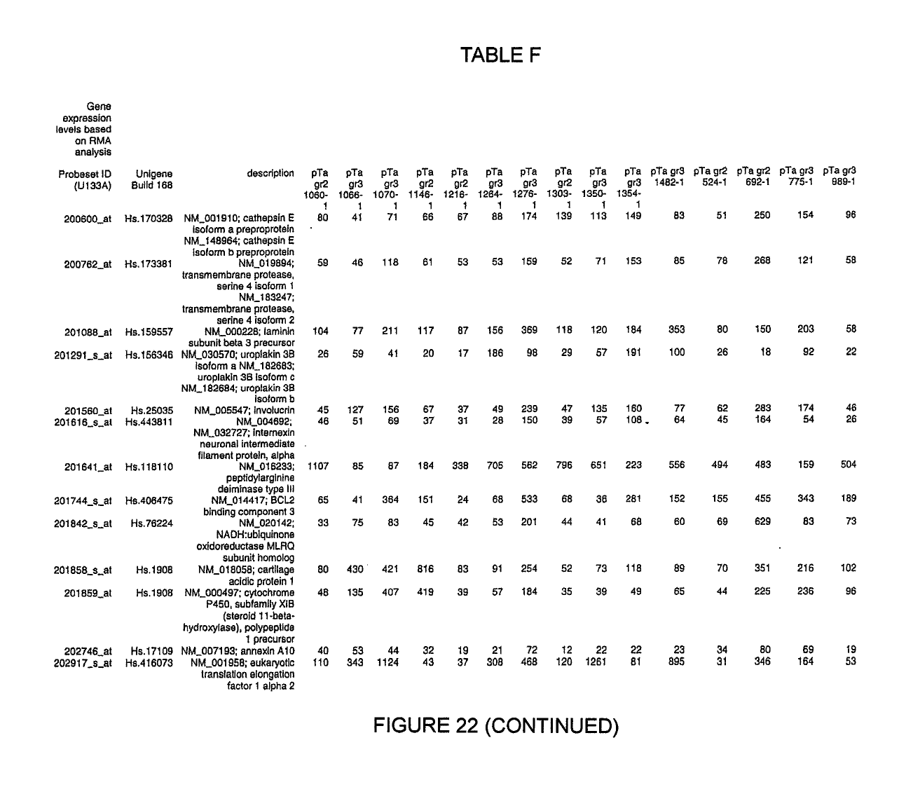

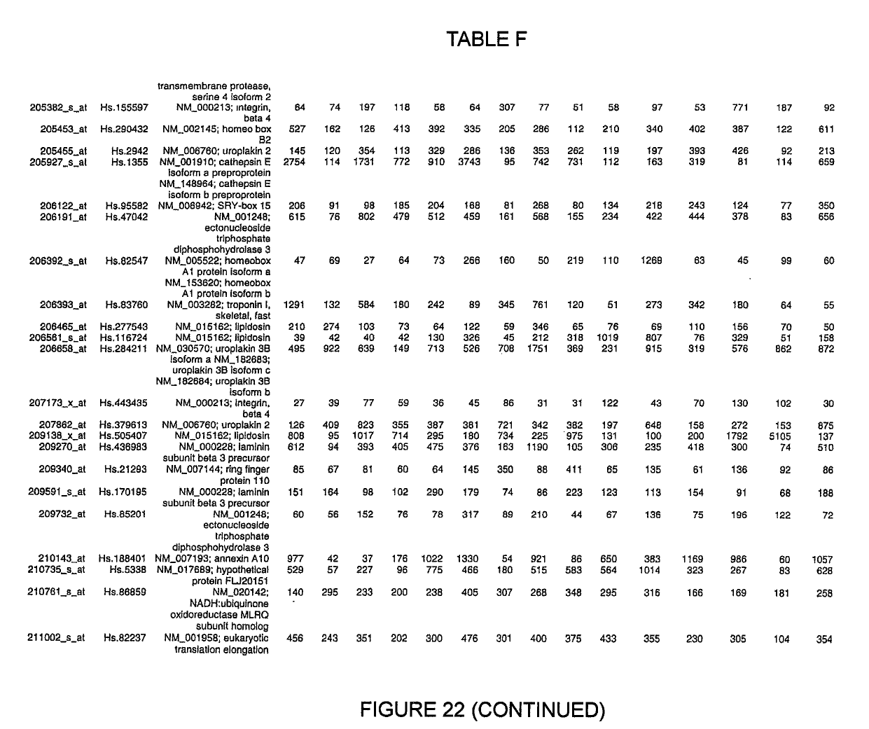

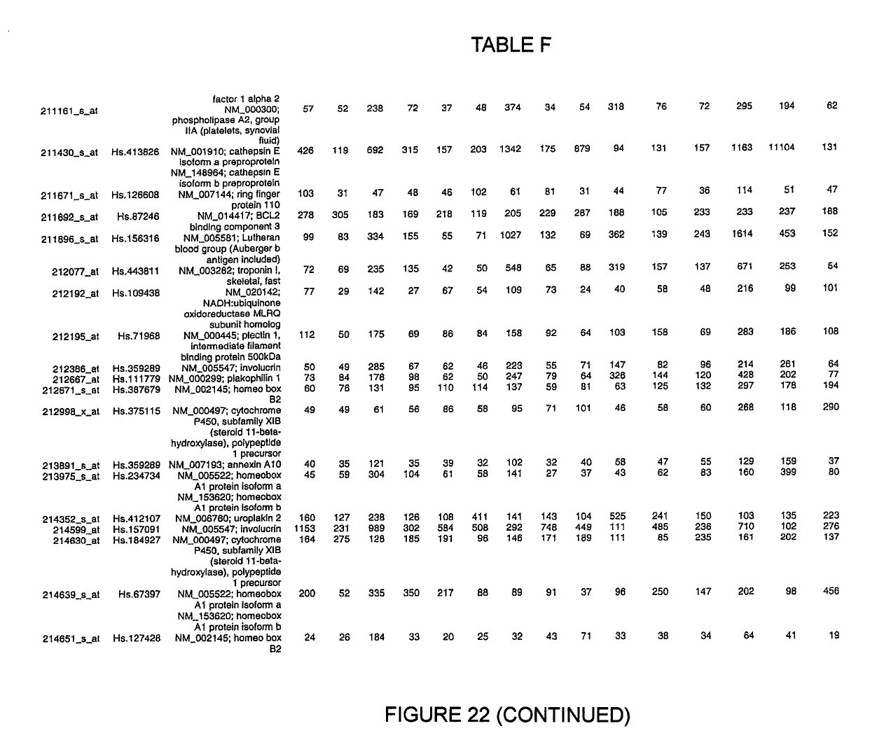

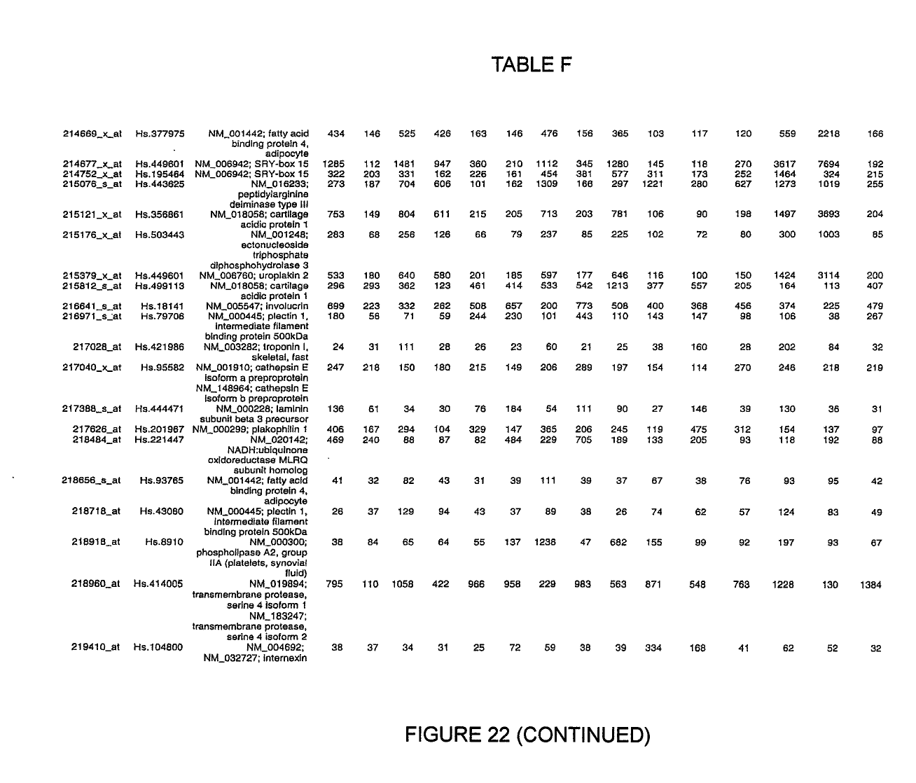

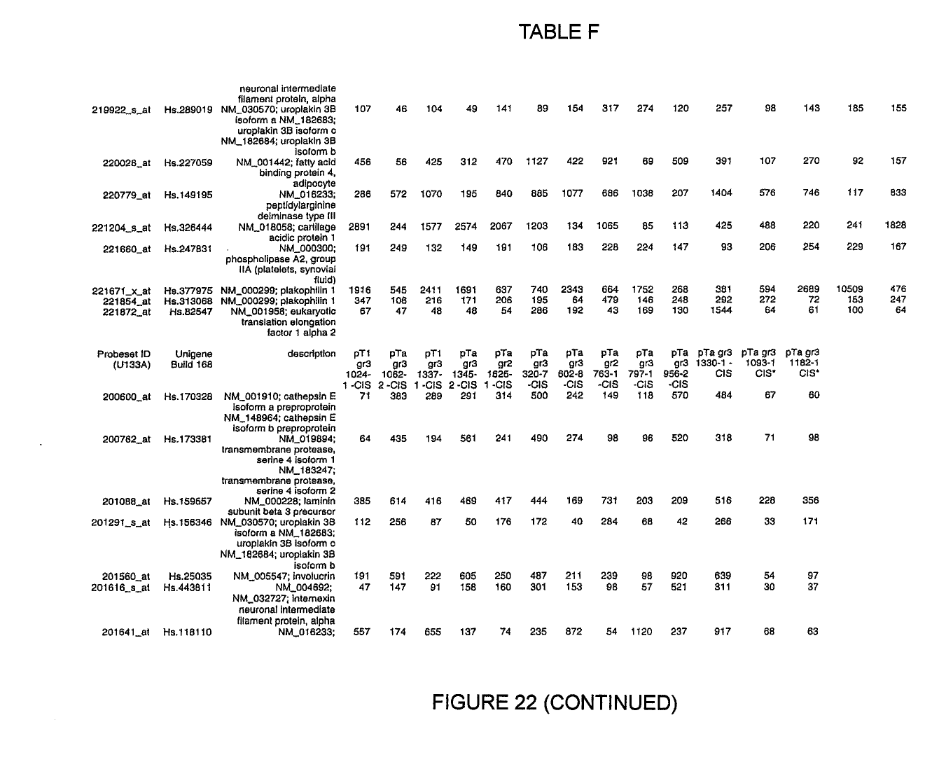

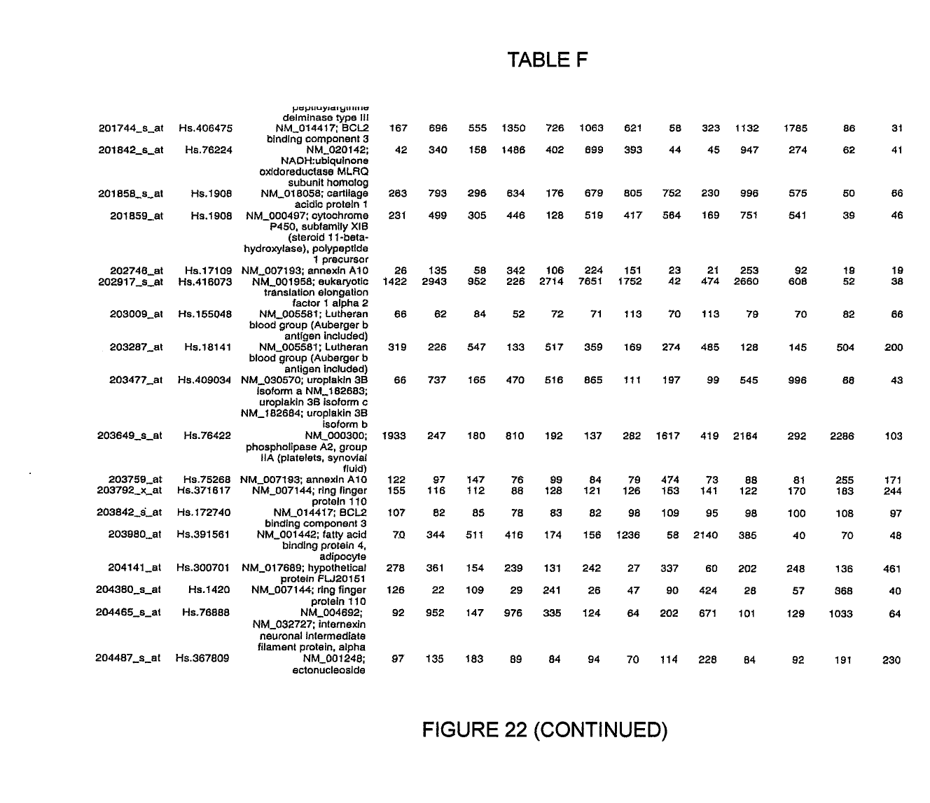

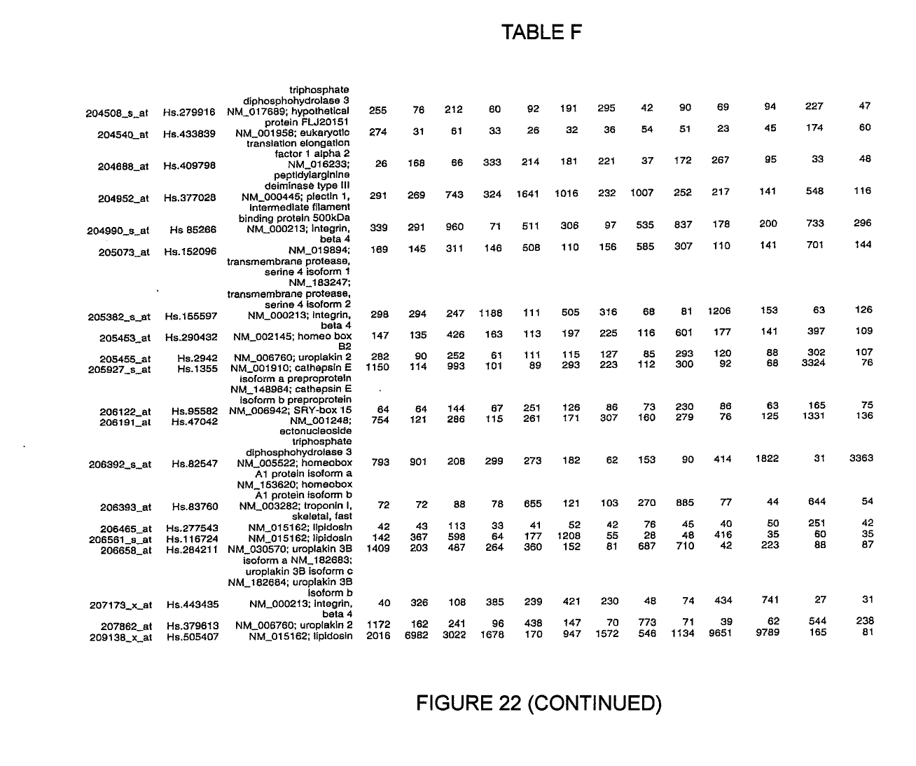

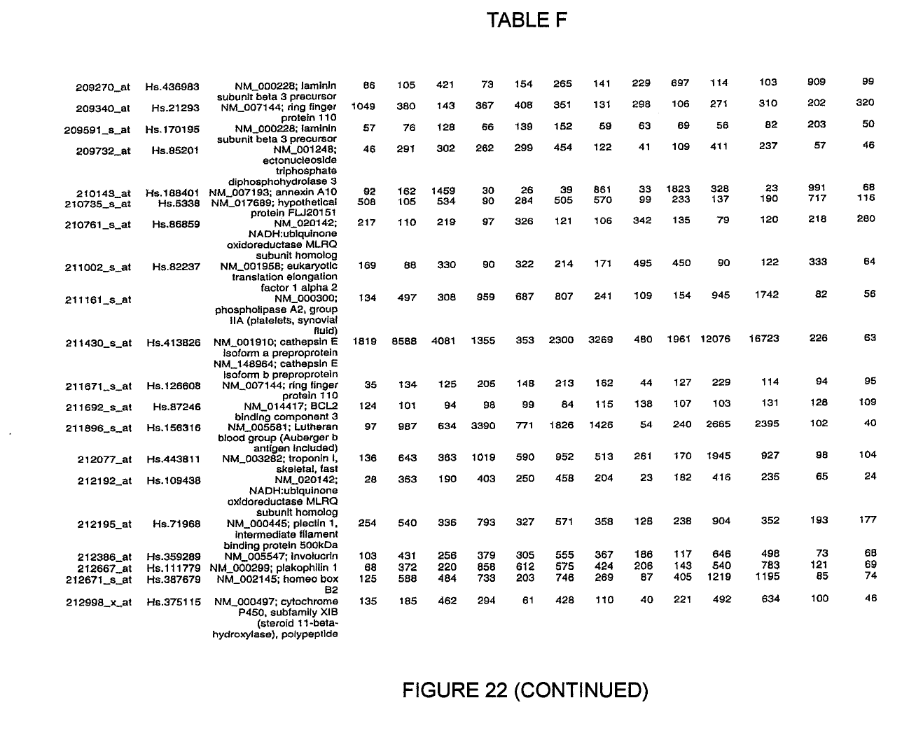

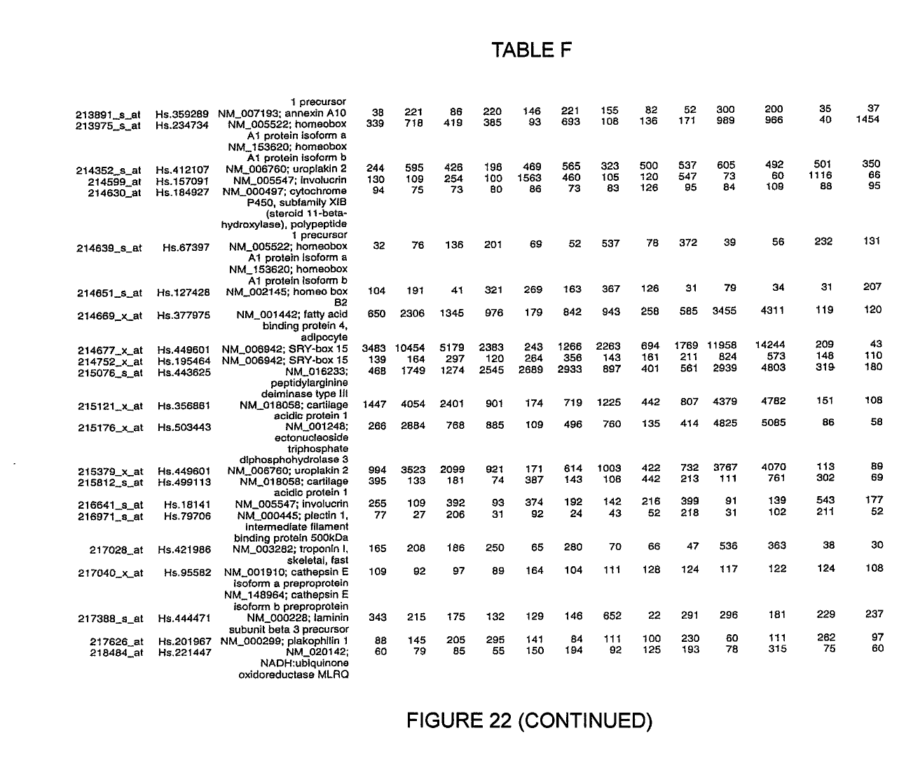

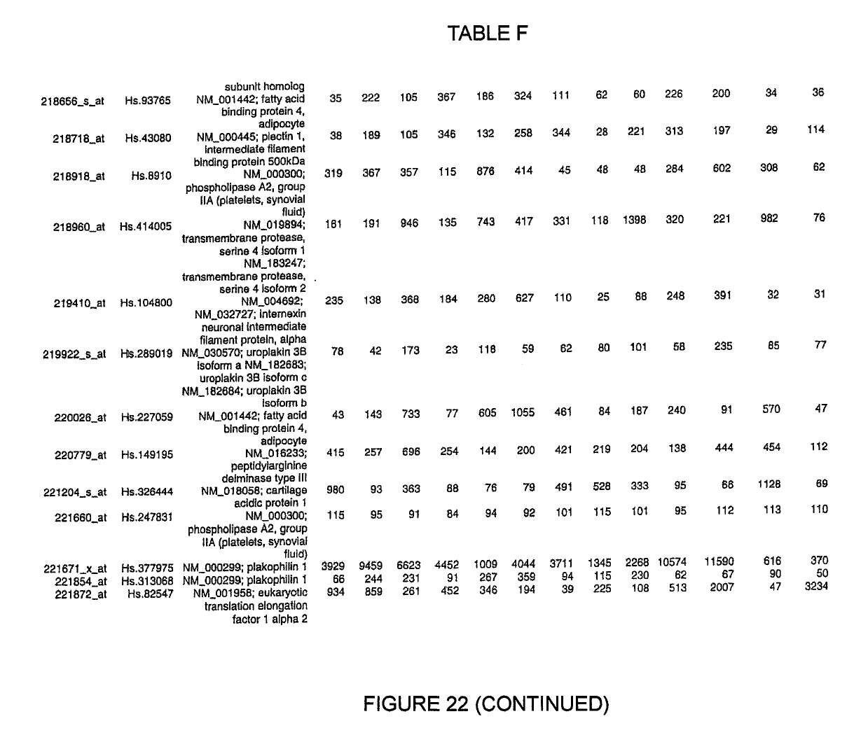

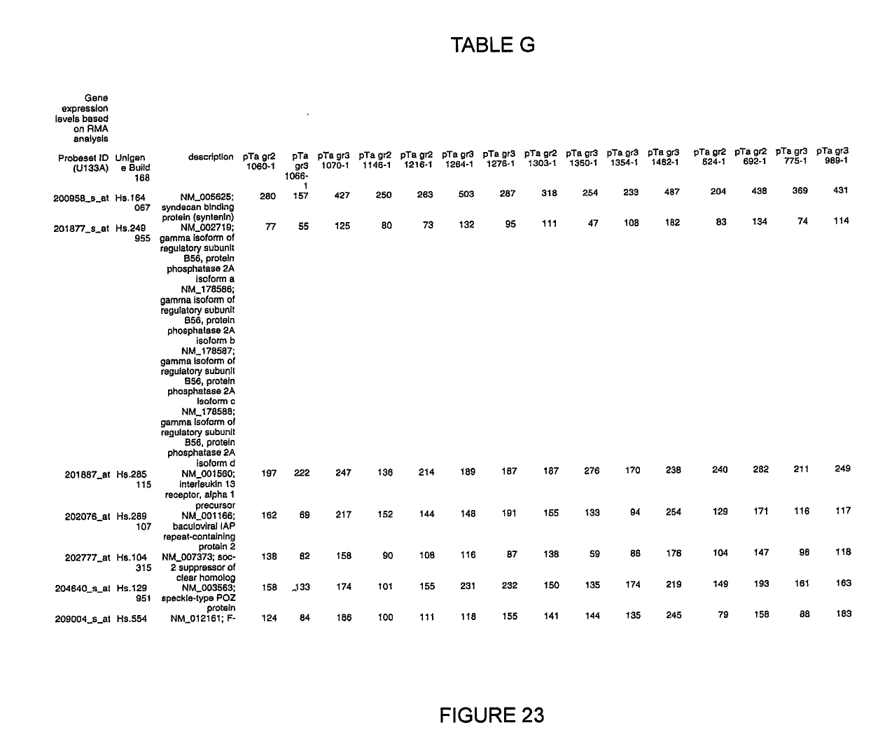

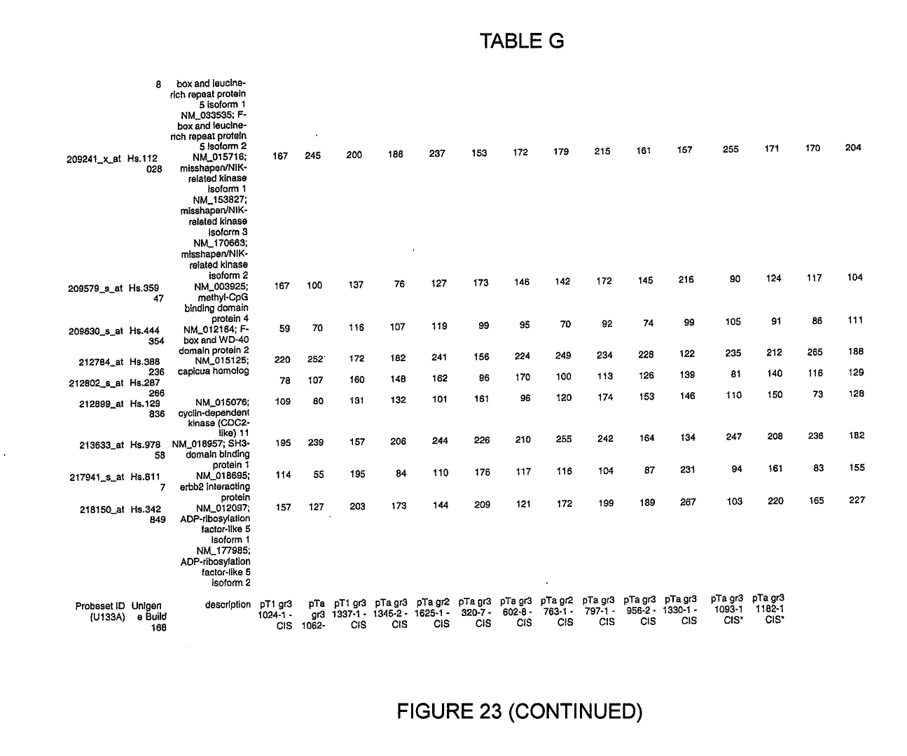

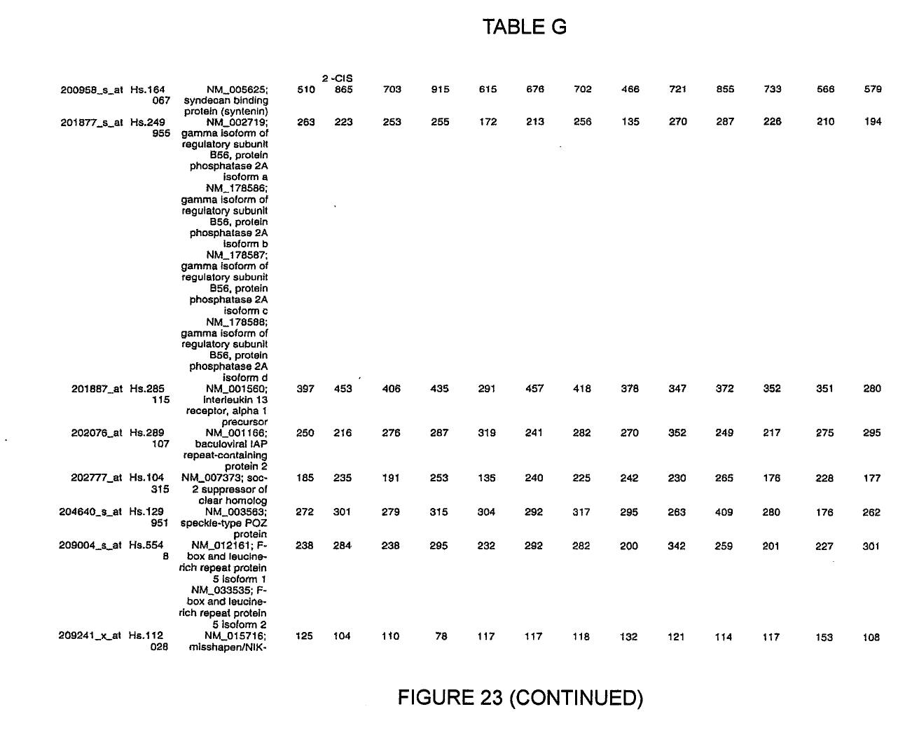

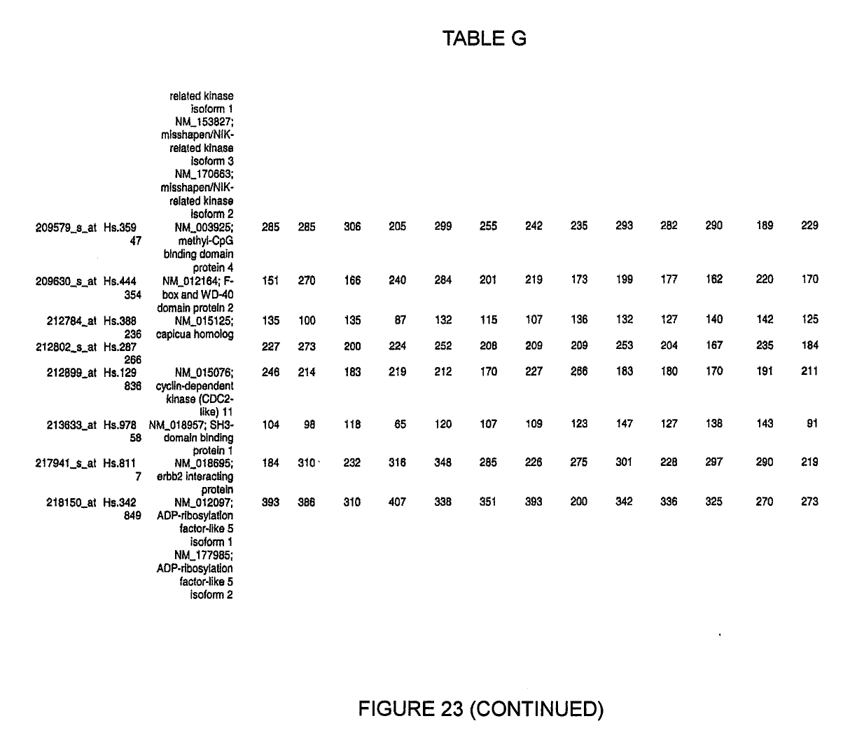

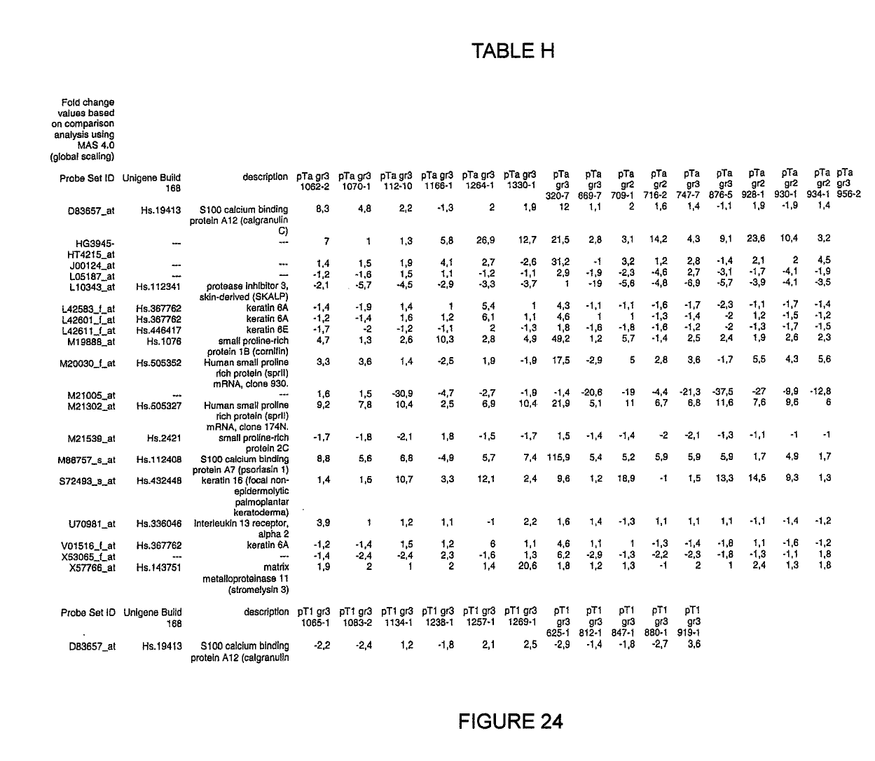

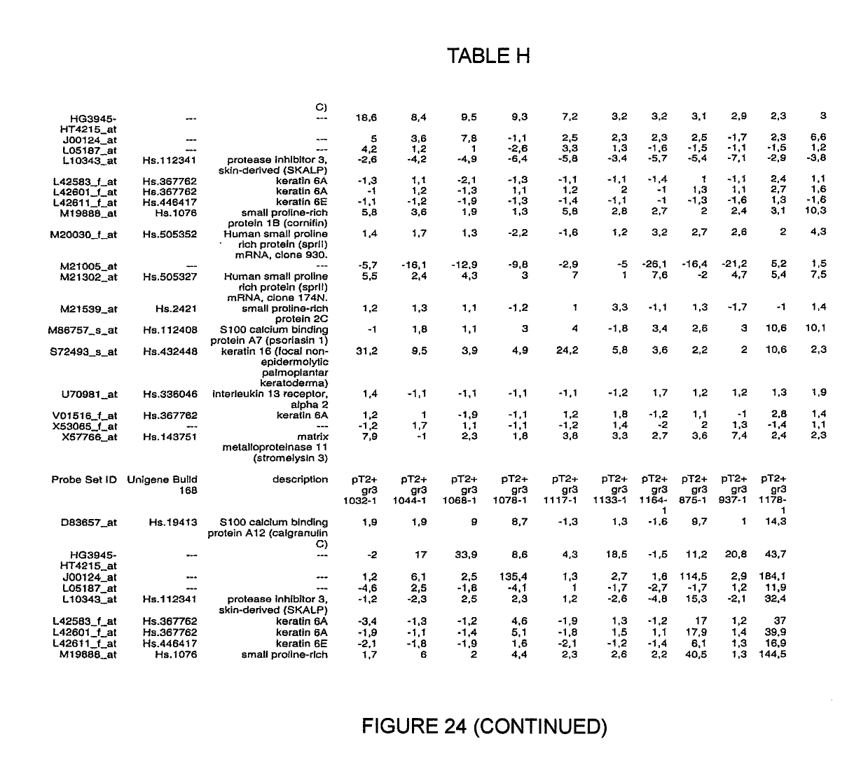

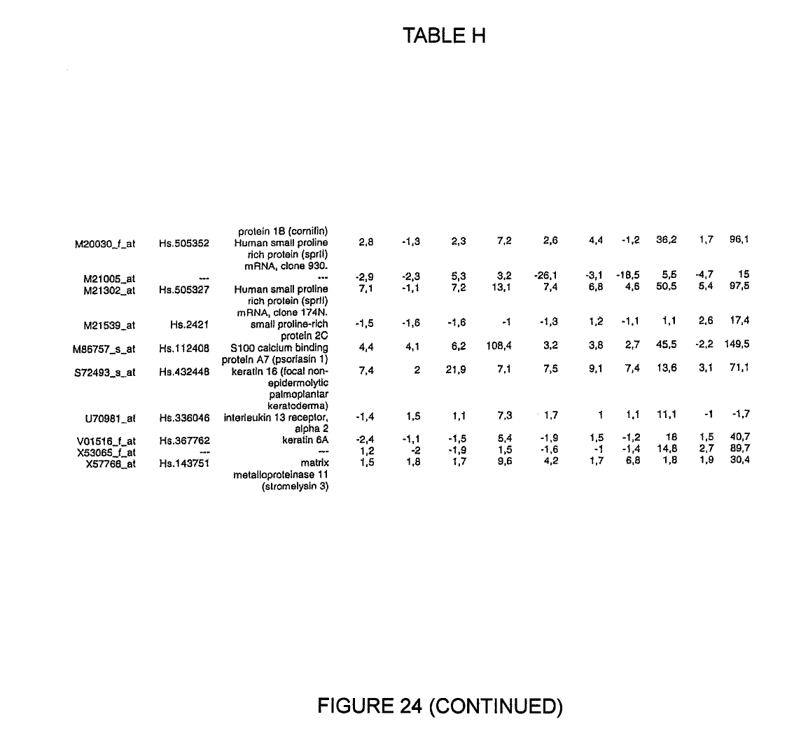

To further explore the strength of classifying CIS we also built a classifier by randomly selecting half of the samples for training and used the other half for testing. Cross validation was used again in the training of this classifier for optimisation of the gene-set for classifying independent samples. Cross-validation with 15 genes showed a good performance (see FIG. 18) and 7 of these genes were included in 70% of the class-validation loops. These 7 genes classified the samples in the test set with one error only—sample 1482-1 (χ²-test, P<0.002). Only two of the genes were also included in the 16-gene classifier, which is understandable considering the number of tests performed and the limitations in sample size. This classification performance is notable considering the small number of samples used for training the classifier.

Grouping of Normal and Cystectomies with CIS

We used hierarchical cluster analysis to group the 9 normal and 10 biopsies from cystectomies with CIS based on the normalised expression profiles of the 16 classifier genes (FIG. 17b). This clustering separated the samples from cystectomies with CIS lesions from the normal samples with only few exceptions as 8 of the 10 biopsies from cystectomies were found in the one main branch of the dendrogram and 8 of the 9 normal biopsies were found on the other main branch (χ²-test, P<0.002).

### Tables

### Table B

### REFERENCES

Pisani, P., Parkin, D. M., Bray, F. & Ferlay, J. Estimates of the worldwide mortality from 25 cancers in 1990. Int J Cancer 83, 18-29 (1999).
Wolf, H. et al. Bladder tumors. Treated natural history. Prog Clin Biol Res 221, 223-55 (1986).
Althausen, A. F., Prout, G. R., Jr., and Daly, J. J. Non-invasive papillary carcinoma of the bladder associated with carcinoma in situ. J Urol, 116:575-580, 1976.
Spruck, C. H., 3rd, Ohneseit, P. F., Gonzalez-Zulueta, M., Esrig, D., Miyao, N., Tsai, Y. C., Lerner, S. P., Schmutte, C., Yang, A. S., Cote, R., and et al. Two molecular pathways to transitional cell carcinoma of the bladder. Cancer Res, 54: 784-788, 1994.
Rosin, M. P., Cairns, P., Epstein, J. I., Schoenberg, M. P., and Sidransky, D. Partial allelotype of carcinoma in situ of the human bladder. Cancer Res, 55: 5213-5216, 1995.
Anderstrom, C., Johansson, S., and Nilsson, S. The significance of lamina propria invasion on the prognosis of patients with bladder tumors. J Urol, 124:23-26, 1980.
Cummings, K. B. Carcinoma of the bladder: predictors. Cancer, 45:1849-1855, 1980.
Cheng, L., Cheville, J. C., Neumann, R. M., Leibovich, B. C., Egan, K. S., Spotts, B. E., and Bostwick, D. G. Survival of patients with carcinoma in situ of the urinary bladder. Cancer, 85:2469-2474, 1999.
Kriegmair, M., Baumgartner, R., Lumper, W., Waidelich, R., and Hofstetter, A. Early clinical experience with 5-aminolevulinic acid for the photodynamic therapy of superficial bladder cancer. Br J Urol, 77: 667-671, 1996.
van't Veer, L. J. et al. Gene expression profiling predicts clinical outcome of breast cancer. Nature 415, 530-6 (2002).
Dyrskjot, L. et al. Identifying distinct classes of bladder carcinoma using microarrays. Nat Genet 33, 90-6 (2003).
van Rhijn, B. W. et al. The fibroblast growth factor receptor 3 (FGFR3) mutation is a strong indicator of superficial bladder cancer with low recurrence rate. Cancer Res 61, 1265-8 (2001).
Yuan, J. et al. Polo-like kinase, a novel marker for cellular proliferation. Am J Pathol 150, 1165-72 (1997).
Galaktionov, K. & Beach, D. Specific activation of cdc25 tyrosine phosphatases by B-type cyclins: evidence for multiple roles of mitotic cyclins. Cell 67, 1181-94 (1991).
Weinstein, J., Jacobsen, F. W., Hsu-Chen, J., Wu, T. & Baum, L. G. A novel mammalian protein, p55CDC, present in dividing cells is associated with protein kinase activity and has homology to the Saccharomyces cerevisiae cell division cycle proteins Cdc20 and Cdc4. Mol Cell Biol 14, 3350-63 (1994).
Hiraiwa, A. et al. Immunolocalization of hCDC47 protein in normal and neoplastic human tissues and its relation to growth. Int J Cancer 74, 180-4 (1997).
Liu, J. & Kern, J. A. Neuregulin-1 activates the JAK-STAT pathway and regulates lung epithelial cell proliferation. Am J Respir Cell Mol Biol 27, 306-13 (2002).
Park, B. H., Vogelstein, B. & Kinzler, K. W. Genetic disruption of PPARdelta decreases the tumorigenicity of human colon cancer cells. Proc Natl Acad Sci USA 98, 2598-603 (2001).
Fauconnet, S. et al. Differential regulation of vascular endothelial growth factor expression by peroxisome proliferator-activated receptors in bladder cancer cells. J Biol Chem 277, 23534-43 (2002).
Golub, T. R. et al. Molecular classification of cancer: class discovery and class prediction by gene expression monitoring. Science 286, 531-7 (1999).
Ambrosini, G., Adida, C. & Altieri, D. C. A novel anti-apoptosis gene, survivin, expressed in cancer and lymphoma. Nat Med 3, 917-21 (1997).
Sidransky, D. et al. Clonal origin bladder cancer. N Engl J Med 326, 737-40 (1992).
Primdahl, H. et al. Allelic imbalances in human bladder cancer: genome-wide detection with high-density single-nucleotide polymorphism arrays. J Natl Cancer Inst 94, 216-23 (2002).
Eaves, I. A. et al. Combining mouse congenic strains and microarray gene expression analyses to study a complex trait: the NOD model of type 1 diabetes. Genome Res 12, 232-43 (2002).
Ghandour, G. & Glynne, R. Method and apparatus for analysis of data from biomolecular arrays. International patent #WO0079465 (2000).
Turkey, J. Exploratory Data Analysis, (Addison-Wesley, Reading, Mass., 1977).
Kruhoffer, M., Magnusson, N. E., Aaboe, M., Dyrskjot, L. & Orntoft, T. F. Microarrays for gene expression profiling: Fabrication of Oligonucleotide microarrays, Isolation of RNA, Fluorescent labelling of cRNA, Hybridisation, and Scanning. in Cell Biology—A laboratory handbook (ed. Celis, J. E.) (To appear in the 4th edition of this book).
Jain, A. N. et al. Fully automatic quantification of microarray image data. Genome Res 12, 325-32 (2002).
Eisen, M. B., Spellman, P. T., Brown, P. O. & Botstein, D. Cluster analysis and display of genome-wide expression patterns. Proc Natl Acad Sci USA 95, 14863-8 (1998).
Seymour, L. Novel anti-cancer agents in development: exciting prospects and new challenges. Cancer Treat. Rev. 25, 301-312 (1999).
Fox, S. B., Gasparini, G., & Harris, A. L. Angiogenesis: pathological, prognostic, and growth-factor pathways and their link to trial design and anticancer drugs. Lancet Oncol. 2, 278-289 (2001).
Shipp, M. A. et al. Diffuse large B-cell lymphoma outcome prediction by gene-expression profiling and supervised machine learning. Nat. Med. 8, 68-74 (2002).
Kerr, J. S., Slee, A. M., & Mousa, S. A. Small molecule alpha(v) integrin antagonists: novel anticancer agents. Expert. Opin. Investig. Drugs 9, 1271-1279 (2000).
Bergkvist, A., Ljungqvist, A., & Moberger, G. Classification of bladder tumours based on the cellular pattern. Preliminary report of a clinical-pathological study of 300 cases with a minimum follow-up of eight years. Acta Chir. Scand. 130, 371-378 (1965).
Li, C. & Hung, W. W. Model-based analysis of oligonucleotide arrays: model validation, design issues and standard error application. Genome Biol. 2, RESEARCH0032 (2001).
Kononen, J. et al. Tissue microarrays for high-throughput molecular profiling of tumor specimens. Nat. Med. 4, 844-847 (1998).

1. A method of predicting the prognosis of a biological condition in animal tissue,
comprising collecting a sample comprising cells from the tissue and/or expression products from the cells,
determining an expression level of at least one gene in the sample, said gene being selected from the group of genes consisting of gene No. 1 to gene No. 562,
correlating the expression level to at least one standard expression level to predict the prognosis of the biological condition in the animal tissue.

2. The method of claim 1, wherein the animal tissue is selected from body organs.

3. The method of claim 2, wherein the animal tissue is selected from epithelial tissue in body organs.

4. The method of claim 3, wherein the animal tissue is selected from epithelial tissue in the urinary bladder.

5. The method of claim 4, wherein the stage of the biological condition is selected from bladder cancer stages Ta, Carcinoma in situ (CIS), T1, T2, T3 and T4.

6. The method of claim 5, comprising determining at least the expression of a Ta stage gene from a Ta stage gene group, at least one T1 stage gene from a T1 stage gene group, at least a T2 stage gene from a T2 stage gene group, at least a T3 stage gene from a T3 stage gene group, at least a T4 stage gene group from a T4 stage gene group, wherein at least one gene from each gene group is expressed in a significantly different amount in that stage than in one of the other stages.

7. The method of claim 4, wherein the stage is bladder cancer stage Ta.

8. The method of claim 4, wherein the animal tissue is mucosa.

9. The method of claim 1, wherein the biological condition is an adenocarcinoma, a carcinoma, a teratoma, a sarcoma, and/or a lymphoma and/or carcinoma-in-situ, and/or dysplasia-in-situ.

10. The method of claim 1, wherein the sample is a biopsy of the tissue or of metastasis originating from said tissue.

11. (canceled)

12. The method of claim 1, wherein the sample comprises substantially only cells from said tissue.

13. The method according to claim 9, wherein the sample comprises substantially only cells from mucosa or tumors derived from said mucosa cells.

14. The method of claim 1, wherein the gene from the group of genes is selected individually from the consisting of gene No. 1 to gene No. 188 (stages).

15. The method of claim 1, wherein the gene from the group of genes is selected individually from the group consisting of gene No. 189 to gene No. 214 (recurrence).

16. The method of claim 1, wherein the gene from the group of genes is selected individually from the group consisting of gene No. 215 to gene No. 232 (SCC).

17. The method of claim 1, wherein the gene from the group of genes is selected individually from the group consisting of gene No. 233 to gene No. 446 (progression).

18. The method of claim 1, wherein the gene from the group of genes is selected individually from the group consisting of gene No. 447 to gene No. 562 (CIS).

19. The method of claim 1, wherein the expression level of at least two genes from the group of genes are determined.

20. The method of claim 1, wherein the expression level of at least three genes from the group of genes are determined.

21-23. (canceled)

24. The method of claim 1, wherein the difference in expression level of a gene from the gene group to the at least one standard expression level is at least two-fold.

25. The method of claim 1, wherein the difference in expression level of a gene from the gene group to the at least one standard expression is at least three-fold.

26. The method of claim 1, wherein the difference in expression level of a gene from the gene group to the at least one standard expression is at least four-fold.

27. The method of claim 1, wherein the expression level is determined by determining the mRNA of the cells.

28. The method of claim 1, wherein the expression level is a) determined by determining expression products in the cells, or b) is determined by determining expression products in a body fluid.

29. (canceled)

30. The method of claim 1, wherein the stage of the biological condition has been determined prior to the prediction of the prognosis.

31. The method claim 30, wherein the stage of the biological condition has been determined by histological examination of the tissue or by genotyping of the tissue.

32. (canceled)

33. The method of claim 31, wherein the stage of the biological condition has been determined by
determining the expression of at least a first stage gene from a first stage gene group and/or at least a second stage gene from a second stage gene group, wherein at least one of said genes is expressed in said first stage of the condition in a higher amount than in said second stage, and the other gene is a expressed in said first stage of the condition in a lower amount than in said second stage of the condition,
correlating the expression level of the assessed genes to a standard level of expression determining the stage of the condition.

34. The method of claim 1, wherein the expression level of at least two genes is determined, by
determining a first expression level of at least one gene from a first gene group, wherein the gene from the first gene group is selected from the group consisting of gene No. 237, 238, 239, 240, 241, 242, 243, 245, 246, 247, 248, 250, 253, 254, 257, 258, 260, 263, 264, 265, 267, 270, 271, 272, 278, 283, 284, 287, 288, 290, 291, 292, 294, 297, 298, 300, 302, 303, 305, 309, 310, 315, 316, 317, 318, 319, 321, 324, 329, 335, 336, 337, 339, 340, 344, 346, 347, 354, 356, 358, 359, 362, 364, 365, 368, 369, 371, 372, 377, 378, 379, 380, 381, 382, 383, 384, 388, 391, 393, 395, 396, 397, 399, 402, 403, 404, 409, 413, 417, 419, 420, 421, 422, 423, 425, 427, 429, 430, 431, 432, 437, and 444 (progressorgener), and
determining a second expression level of at least one gene from a second gene group, wherein the second gene group is selected from the group consisting of genes No. 233, 234, 235, 236, 244, 249, 251, 252, 255, 256, 259, 261, 262, 266, 268, 269, 273, 274, 275, 276, 277, 279, 280, 281, 282, 285, 286, 289, 293, 295, 296, 299, 301, 304, 306, 307, 308, 311, 312, 313, 314, 320, 322, 323, 325, 326, 327, 328, 330, 331, 332, 333, 334, 338, 341, 342, 343, 345, 348, 349, 350, 351, 352, 353, 355, 357, 360, 361, 363, 366, 367, 370, 373, 374, 375, 376, 385, 386, 387, 389, 390, 392, 394, 398, 400, 401, 405, 406, 407, 408, 410, 411, 412, 414, 415, 416, 418, 424, 426, 428, 433, 434, 435, 436, 438, 439, 440, 441, 442, 443, 445, and 446 (non-progressorgener), and
correlating the first expression level to a standard expression level for progressors, and/or the second expression level to a standard expression level for non-progressors to predict the prognosis of the biological condition in the animal tissue.

35. A method of determining the stage of a biological condition in animal tissue, comprising collecting a sample comprising cells from the tissue,
determining an expression level of at least one gene selected from the group of genes consisting of gene No 1 to gene No. 562
correlating the expression level of the assessed genes to at least one standard level of expression determining the stage of the condition.

36. The method of claim 35, wherein the expression level of at least two genes is determined by
determining the expression of at least a first stage gene from a first stage gene group and at least a second stage gene from a second stage gene group, wherein at least one of said genes is expressed in said first stage of the condition in a higher amount than in said second stage, and the other gene is a expressed in said first stage of the condition in a lower amount than in said second stage of the condition, and
correlating the expression level of the assessed genes to a standard level of expression determining the stage of the condition

37. The method of claim 35, wherein the stage is selected from bladder cancer stages Ta, carcinoma in situ (CIS), T1, T2, T3 and T4.

38. The method of claim 37, comprising determining at least the expression of a Ta stage gene from a Ta stage gene group, at least one T1 stage gene from a T1 stage gene group, at least a T2 stage gene from a T2 stage gene group, at least a T3 stage gene from a T3 stage gene group, or at least a T4 stage gene from a T4 stage gene group, wherein at least one gene from each gene group is expressed in a significantly different amount in that stage than in one of the other stages.

39. The method of claim 38, wherein a Ta stage gene is selected individually from the group of Table B1.

40. The method of claim 38, wherein a T1 stage gene is selected individually from the group of Table B2.

41. The method of claim 38, wherein a T2 stage gene is selected individually from the group of Table B3.

42. (canceled)

43. A method of determining an expression pattern of a bladder cell sample, comprising:
collecting sample comprising bladder cells and/or expression products from bladder cells,
determining the expression level of at least one gene in the sample, said gene being selected from the group of genes consisting of gene No. 1 to gene No. 562, and obtaining an expression pattern of the bladder cell sample.

44. The method of 43, wherein the expression level of at least two genes are determined.

45. The method of 43, wherein the expression level of at least three genes are determined.

46-48. (canceled)

49. The method of claim 43, wherein the genes exclude genes which are expressed in the submucosal, muscle, or connective tissue, whereby a pattern of expression is formed for the sample which is independent of the proportion of submucosal, muscle, or connective tissue cells in the sample.

50. The method of claim 49, comprising determining the expression level of one or more genes in the sample comprising predominantly submucosal, muscle, and connective tissue cells, obtaining a second pattern, subtracting said second pattern from the expression pattern of the bladder cell sample, forming a third pattern of expression, said third pattern of expression reflecting expression of the bladder mucosa or bladder cancer cells independent of the proportion of submucosal, muscle, and connective tissue cells present in the sample.

51. The method of claim 43, wherein the sample is a biopsy of the tissue.

52. The method of claim 43, wherein the sample is a cell suspension.

53. The method of claim 43, wherein the sample comprises substantially only cells from said tissue.

54. The method according to claim 53, wherein the sample comprises substantially only cells from mucosa.

55. A method of predicting the prognosis a biological condition in human bladder tissue comprising,
collecting a sample comprising cells from the tissue,
determining an expression pattern of a bladder cell sample, comprising:
collecting sample comprising bladder cells and/or expression products from bladder cells,
determining the expression level of at least one gene in the sample, said gene being selected from the group of genes consisting of gene No. 1 to gene No. 562, and obtaining an expression pattern of the bladder cell sample,
correlating the determined expression pattern to a reference pattern,
predicting the prognosis of the biological condition of said tissue.

56. A method for determining the stage of a biological condition in animal tissue comprising,
collecting a sample comprising cells from the tissue,
determining an expression pattern of a bladder cell sample, comprising:
collecting sample comprising bladder cells and/or expression products from bladder cells,
determining the expression level of at least one gene in the sample, said gene being selected from the group of genes consisting of gene No. 1 to gene No. 562, and obtaining an expression pattern of the bladder cell sample,
correlating the determined expression pattern to a reference pattern,
determining the stage of the biological condition is said tissue.

57-71. (canceled)

72. An assay for predicting the prognosis of a biological condition in animal tissue, comprising
at least one first marker capable of detecting an expression level of at least one gene selected from the group of genes consisting of gene No. 1 to gene No. 562.

73. The assay according to claim 72, wherein the marker is a nucleotide probe.

74. The assay according to claim 72, wherein the marker is an antibody.

75. The assay according to claim 72, comprising at least a first marker and/or a second marker, wherein the first marker is capable of detecting a gene from a first gene group, and/or the second marker is capable of detecting a gene from a second gene group, where the gene from the first group is selected from the group consisting of gene No. 237, 238, 239, 240, 241, 242, 243, 245, 246, 247, 248, 250, 253, 254, 257, 258, 260, 263, 264, 265, 267, 270, 271, 272, 278, 283, 284, 287, 288, 290, 291, 292, 294, 297, 298, 300, 302, 303, 305, 309, 310, 315, 316, 317, 318, 319, 321, 324, 329, 335, 336, 337, 339, 340, 344, 346, 347, 354, 356, 358, 359, 362, 364, 365, 368, 365, 371, 372, 377, 378, 379, 380, 381, 382, 383, 384, 388, 391, 393, 395, 396, 397, 399, 402, 403, 404, 409, 413, 417, 419, 420, 421, 422, 423, 425, 427, 429, 430, 431, 432, 437, and 444 (progressorgener), and
where the gene from the second gene group is selected from the group consisting of genes No. 233, 234, 235, 236, 244, 249, 251, 252, 255, 256, 259, 261, 262, 266, 268, 269, 273, 274, 275, 276, 277, 279, 280, 281, 282, 285, 286, 289, 293, 295, 296, 299, 301, 304, 306, 307, 308, 311, 312, 313, 314, 320, 322, 323, 325, 326, 327, 328, 330, 331, 332, 333, 334, 338, 341, 342, 343, 345, 348, 349, 350, 351, 352, 353, 355, 357, 360, 361, 363, 366, 367, 370, 373, 374, 375, 376, 385, 386, 387, 389, 390, 392, 394, 398, 400, 401, 405, 406, 407, 408, 410, 411, 412, 414, 415, 416, 418, 424, 426, 428, 433, 434, 435, 436, 438, 439, 440, 441, 442, 443, 445, and 446 (non-progressorgener).

76. The assay according to claim 72, said assay further comprising means for correlating the expression level of the at least one gene to a standard expression level and/or a reference expression pattern.
